(19) United States
(12) Patent Application Publication
(10) Pub. No.: US 2023/0159436 A1
(43) Pub. Date: May 25, 2023

(54) APPLICATIONS OF AMIDE ANALOGS OF TRITERPENES IN CURES OF CANCER AND OTHER DISEASES

(71) Applicant: Pacific Arrow Limited, Hong Kong (CN)

(72) Inventors: Pui-Kwong CHAN, Sugarland, TX (US); May Sung MAK, Hong Kong (CN)

(21) Appl. No.: 18/056,265

(22) Filed: Nov. 17, 2022

(30) Foreign Application Priority Data
Nov. 19, 2021 (AU) 2021903740
May 15, 2022 (AU) 2022901291

(51) Int. Cl. C07C 233/36; C07C 231/02; C07C 311/05; C07C 303/38; C07C 275/22; C07C 273/18

(52) U.S. Cl. CPC C07C 233/36; C07C 231/02; C07C 311/05; C07C 303/38; C07C 275/22; C07C 273/1827; C07C 2603/52

(57) ABSTRACT

This invention provides a method of synthesizing new active compounds for pharmaceutical uses including cancer treatment, wherein the cancers comprise breast, leukocytic, liver, ovarian, bladder, prostatic, skin, bone, brain, leukemia, lung, colon, CNS, melanoma, renal, cervical, esophageal, testicular, splenic, kidney, lymphatic, pancreatic, stomach, eye and thyroid cancers. The active compounds are amine, sulfonamides, amide, and urea analogs of triterpene.

### CROSS REFERENCE TO RELATED APPLICATION

This application claims priorities to, and the benefit of, of Australian application AU 2021903740, filed on of 19 Nov. 2021 and Australian application AU 2022901291, filed on 15 May 2022. The contents of the foregoing applications are hereby incorporated in their entireties by reference into this application for all purposes.

### FIELD OF THE INVENTION

This invention provides methods of synthesizing new compounds for pharmaceutical uses.

### BACKGROUND OF THE INVENTION

This invention provides methods of synthesizing new compounds for pharmaceutical uses. This invention provides methods, compounds and compositions for treating cancer, inhibiting cancer invasion, cell invasion, or cancer cell invasion, wherein the cancers comprise breast, leukocytic, liver, ovarian, bladder, prostatic, skin, bone, brain, leukemia, lung, colon, CNS, melanoma, renal, cervical, esophageal, testicular, splenic, kidney, lymphatic, pancreatic, stomach eye and thyroid cancers.

### SUMMARY OF THE INVENTION

This invention provides methods of synthesizing new compounds for pharmaceutical uses. This invention provides compounds, compositions, and methods for treating cancer, inhibiting cancer invasion, cell invasion, macromolecular invasion, cancer cell invasion, and metastasis. The active compounds of this invention are triterpenes in form of amine, sulfonamides, amide, and Urea Analogs, providing extremely stable activity in solution. They prolong the activities and duration of drug in a subject. This invention provides a use of compounds, compositions, for manufacturing medicament for treating cancer, inhibiting cancer invasion, macromolecular invasion, virus invasion and metastasis. This invention provides compounds for use as mediator or inhibitor of adhesion protein or angiopoietin, This invention provides compounds for use in a method of modulating attachment or adhesion of cells or angiogenesis, by modulating or inhibiting adhesion protein macromolecules, or angiopoietin, The compounds comprise the structures selected from the formulae in the present application, wherein the compounds are synthesized or isolated, wherein the compounds comprise the saponins, triterpenes, pentacyclic triterpenes, and compounds selected from formulae in the present application, wherein the cancers comprise breast, leukocytic, liver, ovarian, bladder, prostatic, skin, bone, brain, leukemia, lung, colon, CNS, melanoma, renal, cervical, esophageal, testicular, splenic, kidney, lymphatic, pancreatic, stomach and thyroid cancers. This invention provides compounds for use as a mediator for cell circulating, cell moving cell homing and inflammatory diseases. This invention provides compounds for improving blood circulation; soothing stroke; preventing plaque formation and promote their dissipated; improve blood viscosity; reducing cardiovascular; reducing cerebrovascular; reducing thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, fluttering, Blood circulation, swelling, pain; Treating bronchiectasis, tuberculosis and lung abscess caused by too hemoptysis; reducing bleeding, antitussive, expectorant and analgesic effect, dilate blood vessels; reducing blood pressure and the treatment of cerebral arteriosclerosis; elevating blood lipids and reducing cholesterol.

### BRIEF DESCRIPTION OF THE DRAWINGS

FIG. 1. HPLC profiles of esterification products of E4A with Tigloyl chloride (A) from different times of esterification reaction. Reaction products obtained from each time of reaction (5 sec, 1 min, 2 min, 5 min, and 10 min) were fractionated by HPLC. The profile is plotted according to HPLC elution time and optical density of fractions. Reaction was performed at Room temperature (Top row) and 0 C (bottom row).

FIG. 2. HPLC profiles of esterification products of E4A with 3,3-dimethylacryloly chloride (B) from different times of esterification reaction. Reaction products obtained from each time of reaction (5 sec, 1 min, 2 min, 5 min, and 10 min) were fractionated by HPLC. The profile is plotted according to HPLC elution time and optical density of fractions. Reaction was performed at Room temperature (Top row) and 0 C (bottom row).

FIG. 3. MTT cytotoxic activity of times study at room temperature, A: E4A-Tigloyl(A); B: E4A-3,3-dimethylacryloly(B); C: E4A-4-pentenoyl(C).

FIG. 4. MTT cytotoxic activity of times study at 0 C, A: E4A-Tigloyl(A); B: E4A-3,3-dimethylacryloly(B); C: E4A-4-pentenoyl(C).

FIG. 5. MTT cytotoxic activity of times study, A: E4A-cinnamoyl(J); B: E4A-hexanoyl(D); C: E4A-2-ethylbutyryl(E); and D, controls: Tig control is tigloyl chloride without E4A; AC control is acetyl chloride without E4A; H is acetyl chloride with E4A reaction 1 min.

FIG. 6. MTT cytotoxic activity of times study, A: E4A-acetyl(H); B: E4A-crotonoyl(I)

FIG. 7. HPLC profiles of E4A-Tig in 1 min and 2 hours

FIG. 8. MTT cytotoxic activity of times study for E4A-Tig. Results: E4A-Tigs from reaction of 5 sec to 1 min are most active. Activity decrease after 1 min of reaction. Minimum to no activity was obtained at 10 minutes or longer.

FIG. 9. Results of HPLC profiles of E4A-Tigs: E4A, E4A-ASAP (5 sec), E4A-1 min, E4A-2 min, E4A-5 min, E4A-10 min, E4A-30 min.

FIG. 10. Results of Activity order: M, N, O, P, Q, R, S, T, E4A; M=E4A has no activity.

FIG. 11. (A) The IC50 of Tig-S in KB cells is about 4 ug/ml; and the corresponding IC50 in ES2 cells is less than 1 ug/ml; (B) The IC50 of Tig-S in ES2 cells, MTT assay with low doses of Tig-S, the IC50 of Tig-S in ES2 cells is approximately equal to 0.1 ug/ml

FIG. 12. (A) Results: Swiss3T3 cells are mouse normal fibroblast which were used in this experiment to compare with ES2 (human ovarian cancer) in Tig-R cytotoxicity determination. The preliminary results indicate that the IC50 of Tig-R in SW3T3 cells is above 20 ug/ml while the corresponding IC50 in ES2 cells is about 2.8 ug/ml. (B) Effect of Tig-R on Normal human lung fibroblast (WI38). Results: The IC50 of Tig-R in normal human fibroblast cells (WI38) is about 10-15 ug/ml. This IC50 value is 3 times higher than those in ES2 (3 ug/ml).

FIG. 13. (A) Results: Tig-N, -Q, -R, -T -S and -V do not have hemolytic activity up to 20 ug/ml. The graphs results are overlapped at the bottom of Figure. The original compound ES lyse 100% red blood cells (RBC) at 5 ug/ml. (B) Results: compared to Y3, the ACH-Y3 is less potent in hemolytic activity. Tig-R has no hemolytic activity

FIG. 14. (A) Comparison of potency of compound Y in inhibiting growth of ovarian cancer cells. The IC50 for Compound Y is about 1.5 μ/ml. (B). Hemolytic activity of Xanifolia-Y, B-Escin, Xanifolia-X, ACH-Y and AKOH-Y

FIG. 15. Inhibition of WI38 cells with Tig-S (6 days); Result: IC50=1.5 ug/ml; At 10-20 ug/ml, about negative 10% cell growth.

FIG. 16. Inhibition of ES2 cells with Tig-S (2 days); Results: IC50=0.3 ug/ml; At 5-10 ug/ml, negative 70% cells growth.

FIG. 17. A comparison of non-cancerous WI38 with ES2 cancer cells. MTT OD is proportional to the number of live cells. Here, the MTT OD in the no drug control represents 100% of cell growth. This study shows that at 10 ug/ml of Tig-S, WI38 cells maintain about 55% of the control cell growth, while ES2 cells have only 10% of the control cell growth.

FIG. 18. A comparison of non-cancerous WI38 with cancer cells: In these results, the MTT OD from cells before and after treated with different concentrations of drug was plotted. The result shows that: For WI38 cells, the IC₁₀₀ value is about 10 ug/ml [IC₁₀₀ is defined as the MTT OD value after the drug-treatment equal to the original OD value before the drug-treatment. At this condition (IC₁₀₀), it indicates there is 100% inhibition of growth, but there is no cell lost]. At 20 ug/ml, the OD decrease to about 90% of the original value, indicating there is about 10% cell lost or dead. For ES2 cells, the IC₁₀₀ value is about 0.16-0.3 ug/ml. However, there is a big decrease of OD with higher drug concentrations indicating there are cell dead. At 10 ug/ml, the OD is 12% of the original value, indicating over 90% cells cell lost.

FIG. 19A-B. Tig-S induces cell-death by the apoptosis mechanism.

FIG. 20-21. Leukemia K562 cells were treated with Tig-S for three days; The number inside charts is the Tig-S concentration in ug/ml; The first peak is the intensity of G0/G1 cells. The last peak is the G2/M cells. Between these two peaks represents the intensity of S-phase cells.

FIG. 22. Animal study result shows Group A Mice—Implanted tumor and no drug, Died on day 27; Group B Mice—Implanted tumor and with (Tig-S) drug 100 mg/kg, twice daily, 5 days.

FIG. 23. Animal study result shows Group A Mice—Implanted tumor and no drug; Group B Mice—Implanted tumor and with (Tig-R) drug 100 mg/kg, twice daily, 5 days.

FIG. 24. Animal study result shows Group A Mice—Implanted tumor and no drug; Group B Mice—Implanted tumor and with (Tig-V) drug 50 mg/kg, twice daily, 10 days.

FIG. 25. Inhibition of lung H460 cells growth with Tig-S for one day. IC50=3.4 ug/ml

FIG. 26. Inhibition of lung H460 cells growth with Tig-S for 4 days. IC50=3 ug/ml

FIG. 27. Inhibition of Leukemia K562 cells by Tig-S: Tig-S inhibits Leukemia K562 cells growth with IC50 about 0.6 ug/ml. No grow (IC100) was observed beginning on day 2 at 2.5 ug/ml or higher.

FIG. 28. Inhibition of ovarian cancer (OCAR3) by Tig-s, Tig-S inhibits OCAR3 cells' growth with an IC50 value of 2.5 ug/ml; and inhibition of pancreas cancer (Capan), Tig-S inhibits Capan cells' growth with an IC50 value of about 1 ug/ml.

### DETAILED DESCRIPTION OF THE INVENTION

This invention provides a method of synthesizing new active compounds for pharmaceutical uses. The active compounds of this invention are triterpenes in form of amine, sulfonamides, amide, and urea analogs, providing extremely stable activity in solution They prolong the activities and duration of drug in a subject. This invention provides an anti adhesion therapy which uses the compound as a mediator or inhibitor of adhesion proteins and angiopoietins. It inhibits excessive adhesion and inhibits cell viral and macromolecular attachment. It modulates angiogenesis. The compounds also use as mediator of cell viral and macromolecular adhesion receptor(s).

This invention provides compounds or a composition comprising the compounds of triterpenes in form of amine, sulfonamides, amide, and urea analogs, provided in the invention for treating cancers; for inhibiting cancer growth, for inhibiting viruses; for preventing cerebral aging; for improving memory; improving cerebral functions; for curing enuresis, frequent micturition, urinary incontinence; neurodegenerative diseases, dementia, Alzheimer's disease, autism, brain trauma, Parkinson's disease or other diseases caused by cerebral dysfunctions; for treating arthritis, rheumatism, poor circulation, arteriosclerosis, Raynaud's syndrome, angina pectoris, cardiac disorder, coronary heart disease, headache, dizziness, kidney disorder; cerebrovascular disease; inhibiting NF-kappa B activation; for treating brain edema, severe acute respiratory syndrome, respiratory viral diseases, chronic venous insufficiency, hypertension, chronic venous disease, oedema, inflammation, hemonhoids, peripheral edema formation, varicose vein disease, flu, post traumatic edema and postoperative swelling; for inhibiting blood clots, for inhibiting ethanol absorption; for lowering blood sugar; for regulating adrenocorticotropin and corticosterone levels. This invention provides a composition for Anti-MS, anti-aneurysm, anti-asthmatic, anti-oedematous, anti-inflammatory, anti-bradykinic, anti-capillarihemorrhagic, anti-cephalagic, anti-cervicobrachialgic, anti-eclamptic, anti-edemic, anti-encaphalitic, anti-epiglottitic, anti-exudative, anti-flu, anti-fracture, anti-gingivitic, anti-hematomic, anti-herpetic, anti-histaminic, anti-hydrathritic, anti-meningitic, antioxidant, anti-periodontic, anti-phlebitic, anti-pleuritic, anti-raucedo, anti-rhinitic, anti-tonsilitic, anti-ulcer, anti-varicose, anti-vertiginous, cancerostatic, corticosterogenic, diuretic, fungicide, hemolytic, hyaluronidase inhibitor, lymphagogue, natriuretic, pesticide, pituitary stimulant, thymolytic, vasoprotective, inhibiting leishmaniases, modulating adhesion or angiogenesis of cells, anti-parasitic; increase the expression of the genes: ANGPT2, DDIT3, LIF and NFKB1Z, and manufacturing an adjuvant composition and venotonic treatment.

This invention provides compounds, compositions and methods for treating cancer diseases, inhibiting cancer invasion, for inhibiting cancer growth or for inhibiting cancer metastasis, wherein the compounds comprise the structures selected from the formulae of the present application, wherein the compounds can be synthesized or isolated, wherein the compounds comprise the triterpenes, pentacyclic triterpenes, saponins, and compounds selected from formulae in this application, wherein the cancers comprise breast cancer, leukocytic cancer, liver cancer, ovarian cancer, bladder cancer, prostatic cancer, skin cancer, bone cancer, brain cancer, leukemia cancer, lung cancer, colon cancer, CNS cancer, melanoma cancer, renal cancer, cervical cancer, esophageal cancer, testicular cancer, splenic cancer, kidney cancer, lymphatic cancer, pancreatic cancer, stomach cancer, eye cancer and thyroid cancer; wherein the cells comprise breast cell, leukocytic cell, liver cell, ovarian cell, bladder cell, prostatic cell, skin cell, bone cell, brain cell, leukemia cell, lung cell, colon cell, CNS cell, melanoma cell, renal cell, cervical cell, esophageal cell, testicular cell, splenic cell, kidney cell, lymphatic cell, pancreatic cell, stomach cell and thyroid cell.

This invention provides compounds for improving blood circulation; soothing stroke; preventing plaque formation and promote their dissipated; improve blood viscosity; reducing cardiovascular; reducing cerebrovascular; reducing thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, fluttering, Blood circulation, swelling, pain; Treating bronchiectasis, tuberculosis and lung abscess caused by too hemoptysis; reducing bleeding, antitussive, expectorant and analgesic effect, dilate blood vessels; reducing blood pressure and the treatment of cerebral arteriosclerosis; elevating blood lipids and reducing cholesterol.

This invention shows that the presence of group selected from acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl, C(2-18) Acyl at carbon position 21, 22, 24 and/or 28 of a pentacyclic triterpene, triterpene, triterpeniod, triterpeniod saponin or compound selected from formulae of the present application, produces inhibition of cancer growth, cancer invasion, cells invasion, cancer cell invasion or macromolecular cell invasion. In an embodiment, the presence of group(s) selected from acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl, C(2-18) Acyl at carbon position 2, 3, 8, 15, 21, 22, 23, 24 and/or 28 of a pentacyclic triterpene, triterpeniod, triterpeniod saponin or compound selected from formulae of the present application produces activities including inhibition of cancer growth, cancer invasion, cells invasion, cancer cell invasion, cell adhesion, cell attachment or cell circulating wherein the group may attached with an O, S, NH, CH2O, C(O), C(O)O, N-alkyl, CH2, CH2O, CH2NH, CH2NHCO, CH2NHCONH, or CH2NHSO2 to the carbon of triterpene, triterpeniod, triterpeniod saponin or compound selected from formulae of the present application; The active compounds are triterpene with amine, sulfonamides, amide, or Urea Analogs, providing extremely stable activity in solution. They prolong the activities and duration of drug in a subject. They do not hydrolyzed in plasma by the enzyme, but undergo enzymatic degradation in the liver and excretion in the urine.

In an embodiment, the presence of group at carbon position 24, produces activities. In an embodiment, the presence of group at carbon position 24 and 28 produces activities. In an embodiment, the presence of group at carbon position 24 and 21 produces activities. In an embodiment, the presence of group at carbon position 24, 28 and 21, produces activities. In an embodiment, the presence of group at carbon position 24, 28 and 22 produces activities. In an embodiment, the presence of group at carbon position 24, 28 and 3 produces activities. In an embodiment, the presence of group at carbon position 24, and 3 produces activities. In an embodiment, the presence of group at carbon position 28 and 3 produces activities. In an embodiment, the presence of group at carbon position 3 produces activities. In an embodiment, the presence of group at carbon position 21 and 22 produces activities. In an embodiment, the hemolytic activity of the compound is reduced. In embodiment, the compound is attached a sugar moiety(ies), acid moiety(ies) or alduronic acid. In an embodiment, the presence of group at carbon position 1, produces activities. In an embodiment, the presence of group at carbon position 2, produces activities. In an embodiment, the presence of group at carbon position 3, produces activities. In an embodiment, the presence of group at carbon position 4, produces activities. In an embodiment, the presence of group at carbon position 5, produces activities. In an embodiment, the presence of group at carbon position 6, produces activities. In an embodiment, the presence of group at carbon position 7, produces activities. In an embodiment, the presence of group at carbon position 8, produces activities. In an embodiment, the presence of group at carbon position 9, produces activities. In an embodiment, the presence of group at carbon position 10, produces activities. In an embodiment, the presence of group at carbon position 11, produces activities. In an embodiment, the presence of group at carbon position 12, produces activities. In an embodiment, the presence of group at carbon position 13, produces activities. In an embodiment, the presence of group at carbon position 14, produces activities. In an embodiment, the presence of group at carbon position 15, produces activities. In an embodiment, the presence of group at carbon position 16, produces activities. In an embodiment, the presence of group at carbon position 17, produces activities. In an embodiment, the presence of group at carbon position 18, produces activities. In an embodiment, the presence of group at carbon position 19, produces activities. In an embodiment, the presence of group at carbon position 20, produces activities. In an embodiment, the presence of group at carbon position 21, produces activities. In an embodiment, the presence of group at carbon position 22, produces activities. In an embodiment, the presence of group at carbon position 23, produces activities. In an embodiment, the presence of group at carbon position 24, produces activities. In an embodiment, the presence of group at carbon position 25, produces activities. In an embodiment, the presence of group at carbon position 26, produces activities. In an embodiment, the presence of group at carbon position 27, produces activities. In an embodiment, the presence of group at carbon position 28, produces activities. In an embodiment, the presence of group at carbon position 29, produces activities. In an embodiment, the presence of group at carbon position 30, produces activities. In an embodiment, the activities are for treating cancers, inhibition of cancer growth, cancer invasion, cells invasion, cancer cell invasion; cell adhesion, cell attachment, cell circulating; for treating mad cow disease; treating prion diseases; for inhibiting viruses; for preventing cerebral aging; for improving memory; improving cerebral functions; for curing enuresis, frequent micturition, urinary incontinence; dementia, Alzheimer's disease, autism, brain trauma, Parkinson's disease or other diseases caused by cerebral dysfunctions or neurodegeneration; for treating arthritis, rheumatism, poor circulation, arteriosclerosis, Raynaud's syndrome, angina pectoris, cardiac disorder, coronary heart disease, headache, dizziness, kidney disorder; cerebrovascular diseasea; inhibiting NF-kappa B activation; for treating brain edema, severe acute respiratory syndrome, respiratory viral diseases, chronic venous insufficiency, hypertension, chronic venous disease, oedema, inflammation, hemorrhoids, peripheral edema formation, varicose vein disease, flu, post traumatic edema and postoperative swelling; for inhibiting blood clots, for inhibiting ethanol absorption; for lowering blood sugar; for regulating adrenocorticotropin and corticosterone levels. This invention provides a composition for Anti-MS, anti-aneurysm, anti-asthmatic, anti-oedematous, anti-inflammatory, anti-bradykinic, anti-capillarihemorrhagic, anti-cephalagic, anti-cervicobrachialgic, anti-eclamptic, anti-edemic, anti-encaphalitic, anti-epiglottitic, anti-exudative, anti-flu, anti-fracture, anti-gingivitis, anti-hematomic, anti-herpetic, anti-histaminic, anti-hydrathritic, anti-meningitic, antioxidant, anti-periodontic, anti-phlebitic, anti-pleuritic, anti-raucedo, anti-rhinitic, anti-tonsilitic, anti-ulcer, anti-varicose, anti-vertiginous, cancerostatic, corticosterogenic, diuretic, fungicide, hemolytic, hyaluronidase inhibitor, lymphagogue, natriuretic, pesticide, pituitary stimulant, thymolytic, vasoprotective, inhibiting leishmaniases, modulating adhesion or angiogenesis of cells, anti-parasitic.

In an embodiment, the compound is arresting cells in the S-phase and blocking their entering into the G2/M phase of cell cycle. The compound blocks the DNA synthesis of cancer cell. This application produces synthetic compounds increase the potency and decrease the toxicity. In an embodiment, the compounds are amine, sulfonamides, amide, or urea analogs of triterpene, which provide extremely stable activity in solution They prolong the bio activities and duration of drug in a subject.

Experiments presented in this invention showed that the core compound including E4A, E4D1, E4D which have no effect in inhibiting cancer growth, cancer invasion, cells invasion or cancer cell invasion. The core E4A was obtained by removing the groups from carbon positions 3, 21 and 22 of the active Escin. Further, amination of E4A to give E4D or E4D1 core. The core compound including E4A, E4D, E4D1 E and AKOH have no hemolytic activity and no anti-cancer activity.

This invention showed that functional group attached at carbon position 24 of a pentacyclic triterpene did not produce hemolytic activity, which has bio-activities including inhibiting cancer growth, inhibiting cancer invasion, cells invasion or cancer cell invasion. This invention showed that functional group attached at carbon position 3 of a pentacyclic triterpene did not produce hemolytic activity, which has bio-activities including inhibiting cancer growth, inhibiting cancer invasion, cells invasion or cancer cell invasion. This invention showed that function group(s) attached at carbon position 3 and 1 or 2 or 3 of carbon position 28, 21, 22, 24 of a pentacyclic triterpene did not produce hemolytic activity, which has bio activities including inhibiting cancer growth, inhibiting cancer invasion, cells invasion or cancer cell invasion.

This invention showed that functional group attached at carbon position 2 of a pentacyclic triterpene did not produce hemolytic activity, which has bio activities including inhibiting cancer growth, inhibiting cancer invasion, cells invasion or cancer cell invasion. This invention showed that function group(s) attached at carbon position 2 and 1 or 2 or 3 of carbon position 28, 21, 22, 24 of a pentacyclic triterpene did not produce hemolytic activity, which has bio-activities including inhibiting cancer growth, inhibiting cancer invasion, cells invasion or cancer cell invasion.

This invention provides a pentacyclic triterpene with reduced hemolytic activity for treating diseases, wherein the triterpene comprising a group(s) attached at its core producing bioactivities. This invention provides a pentacyclic triterpene with reduced hemolytic activity comprising a group(s) attached at carbon position 3, or carbon position 24 and 1 or 2 or 3 of of other position(s) of a pantacyclic triterpene, which has bioactivities. This invention provides a triterpene with reduced hemolytic activity comprising a group(s) attached at carbon position 15, 16, 21, 22, 23, 24, 28, 29, 30 and/or 3 of a pentacyclic triterpene, which has bioactivities. This invention provides a composition comprising a triterpene with reduced hemolytic activity comprising a group(s) attached at carbon position 3, or carbon position 24 and 1 or 2 or 3 of of other position(s) of a pentacyclic triterpene, which has bioactivities. This invention provides a method for bio-activities treatment including but not limited to treating cancers, comprising administering to said subject an effective amount of compound, wherein the compound is a pentacyclic triterpene with reduced hemolytic activity comprising a group(s) attached at carbon position 3, or carbon position 24 and 1 or 2 or 3 of of other position(s) of a pentacyclic triterpene, which has bio-activities. The active compounds of this invention are amine, sulfonamides, amide, and urea analogs of triterpene, providing extremely stable activity in solution. They prolong the activities and duration of drug in a subject. They do not hydrolyzed in plasma by the enzyme pseudocholinesterase, but undergo enzymatic degradation in the liver and excretion in the urine.

The compound of the present application can be obtained with the method:
1. Dissolving core compound or triterpenes, hydroxylated triterpenes core in pyridine,
2. Adding acyl chloride,
3. The mixture is stirred for length of time including 5 sec, 10 sec, 20 sec, 30 sec, 40 sec,1 min, 2 min, 5 min, 10 min, 30 min, 1 hr, 2 hrs., 18 hrs., 2 days or 3 days at 0 C, 25 C, 50 C or 75 C,
4. At the end of reaction, an aqueous solution of acid or base, or water is added to the reaction mixture,
5. The solution is then extracted of ethyl acetate and ethyl acetate is removed by evaporation and and lyophilization,
6. Dissolving the reaction product in acetonitrile with Trifluoroacetic acid or DMSO,
7. Testing the reaction product of mixtures and individual fractions with MTT cytotoxic assay,
8. Selecting the HPLC fractions for isolation is according to the cytotoxic activity of the reaction product obtained at a specific reaction time,
9. Purifiing the active esterification products with HPLC,
10. Collecting the products,
11. Testing the products.

The compound of present application, wherein the core compound is terpene, isoprene, or triterpene core; wherein the core compound is hydroxylated; wherein the core compound was dissolved in pyridine; wherein the acyl chloride including Tigloyl chloride, angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, benzoyl chloride and Ethylbutyryl chloride; wherein the reaction time for the mixture is stirred for 5 sec, 1 min, 2 min, 5 min, 10 min, 30 min, 1 hr, 2 hr, 18 hr, 2 days or 3 days; wherein the temperature is 0 C, 25 C, 50 C or 75 C temperature; wherein the acid including HCl or the base is a weak base including NaHCO3 is added to the reaction mixture; wherein the solution is then extracted 3 times with ethyl acetate and lyophilization; wherein the reaction product is dissolved in 80% acetonitrile-0.005% Trifluoroacetic acid or DMSO; wherein selecting the HPLC fractions for isolation is according to the cytotoxic activity of the reaction product obtained at a reaction time of 5 sec, 1 min, 2 min, 5 min, 10 min, 30 min, 1 hr, 2 hr, 18 hr, 2 days or 3 days.

This invention showed that functional group attached at carbon position 23 of a pentacyclic triterpene did not produce hemolytic activity, which has bio-activities including inhibiting cancer growth, inhibiting cancer invasion, cells invasion or cancer cell invasion. This invention showed that function group(s) attached at carbon position 24 and 1 or 2 or 3 of carbon position 28, 21, 22 of a pentacyclic triterpene did not produce hemolytic activity, which has bio-activities including inhibiting cancer growth, inhibiting cancer invasion, cells invasion or cancer cell invasion.

This invention showed that functional group attached at carbon position 24 of a pentacyclic triterpene did not produce hemolytic activity, which has bio-activities including inhibiting cancer growth, inhibiting cancer invasion, cells invasion or cancer cell invasion. This invention showed that function group(s) attached at carbon position 24 and 1 or 2 or 3 of carbon position 28, 21, 22 of a pentacyclic triterpene did not produce hemolytic activity, which has bio-activities including inhibiting cancer growth, inhibiting cancer invasion, cells invasion or cancer cell invasion.

This invention provides a triterpene with reduced hemolytic activity for treating diseases, wherein the triterpene comprising a group(s) attached at its core producing bio-activities. This invention provides a pentacyclic triterpene with reduced hemolytic activity comprising a group(s) attached at carbon position 24, or carbon position 24 and 1 or 2 or 3 of of other position(s) of a pentacyclic triterpene, which has bio-activities. This invention provides a composition comprising a pentacyclic triterpene with reduced hemolytic activity comprising a group(s) attached at carbon position 24, or carbon position 24 and 1 or 2 or 3 of of other position(s) of a pentacyclic triterpene, which has bio-activities. This invention provides a method for bio-activities treatment including but not limited to treating cancers, comprising administering to said subject an effective amount of compound, wherein the compound is a triterpene with reduced hemolytic activity comprising a group(s) attached at carbon position 24, or carbon position 24 and 1 or 2 or 3 of of other position(s) of a pentacyclic triterpene, which has bio-activities.

This invention showed that Tig-N, Tig-Q, Tig-R, Tig-T Tig-S and Tig-V do not have hemolytic activity up to 20 ug/ml. The original compound ES lyse 100% red blood cells (RBC) at 5 ug/ml. Compare to Y3, the ACH-Y3 is less potent in hemolytic activity. Tig-R has no hemolytic activity. This invention showed that Tig-N, Tig-Q, Tig-R, Tig-T Tig-S and Tig-V have anti cancer activities. The E4D1-Tig-R, E4D1-Tig-Q, E4D-Tig-R, E4D1-Tig-T E4D1-Tig-S and E4D1-Tig-V in form of amide provide extremely stable activity in solution. They prolong the bio activities and duration of drug in a subject. They do not hydrolyzed in plasma by the enzyme pseudocholinesterase, but undergo enzymatic degradation in the liver and excretion in the urine.

Many saponins and triterpenes have hemolytic characteristic that damage red blood cells. This severe side effect makes people hesitate to use saponins or triterpenes in medicines. This invention produces sythesised saponins and triterpenes with reduced hemolytic characteristic for use as medicament. This invention produces compounds with reduced hemolytic characteristic for use as medicament. The medicament can be used for treating cancer, inhibiting cancer growth, cancer invasion, cells invasion or cancer cell invasion. This application produces synthetic compounds increase the potency and decrease the toxicity.

A compound which has bio-activities including inhibiting cancer growth, inhibiting cancer invasion, cells invasion or cancer cell invasion is called active compound.

This invention provides a use for compounds, compositions, and methods for manufacturing medicament for treating cancers, inhibition of cancer growth, cancer invasion, cells invasion, cancer cell invasion; cell adhesion, cell attachment, cell circulating, or for inhibiting cancer metastasis, wherein the compounds comprise the structures selected from the formulae of the present application, wherein the compounds can be synthesized or isolated, wherein the compounds comprise the pentacyclic triterpenes, wherein the compounds with reduced hemolytic, wherein the cells comprise cancer cells, wherein the cancers comprise breast cancer, leukocytic cancer, liver cancer, ovarian cancer, bladder cancer, prostatic cancer, skin cancer, bone cancer, brain cancer, leukemia cancer, lung cancer, colon cancer, CNS cancer, melanoma cancer, renal cancer, cervical cancer, esophageal cancer, testicular cancer, splenic cancer, kidney cancer, lymphatic cancer, pancreatic cancer, stomach cancer, eye cancer and thyroid cancer. The method of inhibiting cancer invasion, cells invasion or cancer cell invasion activities uses non-cytotoxic drug concentrations. The method of inhibiting metastasis uses non-cytotoxic drug concentrations. There is no noticeable change in cell morphology.

This invention provides triterpene(s) with reduced hemolytic activity for treating diseases, wherein the triterpene can be a pentacyclic triterpene comprising a group(s) attached at its core producing bio-activities. This invention provides a pentacyclic triterpene with reduced hemolytic effect, comprising a group(s) attached at carbon position 24, or carbon position 24 and 1 or 2 or 3 of of other position(s) of a pentacyclic triterpene, which has bio-activities. This invention provides a composition comprising a triterpene with reduced hemolytic activity comprising a group(s) attached at carbon position 24, or carbon position 24 and 1 or 2 or 3 of of other position(s) of a pentacyclic triterpene, which has bio-activities. This invention provides a method for bio-activities treatment including but not limited to treating cancers, comprising administering to said subject an effective amount of compound, wherein the compound is a triterpene with reduced hemolytic activity, comprising a group(s) attached at carbon position 24, or carbon position 24 and 1 or 2 or 3 of of other position(s) of a triterpene, which has bio-activities, wherein a compound selected from AA1-4, BB1-4, CC1-4, DD1-4, EE1-4, GG1-4, HH1-4, II1-4, JJ1-4, KK1-4, LL1-4, MM1-4, PP1-4, QQ1-4, RR1-4, SS1-4, TT1-4, Tig-Sen-n, Tig-Cro-n, Tig-Acy-n, Tig-Pen-n, Tig-Hex-n, Tig-Cin-n, Tig-Ang-n, Tig-Eth-n, Tig-R-Sen-n, Tig-R-Cro-n, Tig-R-Acy-n, Tig-R-Pen-n, Tig-R-Hex-n, Tig-R-Cin-n, Tig-R-Ang-n, Tig-R-Eth-n, wherein n=1 to 6, and a salt, ester, metabolite thereof, or amine, diamine, amide, sulfonamide, urea thereof; wherein the compound(s) is in form in form of powder, liquid or crystal and the compounds selected from formulae 2A, and K; wherein the compound is selected from Tig-N, Tig-Q, Tig-R, Tig-T Tig-S and Tig-V; wherein the compound is selected from E4D1-Tig-R, E4D1-Tig-Q, E4D-Tig-R, E4D1-Tig-T E4D1-Tig-S and E4D1-Tig-V in form of amide.

This invention provides methods for treating cancers, inhibition of cancer growth, cancer invasion, cells invasion, cancer cell invasion; cell adhesion, cell attachment, cell circulating, migration, metastasis or growth of cancers, wherein the methods comprise affecting gene expression, wherein the methods comprise stimulating gene expression, or wherein the methods comprise inhibiting the gene expression, or wherein the methods comprise administering to a subject an effective amount of compounds, compositions in this application. In an embodiment, the method comprises contacting said cell with a compound selected from AA1-4, BB1-4, CC1-4, DD1-4, EE1-4, GG1-4, HH1-4, II1-4, JJ1-4, KK1-4, Xanifolia Y0, Y1, Y2, Y(Y3), Y5, Y7, Y8, Y9, Y10, Xanifolia (x), M10, Escin(bES), Aescin, ACH-Y(Y3), ACH-Y10, ACH-Y2, ACH-Y8, ACH-Y7, ACH-Y0, ACH-X, ACH-Z4, ACH-Z1, ACH-Escin(bES), ACH-M10, Tig-Sen-n, Tig-Cro-n, Tig-Acy-n, Tig-Pen-n, Tig-Hex-n, Tig-Cin-n, Tig-Ang-n, Tig-Eth-n, Tig-R-Sen-n, Tig-R-Cro-n, Tig-R-Acy-n, Tig-R-Pen-n, Tig-R-Hex-n, Tig-R-Cin-n, Tig-R-Ang-n, Tig-R-Eth-n, wherein n=1 to 6, and a salt, ester, metabolite thereof, amine, diamine, amide, sulfonamide, urea thereof; wherein the compound(s) is in form in form of powder, liquid or crystal and the compounds selected from formulae 2A, and K. In vitro studies show that a compound selected from structure (2A) or (K) inhibits cell adhesion to culture flasks. The compound blocks the function of these adhesive molecules on cells. In an embodiment, the selected compound blocks the function of these adhesive molecules on cells. In an embodiment, the selected compound blocks the function of these adhesive molecules on carcinoma cells. In an embodiment, the selected compound blocks the function of these adhesive molecules on the mesothelial cells. This invention provides an anti adhesion therapy which uses the compound as a mediator or inhibitor of adhesion proteins and angiopoietins. It inhibits excess adhesion and inhibits cell attachment. This invention provides compounds for use as a mediator for cell circulating, cell moving and inflammatory diseases. In an embodiment, the selected compound binds to the adhesive proteins (by masking) on the membrane and inhibits the interaction of adhesion proteins with their receptors. In an embodiment, the selected compound's action on the membrane affects adhesion proteins' function in the membrane. The lost of adhesion activity of cancer cells is result from direct or indirect action of the selected compound on membrane proteins.

This invention provides a use of compounds or methods for inhibiting cancer invasion, cell invasion, cancer cell invasion, macromolecular cell invasion, migration, metastasis or growth of cancers, wherein this invention comprises a process and method for administration of the composition, wherein administration is by intravenous injection, intravenous drip, intraperitoneal injection or oral administration; wherein administration is by intravenous drip: 0.003-0.03 mg/kg body weight of compound dissolved in 250 ml of 10% glucose solution or in 250 ml of 0.9% NaCl solution, or by intravenous injection: 0.003-0.03 mg/kg body weight per day of compound dissolved in 10-20 ml of 10% glucose solution or of 0.9% NaCl solution, or 0.01-0.03 mg/kg body weight of compound dissolved in 250 ml of 10% glucose solution or in 250 ml of 0.9% NaCl solution, or by intravenous injection: 0.01-0.03 mg/kg body weight per day of compound dissolved in 10-20 ml of 10% glucose solution or of 0.9% NaCl solution, or 0.01-0.05 mg/kg body weight of compound dissolved in 250 ml of 10% glucose solution or in 250 ml of 0.9% NaCl solution, or by intravenous injection: 0.01-0.05 mg/kg body weight per day of compound dissolved in 10-20 ml of 10% glucose solution or of 0.9% NaCl solution, or 0.05-0.2 mg/kg body weight of compound dissolved in 250 ml of 10% glucose solution or in 250 ml of 0.9% NaCl solution, or by intravenous injection: 0.05-0.2 mg/kg body weight per day of compound dissolved in 10-20 ml of 10% glucose solution or of 0.9% NaCl solution, or by intravenous drip: 0.1-0.2 mg/kg body weight per day of compound dissolved in 250 ml of 10% glucose solution or in 250 ml of 0.9% NaCl solution, or by intravenous injection: 0.1-0.2 mg/kg body weight per day compound dissolved in 10-20 ml of 10% glucose solution or of 0.9% NaCl solution, or by intraperitoneal injection (I.P.): 2.5 mg/kg body weight per day compound dissolved in 10% glucose solution or of 0.9% NaCl solution, or by oral administration wherein the dosage of mammal is 1-10 mg/kg, 10-30 mg/kg, 30-60 mg/kg, or 60-90 mg/kg body weight of compound, or by intravenous injection or intravenous drip wherein the dosage of mammal is 0.01-0.1 mg/kg body weight, 0.1-0.2 mg/kg, 0.2-0.4 mg/kg body weight, or 0.4-0.6 mg/kg body weight of compound, or by intraperitoneal injection (I.P.) wherein the dosage of mammal is 1-3 mg/kg, 3-5 mg/kg, 4-6 mg/kg, or 6-10 mg/kg body weight of compound, or 10-50 mg/kg body weight of compound, or 50-100 mg/kg body weight of compound, or 30-70 mg/kg body weight of compound or 100-150 mg/kg body weight of compound.

This invention provides a use of compounds or methods for treating cancers, inhibition of cancer growth, cancer invasion, cells invasion, cancer cell invasion; macromolecular cell invasion, cell adhesion, cell attachment, cell circulating, migration, metastasis or growth of cancers, infection or re-infection of virus or infectious macromolecules, and cancer cell fusion, wherein the invention comprises a pharmaceutical composition comprising the compound of this invention or a pharmaceutically acceptable salt thereof, and a pharmaceutically acceptable carrier or diluent, wherein said compound is present in a concentration of 0.01 ug/ml to 65 ug/ml, or wherein said compound is present in a concentration of 0.01 ug/ml to 40 ug/ml, or wherein said compound is present in a concentration of 0.01 ug/ml to 30 ug/ml, or wherein said compound is present in a concentration of 0.01 ug/ml to 10 ug/ml, or wherein said compound is present in a concentration of 0.01 ug/ml to 5 ug/ml, or wherein said compound is present in a concentration of 5 ug/ml to 10 ug/ml, or wherein said compound is present in a concentration of 0.1 ug/ml to 5 ug/ml, or wherein said compound is present in a concentration of 0.1 ug/ml to 7.5 ug/ml, or wherein said compound is present in a concentration of 0.1 ug/ml to 10 ug/ml, or wherein said compound is present in a concentration of 0.1 ug/ml to 15 ug/ml, or wherein said compound is present in a concentration of 0.1 ug/ml to 20 ug/ml, or wherein said compound is present in a concentration of 0.1 ug/ml to 30 ug/ml, or wherein said compound is present in a concentration of 1 ug/ml to 5 ug/ml, or wherein said compound is present in a concentration of 1 ug/ml to 7.5 ug/ml, or wherein said compound is present in a concentration of 1 ug/ml to 10 ug/ml, or wherein said compound is present in a concentration of 1 ug/ml to 15 ug/ml, or wherein said compound is present in a concentration of 1 ug/ml to 20 ug/ml, or wherein said compound is present in a concentration of 1 ug/ml to 30 ug/ml, or wherein said compound is present in a concentration of 3 ug/ml to 5 ug/ml, or wherein said compound is present in a concentration of 3 ug/ml to 7.5 ug/ml, or wherein said compound is present in a concentration of 3 ug/ml to 10 ug/ml, or wherein said compound is present in a concentration of 3 ug/ml to 15 ug/ml, or wherein said compound is present in a concentration of 3 ug/ml to 20 ug/ml, or wherein said compound is present in a concentration of 3 ug/ml to 30 ug/ml, or wherein said compound is present in a concentration of 4 ug/ml to 5 ug/ml, or wherein said compound is present in a concentration of 4 ug/ml to 7.5 ug/ml, or wherein said compound is present in a concentration of 4 ug/ml to 10 ug/ml, or wherein said compound is present in a concentration of 4 ug/ml to 15 ug/ml, or wherein said compound is present in a concentration of 4 ug/ml to 20 ug/ml, or wherein said compound is present in a concentration of 4 ug/ml to 30 ug/ml, or wherein said compound is present in a concentration of 5 ug/ml to 8 ug/ml, or wherein said compound is present in a concentration of 5 ug/ml to 9 ug/ml, or wherein said compound is present in a concentration of 5 ug/ml to 10 ug/ml, or wherein said compound is present in a concentration of 5 ug/ml to 15 ug/ml, or wherein said compound is present in a concentration of 5 ug/ml to 20 ug/ml, or wherein said compound is present in a concentration of 5 ug/ml to 30 ug/ml, or wherein said compound is present in a concentration of 7 ug/ml to 8 ug/ml, or wherein said compound is present in a concentration of 7 ug/ml to 9 ug/ml, or wherein said compound is present in a concentration of 7 ug/ml to 10 ug/ml, or wherein said compound is present in a concentration of 7 ug/ml to 15 ug/ml, or wherein said compound is present in a concentration of 7 ug/ml to 20 ug/ml, or wherein said compound is present in a concentration of 7 ug/ml to 30 ug/ml, or wherein said compound is present in a concentration of 30 ug/ml to 70 ug/ml or wherein said compound is present in a concentration of 70 ug/ml to 100 ug/ml or wherein said compound is present in a concentration of 100 ug/ml to 150 ug/ml. In an embodiment, the compound(s) is(are) in form in form of powder, liquid or crystal.

This invention provides a use of compounds or methods for treating cancers, inhibition of cancer growth, cancer invasion, cells invasion, cancer cell invasion; macromolecular cell invasion, cell adhesion, cell attachment, cell circulating, migration, metastasis or growth of cancers, infection or re-infection of virus or infectious macromolecules, and cancer cell fusion, wherein the invention comprises a pharmaceutical composition comprising the compound of this invention or a pharmaceutically acceptable salt thereof, and a pharmaceutically acceptable carrier or diluent, wherein said compound is present in a concentration of 0.008 uM to 80 uM, or wherein said compound is present in a concentration of 0.01 uM to 60 uM, or wherein said compound is present in a concentration of 0.01 uM to 50 uM, or wherein said compound is present in a concentration of 0.01 uM to 40 uM, or wherein said compound is present in a concentration of 0.01 uM to 30 uM, or wherein said compound is present in a concentration of 0.01 uM to 20 uM, or wherein said compound is present in a concentration of 0.01 uM to 10 uM, or wherein said compound is present in a concentration of 5 uM to 10 uM, or wherein said compound is present in a concentration of 0.1 uM to 5 uM, or wherein said compound is present in a concentration of 0.1 uM to 7.5 uM, or wherein said compound is present in a concentration of 0.1 uM to 10 uM, or wherein said compound is present in a concentration of 0.1 uM to 15 uM, or wherein said compound is present in a concentration of 0.1 uM to 20 uM, or wherein said compound is present in a concentration of 0.1 uM to 30 uM or wherein said compound is present in a concentration of 0.1 uM to 40 uM, or wherein said compound is present in a concentration of 0.1 uM to 50 uM or wherein said compound is present in a concentration of 0.1 uM to 60 uM, or wherein said compound is present in a concentration of 0.1 uM to 80 uM, or wherein said compound is present in a concentration of 1 uM to 5 uM, or wherein said compound is present in a concentration of 1 uM to 7.5 uM, or wherein said compound is present in a concentration of 1 uM to 10 uM, or wherein said compound is present in a concentration of 1 uM to 15 uM, or wherein said compound is present in a concentration of 1 uM to 20 uM, or wherein said compound is present in a concentration of 1 uM to 30 uM or wherein said compound is present in a concentration of 1 uM to 40 uM, or wherein said compound is present in a concentration of 1 uM to 50 uM or wherein said compound is present in a concentration of 1 uM to 60 uM, or wherein said compound is present in a concentration of 1 uM to 80 uM, or wherein said compound is present in a concentration of 3 uM to 5 uM, or wherein said compound is present in a concentration of 3 uM to 7.5 uM, or wherein said compound is present in a concentration of 3 uM to 10 uM, or wherein said compound is present in a concentration of 3 uM to 15 uM, or wherein said compound is present in a concentration of 3 uM to 20 uM, or wherein said compound is present in a concentration of 3 uM to 30 uM or wherein said compound is present in a concentration of 3 uM to 40 uM, or wherein said compound is present in a concentration of 3 uM to 50 uM or wherein said compound is present in a concentration of 3 uM to 60 uM, or wherein said compound is present in a concentration of 3 uM to 80 uM, or wherein said compound is present in a concentration of 5 uM to 8 uM, or wherein said compound is present in a concentration of 5 uM to 10 uM, or wherein said compound is present in a concentration of 5 uM to 15 uM, or wherein said compound is present in a concentration of 5 uM to 20 uM, or wherein said compound is present in a concentration of 5 uM to 30 uM or wherein said compound is present in a concentration of 5 uM to 40 uM, or wherein said compound is present in a concentration of 5 uM to 50 uM or wherein said compound is present in a concentration of 5 uM to 60 uM, or wherein said compound is present in a concentration of 5 uM to 80 uM. or wherein said compound is present in a concentration of 7 uM to 8 uM, or wherein said compound is present in a concentration of 7 uM to 10 uM, or wherein said compound is present in a concentration of 7 uM to 15 uM, or wherein said compound is present in a concentration of 7 uM to 20 uM, or wherein said compound is present in a concentration of 7 uM to 30 uM or wherein said compound is present in a concentration of 7 uM to 40 uM, or wherein said compound is present in a concentration of 7 uM to 50 uM or wherein said compound is present in a concentration of 7 uM to 60 uM, or wherein said compound is present in a concentration of 7 uM to 80 uM or wherein said compound is present in a concentration of 70 uM to 100 uM, or wherein said compound is present in a concentration of 90 uM to 120 uM.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Disclosures of these references or publications in their entireties are hereby incorporated by reference into this application in order to more fully describe the state of the art to which this invention pertains.

It is to be noted that the transitional term “comprising”, which is synonymous with “including”, “containing” or “characterized by”, is inclusive or open-ended and does not exclude additional, un-recited elements or method steps.

### EXAMPLE 1

### Tablet for Dose Containing 10 mg, 20 mg 30 mg of Active Compound

| | | | | | | | | | | |
|---|---|---|---|---|---|---|---|---|---|---|
| Active | 1 | mg | 5 | mg | 10 | mg | 20 | mg | 30 | mg |
| compound | | | | | | | | | | |
| Micro- | 20 | mg | 20 | mg | 19.75 | mg | 60 | mg | 100 | mg |
| crystalline | | | | | | | | | | |
| cellulose | | | | | | | | | | |
| Corn starch | 29 | mg | 24.5 | mg | 19.75 | mg | 19.25 | mg | 18.5 | mg |
| Magnesium | 0 | mg | 0.5 | mg | 0.5 | mg | 0.75 | mg | 1.5 | mg |
| stearate | | | | | | | | | | |

The active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 1, 5, 10, 20, 30 mg, respectively of active ingredient per tablet.

### EXAMPLE 2

### Intravenous Solution Preparation

An intravenous dosage form of the active compound is prepared as follows:

Active compound 1-10 ug

Sodium citrate 5-50 mg

Citric acid 1-15 mg

Sodium chloride 1-8 mg

Water for injection (USP) q.s. to 1 mL

Utilizing the above quantities, the active compound is dissolved at room temperature in a prepared solution of sodium chloride, citric acid, and sodium citrate in water for injection.

### EXAMPLE 3

### Intravenous Drip Preparation

0.25-2.5 mg compound dissolved in 250 ml of 10% glucose solution or in 250 ml of 0.9% NaCl solution.

Intravenous drip preparation: 1-2. mg compound dissolved in 250 ml of 10% glucose solution or in 250 ml of 0.9% NaCl solution

Treatment of angelic acid with one of the many standard chlorinating reagents including phosphorus ocychloride, phosphorus trichloride and thionyl chloride produces tigloyl chloride. Oxalyl chloride produces a 2:1 ratio of angeloyl chloride to tigloyl chloride. Treatment of potassium salt in diethyl ether with oxalyl chloride and catalytic DMF for 2 hr at 0 C produces pure angeloyl chloride.

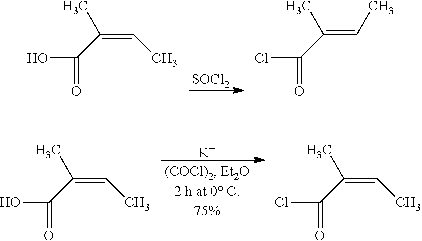

Acid Hydrolysis of the Following Compounds:

a) Xanifolia (Y),

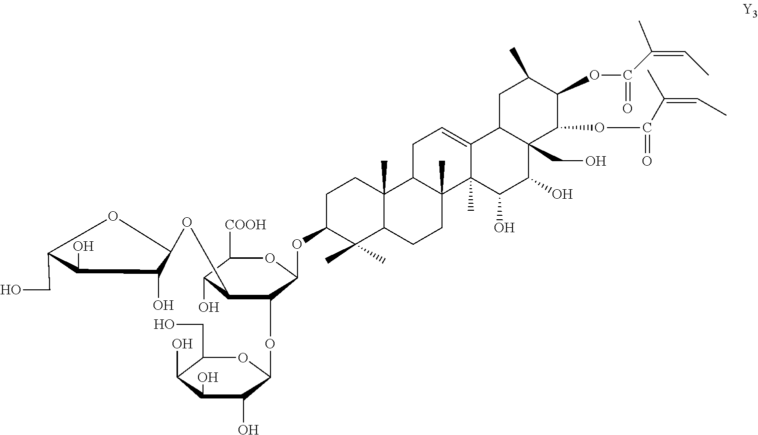

or chemical name: 3-O-[β-D-galactopyranosyl (1→2)]-α-L-arabinofuranosy (1→3)-β-D-glucuronopyranosyl-21,22-O-diangeloyl-3β,15α,16α,21β,22α,28-hexahydroxyolean-12-ene;

c) Xanifolia (Y2),

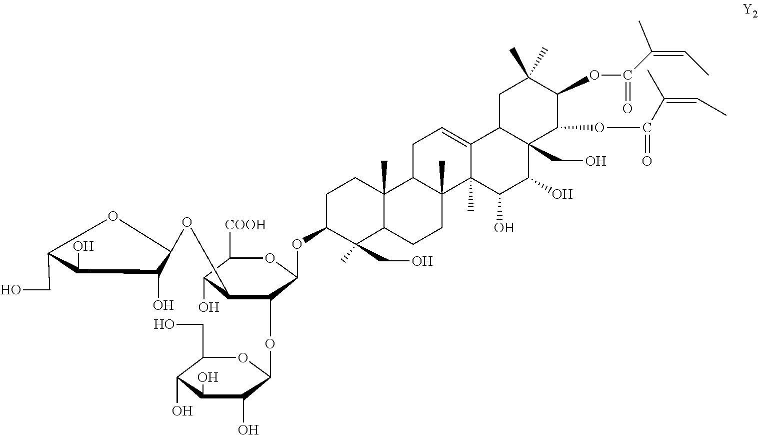

or chemical name: 3-O-[β-D-glucopyranosyl-(1→2)]-α-L-arabinofuranosy (1→3)-β-D-glucuronopyranosyl-21,22-O-diangeloyl-3β,15α,16α,21β,22α,24β,28-heptahydroxyolean-12-ene;

d) Xanifolia (Y8),

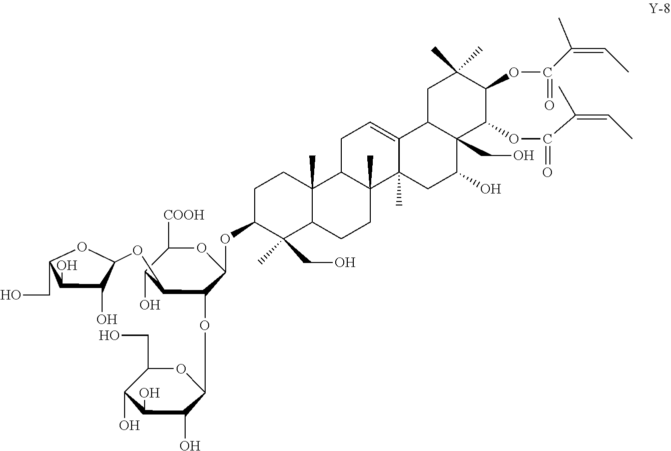

or chemical name: 3-O-[β-glucopyranosyl (1→2)]-α-arabinofuranosyl (1→3)-β-glucuronopyranosyl-21,22-O-diangeloyl-3β,16α,21β,22α,24β,28-hexahydroxyolean-12-ene;

m) structure (bES):

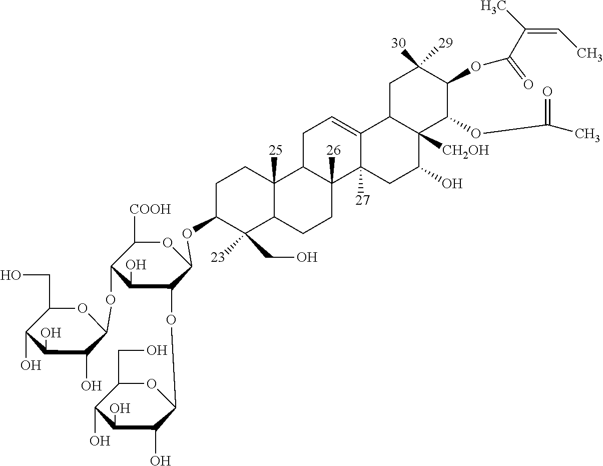

After acid hydrolysis of the above, an isolated, purified or synthesized compound is produced having a structure (ACH) selected from following:

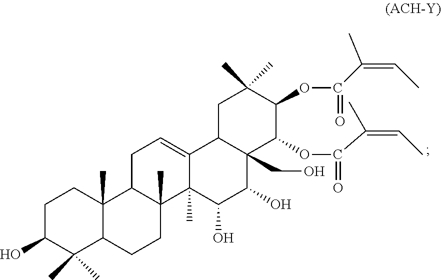

### ACH-Y10;

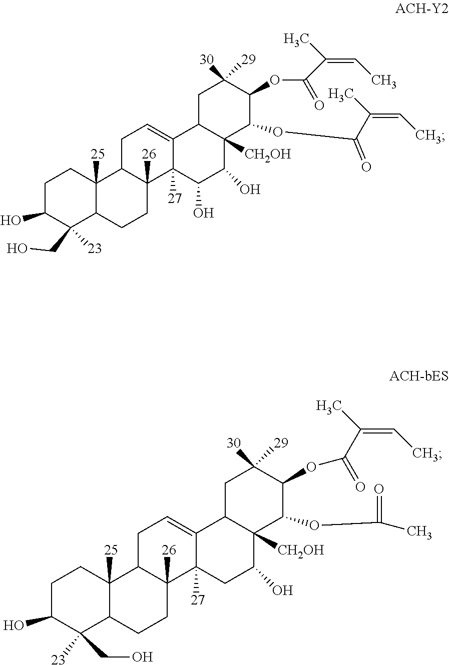 [0090] ACH-M10; ACH-n2

ACH-n3; ACH-n3

The composition comprises bioactive compounds from natural plants or synthesis.

The program is based on the purification methods and biological assays including the MTT assay as described in International Application No. PCT/US05/31900, filed Sep. 7, 2005, U.S. Ser. No. 11/289,142, filed Nov. 28, 2005, and U.S. Ser. No. 11/131,551, filed May 17, 2005, and PCT/US2008/002086, 1188-ALA-PCT, filed Feb. 15, 2008, Ser. No. 12/344,682, 1020-B1-US, filed Dec. 29, 2008, US009499577B2, filed Jun. 24, 2014. The details of Analysis of gene expression of ES2 cells after Y-treatment by Microarray, Data Analysis Methods and Western blot in PCT/US2008/002086, 1188-ALA-PCT, filed Feb. 15, 2008, and the cell invasion experiments methods in International Application PCT/US2010/0042240, filed Jul. 16, 2010.

The Haemolytic Assay

Erythrocytes (RBC) were isolated from human blood (EDTA whole blood, collected randomly). 50 ul of the 10% RBC suspension (in PBS) was added to 2 ml of sample solutions (concentration range from 0.1 ug/ml to 400 ug/ml) in PBS. The mixture was vortexed briefly and sat for 60 min at room temperature. The mixture was spun at 3K for 10 min and the relative amounts of lysed hemoglobin in the supernatant were measured at 540 nm. The synthetic compounds of present application were tested with this method.

Acid Hydrolysis of Saponin

15 mg Xanifolia-Y was dissolved in 1 ml of methanol. 1 ml of 2N HCl was then added. The mixture was refluxed in 80 C water bath for 5 hours. The solution was then neutralized by adding 2 ml of 1N NaOH (to final pH 4-6). The aglycone was then extracted with ethylacetate 3 ml×2. The extracts were collected and pooled. Further isolation of aglycone (ACH-Y) was achieved by HPLC with isocratic elution of 80-100% acetonitrile. Repeating the experiment with compounds Z4, Y10, Y2, Y8, Y7, Y0, X, M10 and ESCIN (bES) gives the following compounds respectively: ACH-Z4, ACH-Y10, ACH-Y2, ACH-Y8, ACH-Y7, ACH-Y0, ACH-X, ACH-E, ACH-Z5, ACH-M10 and ACH-bES.

Removal of the Acyl Group by Alkaline Hydrolysis

20 mg of Xanifolia-Y was dissolved in 0.5 ml of 1N NaOH. The solution was incubated in 80 C water bath for 4 hours. It was cooled to room temperature before being neutralized with 0.5 ml 1N HCl (adjust pH to about 3). The mixture was extracted with 2 ml 1-butanol 3 times. The butanol fractions were collected and lyophilized. The hydrolyzed saponin was further purified with HPLC in a C-18 column eluted with 25% acetonitrile.

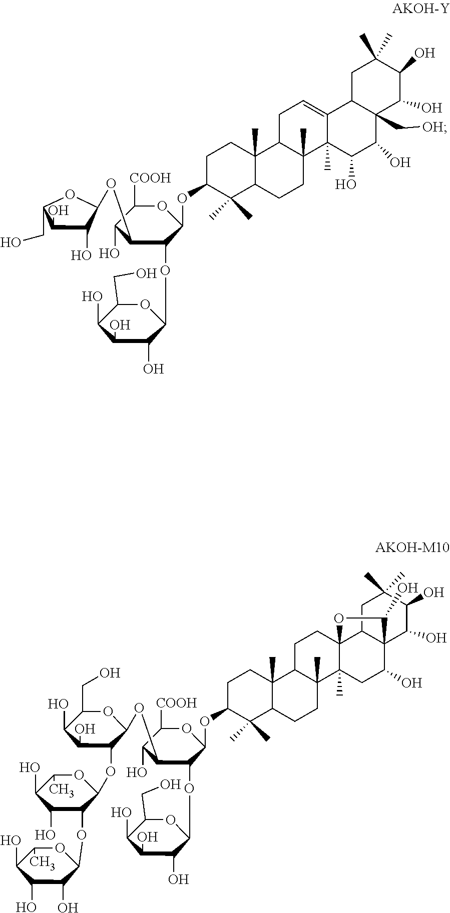

Compounds AKOH-Y and AKOH-M10 do not show the ability to inhibit cancer growth, cancer invasion, cells invasion or cancer cell invasion.

Core Compound

A core compound or pentacyclic triterpenes, hydroxylated triterpenes is obtained by acid and alkaline hydroysis of saponin from natural sources. A pentacyclic triterpene can also be obtained by synthetic methods. A method for synthesizing the core compound is as follows:

Beta-Escin, dissolved in 1M NaOH (20 mg/ml) was incubated at 70 C for 5 hours. The hydrolyzed solution was neutralized with HCl and the water was evaporated by lyophilization. The product was dissolved in 50% methanol and 1N HCl. The mixture was incubated at 70 C for 5 hours. The solution was neutralized with NaOH. The hydrolyzed product was extracted with ethylacetate, which was subsequently removed by evaporation. Further purification of the hydrolyzed product of core compounds including (E4A) were archived with FPLC chromatography in a C18 column equilibrated with 70% acetonitrile/TFA at the flow rate of 1 ml/min. The core compound are obtained.

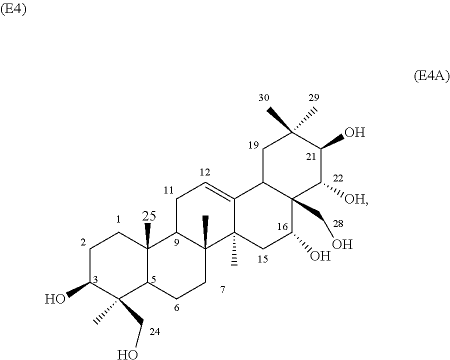

To a solution of compound E4A (2 mmol) in tetrahydrofuran (THF, 10 mL) were added methanesulfonyl chloride (Ms-Cl, 2.2 mmol) and triethylamine (TEA, 3 mmol) at 0° C. and the resulting mixture was stirred for 2 hours. The reaction mixture was concentrated under reduced pressure, and the residue was dissolved in ethyl acetate (EtOAc) and washed with water. The organic layer was dried over anhydrous magnesium sulfate, filtered and concentrated under reduced pressure. The residue was dissolved in 10 mL of dimethylformamide (DMF), and then sodium azide (NaN3, 6 mmol) was added. After overnight stirring at 60° C., the reaction mixture was diluted with ethyl acetate and washed with water. The organic layer was dried over anhydrous magnesium sulfate, filtered and concentrated under reduced pressure. Then, the obtained compound was dissolved in methanol (MeOH), and 10% palladium on carbon (Pd—C, 0.2 mmol) was added. After overnight stirring under hydrogen atmosphere, the reaction mixture was filtered, washed with methanol and concentrated under reduced pressure to provide the desired 24,28-diamine (E4D).

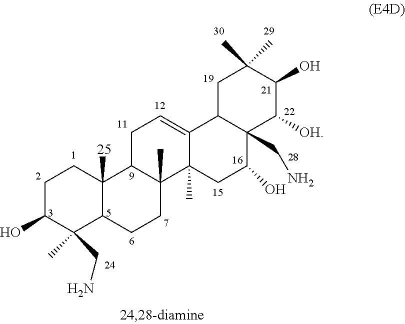

Further, Amination primary and secondary alcohol of core compound E4A to give E4D1

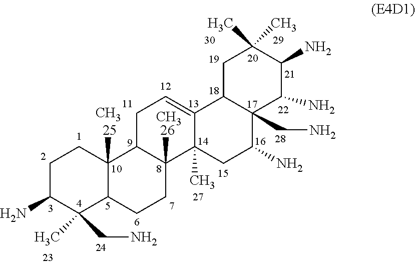

The core compounds do not show the ability to inhibit cancer growth, cancer invasion, or cell adhesion. The structures of core compounds:

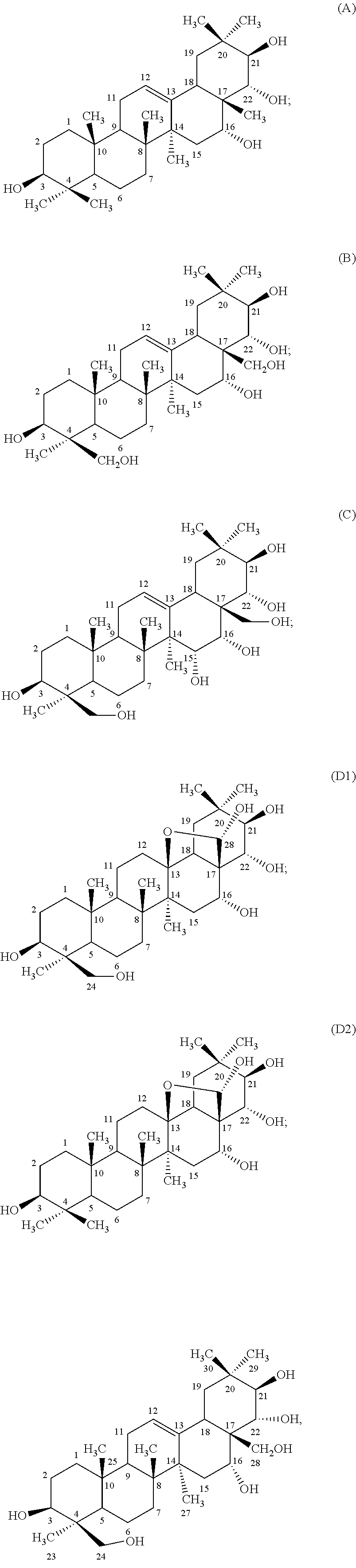

also named as bES-core, E IV A, ES4A, E4A or (E4);

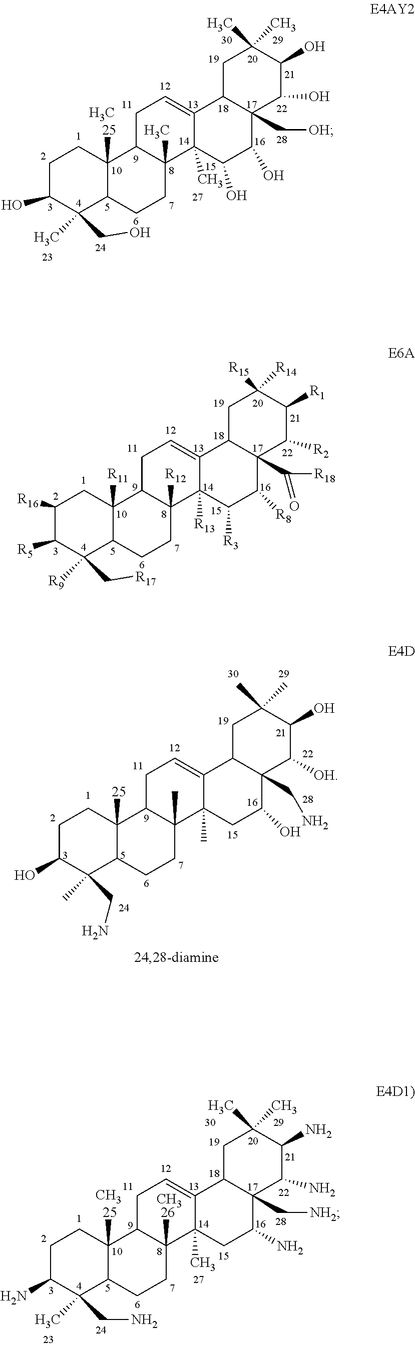

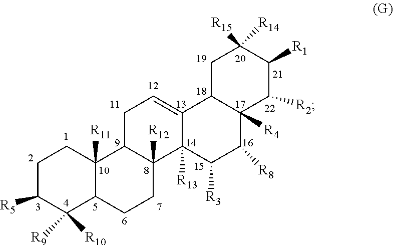

wherein R1, R2, R5, R8 represent OH; R3 represents OH, H or absent; R4, R10 represent CH3 or CH2OH; R9, R11, R12, R13, R14, R15 represent CH3;

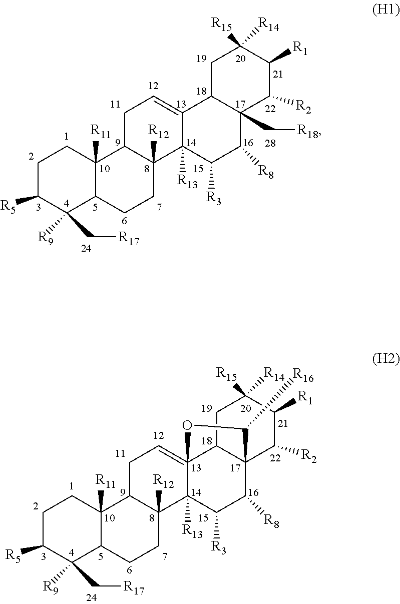

wherein R1, R2, R5, R8, R17, R18 represent OH or NH2; R3 represents NH2, OH, H or absent; R9, R11, R12, R13, R14, R15 represent CH3.

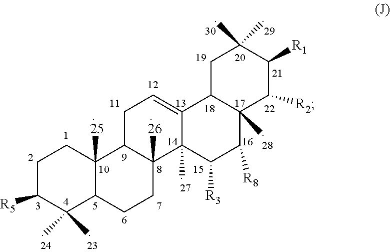

A typical numbering 1 to 30 of carbon positions of a pentacyclic triterpene.

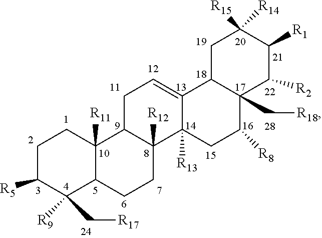

wherein R1, R2, R5, R8, R17, R18 represent OH; R9, R11, R12, R13, R14, R15 represent CH3, also named E4A or (E).

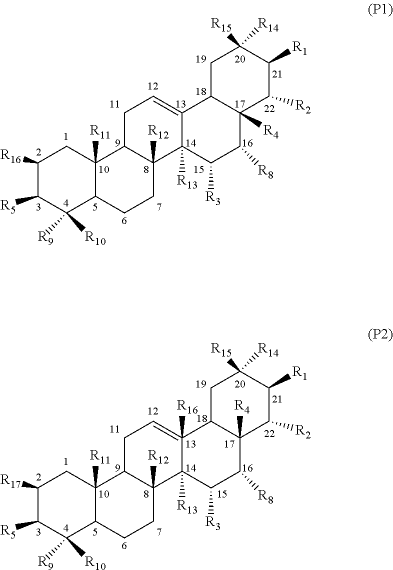

wherein R1, R2, R3, R4, R5, R8, R9, R10, R11, R12, R13, R14, R15, R16 R17 represent H, OH, CH2OH, COOH, OR CH3, named as (P1), (P2).

This invention provides a method of synthesizing new active compounds. A method of attaching functional groups to the core compounds including but not limited to (A), (B), (C), (D1), (D2), (E), (F), (G), (H1), (H2), (J), E4A, E4A2Y, E6A, (P1), P(2),] involves esterification or methods of present invention of core compounds with acyl halide, wherein the halide including chloride, bromide, fluoride and iodide, wherein the acyl halide comprises acyl chloride, wherein acyl chloride including but not limited to Tigloyl chloride, angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, benzoyl chloride, Ethylbutyryl chloride, Propionyl chloride, 2-Propenoyl chloride, Isobutyryl chloride, Butyryl chloride, (2E)-2-pentenoyl chloride, 4-Pentenoyl chloride, 5-Hexenoyl chloride, Heptanoyl chloride, Octanoyl chloride, Nonanoyl chloride, Decanoyl chloride, Lauroyl chloride, Myristoyl chloride, Oleoyl chloride for 5 sec, 1 min, 2 min, 5 min, 10 min, 30 min, 1 hr, 2 hr, 18 hr, 2 days or 3 days at 0 C, 25 C or 75 C temperature. At the end of reaction, 5 ml of 2N HCl or 1M NaHCO3 is added to the reaction mixture. The solution is then extracted 3 times with 10 ml of ethyl acetate which is then evaporated under vacuum and at 45 C and lyophilization. The reaction product is dissolved in 80% acetonitrile-0.005% Trifluoroacetic acid. The active esterification products are purified with HPLC. MTT activity was performed to test the activity of acyl chloride, solution after the reaction, individual fractions, and individual compounds. The core compounds are synthetic, semi synthetic or from natural source. The core compounds are including terpene, isoprene, triterpenes, and hydroxylated triterpenes.

MTT activity of acylation of core compounds in different reaction time period of (ASAP) 5 sec, 1 min, 2 min, 5 min, 10 min, 30 min, 1 hr, 2 hrs., 18 hrs., 2 days or 3 days at 0 C, 25 C or 75 C temperature were studied. HPLC profiles of esterification or methods of present application, products of core compound E4A with acyl halide, wherein the halide comprise chloride, bromide, fluoride and iodide, wherein the acyl halide comprise acyl chloride, wherein acyl chloride comprise tigloyl chloride, angeloyl chloride, acetyl chloride, crotonoyl chloride, 3,3-dimethylartyloyl chloride, senecioyl chloride, cinnamoyl chloride, pentenoyl chloride, hexanoyl chloride, benzoyl chloride, ethylbutyryl chloride, propionyl chloride, 2-propenoyl chloride, isobutyryl chloride, butyryl chloride, (2E) pentenoyl chloride, 4-Pentenoyl chloride, 5-hexenoyl chloride, heptanoyl chloride, octanoyl chloride, nonanoyl chloride, decanoyl chloride, Lauroyl chloride, myristoyl chloride, oleoyl chloride show that the compounds vary in composition when the time or temperature of the reaction is changed.

The peaks, fractions and compounds are selected according to the activities of times studies and the changes of peaks. Selecting the HPLC fractions for isolation is according to the cytotoxic activity of the reaction product obtained at a specific time. The compounds having strong to weak activities are selected and isolated. Selecting the HPLC fractions for isolation may be according to the cytotoxic activity of times studies and the change of peaks. The anti cancer activities are the MTT studies of bone (U2OS), lung (H460), bladder (HTB-9), ovary (ES2), colon (HCT116), pancreas (Capan), ovary (OVCAR3), prostate (DU145), skin (SK-Mel-5), mouth (KB), kidney (A498), breast (MCF-7), liver (HepG2), brain (T98G), luekemia (K562), cervix (HeLa).

Amidation of core compound E4D or E4D1 with Tigloyl or Tigloyl chloride and isolation of the compounds with HPLC give the following compounds: wherein Tig=Tigloyl

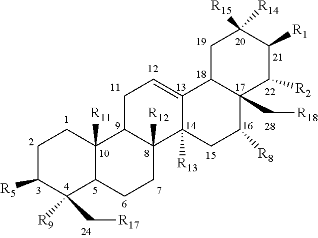

| | R1 | R2 | R5 | R8 | R17 | R18 | Cytotoxicity activity |
|---|---|---|---|---|---|---|---|
| E4D | OH | OH | OH | OH | NH2 | NH2 | none |
| E4D1 | NH2 | NH2 | NH2 | NH2 | NH2 | NH2 | none |
| AA1 | HN2 | NH2 | NH2 | NH2 | NH-Tig | NH-Tig | moderate |
| AA2 | OH | OH | OH | OH | NH -Tig | NH-Tig | strong |
| AA3 | NH-Tig | NH | NH | NH | NH-Tig | NH2 | moderate |
| AA4 | NH-Tig | NH2 | NH2 | NH2 | NH-Tig | NH-Tig | weak |

Amidation of core compound E4D or E4D1 with Angeloyl or Angeloyl chloride and isolation of the compounds with HPLC give the following compounds: wherein Ang=Angeloyl

| | R1 | R2 | R5 | R8 | R17 | R18 | Cytotoxicity activity |
|---|---|---|---|---|---|---|---|
| E4D | OH | OH | OH | OH | NH2 | NH2 | none |
| E4D1 | NH2 | NH2 | NH2 | NH2 | NH2 | NH2 | none |
| GG1 | HN2 | NH2 | NH2 | NH2 | NH-Ang | NH-Ang | moderate |
| GG2 | OH | OH | OH | OH | NH -Ang | NH-Ang | strong |
| GG3 | NH-Ang | NH | NH | NH | NH-Ang | NH2 | moderate |
| GG4 | NH-Ang | NH2 | NH2 | NH2 | NH-Ang | NH-Ang | weak |

Amidation of core compound E4D or E4D1 with senecioyl or senecioyl chloride and isolation of the compounds with HPLC give the following compounds:

Wherein Sen=senecioyl

| | R1 | R2 | R5 | R8 | R17 | R18 | Cytotoxicity activity |
|---|---|---|---|---|---|---|---|
| E4D | OH | OH | OH | OH | NH2 | NH2 | none |
| E4D1 | NH2 | NH2 | NH2 | NH2 | NH2- | NH2 | moderate |
| BB1 | NH2 | NH2 | NH2 | NH2 | NH-Sen | NH-Sen | moderate |
| BB2 | OH | OH | OH | OH | NH-Sen | NH-Sen | strong |
| BB3 | NH-Sen | NH2 | NH2 | NH2 | NH-Sen | NH2 | moderate |
| BB4 | NH-Sen | NH2 | NH2 | NH2 | NH-Sen | NH-Sen | weak |

Amidation of core compound E4D, or E4D1 with Pentenoyl or 4-Pentenoyl chloride and isolation of the compounds with HPLC give the following compounds: wherein Pen=4-Pentenoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | NONE |
| CC1 | NH2 | NH2 | NH2 | NH2 | NH-Pen | NH-Pen | moderate |
| CC2 | OH | OH | OH | ON | NH-Pen | NH-Pen | strong |
| CC3 | NH-Pen | NH2 | NH2 | NH2 | NH-Pen | NH2 | moderate |
| CC4 | NH-Pen | NH2 | NH2 | NH2 | NH-Pen | NH-Pen | weak |

Amidation of core compound E4D, or E4D1 with Hexanoyl or Hexanoyl chloride and isolation of the compounds with HPLC give the following compounds: wherein Hex=Hexanoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | moderate |
| DD1 | NH2 | NH2 | NH2 | NH2 | NH- | NH- | moderate |
| | | | | | Hex | Hex | |
| DD2 | OH | OH | OH | OH | NH- | NH- | strong |
| | | | | | Hex | Hex | |
| DD3 | NH- | NH2 | NH2 | NH2 | NH- | NH2 | moderate |
| | Hex | | | | Hex | | |
| DD4 | NH- | NH2 | NH2 | NH2 | NH- | NH- | weak |
| | Hex | | | | Hex | Hex | |

Amidation of core compound E4D or E4D1 with2-Ethylbutyryl or 2-Ethylbutyryl chloride and isolation of the compounds with HPLC give the following compounds: wherein Eth=2-Ethylbutyryl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E1E4D1 | NH2 | NH2 | NH2 | NH2 | NH2 | NH2 | none |
| EE1 | NH | NH | NH | NH | NH- | NH- | moderate |
| | | | | | ETH | Eth | |
| EE2 | OH | OH | OH | OH | NH- | NH- | strong |
| | | | | | Eth | Eth | |
| EE3 | NH- | NH | NH | NH | NH- | NH | moderate |
| | Eth | | | | Eth | | |
| EE4 | NH- | NH | NH | NH | NH- | NH- | weak |
| | Eth | | | | Eth | Eth | |

Amidation of core compound E4D or E4D1 with Acetyl chloride (H) and isolation of the compounds with HPLC give the following compounds: wherein Acy=Acetyl

| | | | | | | | Cytotoxicity |
|---|---|---|---|---|---|---|---|
| | R1 | R2 | R5 | R8 | R17 | R18 | activity |
| E4D | OH | OH | OH | OH | NH | NH | none |
| E4D1 | NH2 | NH2 | NH2 | NH2 | NH2 | NH2 | none |
| HH1 | NH2 | NH2 | NH2 | NH2 | NH- | NH- | moderate |
| | | | | | Acy | Acy | |
| HH2 | OH | OH | OH | OH | NH- | NH- | strong |
| | | | | | Acy | Acy | |
| HH3 | NH- | NH2 | NH2 | NH2 | NH- | NH2 | moderate |
| | Acy | | | | Acy | | |
| HH4 | NH- | NH2 | NH2 | NH2 | NH- | NH- | weak |
| | Acy | | | | Acy | Acy | |

Amidation of core compound E4D or E4D1 with Crotonoyl or Crotonoyl chloride and isolation of the compounds with HPLC give the following compounds: wherein Cro=Crotonoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| I 1E4D1 | NH2 | NH2 | NH2 | NH2 | NH2 | NH2 | none |
| II 1 | NH | NH | NH | NH | NH- | NH- | moderate |
| | | | | | Cro | Cro | |
| I I2 | OH | OH | OH | OH | NH- | NH- | strong |
| | | | | | Cro | Cro | |
| I I3 | NH- | NH2 | NH2 | NH2 | NH- | NH2 | moderate |
| | Cro | | | | Cro | | |
| II 4 | NH- | NH2 | NH2 | NH2 | NH- | NH- | weak |
| | Cro | | | | Cro | Cro | |

Amidation of core compound E4D or E4D1 with Cinnamoyl or Cinnamoyl chloride and isolation of the compounds with HPLC give the following compounds: wherein Cin=Cinnamoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| JJ1 | NH | NH | NH | NH | NH-Cin | NH-Cin | moderate |
| JJ2 | OH | OH | OH | OH | NH-Cin | NH-Cin | strong |
| JJ3 | NH-Cin | NH | NH | NH | NH-Cin | NH | moderate |
| JJ4 | NH-Cin | NH | NH | NH | NH-Cin | NH-Cin | weak |

Amidation of core compound E4D or E4D1 with benzoyl or benzoyl chloride and isolation of the compounds with HPLC give the following compounds: wherein Ben=benzoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | None |
| E4D1 | NH2 | NH2 | NH2 | NH2 | NH2 | NH2 | none |
| KK1 | NH2 | NH2 | NH2 | NH2 | NH-Ben | N-Ben | moderate |
| KK2 | OH | OH | OH | OH | NH-Ben | NH-Ben | strong |
| KK3 | NH-Ben | NH2 | NH2 | NH2 | NH-Ben | NH2 | moderate |
| KK4 | NH-Ben | NH2 | NH2 | NH2 | NH-Ben | NH-Ben | weak |

Amidation of core compound E4D OR E4D1 with Propionyl or Propionyl chloride and isolation of the compounds with HPLC give the following compounds: wherein Ppi=Propionyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| LL1 | NH2 | NH2 | NH2 | NH2 | NH-Ppi | NH-Ppi | moderate |
| LL2 | OH | OH | OH | OH | NH-Ppi | NH-Ppi | strong |
| LL3 | NH-Ppi | NH2 | NH2 | NH2 | N-Ppi | NH2 | moderate |
| LL4 | NH-Ppi | NH2 | NH2 | NH2 | NH-Ppi | NH-Ppi | weak |

Amidation of core compound E4D OR E4D1 with 2-propenoyl or 2-propenoyl chloride and isolation of the compounds with HPLC give the following compounds: wherein Ppe=Propenoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| MM1 | NH | NH | NH | NH | NH-Ppe | NH-Ppe | moderate |
| MM2 | OH | OH | OH | OH | NH-Ppe | NH-Ppe | strong |
| MM3 | NH-Ppe | NH2 | NH2 | NH2 | NH-Ppe | NH2 | moderate |
| MM4 | NH-Ppe | NH2 | NH2 | NH2 | NH-Ppe | NH-Ppe | weak |

Amidation of core compound E4D or E4D1 with Isobutyryl or Isobutyryl chloride and isolation of the compounds with HPLC give the following compounds: wherein Iso=Isobutyryl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| NN1 | NH2 | NH2 | NH2 | NH2 | NH-Iso | NH-Iso | moderate |
| NN2 | OH | OH | OH | OH | NH-Iso | NH-Iso | strong |
| NN3 | NH-Iso | NH2 | NH2 | NH2 | N-Iso | NH2 | moderate |
| NN4 | NH-Iso | NH2 | NH2 | NH2 | NH-Iso | NH-Iso | weak |

Amidation of core compound E4D or E4D1 with Butyryl or Butyryl chloride and isolation of the compounds with HPLC give the following compounds: wherein But=Butyryl

| | | | | | | | Cytotoxicity |
|---|---|---|---|---|---|---|---|
| | R1 | R2 | R5 | R8 | R17 | R18 | activity |
| E4D | OH | OH | OH | OH | ON2 | NH2 | none |
| E4D1 | NH2 | NH2 | NH2 | NH2 | NH2-But | NH2 | none |
| PP1 | NH2 | NH2 | NH2 | NH2 | NH-But | NH-But | moderate |
| PP2 | OH | OH | OH | OH | NH-But | NH-But | strong |
| PP3 | NH-But | NH2 | NH2 | NH2 | NH-But | NH2 | moderate |
| PP4 | NH-But | NH2 | NH2 | NH2 | NH-But | NH-But | weak |

Amidation of core compound E4D or E4D1 with(2E)-2-pentenoyl or (2E)-2-pentenoyl chloride and isolation of the compounds with HPLC give the following compounds: wherein 2pe=2-pentenoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| QQ1 | NH2 | NH2 | NH2 | NH2 | NH-2Pe | NH-2pe | moderate |
| QQ2 | OH | OH | OH | OH | NH-2pe | NH-2pe | strong |
| QQ3 | NH-2pe | NH2 | NH2 | NH2 | NH-2pe | NH2 | moderate |
| QQ4 | NH-2pe | NH2 | NH2 | NH2 | NH-2pe | NH-2pe | weak |

Amidation of core compound E4D of E4D1 with Octanoyl or Octanoyl chloride and isolation of the compounds with HPLC give the following compounds: wherein Oct=Octanoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| RR1 | NH2 | NH2 | NH2 | NH2 | NH-Oct | NH-Oct | moderate |
| RR2 | OH | OH | OH | OH | NH-Oct | NH-Oct | strong |
| RR3 | NH-Oct | NH2 | NH2 | NH2 | NH-Oct | NH2 | moderate |
| RR4 | NH-Oct | NH2 | NH2 | NH2 | NH-Oct | NH-Oct | weak |

Amidation of core compound E4D or E4D1 with Decanoyl or Decanoyl chloride and isolation of the compounds with HPLC give the following compounds: wherein Dec=Decanoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| SS1 | NH2 | NH2 | NH2 | NH2 | NH-Dec | NH-Dec | moderate |
| SS2 | OH | OH | OH | OH | NH-Dec | NH-Dec | strong |
| SS3 | NH-Dec | NH2 | NH2 | NH2 | NH-Dec | NH | moderate |
| SS4 | NH-Dec | NH2 | NH2 | NH2 | NH-Dec | NH-Dec | weak |

Amidation of core compound E4D or E4D1 with Myristoyl or Myristoyl chloride and isolation of the compounds with HPLC give the following compounds: wherein Myr=Myristoyl

| | | | | | | | Cytotoxicity |
|---|---|---|---|---|---|---|---|
| | R1 | R2 | R5 | R8 | R17 | R18 | activity |
| E4D | OH | OH | OH | OH | NH2 | NH2 | none |
| E4D1 | NH2 | NH2 | NH2 | NH2 | NH2 | NH2 | none |
| TT1 | NH2 | NH2 | NH2 | NH2 | NH- | NH- | moderate |
| | | | | | Myr | Myr | |
| TT2 | OH | OH | OH | OH | NH- | NH- | strong |
| | | | | | Myr | Myr | |
| TT3 | NH- | NH2 | NH2 | NH2 | NH- | NH2 | moderate |
| | Myr | | | | Myr | | |
| TT4 | NH- | NH2 | NH2 | NH2 | NH- | NH- | weak |
| | Myr | | | | Myr | Myr | |

Esterification, amidation, amination, or sulfonamidation of compound (A), (B), (C), (D1), (D2), (E), (F), (G), (H1), (H2), E4A, E4A2Y, E4D, E4D1, (P1), P(2), terpene, isoprene, triterpenes, hydroxylated triterpenes, with acyl halide, wherein the halide comprise chloride, bromide, fluoride and iodide, wherein the acyl halide comprise acyl chloride, wherein acyl chloride comprise tigloyl chloride, angeloyl chloride, acetyl chloride, crotonoyl chloride, 3,3-dimethylartyloyl chloride, senecioyl chloride, cinnamoyl chloride, pentenoyl chloride, hexanoyl chloride, benzoyl chloride, ethylbutyryl chloride, propionyl chloride, 2-propenoyl chloride, isobutyryl chloride, butyryl chloride, (2E)-2-pentenoyl chloride, 4-Pentenoyl chloride, 5-hexenoyl chloride, heptanoyl chloride, octanoyl chloride, nonanoyl chloride, decanoyl chloride, Lauroyl chloride, myristoyl chloride, oleoyl chloride. The compounds vary in composition when the time or temperature of the reaction is changed. The peaks, fractions and compounds are selected according to the activities of times studies and the changes of peaks. The compounds having strong to weak activities are selected and isolated. The anti cancer activities (Cytotoxic Assay) are the MTT studies of bone (U2OS), lung (H460), bladder (HTB-9), ovary (ES2), colon (HCT116), pancreas (Capan), ovary (OVCAR3), prostate (DU145), skin (SK-Mel-5), mouth (KB), kidney (A498), breast (MCF-7), liver (HepG2), brain (T98G), luekemia (K562), cervix (HeLa). The active esterification, amidation, amination, or sulfonamidation products are purified with HPLC. The reaction product of mixtures and individual compounds are tested with MTT Cytotoxic Assay. Details of method are in Experiment 3 of the present application. A second esterification of compound can be selected from the above experiment results to produce new active compounds. A partial esterification compound is selected from the above experiments to perform a second or repeated with a third esterification with different acyl chloride in order to produce new active compounds with the experiments in the present application. The active compounds of this invention are triterpenes in form of amine, sulfonamides, amide, and urea analogs, providing extremely stable activity in solution. They prolong the activities and duration of drug in a subject.

A method is 1) Dissolving core compound or triterpenes core, hydroxylated triterpenes core, in pyridine; 2) Adding acyl halide or acyl chloride; 3, The mixture is stirred for length of time including 5 sec, 10 sec, 20 sec, 30 sec, 40 sec, 1 min, 2 min, 5 min, 10 min, 30 min, 1 hr, 2 hr, 18 hr, 2 days or 3 days at different temperature; 4) At the end of reaction, aqueous solution of acid or weak base, or water is added to the reaction mixture; 5) The solution is then extracted of ethyl acetate and ethyl acetate is removed by evaporation and lyophilization; 6) Dissolving the reaction product in acetonitrile with Trifluoroacetic acid or DMSO; 7) Testing the reaction product of mixtures and individual fractions with MTT cytotoxic assay; 8) Selecting the HPLC fractions for isolation is according to the cytotoxic activity of the reaction product obtained at a specific reaction time; 10) Purifiing the active esterification products with HPLC; 11) Collecting the products; 12) Testing the products; wherein the core compound is terpene, isoprene, or triterpene core or hydroxylated triterpenes core; wherein the core compound was dissolved in pyridine; wherein the acyl chloride including Tigloyl chloride, angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, benzoyl chloride, Ethylbutyryl chloride, Propionyl chloride, 2-Propenoyl chloride, Isobutyryl chloride, Butyryl chloride, (2E)-2-pentenoyl chloride, 4-Pentenoyl chloride, 5-Hexenoyl chloride, Heptanoyl chloride, Octanoyl chloride, Nonanoyl chloride, Decanoyl chloride, Lauroyl chloride, Myristoyl chloride, and Oleoyl chloride; wherein the reaction time for the mixture is stirred for 5 sec, 10 sec, 20 sec, 30 sec, 40 sec, 1 min, 2 min, 5 min, 10 min, 30 min, 1 hr, 2 hrs., 18 hrs., 2 days or 3 days; wherein the temperature is 0 C, 25 C, 50 C or 75 C temperature; wherein the acid including HCl or the base including NaHCO3 is added to the reaction mixture; wherein the solution is then extracted 3 times with ethyl acetate and lyophilization; wherein the reaction product is dissolved in 80% acetonitrile-0.005% Trifluoroacetic acid or DMSO; wherein selecting the HPLC fractions for isolation is according to the cytotoxic activity of the reaction product obtained at a reaction time of 5 sec, 10 sec, 20 sec, 30 sec, 40 sec, 1 min, 2 min, 5 min, 10 min, 30 min, 1 hr, 2 hr, 18 hr, 2 days or 3 days. In an embodiment, the reaction time may be over 3 days. In an embodiment, the experiment may be performed under 0 C. In an embodiment, the experiment may be performed over 75 C.

The anti-cancer activities of Tig-R compound: IC50 of bone (U2OS) is 4.5 ug/ml, lung (H460) is 4.8 ug/ml, bladder (HTB-9) is 2.5 ug/ml, ovary (ES2) is 2.8 ug/ml, colon (HCT116) is 5.2 ug/ml, pancreas (Capan) 2.4 ug/ml, ovary (OVCAR3) is 5.8, prostate (DU145) is 3.6 ug/ml, skin (SK-Mel-5) is 5.1 ug/ml, mouth (KB) is 3 ug/ml, kidney (A498) is 3.5 ug/ml, breast (MCF-7) is 4.5 ug/ml, liver (HepG2) is 6 ug/ml, brain (T98G) is 8 ug/ml), leukemia (K562) is 2 ug/ml, cervix (HeLa) is 5 ug/ml.

The anti-cancer activities of Tig-V compound: IC50 of bone (U2OS) is 7 ug/ml, lung (H460) is 6.8 ug/ml, bladder (HTB-9) is 4 ug/ml, ovary (ES2) is 2 ug/ml, colon (HCT116) is 8 ug/ml, pancreas (Capan) 5 ug/ml, ovary (OVCAR3) is 9, prostate (DU145) is 4 ug/ml, skin (SK-Mel-5) is 6 ug/ml, mouth (KB) is 4.5 ug/ml, kidney (A498) is 4.8 ug/ml, breast (MCF-7) is 9 ug/ml, liver (HepG2) is 12 ug/ml, brain (T98G) is 14 ug/ml), leukemia (K562) is 4 ug/ml, cervix (HeLa) is 7 ug/ml.

The anti-cancer activities of Tig-N compound: IC50 of bone (U2OS) is 15 ug/ml, lung (H460) is 13 ug/ml, bladder (HTB-9) is 7.5 ug/ml, ovary (ES2) is 9 ug/ml, colon (HCT116) is 15 ug/ml, pancreas (Capan) 8 ug/ml, ovary (OVCAR3) is 18, prostate (DU145) is 4.8 ug/ml, skin (SK-Mel-5) is 15 ug/ml, mouth (KB) is 9 ug/ml, kidney (A498) is 11 ug/ml, breast (MCF-7) is 13 ug/ml, liver (HepG2) is 18 ug/ml, brain (T98G) is 19 ug/ml), leukemia (K562) is 6 ug/ml, cervix (HeLa) is 15 ug/ml.

The anti-cancer activities of Tig-Q compound: IC50 of bone (U2OS) is 20 ug/ml, lung (H460) is 18 ug/ml, bladder (HTB-9) is 10 ug/ml, ovary (ES2) is 12 ug/ml, colon (HCT116) is 22 ug/ml, pancreas (Capan) 9 ug/ml, ovary (OVCAR3) is 23, prostate (DU145) is 15 ug/ml, skin (SK-Mel-5) is 20 ug/ml, mouth (KB) is 12 ug/ml, kidney (A498) is 13 ug/ml, breast (MCF-7) is 18 ug/ml, liver (HepG2) is 24 ug/ml, brain (T98G) is 29 ug/ml), leukemia (K562) is 6 ug/ml, cervix (HeLa) is 20 ug/ml.

The anti-cancer activities of Tig-T compound: IC50 of bone (U2OS) is 20 ug/ml, lung (H460) is 21 ug/ml, bladder (HTB-9) is 12 ug/ml, ovary (ES2) is 14 ug/ml, colon (HCT116) is 23 ug/ml, pancreas (Capan) 10 ug/ml, ovary (OVCAR3) is 25, prostate (DU145) is 16 ug/ml, skin (SK-Mel-5) is 22 ug/ml, mouth (KB) is 13 ug/ml, kidney (A498) is 15 ug/ml, breast (MCF-7) is 20 ug/ml, liver (HepG2) is 26 ug/ml, brain (T98G) is 26 ug/ml), leukemia (K562) is 9 ug/ml, cervix (HeLa) is 18 ug/ml.

The anti-cancer activities of Tig-S compound: IC50 of bone (U2OS) is 5.2 ug/ml, lung (H460) is 5.6 ug/ml, bladder (HTB-9) is 3.5 ug/ml, ovary (ES2) is 0.1 ug/ml, colon (HCT116) is 6.6 ug/ml, pancreas (Capan) 2.9 ug/ml, ovary (OVCAR3) is 6.5, prostate (DU145) is 4.3 ug/ml, skin (SK-Mel-5) is 5.8 ug/ml, mouth (KB) is 4 ug/ml, kidney (A498) is 4.8 ug/ml, breast (MCF-7) is 6.3 ug/ml, liver (HepG2) is 8.5 ug/ml, brain (T98G) is 9 ug/ml), leukemia (K562) is 4.3 ug/ml, cervix (HeLa) is 7 ug/ml.

The anti-cancer activities of Tig-U compound: IC50 of bone (U2OS) is 23 ug/ml, lung (H460) is 19 ug/ml, bladder (HTB-9) is 15 ug/ml, ovary (ES2) is 17 ug/ml, colon (HCT116) is 26 ug/ml, pancreas (Capan) 9 ug/ml, ovary (OVCAR3) is 27, prostate (DU145) is 15 ug/ml, skin (SK-Mel-5) is 24 ug/ml, mouth (KB) is 16 ug/ml, kidney (A498) is 18 ug/ml, breast (MCF-7) is 25 ug/ml, liver (HepG2) is 23 ug/ml, brain (T98G) is 22 ug/ml), leukemia (K562) is 10 ug/ml, cervix (HeLa) is 17 ug/ml.

The IC50 of Tig-R in normal human fibroblast cells (WI38) is about 10-15 ug/ml. This IC50 value is 3 times higher than those in ovary ES2 (2.8 ug/ml) and lung (H460) is 4.8 ug/ml.

Swiss3T3 cells are mouse normal fibroblast which were used in this experiment to compare with ES2 (human ovarian cancer) in Tig-R cytotoxicity determination. The preliminary results indicate that the IC50 of Tig-R in SW3T3 cells is above 20 ug/ml while the corresponding IC50 in ES2 cells is about 2.8 ug/ml.

This invention provides compounds, methods, or uses of a compound for the manufacture of a medicament, or uses of a compound for medicament selected from formula (2A), K, 3K, 3K2, T1, T2, T3, T4, T5, T6, T7, T8, T9,T10, T11, T12, T13, T14, T15, T16, T17, T18, T19, T20, T21, T22, T23, T24, T25, T26, T27 for treating cancers, inhibition of cancer growth, cancer invasion, cells invasion, cancer cell invasion; cell adhesion, cell attachment, cell circulating; for treating mad cow disease; treating prion diseases; for inhibiting viruses; for preventing cerebral aging; for improving memory; improving cerebral functions; for curing enuresis, frequent micturition, urinary incontinence; dementia, Alzheimer's disease, autism, brain trauma, Parkinson's disease or other diseases caused by cerebral dysfunctions or neurodegeneration; for treating arthritis, rheumatism, poor blood circulation, arteriosclerosis, Raynaud's syndrome, angina pectoris, cardiac disorder, coronary heart disease, headache, dizziness, kidney disorder; cerebrovascular diseases; inhibiting NF-kappa B activation; for treating brain edema, severe acute respiratory syndrome, respiratory viral diseases, chronic venous insufficiency, hypertension, chronic venous disease, oedema, inflammation, hemorrhoids, peripheral edema formation, varicose vein disease, flu, post traumatic edema and postoperative swelling; for inhibiting blood clots, for inhibiting ethanol absorption; for lowering blood sugar; for regulating adrenocorticotropin and corticosterone levels; for Anti-MS, anti-aneurysm, anti-asthmatic, anti-oedematous, anti-inflammatory, anti-bradykinic, anti-capillarihemorrhagic, anti-cephalagic, anti-cervicobrachialgic, anti-eclamptic, anti-edemic, anti-encaphalitic, anti-epiglottitic, anti-exudative, anti-flu, anti-fracture, anti-gingivitic, anti-hematomic, anti-herpetic, anti-histaminic, anti-hydrathritic, anti-meningitic, antioxidant, anti-periodontic, anti-phlebitic, anti-pleuritic, anti-raucedo, anti-rhinitic, anti-tonsilitic, anti-ulcer, anti-varicose, anti-vertiginous, cancerostatic, corticosterogenic, diuretic, fungicide, hemolytic, hyaluronidase inhibitor, lymphagogue, natriuretic, pesticide, pituitary stimulant, thymolytic, vasoprotective, inhibiting leishmaniases, modulating adhesion or angiogenesis of cells, anti-parasitic; for improving blood circulation; soothing stroke; preventing plaque formation and promote their dissipated; improve blood viscosity; reducing cardiovascular; reducing cerebrovascular; reducing thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, fluttering, Blood circulation, swelling, pain; treating bronchiectasis, tuberculosis and lung abscess caused by too hemoptysis; reducing bleeding; antitussive; reducing expectorant; reducing analgesic effect; dilate blood vessels; reducing blood pressure; treatment of cerebral arteriosclerosis; elevating blood lipids; reducing cholesterol; manufacturing an adjuvant composition for treatment.

In an embodiment, the cancers comprise breast cancer, leukocytic cancer, liver cancer, ovarian cancer, bladder cancer, prostatic cancer, skin cancer, bone cancer, brain cancer, leukemia cancer, lung cancer, colon cancer, CNS cancer, melanoma cancer, renal cancer, cervical cancer, esophageal cancer, testicular cancer, splenic cancer, kidney cancer, lymphatic cancer, pancreatic cancer, stomach cancer, eye cancer and thyroid cancer; wherein the cells comprise breast cell, leukocytic cell, liver cell, ovarian cell, bladder cell, prostatic cell, skin cell, bone cell, brain cell, leukemia cell, lung cell, colon cell, CNS cell, melanoma cell, renal cell, cervical cell, esophageal cell, testicular cell, splenic cell, kidney cell, lymphatic cell, pancreatic cell, stomach cell and thyroid cell.

In an embodiment, the compound is selected from the structure:

With the experiments in present application, the compound is selected from the structures:

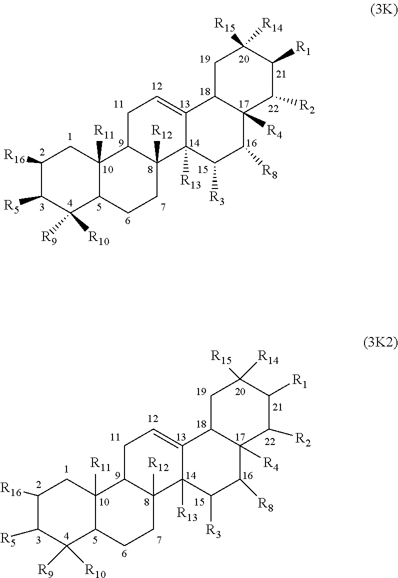

R1, R2, R3, R4, R5, R8, R9, R10, R11, R12, R13, R14, R15, R16 are independently selected from the group of H, O, OH, NH2, CH2NH2, NH-angeloyl, CH2NH-angeloyl, CH2NHCO-angeloyl, CH2NHCONH-angeloyl, CH2NHSO2-angeloyl, CH3, CH2OH, COOH, hydrogen, hydroxyl, methyl, O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-alkane, O-alkene, O-sugar moiety, O-acid moiety, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, -Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, COO-angeloyl, COO-tigloyl, COO-senecioyl, COO-acetyl, COO-Crotonoyl, COO-3,3-Dimethylartyloyl, COO-Cinnamoyl, COO-Pentenoyl, COO-Hexanoyl, COO-benzoyl, COO-Ethylbutyryl, COO-alkyl, COO-dibenzoyl, COO-benzoyl, COO-alkanoyl, CH2O-alkenoyl, COO-benzoyl alkyl substituted O-alkanoyl, COO-alkanoyl substituted phenyl, COO-alkenoyl substituted phenyl, COO-aryl, COO-acyl, COO-heterocylic, COO-heteroraryl, COO-alkenylcarbonyl, COO-alkane, COO-alkene, COO-sugar moiety, COO-acid moiety, COO-ethanoyl, COO-propanoyl, COO-propenoyl, COO-butanoyl, COO-butenoyl, COO-pentanoyl, COO-hexenoyl, COO-heptanoyl, COO-heptenoyl, COO-octanoyl, COO-octenoyl, COO-nonanoyl, COO-nonenoyl, COO-decanoyl, COO-decenoyl, COO-propionyl, COO-2-propenoyl, COO-2-butenoyl, COO-Isobutyryl, COO-2-methylpropanoyl, COO-2-ethylbutyryl, COO-ethylbutanoyl, COO-2-ethylbutanoyl, COO-butyryl, COO-(E)-2,3-Dimethylacryloyl, COO-(E)-2-Methylcrotonoyl, COO-3-cis-Methyl-methacryloyl, COO-3-Methyl-2-butenoyl, COO-3-Methylcrotonoyl, COO-4-Pentenoyl, COO-(2E)-2-pentenoyl, COO-Caproyl, COO-5-Hexenoyl, COO-Capryloyl, COO-Lauroyl, COO-Dodecanoyl, COO-Myristoyl, COO-Tetradecanoyl, COO-Oleoyl, COO—C(2-18) Acyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-alkane, CH2O-alkene and CH2O-sugar moiety, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl, (CnH2n)O-angeloyl, (CnH2n)O-tigloyl, (CnH2n)O-senecioyl, (CnH2n)O-acetyl, (CnH2n)O-Crotonoyl, (CnH2n)O-3,3-Dimethylartyloyl, (CnH2n)O-Cinnamoyl, (CnH2n)O-Pentenoyl, (CnH2n)O-Hexanoyl, (CnH2n)O-benzoyl, (CnH2n)O-Ethylbutyryl, (CnH2n)O-alkyl, (CnH2n)O-dibenzoyl, (CnH2n)O-benzoyl, (CnH2n)O-alkanoyl, (CnH2n)O-alkenoyl, (CnH2n)O-benzoyl alkyl substituted O-alkanoyl, (CnH2n)O-alkanoyl substituted phenyl, (CnH2n)O-alkenoyl substituted phenyl, (CnH2n)O-aryl, (CnH2n)O-acyl, (CnH2n)O-heterocylic, (CnH2n)O-heteroraryl, (CnH2n)O-alkenylcarbonyl, (CnH2n)O-alkane, (CnH2n)O-alkene and (CnH2n)O-sugar moiety and (CnH2n)O-acid moiety, wherein n is 1 or 2 or 3 or 4 or over 5 or derivatives thereof; wherein the sugar moiety(ies) is/are selected from a group of glucose, galactose, rhamnose, arabinose, xylose, fucose, allose, altrose, gulose, idose, lyxose, mannose, psicose, ribose, sorbose, tagatose, talose, fructose, alduronic acid, glucuronic acid, galacturonic acid, and derivatives or combinations thereof; or wherein any 1 or 2 or 3 or 4 of R1, R2, R3, R4, R5, R8, R16 and R10 are independently attached an O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl;

NH-tigloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl, NH-alkyl, NH-dibenzoyl, NH-benzoyl, NH-alkanoyl, NH-alkenoyl, NH-benzoyl alkyl substituted NH-alkanoyl, NH-alkanoyl substituted phenyl, NH-alkenoyl substituted phenyl, NH-aryl, NH-acyl, NH-heterocylic, NH-heteroraryl, NH-alkenylcarbonyl, NH-alkane, NH-alkene, NH-sugar moiety, NH-acid moiety, NH-ethanoyl, NH-propanoyl, NH-propenoyl, NH-butanoyl, NH-butenoyl, NH-pentanoyl, NH-hexenoyl, NH-heptanoyl, NH-heptenoyl, NH-octanoyl, H-octenoyl, NH-nonanoyl, NH-nonenoyl, NH-decanoyl, NH-decenoyl, NH-propionyl, NH-2-propenoyl, NH-2-butenoyl, NH-Isobutyryl, NH-2-methylpropanoyl, NH-2-ethylbutyryl, NH-ethylbutanoyl, NH-2-ethylbutanoyl, NH-butyryl, NH-(E)-2,3-Dimethylacryloyl, NH-(E)-2-Methylcrotonoyl, NH-3-cis-Methyl-methacryloyl, NH-3-Methyl-2-butenoyl, NH-3-Methylcrotonoyl, NH-4-Pentenoyl, NH-(2E)-2-pentenoyl, NH-Caproyl, NH-5-Hexenoyl, NH-Capryloyl, NH-Lauroyl, NH-Dodecanoyl, NH-Myristoyl, NH-Tetradecanoyl, NH-Oleoyl, NH—C(2-18) Acyl, NH-ethanyl, NH-propanyl, NH-propenyl, NH-butanyl, NH-butenyl, NH-pentanyl, NH-hexenyl, NH-heptanyl, NH-heptenyl, NH-octanyl, NH-octenyl, NH-nonanyl, NH-nonenyl, NH-decanyl, NH-decenyl, NH-alkyl, NH-haloalkyl, NH-alkenyl, NH-alkynyl, NH-hydroxyalkyl, NH-alkylene-O-alkyl, NH-aryl, NH-alkylene-aryl, NH-heteroaryl, NH-alkylene-heteroaryl, NH-cycloalkyl, NH-heterocyclyl, and NH-alkylene-heterocyclyl,

CH2NH-tigloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl, CH2NH-ethanyl, CH2NH-propanyl, CH2NH-propenyl, CH2NH-butanyl, CH2NH-butenyl, CH2NH-pentanyl, CH2NH-hexenyl, CH2NH-heptanyl, CH2NH-heptenyl, CH2NH-octanyl, CH2NH-octenyl, CH2NH-nonanyl, CH2NH-nonenyl, CH2NH-decanyl, CH2NH-decenyl, CH2NH-alkyl, CH2NH-haloalkyl, CH2NH-alkenyl, CH2NH-alkynyl, CH2NH-hydroxyalkyl, CH2NH-alkylene-O-alkyl, CH2NH-aryl, CH2NH-alkylene-aryl, CH2NH-heteroaryl, CH2NH-alkylene-heteroaryl, CH2NH-cycloalkyl, CH2NH-heterocyclyl, and CH2NH-alkylene-heterocyclyl, CH2NHCH2-tigloyl, CH2NHCH2-angeloyl, CH2NHCH2-senecioyl, CH2NHCH2-acetyl, CH2NHCH2-Crotonoyl, CH2NHCH2-3,3-Dimethylartyloyl, CH2NHCH2-Cinnamoyl, CH2NHCH2-Pentenoyl, CH2NHCH2-Hexanoyl, CH2NHCH2-benzoyl, CH2NHCH2-Ethylbutyryl, CH2NHCH2-alkyl, CH2NHCH2-dibenzoyl, CH2NHCH2-benzoyl, CH2NHCH2-alkanoyl, CH2NHCH2-alkenoyl, CH2NHCH2-benzoyl alkyl substituted CH2NHCH2-alkanoyl, CH2NHCH2-alkanoyl substituted phenyl, CH2NHCH2-alkenoyl substituted phenyl, CH2NHCH2-aryl, CH2NHCH2-acyl, CH2NHCH2-heterocylic, CH2NHCH2-heteroraryl, CH2NHCH2-alkenylcarbonyl, CH2NHCH2-alkane, CH2NHCH2-alkene, CH2NHCH2-sugar moiety, CH2NHCH2-acid moiety, CH2NHCH2-ethanoyl, CH2NHCH2-propanoyl, CH2NHCH2-propenoyl, CH2NHCH2-butanoyl, CH2NHCH2-butenoyl, CH2NHCH2-pentanoyl, CH2NHCH2-hexenoyl, CH2NHCH2-heptanoyl, CH2NHCH2-heptenoyl, CH2NHCH2-octanoyl, CH2NHCH2-octenoyl, CH2NHCH2-nonanoyl, CH2NHCH2-nonenoyl, CH2NHCH2-decanoyl, CH2NH-decenoyl, CH2NHCH2-propionyl, CH2NHCH2-2-propenoyl, CH2NHCH2-2-butenoyl, CH2NHCH2-Isobutyryl, CH2NHCH2-2-methylpropanoyl, CH2NHCH2-2-ethylbutyryl, CH2NHCH2-ethylbutanoyl, CH2NHCH2-2-ethylbutanoyl, CH2NHCH2-butyryl, CH2NHCH2-(E)-2,3-Dimethylacryloyl, CH2NHCH2-(E)-2-Methylcrotonoyl, CH2NHCH2-3-cis-Methyl-methacryloyl, CH2NHCH2-3-Methyl-2-butenoyl, CH2NHCH2-3-Methylcrotonoyl, CH2NHCH2-4-Pentenoyl, CH2NHCH2-(2E)-2-pentenoyl, CH2NHCH2-Caproyl, CH2NHCH2-5-Hexenoyl, CH2NHCH2-Capryloyl, CH2NHCH2-Lauroyl, CH2NHCH2-Dodecanoyl, CH2NHCH2-Myristoyl, CH2NHCH2-Tetradecanoyl, CH2NHCH2-Oleoyl, CH2NHCH2-C(2-18) Acyl, CH2NHCH2-ethanyl, CH2NHCH2-propanyl, CH2NHCH2-propenyl, CH2NHCH2-butanyl, CH2NHCH2-butenyl, CH2NHCH2-pentanyl, CH2NHCH2-hexenyl, CH2NHCH2-heptanyl, CH2NHCH2-heptenyl, CH2NHCH2-octanyl, CH2NHCH2-octenyl, CH2NHCH2-nonanyl, CH2NHCH2-nonenyl, CH2NHCH2-decanyl, CH2NHCH2-decenyl, CH2NHCH2-alkyl, haloalkyl, CH2NHCH2-alkenyl, CH2NHCH2-alkynyl, CH2NHCH2-hydroxyalkyl, -alkylene-O-alkyl, CH2NHCH2-aryl, CH2NHCH2-alkylene-aryl, CH2NHCH2-heteroaryl, CH2NHCH2-alkylene-heteroaryl, CH2NHCH2-cycloalkyl, CH2NHCH2-heterocyclyl, and CH2NHCH2-alkylene-heterocyclyl,

CH2NHCO-tigloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl, CH2NHCO-alkyl, CH2NHCO-dibenzoyl, CH2NHCO-benzoyl, CH2NHCO-alkanoyl, CH2NHCO-alkenoyl, CH2NHCO-benzoyl alkyl substituted CH2NHCO-alkanoyl, CH2NHCO-alkanoyl substituted phenyl, CH2NHCO-alkenoyl substituted phenyl, CH2NHCO-aryl, CH2NHCO-acyl, CH2NHCO-heterocylic, CH2NHCO-heteroraryl, CH2NHCO-alkenylcarbonyl, CH2NHCO-alkane, CH2NHCO-alkene, CH2NHCO-sugar moiety, CH2NHCO-acid moiety, CH2NHCO-ethanoyl, CH2NHCO-propanoyl, CH2NHCO-propenoyl, CH2NHCO-butanoyl, CH2NHCO-butenoyl, CH2NHCO-pentanoyl, CH2NHCO-hexenoyl, CH2NHCO-heptanoyl, CH2NHCO-heptenoyl, CH2NHCO-octanoyl, CH2NHCO-octenoyl, CH2NHCO-nonanoyl, CH2N HCO-nonenoyl, CH2NHCO-decanoyl, CH2NHCO-decenoyl, CH2NHCO-propionyl, CH2NHCO-2-propenoyl, CH2NHCO-2-butenoyl, CH2NHCO-Isobutyryl, CH2NHCO-2-methylpropanoyl, CH2NHCO-2-ethylbutyryl, CH2NHCO-ethylbutanoyl, CH2NHCO-2-ethylbutanoyl, CH2NHCO-butyryl, CH2NHCO-(E)-2,3-Dimethylacryloyl, CH2NHCO-(E)-2-Methylcrotonoyl, CH2NHCO-3-cis-Methyl-methacryloyl, CH2NHCO-3-Methyl-2-butenoyl, CH2NHCO-3-Methylcrotonoyl, CH2NHCO-4-Pentenoyl, CH2NHCO-(2E)-2-pentenoyl, CH2NHCO-Caproyl, CH2NHCO-5-Hexenoyl, CH2NHCO-Capryloyl, CH2NHCO-Lauroyl, CH2NHCO-Dodecanoyl, CH2NHCO-Myristoyl, CH2NHCO-Tetradecanoyl, CH2NHCO-Oleoyl, CH2NHCO—C(2-18) Acyl, CH2NHCO-ethanyl, CH2NHCO-propanyl, CH2NHCO-propenyl, CH2NHCO-butanyl, CH2NHCO-butenyl, CH2NHCO-pentanyl, CH2NHCO-hexenyl, CH2NHCO-heptanyl, CH2NHCO-heptenyl, CH2NHCO-octanyl, CH2NHCO-octenyl, CH2NHCO-nonanyl, CH2NHCO-nonenyl, CH2NHCO-decanyl, CH2NHCO-decenyl, CH2NHCO-alkyl, CH2NHCO-haloalkyl, alkenyl, CH2NHCO-alkynyl, CH2NHCO-hydroxyalkyl, CH2NHCO-alkylene-O-alkyl, CH2NHCO-aryl, CH2NHCO-alkylene-aryl, CH2NHCO-heteroaryl, CH2NHCO-alkylene-heteroaryl, CH2NHCO-cycloalkyl, CH2NHCO-heterocyclyl, and CH2NHCO-alkylene-heterocyclyl,

CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl, CH2NHCONH-alkyl, CH2NHCONH-dibenzoyl, CH2NHCONH-benzoyl, CH2NHCONH-alkanoyl, CH2NHCONH-alkenoyl, CH2NHCONH-benzoyl alkyl substituted CH2NHCONH-alkanoyl, CH2NHCONH-alkanoyl substituted phenyl, CH2NHCONH-alkenoyl substituted phenyl, CH2NHCONH-aryl, CH2NHCONH-acyl, CH2NHCONH-heterocylic, CH2NHCONH-heteroraryl, CH2NHCONH-alkenylcarbonyl, CH2NHCONH-alkane, CH2NHCONH-alkene, CH2NHCONH-sugar moiety, CH2NHCONH-acid moiety, CH2NHCONH-ethanoyl, CH2NHCONH-propanoyl, CH2NHCONH-propenoyl, CH2NHCONH-butanoyl, CH2NHCONH-butenoyl, CH2NHCONH-pentanoyl, CH2NHCONH-hexenoyl, CH2NHCONH-heptanoyl, CH2NHCONH-heptenoyl, CH2NHCONH-octanoyl, CH2NHCONH-octenoyl, CH2NHCONH-nonanoyl, CH2NHCONH-nonenoyl, CH2NHCONH-decanoyl, CH2NHCONH-decenoyl, CH2NHCONH-propionyl, CH2NHCONH-2-propenoyl, CH2NHCONH-2-butenoyl, CH2NHCONH-Isobutyryl, CH2NHCONH-2-methylpropanoyl, CH2NHCONH-2-ethylbutyryl, CH2NHCONH-ethylbutanoyl, CH2NHCONH-2-ethylbutanoyl, CH2NHCONH-butyryl, CH2NHCONH-(E)-2,3-Dimethylacryloyl, CH2NHCONH-(E)-2-Methylcrotonoyl, CH2NHCONH-3-cis-Methyl-methacryloyl, CH2NHCONH-3-Methyl-2-butenoyl, CH2NHCONH-3-Methylcrotonoyl, CH2NHCONH-4-Pentenoyl, CH2NHCONH-(2E)-2-pentenoyl, CH2NHCONH-Caproyl, CH2NHCONH-5-Hexenoyl, CH2NHCONH-Capryloyl, CH2NHCONH-Lauroyl, CH2NHCONH-Dodecanoyl, CH2NHCONH-Myristoyl, CH2NHCONH-Tetradecanoyl, CH2NHCONH-Oleoyl, CH2NHCONH—C(2-18) Acyl, CH2NHCONH-ethanyl, CH2NHCONH-propanyl, CH2NHCONH-propenyl, CH2NHCONH-butanyl, CH2NHCONH-butenyl, CH2NHCONH-pentanyl, CH2NHCONH-hexenyl, CH2NHCONH-heptanyl, CH2NHCONH-heptenyl, CH2NHCONH-octanyl, CH2NHCONH-octenyl, CH2NHCONH-nonanyl, CH2NHCONH-nonenyl, CH2NHCONH-decanyl, CH2NHCONH-decenyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl),

CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl, CH2NHSO2-alkyl, CH2NHSO2-dibenzoyl, CH2NHSO2-benzoyl, CH2NHSO2-alkanoyl, CH2NHSO2-alkenoyl, CH2NHSO2-benzoyl alkyl substituted CH2NHSO2-alkanoyl, CH2NHSO2-alkanoyl substituted phenyl, CH2NHSO2-alkenoyl substituted phenyl, CH2NHSO2-aryl, CH2NHSO2-acyl, CH2NHSO2-heterocylic, CH2NHSO2-heteroraryl, CH2NHSO2-alkenylcarbonyl, CH2NHSO2-alkane, CH2NHSO2-alkene, CH2NHSO2-sugar moiety, CH2NHSO2-acid moiety, CH2NHSO2-ethanoyl, CH2NHSO2-propanoyl, CH2NHSO2-propenoyl, CH2NHSO2-butanoyl, CH2NHSO2-butenoyl, CH2NHSO2-pentanoyl, CH2NHSO2-hexenoyl, CH2NHSO2-heptanoyl, CH2NHSO2-heptenoyl, CH2NHSO2-octanoyl, CH2NHSO2-octenoyl, CH2NHSO2-nonanoyl, CH2NHSO2-nonenoyl, CH2NHSO2-decanoyl, CH2NHSO2-decenoyl, CH2NHSO2-propionyl, CH2NHSO2-2-propenoyl, CH2NHSO2-2-butenoyl, CH2NHSO2-Isobutyryl, CH2NHSO2-2-methylpropanoyl, CH2NHSO2-2-ethylbutyryl, CH2NHSO2-ethylbutanoyl, CH2NHSO2-2-ethylbutanoyl, CH2NHSO2-butyryl, CH2NHSO2-(E)-2,3-Dimethylacryloyl, CH2NHSO2-(E)-2-Methylcrotonoyl, CH2NHSO2-3-cis-Methyl-methacryloyl, CH2NHSO2-3-Methyl-2-butenoyl, CH2NHSO2-3-Methylcrotonoyl, CH2NHSO2-4-Pentenoyl, CH2NHSO2-(2E)-2-pentenoyl, CH2NHSO2-Caproyl, CH2NHSO2-5-Hexenoyl, CH2NHSO2-Capryloyl, CH2NHSO2-Lauroyl, CH2NHSO2-Dodecanoyl, CH2NHSO2-Myristoyl, CH2NHSO2-Tetradecanoyl, CH2NHSO2-Oleoyl, CH2NHSO2-C(2-18) Acyl, CH2NHSO2-ethanyl, CH2NHSO2-propanyl, CH2NHSO2-propenyl, CH2NHSO2-butanyl, CH2NHSO2-butenyl, CH2NHSO2-pentanyl, CH2NHSO2-hexenyl, CH2NHSO2-heptanyl, CH2NHSO2-heptenyl, CH2NHSO2-octanyl, CH2NHSO2-octenyl, CH2NHSO2-nonanyl, CH2NHSO2-nonenyl, CH2NHSO2-decanyl, CH2NHSO2-decenoyl, CH2NHSO2-alkyl, CH2NHSO2-haloalkyl, CH2NHSO2-alkenyl, CH2NHSO2-alkynyl, CH2NHSO2-hydroxyalkyl, CH2NHSO2-alkylene-O-alkyl, CH2NHSO2-aryl, CH2NHSO2-alkylene-aryl, CH2NHSO2-heteroaryl, CH2NHSO2-alkylene-heteroaryl, CH2NHSO2-cycloalkyl, CH2NHSO2-heterocyclyl, and CH2NHSO2-alkylene-heterocyclyl, CH2NHSO2-ethyl, CH2NHSO2-(Z)-(2-methylbut-2-en-1-yl), CH2NHSO2-(E)-prop-1-enyl, CH2NHSO2-(E)-2-phenylethenyl, CH2NHSO2-but-3-enyl;

NH-ethyl, NH—(Z)-(2-methylbut-2-en-1-yl), NH-(E)-(2-methylbut-2-en-1-yl), NH-(3-methylbut-2-en-1-yl, NH-(E)-(but-2-en-1-yl, NH-cinnamyl, NH-pent-4-en-1-yl, NH-(E)-3-((4-(dimethylamino)but-2-en-1-yl, NH-acetyl, NH-angeloyl, NH-tigloyl, NH-senecioyl, NH-Crotonoyl, NH-Cinnamoyl, NH-Pentenoyl, NH-4-(dimethylamino)-2-methylbut-2-enoyl, NH-4-[(2-methoxyethyl)amino]-2-methyl-4oxobut-2-enoyl, NHSO2-ethyl, NHSO2-(Z)-(2-methylbut-2-en-1-yl), NHSO2-(E)-prop-1-enyl, NHSO2-(E)-2-phenylethenyl, NHSO2-but-3-enyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl);

or wherein R9, R11, R12, R13, R14, R15, are independently attached a CH3; or wherein R10 is attached CH2NH-tigloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl,

or wherein R4 and/or R10 are independently attached an O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methyl propanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl;

NH-tigloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl, NH-alkyl, NH-dibenzoyl, NH-benzoyl, NH-alkanoyl, NH-alkenoyl, NH-benzoyl alkyl substituted NH-alkanoyl, NH-alkanoyl substituted phenyl, NH-alkenoyl substituted phenyl, NH-aryl, NH-acyl, NH-heterocylic, NH-heteroraryl, NH-alkenylcarbonyl, NH-alkane, NH-alkene, NH-sugar moiety, NH-acid moiety, NH-ethanoyl, NH-propanoyl, NH-propenoyl, NH-butanoyl, NH-butenoyl, NH-pentanoyl, NH-hexenoyl, NH-heptanoyl, NH-heptenoyl, NH-octanoyl, NH-octenoyl, NH-nonanoyl, NH-nonenoyl, NH-decanoyl, NH-decenoyl, NH-propionyl, NH-2-propenoyl, NH-2-butenoyl, NH-Isobutyryl, NH-2-methylpropanoyl, NH-2-ethylbutyryl, NH-ethylbutanoyl, NH-2-ethylbutanoyl, NH-butyryl, NH-(E)-2,3-Dimethylacryloyl, NH-(E)-2-Methylcrotonoyl, NH-3-cis-Methyl-methacryloyl, NH-3-Methyl-2-butenoyl, NH-3-Methylcrotonoyl, NH-4-Pentenoyl, NH-(2E)-2-pentenoyl, NH-Caproyl, NH-5-Hexenoyl, NH-Capryloyl, NH-Lauroyl, NH-Dodecanoyl, NH-Myristoyl, NH-Tetradecanoyl, NH-Oleoyl, NH—C(2-18) Acyl, NH-ethanyl, NH-propanyl, NH-propenyl, NH-butanyl, NH-butenyl, NH-pentanyl, NH-hexenyl, NH-heptanyl, NH-heptenyl, NH-octanyl, NH-octenyl, NH-nonanyl, NH-nonenyl, NH-decanyl, NH-decenyl, NH-alkyl, NH-haloalkyl, NH-alkenyl, NH-alkynyl, NH-hydroxyalkyl, NH-alkylene-O-alkyl, NH-aryl, NH-alkylene-aryl, NH-heteroaryl, NH-alkylene-heteroaryl, NH-cycloalkyl, NH-heterocyclyl, and NH-alkylene-heterocyclyl,

CH2NH-tigloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl, CH2NH-ethanyl, CH2NH-propanyl, CH2NH-propenyl, CH2NH-butanyl, CH2NH-butenyl, CH2NH-pentanyl, CH2NH-hexenyl, CH2NH-heptanyl, CH2NH-heptenyl, CH2NH-octanyl, CH2NH-octenyl, CH2NH-nonanyl, CH2NH-nonenyl, CH2NH-decanyl, CH2NH-decenyl, CH2NH-alkyl, CH2NH-haloalkyl, CH2NH-alkenyl, CH2NH-alkynyl, CH2NH-hydroxyalkyl, CH2NH-alkylene-O-alkyl, CH2NH-aryl, CH2NH-alkylene-aryl, CH2NH-heteroaryl, CH2NH-alkylene-heteroaryl, CH2NH-cycloalkyl, CH2NH-heterocyclyl, and CH2NH-alkylene-heterocyclyl,

CH2NHCH2-tigloyl, CH2NHCH2-angeloyl, CH2NHCH2-senecioyl, CH2NHCH2-acetyl, CH2NHCH2-Crotonoyl, CH2NHCH2-3,3-Dimethylartyloyl, CH2NHCH2-Cinnamoyl, CH2NHCH2-Pentenoyl, CH2NHCH2-Hexanoyl, CH2NHCH2-benzoyl, CH2NHCH2-Ethylbutyryl, CH2NHCH2-alkyl, CH2NHCH2-dibenzoyl, CH2NHCH2-benzoyl, CH2NHCH2-alkanoyl, CH2NHCH2-alkenoyl, CH2NHCH2-benzoyl alkyl substituted CH2NHCH2-alkanoyl, CH2NHCH2-alkanoyl substituted phenyl, CH2NHCH2-alkenoyl substituted phenyl, CH2NHCH2-aryl, CH2NHCH2-acyl, CH2NHCH2-heterocylic, CH2NHCH2-heteroraryl, CH2NHCH2-alkenylcarbonyl, CH2NHCH2-alkane, CH2NHCH2-alkene, CH2NHCH2-sugar moiety, CH2NHCH2-acid moiety, CH2NHCH2-ethanoyl, CH2NHCH2-propanoyl, CH2NHCH2-propenoyl, CH2NHCH2-butanoyl, CH2NHCH2-butenoyl, CH2NHCH2-pentanoyl, CH2NHCH2-hexenoyl, CH2NHCH2-heptanoyl, CH2NHCH2-heptenoyl, CH2NHCH2-octanoyl, CH2NHCH2-octenoyl, CH2NHCH2-nonanoyl, CH2NHCH2-nonenoyl, CH2NHCH2-decanoyl, CH2NH-decenoyl, CH2NHCH2-propionyl, CH2NHCH2-2-propenoyl, CH2NHCH2-2-butenoyl, CH2NHCH2-Isobutyryl, CH2NHCH2-2-methylpropanoyl, CH2NHCH2-2-ethylbutyryl, CH2NHCH2-ethylbutanoyl, CH2NHCH2-2-ethylbutanoyl, CH2NHCH2-butyryl, CH2NHCH2-(E)-2,3-Dimethylacryloyl, CH2NHCH2-(E)-2-Methylcrotonoyl, CH2NHCH2-3-cis-Methyl-methacryloyl, CH2NHCH2-3-Methyl-2-butenoyl, CH2NHCH2-3-Methylcrotonoyl, CH2NHCH2-4-Pentenoyl, CH2NHCH2-(2E)-2-pentenoyl, CH2NHCH2-Caproyl, CH2NHCH2-5-Hexenoyl, CH2NHCH2-Capryloyl, CH2NHCH2-Lauroyl, CH2NHCH2-Dodecanoyl, CH2NHCH2-Myristoyl, CH2NHCH2-Tetradecanoyl, CH2NHCH2-Oleoyl, CH2NHCH2-C(2-18) Acyl, CH2NHCH2-ethanyl, CH2NHCH2-propanyl, CH2NHCH2-propenyl, CH2NHCH2-butanyl, CH2NHCH2-butenyl, CH2NHCH2-pentanyl, CH2NHCH2-hexenyl, CH2NHCH2-heptanyl, CH2NHCH2-heptenyl, CH2NHCH2-octanyl, CH2NHCH2-octenyl, CH2NHCH2-nonanyl, CH2NHCH2-nonenyl, CH2NHCH2-decanyl, CH2NHCH2-decenyl, CH2NHCH2-alkyl, haloalkyl, CH2NHCH2-alkenyl, CH2NHCH2-alkynyl, CH2NHCH2-hydroxyalkyl, -alkylene-O-alkyl, CH2NHCH2-aryl, CH2NHCH2-alkylene-aryl, CH2NHCH2-heteroaryl, CH2NHCH2-alkylene-heteroaryl, CH2NHCH2-cycloalkyl, CH2NHCH2-heterocyclyl, and CH2NHCH2-alkylene-heterocyclyl,

CH2NHCO-tigloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl, CH2NHCO-alkyl, CH2NHCO-dibenzoyl, CH2NHCO-benzoyl, CH2NHCO-alkanoyl, CH2NHCO-alkenoyl, CH2NHCO-benzoyl alkyl substituted CH2NHCO-alkanoyl, CH2NHCO-alkanoyl substituted phenyl, CH2NHCO-alkenoyl substituted phenyl, CH2NHCO-aryl, CH2NHCO-acyl, CH2NHCO-heterocylic, CH2NHCO-heteroraryl, CH2NHCO-alkenylcarbonyl, CH2NHCO-alkane, CH2NHCO-alkene, CH2NHCO-sugar moiety, CH2NHCO-acid moiety, CH2NHCO-ethanoyl, CH2NHCO-propanoyl, CH2NHCO-propenoyl, CH2NHCO-butanoyl, CH2NHCO-butenoyl, CH2NHCO-pentanoyl, CH2NHCO-hexenoyl, CH2NHCO-heptanoyl, CH2NHCO-heptenoyl, CH2NHCO-octanoyl, CH2NHCO-octenoyl, CH2NHCO-nonanoyl, CH2NHCO-nonenoyl, CH2NHCO-decanoyl, CH2NHCO-decenoyl, CH2NHCO-propionyl, CH2NHCO-2-propenoyl, CH2NHCO-2-butenoyl, CH2NHCO-Isobutyryl, CH2NHCO-2-methylpropanoyl, CH2NHCO-2-ethylbutyryl, CH2NHCO-ethylbutanoyl, CH2NHCO-2-ethylbutanoyl, CH2NHCO-butyryl, CH2NHCO-(E)-2,3-Dimethylacryloyl, CH2NHCO-(E)-2-Methylcrotonoyl, CH2NHCO-3-cis-Methyl-methacryloyl, CH2NHCO-3-Methyl-2-butenoyl, CH2NHCO-3-Methylcrotonoyl, CH2NHCO-4-Pentenoyl, CH2NHCO-(2E)-2-pentenoyl, CH2NHCO-Caproyl, CH2NHCO-5-Hexenoyl, CH2NHCO-Capryloyl, CH2NHCO-Lauroyl, CH2NHCO-Dodecanoyl, CH2NHCO-Myristoyl, CH2NHCO-Tetradecanoyl, CH2NHCO-Oleoyl, CH2NHCO—C(2-18) Acyl, CH2NHCO-ethanyl, CH2NHCO-propanyl, CH2NHCO-propenyl, CH2NHCO-butanyl, CH2NHCO-butenyl, CH2NHCO-pentanyl, CH2NHCO-hexenyl, CH2NHCO-heptanyl, CH2NHCO-heptenyl, CH2NHCO-octanyl, CH2NHCO-octenyl, CH2NHCO-nonanyl, CH2NHCO-nonenyl, CH2NHCO-decanyl, CH2NHCO-decenyl, CH2NHCO-alkyl, CH2NHCO-haloalkyl, alkenyl, CH2NHCO-alkynyl, CH2NHCO-hydroxyalkyl, CH2NHCO-alkylene-O-alkyl, CH2NHCO-aryl, CH2NHCO-alkylene-aryl, CH2NHCO-heteroaryl, CH2NHCO-alkylene-heteroaryl, CH2NHCO-cycloalkyl, CH2NHCO-heterocyclyl, and CH2NHCO-alkylene-heterocyclyl,

CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl, CH2NHCONH-alkyl, CH2NHCONH-dibenzoyl, CH2NHCONH-benzoyl, CH2NHCONH-alkanoyl, CH2NHCONH-alkenoyl, CH2NHCONH-benzoyl alkyl substituted CH2NHCONH-alkanoyl, CH2NHCONH-alkanoyl substituted phenyl, CH2NHCONH-alkenoyl substituted phenyl, CH2NHCONH-aryl, CH2NHCONH-acyl, CH2NHCONH-heterocylic, CH2NHCONH-heteroraryl, CH2NHCONH-alkenylcarbonyl, CH2NHCONH-alkane, CH2NHCONH-alkene, CH2NHCONH-sugar moiety, CH2NHCONH-acid moiety, CH2NHCONH-ethanoyl, CH2NHCONH-propanoyl, CH2NHCONH-propenoyl, CH2NHCONH-butanoyl, CH2NHCONH-butenoyl, CH2NHCONH-pentanoyl, CH2NHCONH-hexenoyl, CH2NHCONH-heptanoyl, CH2NHCONH-heptenoyl, CH2NHCONH-octanoyl, CH2NHCONH-octenoyl, CH2NHCONH-nonanoyl, CH2NHCONH-nonenoyl, CH2NHCONH-decanoyl, CH2NHCONH-decenoyl, CH2NHCONH-propionyl, CH2NHCONH-2-propenoyl, CH2NHCONH-2-butenoyl, CH2NHCONH-Isobutyryl, CH2NHCONH-2-methylpropanoyl, CH2NHCONH-2-ethylbutyryl, CH2NHCONH-ethylbutanoyl, CH2NHCONH-2-ethylbutanoyl, CH2NHCONH-butyryl, CH2NHCONH-(E)-2,3-Dimethylacryloyl, CH2NHCONH-(E)-2-Methylcrotonoyl, CH2NHCONH-3-cis-Methyl-methacryloyl, CH2NHCONH-3-Methyl-2-butenoyl, CH2NHCONH-3-Methylcrotonoyl, CH2NHCONH-4-Pentenoyl, CH2NHCONH-(2E)-2-pentenoyl, CH2NHCONH-Caproyl, CH2NHCONH-5-Hexenoyl, CH2NHCONH-Capryloyl, CH2NHCONH-Lauroyl, CH2NHCONH-Dodecanoyl, CH2NHCONH-Myristoyl, CH2NHCONH-Tetradecanoyl, CH2NHCONH-Oleoyl, CH2NHCONH—C(2-18) Acyl, CH2NHCONH-ethanyl, CH2NHCONH-propanyl, CH2NHCONH-propenyl, CH2NHCONH-butanyl, CH2NHCONH-butenyl, CH2NHCONH-pentanyl, CH2NHCONH-hexenyl, CH2NHCONH-heptanyl, CH2NHCONH-heptenyl, CH2NHCONH-octanyl, CH2NHCONH-octenyl, CH2NHCONH-nonanyl, CH2NHCONH-nonenyl, CH2NHCONH-decanyl, CH2NHCONH-decenyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl);

CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl, CH2NHSO2-alkyl, CH2NHSO2-dibenzoyl, CH2NHSO2-benzoyl, CH2NHSO2-alkanoyl, CH2NHSO2-alkenoyl, CH2NHSO2-benzoyl alkyl substituted CH2NHSO2-alkanoyl, CH2NHSO2-alkanoyl substituted phenyl, CH2NHSO2-alkenoyl substituted phenyl, CH2NHSO2-aryl, CH2NHSO2-acyl, CH2NHSO2-heterocylic, CH2NHSO2-heteroraryl, CH2NHSO2-alkenylcarbonyl, CH2NHSO2-alkane, CH2NHSO2-alkene, CH2NHSO2-sugar moiety, CH2NHSO2-acid moiety, CH2NHSO2-ethanoyl, CH2NHSO2-propanoyl, CH2NHSO2-propenoyl, CH2NHSO2-butanoyl, CH2NHSO2-butenoyl, CH2NHSO2-pentanoyl, CH2NHSO2-hexenoyl, CH2NHSO2-heptanoyl, CH2NHSO2-heptenoyl, CH2NHSO2-octanoyl, CH2NHSO2-octenoyl, CH2NHSO2-nonanoyl, CH2NHSO2-nonenoyl, CH2NHSO2-decanoyl, CH2NHSO2-decenoyl, CH2NHSO2-propionyl, CH2NHSO2-2-propenoyl, CH2NHSO2-2-butenoyl, CH2NHSO2-Isobutyryl, CH2NHSO2-2-methylpropanoyl, CH2NHSO2-2-ethylbutyryl, CH2NHSO2-ethylbutanoyl, CH2NHSO2-2-ethylbutanoyl, CH2NHSO2-butyryl, CH2NHSO2-(E)-2,3-Dimethylacryloyl, CH2NHSO2-(E)-2-Methylcrotonoyl, CH2NHSO2-3-cis-Methyl-methacryloyl, CH2NHSO2-3-Methyl-2-butenoyl, CH2NHSO2-3-Methylcrotonoyl, CH2NHSO2-4-Pentenoyl, CH2NHSO2-(2E)-2-pentenoyl, CH2NHSO2-Caproyl, CH2NHSO2-5-Hexenoyl, CH2NHSO2-Capryloyl, CH2NHSO2-Lauroyl, CH2NHSO2-Dodecanoyl, CH2NHSO2-Myristoyl, CH2NHSO2-Tetradecanoyl, CH2NHSO2-Oleoyl, CH2NHSO2-C(2-18) Acyl, CH2NHSO2-ethanyl, CH2NHSO2-propanyl, CH2NHSO2-propenyl, CH2NHSO2-butanyl, CH2NHSO2-butenyl, CH2NHSO2-pentanyl, CH2NHSO2-hexenyl, CH2NHSO2-heptanyl, CH2NHSO2-heptenyl, CH2NHSO2-octanyl, CH2NHSO2-octenyl, CH2NHSO2-nonanyl, CH2NHSO2-nonenyl, CH2NHSO2-decanyl, CH2NHSO2-decenoyl, CH2NHSO2-alkyl, CH2NHSO2-haloalkyl, CH2NHSO2-alkenyl, CH2NHSO2-alkynyl, CH2NHSO2-hydroxyalkyl, CH2NHSO2-alkylene-O-alkyl, CH2NHSO2-aryl, CH2NHSO2-alkylene-aryl, CH2NHSO2-heteroaryl, CH2NHSO2-alkylene-heteroaryl, CH2NHSO2-cycloalkyl, CH2NHSO2-heterocyclyl, and CH2NHSO2-alkylene-heterocyclyl, CH2NHSO2-ethyl, CH2NHSO2-(Z)-(2-methylbut-2-en-1-yl), CH2NHSO2-(E)-prop-1-enyl, CH2NHSO2-(E)-2-phenylethenyl, CH2NHSO2-but-3-enyl,

wherein R3 is OH or H or NH2 or others; wherein R1, R2, R3, R5, R8, R16 are OH or H or NH2 or others; or wherein an O were attached to the above structures with double bond; wherein R9, R11, R12, R13, R14, and R15 are CH3; or wherein R1, R2, R5, R8 represent NH2 or OH; R3 represents NH2 or OH, H or absent; or wherein R4, R10 represent CH2Oangeloyl or CH2NH-angeloyl or CH2NH-tigloyl; R9, R11, R12, R13, R14, R15 represent CH3; or wherein R1, R2, R5, R8 represent OH, NH2, NH-tigloyl, or O-tigloyl; R3 represents OH, H, or absent; or wherein R4, R10 represent NH-tigloyl or CH2O tigloyl; R9, R11, R12, R13, R14, R15 represent CH3; wherein the group attaching to the core compound selected from acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl, C(2-18) Acyl are interchangeable; wherein the attached group can be the same group or in combination thereof; wherein the connecting group between the core compound and attached group may be O, S, S(O), S(O)2, C(O), C(O)O, NH, N-alkyl, CH2 or CH2O. In an embodiment, R4 is attached an O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl, In an embodiment, the connecting group between the functional group of angeloyl, tigloyl, senecioyl, acetyl, Crotonoyl, 3,3-Dimethylartyloyl, Cinnamoyl, Pentenoyl, Hexanoyl, benzoyl, Ethylbutyryl, alkyl, dibenzoyl, benzoyl, alkanoyl, alkenoyl, benzoyl alkyl substituted alkanoyl, alkanoyl substituted phenyl, alkenoyl substituted phenyl, aryl, acyl, heterocylic, heteroraryl, and alkenylcarbonyl ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl, or C(2-18) Acyl, can be O, S, S(O), S(O)2, C(O), C(O)O, NH, N-alkyl, CH2 or CH2O. In an embodiment, the compound(s) is(are) in form in form of powder, liquid or crystal.

In an embodiment, any 1 or 2 or 3 or 4 or 5 or 6 of R1, R2, R3, R4, R5, R8, R9, R10, R11, R12, R13, R14, R15, R16 are independently selected from the group of A-B, wherein A can be O, S, S(O), S(O)2, C(O), C(O)O, NH, N-alkyl, CH2 or CH2O, CH2NH, CH2NHCH2, CH2NHCO, CH2NHCONH, CH2NHSO2;

wherein B is selected from the group of acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl and C(2-18) Acyl. In an embodiment, R1 is A-B. In an embodiment, R2 is A-B. In an embodiment, R3 is A-B. In an embodiment, R4 is A-B. In an embodiment, R5 is A-B. In an embodiment, R6 is A-B. In an embodiment, R7 is A-B. In an embodiment, R8 is A-B. In an embodiment, R9 is A-B. In an embodiment, R10 is A-B. In an embodiment, R11 is A-B. In an embodiment, R12 is A-B. In an embodiment, R13 is A-B. In an embodiment, R14 is A-B. In an embodiment, R15 is A-B. In an embodiment, the compound(s) is(are) in form in form of powder, liquid or crystal. In an embodiment, this invention provides compounds of the above to improve blood circulation; soothing stroke; Prevent plaque formation and promote their dissipated; improve blood viscosity; reduce cardiovascular; reduce cerebrovascular; reduce thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, flutter, Blood circulation, swelling, pain; Treating bronchiectasis, tuberculosis and lung abscess caused by too hemoptysis; reducing bleeding, antitussive, expectorant and analgesic effect, dilate blood vessels; reducing blood pressure and the treatment of cerebral arteriosclerosis; elevated blood lipids and reduced cholesterol. The R1, R2, R3, R4, R5, R8, R9, R10, R11, R12, R13, R14, R15, R16 bonds of (3K2) can be alpha or beta.

R1, R2, R3, R4, R5, R8, R9, R10, R11, R12, R13, R14, R15, R16 are independently selected from the group of O, hydrogen, hydroxyl, methyl, O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl In an embodiment, the compound(s) is(are) in form in form of powder, liquid or crystal.

In embodiment the core having structures:

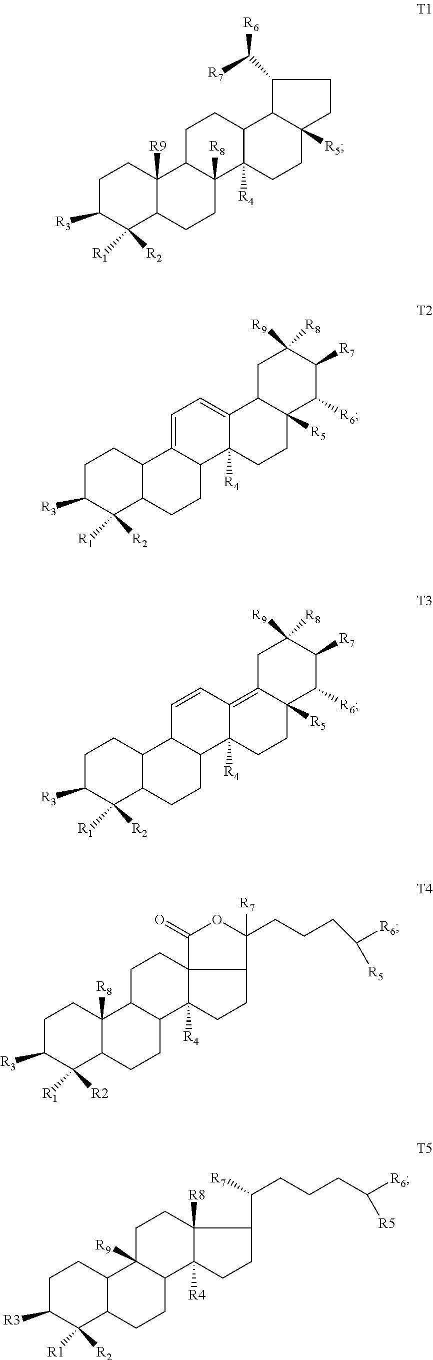

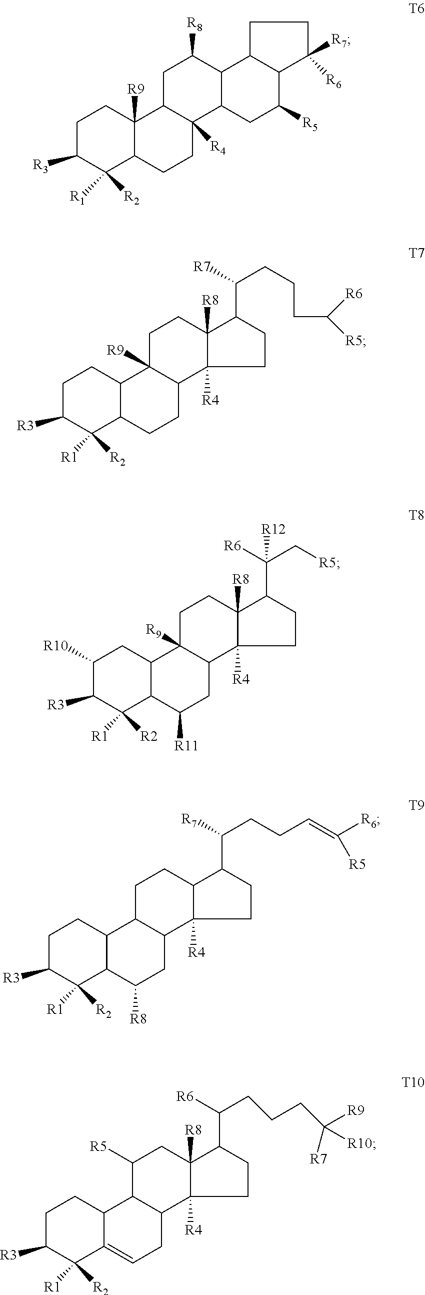

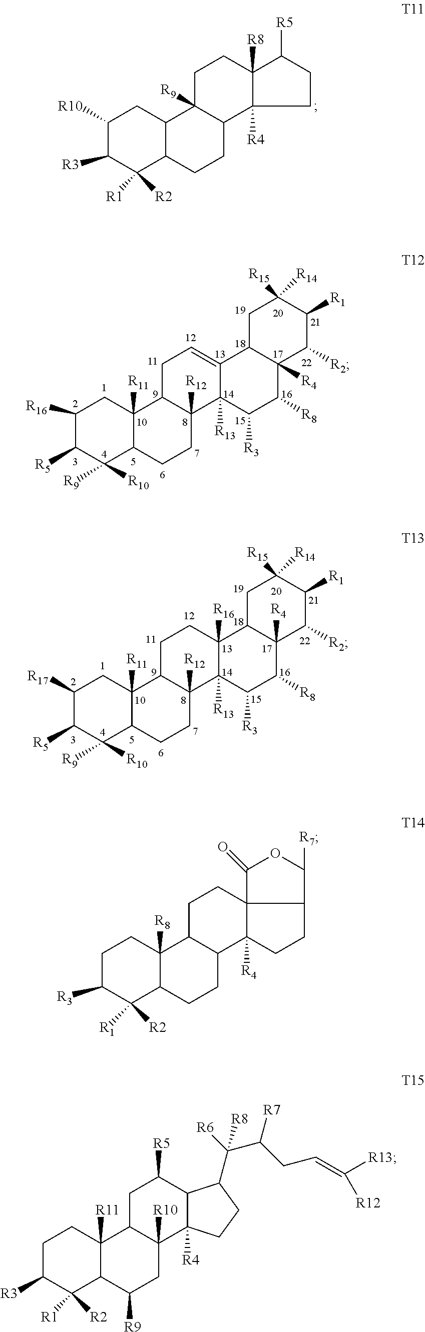

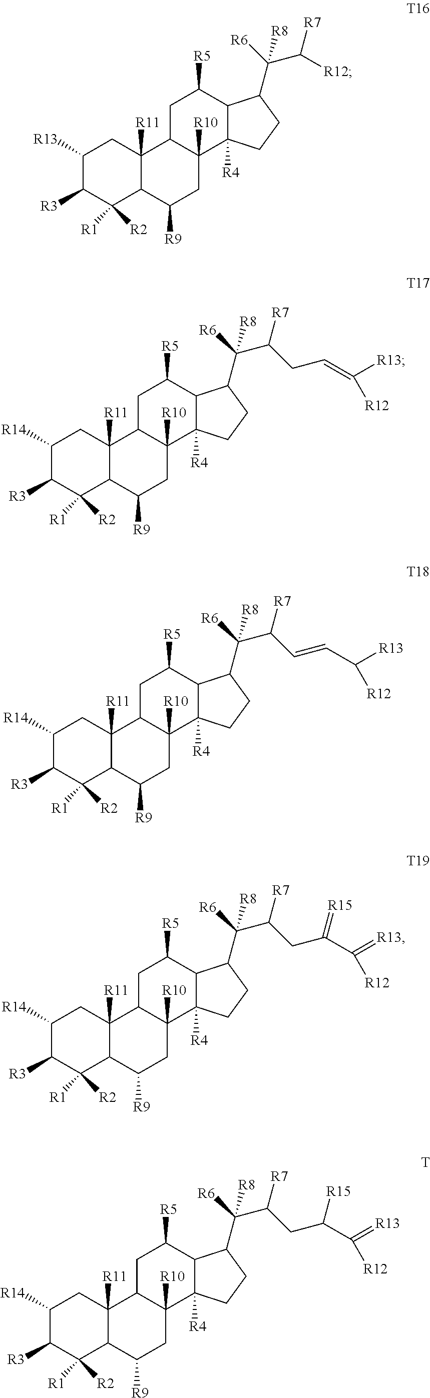

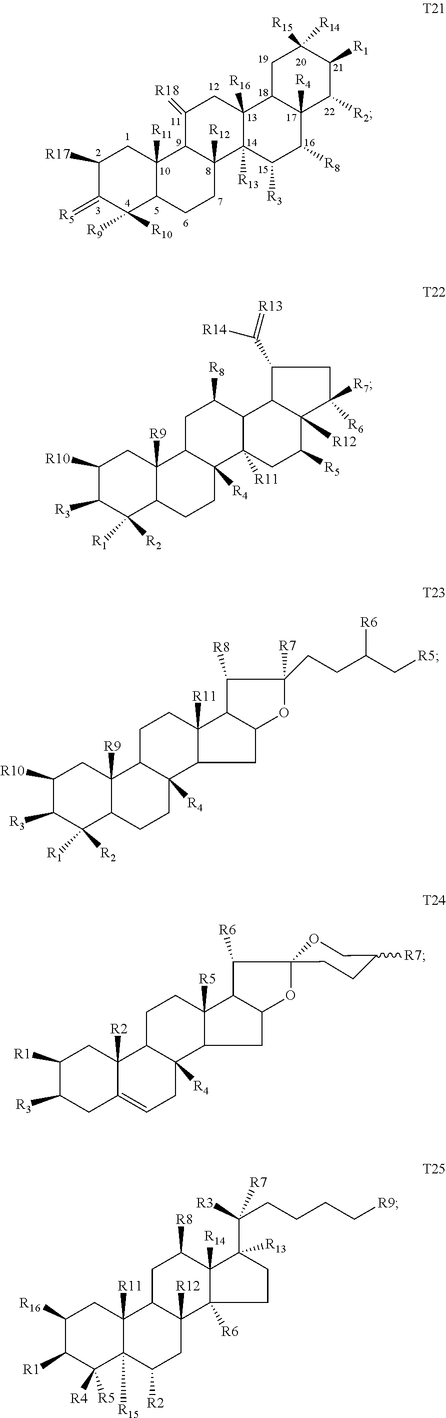

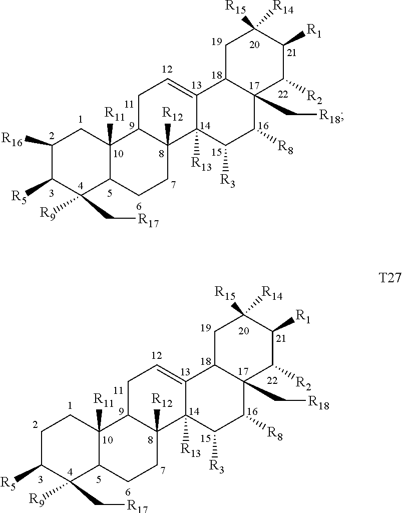

wherein R1, R2, R3, R4, R5, R8, R9, R10, R11, R12, R13, R14, R15, R16, R17, R18, of T1, T2, T3, T4, T5, T6, T7, T8, T9,T10, T11, T12, T13, T14, T15, T16, T17, T18, T19, T20, T21, T22, T23, T24, T25, T26, T27 represent H, OH, O, CH2OH, COOH or CH3. The bonds can be in forms of alpha or beta or in combinations. In an embodiment, the compound(s) is in form in form of powder, liquid or crystal

With experiments and methods in present application or esterification the above core compound with acyl halide, wherein the halide comprise chloride, bromide, fluoride and iodide, wherein the acyl halide comprise acyl chloride, wherein acyl chloride comprise tigloyl chloride, angeloyl chloride, acetyl chloride, crotonoyl chloride, 3,3-dimethylartyloyl chloride, senecioyl chloride, cinnamoyl chloride, pentenoyl chloride, hexanoyl chloride, benzoyl chloride, ethylbutyryl chloride, propionyl chloride, 2-propenoyl chloride, isobutyryl chloride, butyryl chloride, (2E)-2-pentenoyl chloride, 4-Pentenoyl chloride, 5-hexenoyl chloride, heptanoyl chloride, octanoyl chloride, nonanoyl chloride, decanoyl chloride, Lauroyl chloride, myristoyl chloride, oleoyl chloride. The compounds vary in composition when the time or temperature of the reaction is changed. The peaks, fractions and compounds are selected according to the activities of times studies and the changes of peaks. The compounds having strong to weak activities are selected and isolated. The anti cancer activities (Cytotoxic Assay) are the MTT studies of bone (U2OS), lung (H460), bladder (HTB-9), ovary (ES2), colon (HCT116), pancreas (Capan), ovary (OVCAR3), prostate (DU145), skin (SK-Mel-5), mouth (KB), kidney (A498), breast (MCF-7), liver (HepG2), brain (T98G), luekemia (K562), cervix (HeLa). The active esterification products are purified with HPLC. The reaction product of mixtures and individual compounds are tested with MTT Cytotoxic Assay. Details of method are in Experiment 3 of the present application. A second esterification of compound can be selected from the above experiment results to produce new active compounds. A partial esterification compound is selected from the above experiments to perform a second or repeated with a third esterification with different acyl chloride in order to produce new active compounds with the experiments in the present application.

A method is 1) Dissolving core compound or triterpenes core, hydroxylated triterpenes core, in pyridine; 2) Adding acyl halide or acyl chloride; 3, The mixture is stirred for length of time including 5 sec, 10 sec, 20 sec, 30 sec, 40 sec, 1 min, 2 min, 5 min, 10 min, 30 min, 1 hr, 2 hr, 18 hr, 2 days or 3 days at different temperature; 4) At the end of reaction, aqueous solution of acid or weak base, or water is added to the reaction mixture; 5) The solution is then extracted of ethyl acetate and ethyl acetate is removed by evaporation and lyophilization; 6) Dissolving the reaction product in acetonitrile with Trifluoroacetic acid or DMSO; 7) Testing the reaction product of mixtures and individual fractions with MTT cytotoxic assay; 8) Selecting the HPLC fractions for isolation is according to the cytotoxic activity of the reaction product obtained at a specific reaction time; 10) Purifying the active esterification products with HPLC; 11) Collecting the products; 12) Testing the products; wherein the core compound is terpene, isoprene, or triterpene core or hydroxylated triterpenes core; wherein the core compound was dissolved in pyridine; wherein the acyl chloride including Tigloyl chloride, angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, benzoyl chloride, Ethylbutyryl chloride, Propionyl chloride, 2-Propenoyl chloride, Isobutyryl chloride, Butyryl chloride, (2E)-2-pentenoyl chloride, 4-Pentenoyl chloride, 5-Hexenoyl chloride, Heptanoyl chloride, Octanoyl chloride, Nonanoyl chloride, Decanoyl chloride, Lauroyl chloride, Myristoyl chloride, and Oleoyl chloride; wherein the reaction time for the mixture is stirred for 5 sec, 10 sec, 20 sec, 30 sec, 40 sec, 1 min, 2 min, 5 min, 10 min, 30 min, 1 hr, 2 hr, 18 hr, 2 days or 3 days; wherein the temperature is 0 C, 25 C, 50 C or 75 C temperature; wherein the acid including HCl or the base including NaHCO3 is added to the reaction mixture; wherein the solution is then extracted 3 times with ethyl acetate and lyophilization; wherein the reaction product is dissolved in 80% acetonitrile-0.005% Trifluoroacetic acid or DMSO; wherein selecting the HPLC fractions for isolation is according to the cytotoxic activity of the reaction product obtained at a reaction time of 5 sec, 10 sec, 20 sec, 30 sec, 40 sec, 1 min, 2 min, 5 min, 10 min, 30 min, 1 hr, 2 hrs, 18 hrs, 2 days or 3 days. In an embodiment, the reaction time may be over 3 days. In an embodiment, the experiment may be performed under 0 C. In an embodiment, the experiment may be performed over 75° C.

In embodiment, the attachment of sugar moiety(ies) can be biosynthesized.

A method is to a solution of core (2 mmol) in THF (10 mL) were added Ms-Cl (2.2 mmol) and triethylamine (3 mmol) at 0° C. and the resulting mixture was stirred for 2 hours. The reaction mixture was concentrated under reduced pressure, and the residue was dissolved in ethyl acetate and washed with water. The organic layer was dried over anhydrous magnesium sulfate, filtered and concentrated under reduced pressure. The residue was dissolved in 10 mL of DMF, and then NaN3 (6 mmol) was added. After overnight stirring at 60° C., the reaction mixture was diluted with ethyl acetate and washed with water. The organic layer was dried over anhydrous magnesium sulfate, filtered and concentrated under reduced pressure. Then, the obtained compound was dissolved in methanol, and 10% Pd—C (0.2 mmol) was added. After overnight stirring under hydrogen atmosphere, the reaction mixture was filtered, washed with methanol and concentrated under reduced pressure to provide products.

This invention provide the compound having structures of T1, T2, T3, T4, T5, T6, T7, T8, T9, T10, T11, T12, T13, T14, T15, T16, T17; T18, T19, T20, T21, T22, T23, T24, T25, T26, T27 wherein the R1, R2, R3, R4, R5, R8, R9, R10, R11, R12, R13, R14, R15, R16, R17, R18 are independently selected from the group of hydrogen, hydroxyl, methyl, O,O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methyl propanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, O-4-(dimethylamino)-2-methylbut-2-enoyl, O-4-[(2-methoxyethyl)amino]-2-methyl-4oxobut-2-enoyl, O-sugar moiety(ies), O-acid moiety(ies), CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl, CH2O-4-(dimethylamino)-2-methylbut-2-enoyl, CH2O-4-[(2-methoxyethyl)amino]-2-methyl-4oxobut-2-enoyl, alkane, alkene and derivatives thereof. In an embodiment, the compound(s) is(are) in form in form of powder, liquid or crystal.

In an embodiment, the compound is attached a sugar moiety(ies), acid moiety(ies) or alduronic acid, wherein the sugar moiety(ies) is/are selected from a group of glucose, galactose, rhamnose, arabinose, xylose, fucose, allose, altrose, gulose, idose, lyxose, mannose, psicose, ribose, sorbose, tagatose, talose, fructose, alduronic acid, glucuronic acid, galacturonic acid, and derivatives or combinations thereof. In an embodiment, the compound is attached a group selected from (CnH2n)O-angeloyl, (CnH2n)O-tigloyl, (CnH2n)O-senecioyl, (CnH2n)O-acetyl, (CnH2n)O-Crotonoyl, (CnH2n)O-3,3-Dimethylartyloyl, (CnH2n)O-Cinnamoyl, (CnH2n)O-Pentenoyl, (CnH2n)O-Hexanoyl, (CnH2n)O-benzoyl, (CnH2n)O-Ethylbutyryl, (CnH2n)O-alkyl, (CnH2n)O-dibenzoyl, (CnH2n)O-benzoyl, (CnH2n)O-alkanoyl, (CnH2n)O-alkenoyl, (CnH2n)O-benzoyl alkyl substituted O-alkanoyl, (CnH2n)O-alkanoyl substituted phenyl, (CnH2n)O-alkenoyl substituted phenyl, (CnH2n)O-aryl, (CnH2n)O-acyl, (CnH2n)O-heterocylic, (CnH2n)O-heteroraryl, (CnH2n)O-alkenylcarbonyl, (CnH2n)O-alkane, (CnH2n)O-alkene and (CnH2n)O-sugar moiety and (CnH2n)O-acid moiety; wherein the sugar moiety(ies) is/are included but not limited to a group of glucose, galactose, rhamnose, arabinose, xylose, fucose, allose, altrose, gulose, idose, lyxose, mannose, psicose, ribose, sorbose, tagatose, talose, fructose, alduronic acid, glucuronic acid, galacturonic acid, and derivatives or combinations thereof; wherein n is 1 or 2 or 3 or 4 or over 5.

In an embodiment with experiments in the invention, any 1 or 2 or 3 or 4 or 5 or 6 of R1, R2, R3, R4, R5, R8, R9, R10, R11, R12, R13, R14, R15, R16, R17, R18 are independently selected from the group of A-B, wherein A can be O, S, S(O), S(O)2, C(O), C(O)O, NH, N-alkyl, CH2, CH2O, CH2NH, CH2NHCH2, CH2NHCO, CH2NHCONH, or CH2NHSO2;

wherein B is selected from the group of H, acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl and C(2-18) Acyl.

NH-tigloyl, NH-angeloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl, NH-alkyl, NH-dibenzoyl, NH-benzoyl, NH-alkanoyl, NH-alkenoyl, NH-benzoyl alkyl substituted NH-alkanoyl, NH-alkanoyl substituted phenyl, NH-alkenoyl substituted phenyl, NH-aryl, NH-acyl, NH-heterocylic, NH-heteroraryl, NH-alkenylcarbonyl, NH-alkane, NH-alkene, NH-sugar moiety, NH-acid moiety, NH-ethanoyl, NH-propanoyl, NH-propenoyl, NH-butanoyl, NH-butenoyl, NH-pentanoyl, NH-hexenoyl, NH-heptanoyl, NH-heptenoyl, NH-octanoyl, NH-octenoyl, NH-nonanoyl, NH-nonenoyl, NH-decanoyl, NH-decenoyl, NH-propionyl, NH-2-propenoyl, NH-2-butenoyl, NH-Isobutyryl, NH-2-methylpropanoyl, NH-2-ethylbutyryl, NH-ethylbutanoyl, NH-2-ethylbutanoyl, NH-butyryl, NH-(E)-2,3-Dimethylacryloyl, NH-(E)-2-Methylcrotonoyl, NH-3-cis-Methyl-methacryloyl, NH-3-Methyl-2-butenoyl, NH-3-Methylcrotonoyl, NH-4-Pentenoyl, NH-(2E)-2-pentenoyl, NH-Caproyl, NH-5-Hexenoyl, NH-Capryloyl, NH-Lauroyl, NH-Dodecanoyl, NH-Myristoyl, NH-Tetradecanoyl, NH-Oleoyl, NH—C(2-18) Acyl,

CH2NH-tigloyl, CH2NH-angeloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl,

CH2NHCH2-tigloyl, CH2NHCH2-angeloyl, CH2NHCH2-senecioyl, CH2NHCH2-acetyl, CH2NHCH2-Crotonoyl, CH2NHCH2-3,3-Dimethylartyloyl, CH2NHCH2-Cinnamoyl, CH2NHCH2-Pentenoyl, CH2NHCH2-Hexanoyl, CH2NHCH2-benzoyl, CH2NHCH2-Ethylbutyryl, CH2NHCH2-alkyl, CH2NHCH2-dibenzoyl, CH2NHCH2-benzoyl, CH2NHCH2-alkanoyl, CH2NHCH2-alkenoyl, CH2NHCH2-benzoyl alkyl substituted CH2NHCH2-alkanoyl, CH2NHCH2-alkanoyl substituted phenyl, CH2NHCH2-alkenoyl substituted phenyl, CH2NHCH2-aryl, CH2NHCH2-acyl, CH2NHCH2-heterocylic, CH2NHCH2-heteroraryl, CH2NHCH2-alkenylcarbonyl, CH2NHCH2-alkane, CH2NHCH2-alkene, CH2NHCH2-sugar moiety, CH2NHCH2-acid moiety, CH2NHCH2-ethanoyl, CH2NHCH2-propanoyl, CH2NHCH2-propenoyl, CH2NHCH2-butanoyl, CH2NHCH2-butenoyl, CH2NHCH2-pentanoyl, CH2NHCH2-hexenoyl, CH2NHCH2-heptanoyl, CH2NHCH2-heptenoyl, CH2NHCH2-octanoyl, CH2NHCH2-octenoyl, CH2NHCH2-nonanoyl, CH2NHCH2-nonenoyl, CH2NHCH2-decanoyl, CH2NH-decenoyl, CH2NHCH2-propionyl, CH2NHCH2-2-propenoyl, CH2NHCH2-2-butenoyl, CH2NHCH2-Isobutyryl, CH2NHCH2-2-methylpropanoyl, CH2NHCH2-2-ethylbutyryl, CH2NHCH2-ethylbutanoyl, CH2NHCH2-2-ethylbutanoyl, CH2NHCH2-butyryl, CH2NHCH2-(E)-2,3-Dimethylacryloyl, CH2NHCH2-(E)-2-Methylcrotonoyl, CH2NHCH2-3-cis-Methyl-methacryloyl, CH2NHCH2-3-Methyl-2-butenoyl, CH2NHCH2-3-Methylcrotonoyl, CH2NHCH2-4-Pentenoyl, CH2NHCH2-(2E)-2-pentenoyl, CH2NHCH2-Caproyl, CH2NHCH2-5-Hexenoyl, CH2NHCH2-Capryloyl, CH2NHCH2-Lauroyl, CH2NHCH2-Dodecanoyl, CH2NHCH2-Myristoyl, CH2NHCH2-Tetradecanoyl, CH2NHCH2-Oleoyl, CH2NHCH2-C(2-18) Acyl,

CH2NHCO-tigloyl, CH2NHCO-angeloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl, CH2NHCO-alkyl, CH2NHCO-dibenzoyl, CH2NHCO-benzoyl, CH2NHCO-alkanoyl, CH2NHCO-alkenoyl, CH2NHCO-benzoyl alkyl substituted CH2NHCO-alkanoyl, CH2NHCO-alkanoyl substituted phenyl, CH2NHCO-alkenoyl substituted phenyl, CH2NHCO-aryl, CH2NHCO-acyl, CH2NHCO-heterocylic, CH2NHCO-heteroraryl, CH2NHCO-alkenylcarbonyl, CH2NHCO-alkane, CH2NHCO-alkene, CH2NHCO-sugar moiety, CH2NHCO-acid moiety, CH2NHCO-ethanoyl, CH2NHCO-propanoyl, CH2NHCO-propenoyl, CH2NHCO-butanoyl, CH2NHCO-butenoyl, CH2NHCO-pentanoyl, CH2NHCO-hexenoyl, CH2NHCO-heptanoyl, CH2NHCO-heptenoyl, CH2NHCO-octanoyl, CH2NHCO-octenoyl, CH2NHCO-nonanoyl, CH2NHCO-nonenoyl, CH2NHCO-decanoyl, CH2NHCO-decenoyl, CH2NHCO-propionyl, CH2NHCO-2-propenoyl, CH2NHCO-2-butenoyl, CH2NHCO-Isobutyryl, CH2NHCO-2-methylpropanoyl, CH2NHCO-2-ethylbutyryl, CH2NHCO-ethylbutanoyl, CH2NHCO-2-ethylbutanoyl, CH2NHCO-butyryl, CH2NHCO-(E)-2,3-Dimethylacryloyl, CH2NHCO-(E)-2-Methylcrotonoyl, CH2NHCO-3-cis-Methyl-methacryloyl, CH2NHCO-3-Methyl-2-butenoyl, CH2NHCO-3-Methylcrotonoyl, CH2NHCO-4-Pentenoyl, CH2NHCO-(2E)-2-pentenoyl, CH2NHCO-Caproyl, CH2NHCO-5-Hexenoyl, CH2NHCO-Capryloyl, CH2NHCO-Lauroyl, CH2NHCO-Dodecanoyl, CH2NHCO-Myristoyl, CH2NHCO-Tetradecanoyl, CH2NHCO-Oleoyl, CH2NHCO—C(2-18) Acyl,

CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl, CH2NHCONH-alkyl, CH2NHCONH-dibenzoyl, CH2NHCONH-benzoyl, CH2NHCONH-alkanoyl, CH2NHCONH-alkenoyl, CH2NHCONH-benzoyl alkyl substituted CH2NHCONH-alkanoyl, CH2NHCONH-alkanoyl substituted phenyl, CH2NHCONH-alkenoyl substituted phenyl, CH2NHCONH-aryl, CH2NHCONH-acyl, CH2NHCONH-heterocylic, CH2NHCONH-heteroraryl, CH2NHCONH-alkenylcarbonyl, CH2NHCONH-alkane, CH2NHCONH-alkene, CH2NHCONH-sugar moiety, CH2NHCONH-acid moiety, CH2NHCONH-ethanoyl, CH2NHCONH-propanoyl, CH2NHCONH-propenoyl, CH2NHCONH-butanoyl, CH2NHCONH-butenoyl, CH2NHCONH-pentanoyl, CH2NHCONH-hexenoyl, CH2NHCONH-heptanoyl, CH2NHCONH-heptenoyl, CH2NHCONH-octanoyl, CH2NHCONH-octenoyl, CH2NHCONH-nonanoyl, CH2NHCONH-nonenoyl, CH2NHCONH-decanoyl, CH2NHCONH-decenoyl, CH2NHCONH-propionyl, CH2NHCONH-2-propenoyl, CH2NHCONH-2-butenoyl, CH2NHCONH-Isobutyryl, CH2NHCONH-2-methylpropanoyl, CH2NHCONH-2-ethylbutyryl, CH2NHCONH-ethylbutanoyl, CH2NHCONH-2-ethylbutanoyl, CH2NHCONH-butyryl, CH2NHCONH-(E)-2,3-Dimethylacryloyl, CH2NHCONH-(E)-2-Methylcrotonoyl, CH2NHCONH-3-cis-Methyl-methacryloyl, CH2NHCONH-3-Methyl-2-butenoyl, CH2NHCONH-3-Methylcrotonoyl, CH2NHCONH-4-Pentenoyl, CH2NHCONH-(2E)-2-pentenoyl, CH2NHCONH-Caproyl, CH2NHCONH-5-Hexenoyl, CH2NHCONH-Capryloyl, CH2NHCONH-Lauroyl, CH2NHCONH-Dodecanoyl, CH2NHCONH-Myristoyl, CH2NHCONH-Tetradecanoyl, CH2NHCONH-Oleoyl, CH2NHCONH—C(2-18) Acyl,

NHSO2-tigloyl, NHSO2-senecioyl, NHSO2-acetyl, NHSO2-Crotonoyl, NHSO2-3,3-Dimethylartyloyl, NHSO2-Cinnamoyl, NHSO2-Pentenoyl, NHSO2-Hexanoyl, NHSO2-benzoyl, NHSO2-Ethylbutyryl, NHSO2-alkyl, NHSO2-dibenzoyl, NHSO2-benzoyl, NHSO2-alkanoyl, NHSO2-alkenoyl, NHSO2-benzoyl alkyl substituted NHSO2-alkanoyl, NHSO2-alkanoyl substituted phenyl, NHSO2-alkenoyl substituted phenyl, NHSO2-aryl, NHSO2-acyl, NHSO2-heterocylic, NHSO2-heteroraryl, NHSO2-alkenylcarbonyl, NHSO2-alkane, NHSO2-alkene, NHSO2-sugar moiety, NHSO2-acid moiety, NHSO2-ethanoyl, NHSO2-propanoyl, NHSO2-propenoyl, NHSO2-butanoyl, NHSO2-butenoyl, NHSO2-pentanoyl, NHSO2-hexenoyl, NHSO2-heptanoyl, NHSO2-heptenoyl, NHSO2-octanoyl, NHSO2-octenoyl, NHSO2-nonanoyl, NHSO2-nonenoyl, NHSO2-decanoyl, NHSO2-decenoyl, NHSO2-propionyl, NHSO2-2-propenoyl, NHSO2-2-butenoyl, NHSO2-Isobutyryl, NHSO2-2-methylpropanoyl, NHSO2-2-ethylbutyryl, NHSO2-ethylbutanoyl, NHSO2-2-ethylbutanoyl, NHSO2-butyryl, NHSO2-(E)-2,3-Dimethylacryloyl, NHSO2-(E)-2-Methylcrotonoyl, NHSO2-3-cis-Methyl-methacryloyl, NHSO2-3-Methyl-2-butenoyl, NHSO2-3-Methylcrotonoyl, NHSO2-4-Pentenoyl, NHSO2-(2E)-2-pentenoyl, NHSO2-Caproyl, NHSO2-5-Hexenoyl, NHSO2-Capryloyl, NHSO2-Lauroyl, NHSO2-Dodecanoyl, NHSO2-Myristoyl, NHSO2-Tetradecanoyl, NHSO2-Oleoyl, NHSO2-C(2-18) Acyl,

CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl, CH2NHSO2-alkyl, CH2NHSO2-dibenzoyl, CH2NHSO2-benzoyl, CH2NHSO2-alkanoyl, CH2NHSO2-alkenoyl, CH2NHSO2-benzoyl alkyl substituted CH2NHSO2-alkanoyl, CH2NHSO2-alkanoyl substituted phenyl, CH2NHSO2-alkenoyl substituted phenyl, CH2NHSO2-aryl, CH2NHSO2-acyl, CH2NHSO2-heterocylic, CH2NHSO2-heteroraryl, CH2NHSO2-alkenylcarbonyl, CH2NHSO2-alkane, CH2NHSO2-alkene, CH2NHSO2-sugar moiety, CH2NHSO2-acid moiety, CH2NHSO2-ethanoyl, CH2NHSO2-propanoyl, CH2NHSO2-propenoyl, CH2NHSO2-butanoyl, CH2NHSO2-butenoyl, CH2NHSO2-pentanoyl, CH2NHSO2-hexenoyl, CH2NHSO2-heptanoyl, CH2NHSO2-heptenoyl, CH2NHSO2-octanoyl, CH2NHSO2-octenoyl, CH2NHSO2-nonanoyl, CH2NHSO2-nonenoyl, CH2NHSO2-decanoyl, CH2NHSO2-decenoyl, CH2NHSO2-propionyl, CH2NHSO2-2-propenoyl, CH2NHSO2-2-butenoyl, CH2NHSO2-Isobutyryl, CH2NHSO2-2-methylpropanoyl, CH2NHSO2-2-ethylbutyryl, CH2NHSO2-ethylbutanoyl, CH2NHSO2-2-ethylbutanoyl, CH2NHSO2-butyryl, CH2NHSO2-(E)-2,3-Dimethylacryloyl, CH2NHSO2-(E)-2-Methylcrotonoyl, CH2NHSO2-3-cis-Methyl-methacryloyl, CH2NHSO2-3-Methyl-2-butenoyl, CH2NHSO2-3-Methylcrotonoyl, CH2NHSO2-4-Pentenoyl, CH2NHSO2-(2E)-2-pentenoyl, CH2NHSO2-Caproyl, CH2NHSO2-5-Hexenoyl, CH2NHSO2-Capryloyl, CH2NHSO2-Lauroyl, CH2NHSO2-Dodecanoyl, CH2NHSO2-Myristoyl, CH2NHSO2-Tetradecanoyl, CH2NHSO2-Oleoyl, CH2NHSO2-C(2-18) Acyl In an embodiment, the compound(s) is(are) in form in form of powder, liquid or crystal. The active compounds of triterpenes in form of amine, sulfonamides, amide, and Urea Analogs, providing extremely stable activity in solution. They prolong the activities and duration of drug in a subject.

This invention provide the compound having structures of T1, T2, T3, T4, T5, T6, T7, T8, T9, T10, T11, T12, T13, T14, T15, T16, T17; T18, T19, T20, T21, T22, T23, T24, T25. T26 provided for treating cancers, inhibition of cancer growth, cancer invasion, cells invasion, cancer cell invasion; cell adhesion, cell attachment, cell circulating; for treating mad cow disease; treating prion diseases; for treating diabetes; for inhibiting viruses; for preventing cerebral aging; for improving memory; improving cerebral functions; for curing enuresis, frequent micturition, urinary incontinence; dementia, Alzheimer's disease, autism, brain trauma, Parkinson's disease or other diseases caused by cerebral dysfunctions or neurodegeneration; for treating arthritis, rheumatism, poor circulation, arteriosclerosis, Raynaud's syndrome, angina pectoris, cardiac disorder, coronary heart disease, headache, dizziness, kidney disorder; cerebrovascular diseasea; inhibiting NF-kappa B activation; for treating brain edema, severe acute respiratory syndrome, respiratory viral diseases, chronic venous insufficiency, hypertension, chronic venous disease, oedema, inflammation, hemorrhoids, peripheral edema formation, varicose vein disease, flu, post traumatic edema and postoperative swelling; for inhibiting blood clots, for inhibiting ethanol absorption; for lowering blood sugar; for regulating adrenocorticotropin and corticosterone levels. This invention provides a composition for Anti-MS, anti-aneurysm, anti-asthmatic, anti-oedematous, anti-inflammatory, anti-bradykinic, anti-capillarihemorrhagic, anti-cephalagic, anti-cervicobrachialgic, anti-eclamptic, anti-edemic, anti-encaphalitic, anti-epiglottitic, anti-exudative, anti-flu, anti-fracture, anti-gingivitic, anti-hematomic, anti-herpetic, anti-histaminic, anti-hydrathritic, anti-meningitic, antioxidant, anti-periodontic, anti-phlebitic, anti-pleuritic, anti-raucedo, anti-rhinitic, anti-tonsilitic, anti-ulcer, anti-varicose, anti-vertiginous, cancerostatic, corticosterogenic, diuretic, fungicide, hemolytic, hyaluronidase inhibitor, lymphagogue, natriuretic, pesticide, pituitary stimulant, thymolytic, vasoprotective, inhibiting leishmaniases, modulating adhesion or angiogenesis of cells, anti-parasitic; increase the expression of the genes: ANGPT2, DDIT3, LIF and NFKB1Z, and manufacturing an adjuvant composition and venotonic treatment. The compound blocks the DNA synthesis of cancer cell; wherein the cancers comprise breast cancer, leukocytic cancer, liver cancer, ovarian cancer, bladder cancer, prostatic cancer, skin cancer, bone cancer, brain cancer, leukemia cancer, lung cancer, colon cancer, CNS cancer, melanoma cancer, renal cancer, cervical cancer, esophageal cancer, testicular cancer, splenic cancer, kidney cancer, lymphatic cancer, pancreatic cancer, stomach cancer, eye cancer and thyroid cancer. This invention provides compounds to improve blood circulation; soothing stroke; Prevent plaque formation and promote their dissipated; improve blood viscosity; reduce cardiovascular; reduce cerebrovascular; reduce thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, flutter, Blood circulation, swelling, pain; Treating bronchiectasis, tuberculosis and lung abscess caused by too hemoptysis; reducing bleeding, antitussive, expectorant and analgesic effect, dilate blood vessels; reducing blood pressure and the treatment of cerebral arteriosclerosis; elevated blood lipids and reduced cholesterol, Inducing or Increasing the number of Tumour Necrosis Factor (TNF) receptors (RELL1), DR1, DR4, or DR5.

Liposome is artificially prepared vesicles which made up of a lipid bilayer. Certain sizes of liposome can enter tumour sites from blood due to the enhanced permeability and retention effect. While human blood vessels are all surrounded by endothelial cells bound by tight junctions, those tight junctions binding tumour vessels are leakier than those binding other vessels and thus liposomes are able to enter these vessels to enhance the delivery, efficacy, bioavailability and absorption of liposome enclosed drug. This invention provides methods to use liposomes or nanoparticles capsules as a carrier delivering the compound as medicament, wherein the size of liposomes or nanoparticles capsules is less than 200 nm or 100-200 nm or 50-100 nm or 5-50 nm or less than 50 nm, wherein the medicament is included but not limited for treating cancer, inhibiting cancer growth, inhibiting cancer invasion, inhibiting cancer metastasis, modulating cell adhesion, modulating cell attachment, wherein the compound is selected from formula (2A) or formula (K) at the above. In an embodiment, the compound(s) is(are) in form in form of powder, liquid or crystal.

Substitution, deletion and/or addition of any group in the above-described compounds by other group(s) will be apparent to one of ordinary skill in the art based on the teachings of this application. In a further embodiment, the substitution, deletion and/or addition of the group(s) in the compound of the invention does not substantially affect the biological function of the compound is included in the invention.

In an embodiment, the compound is selected from the structures:

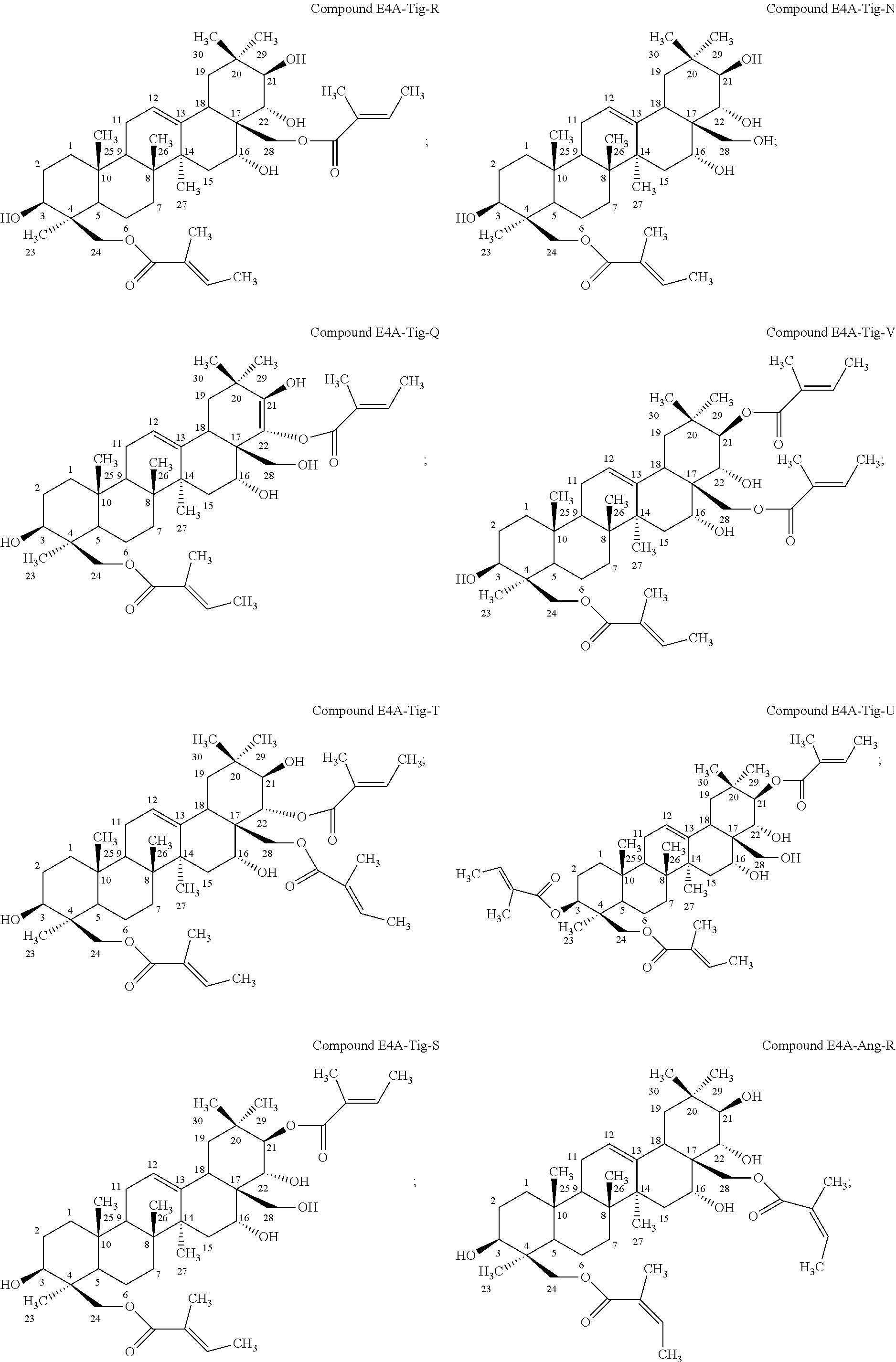

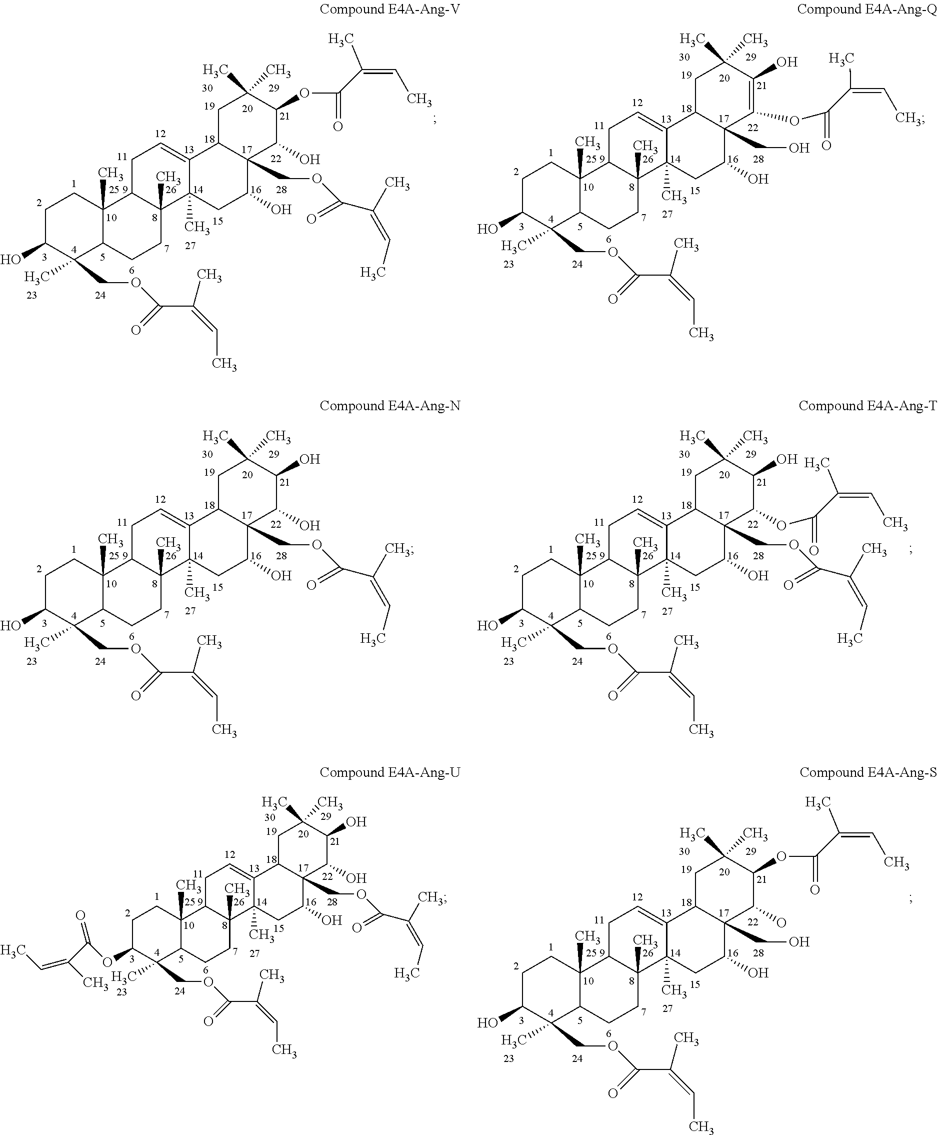

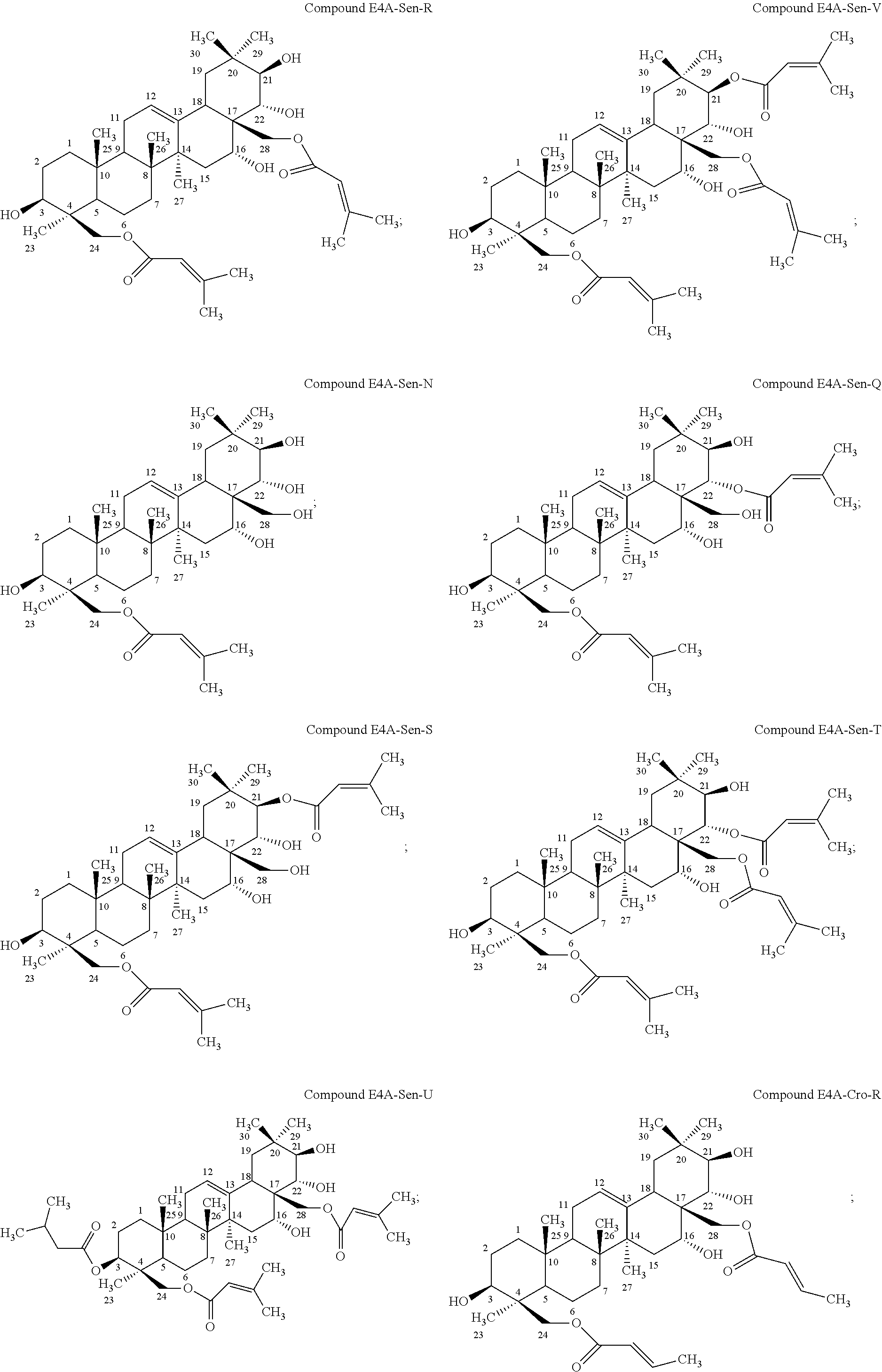

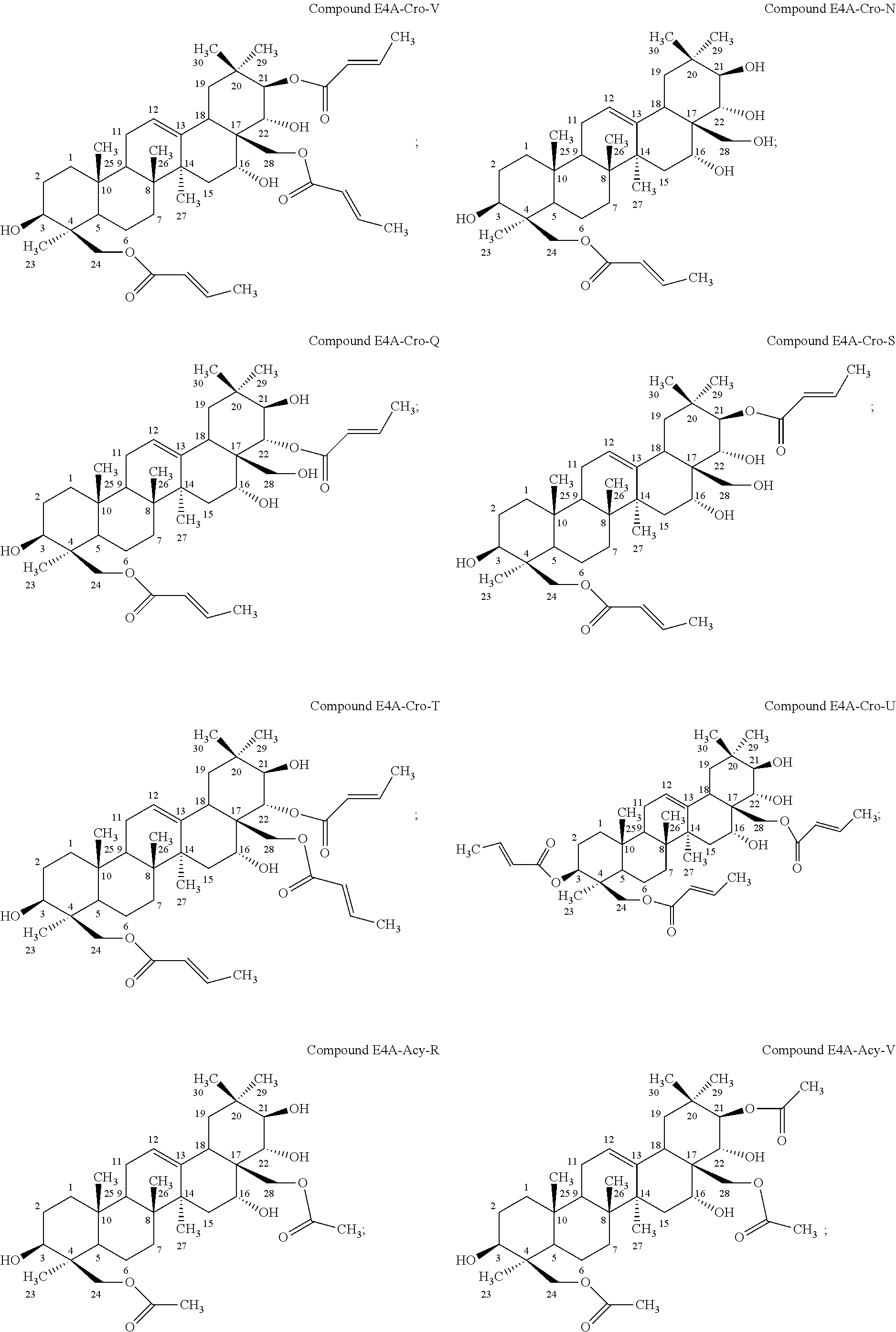

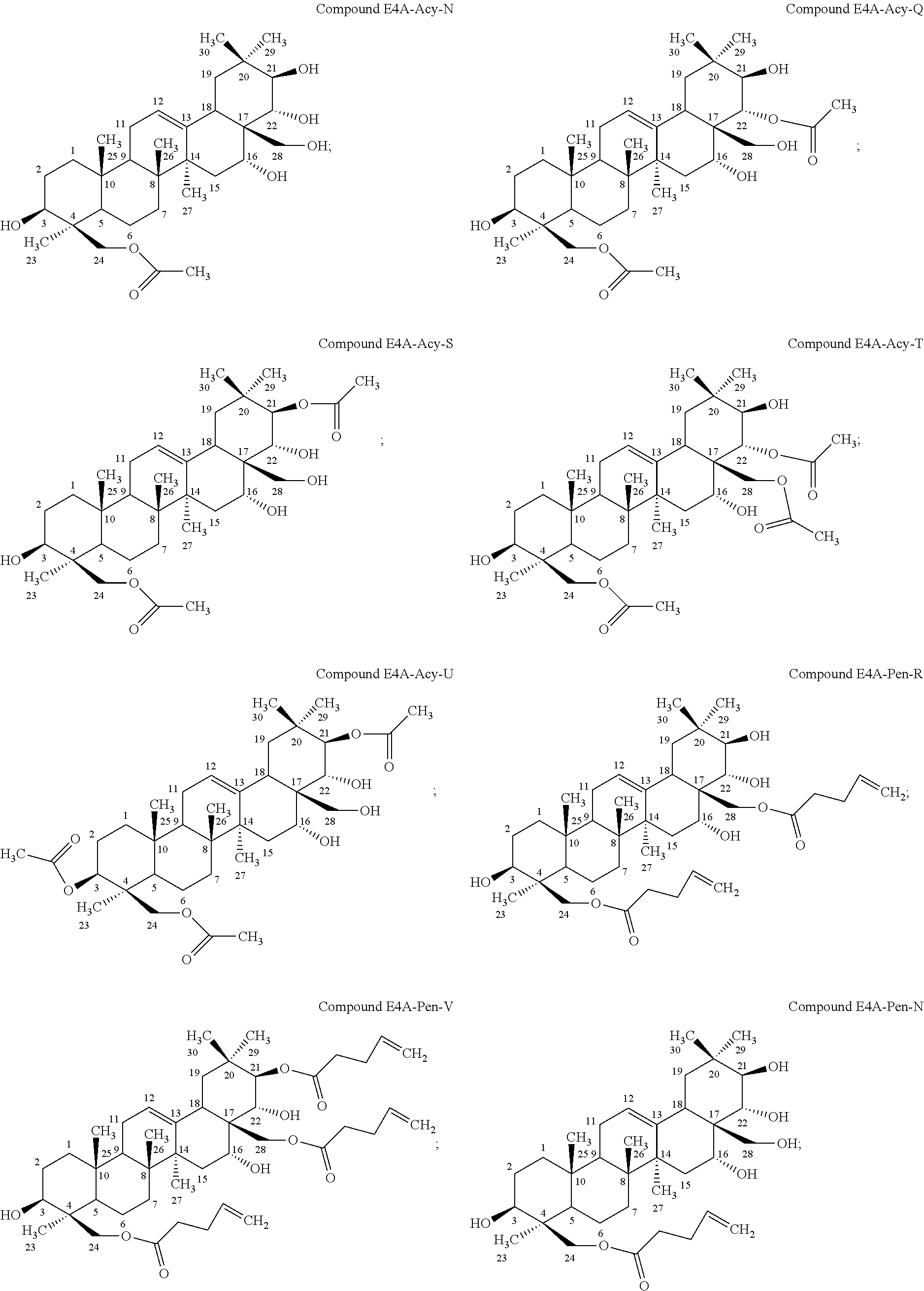

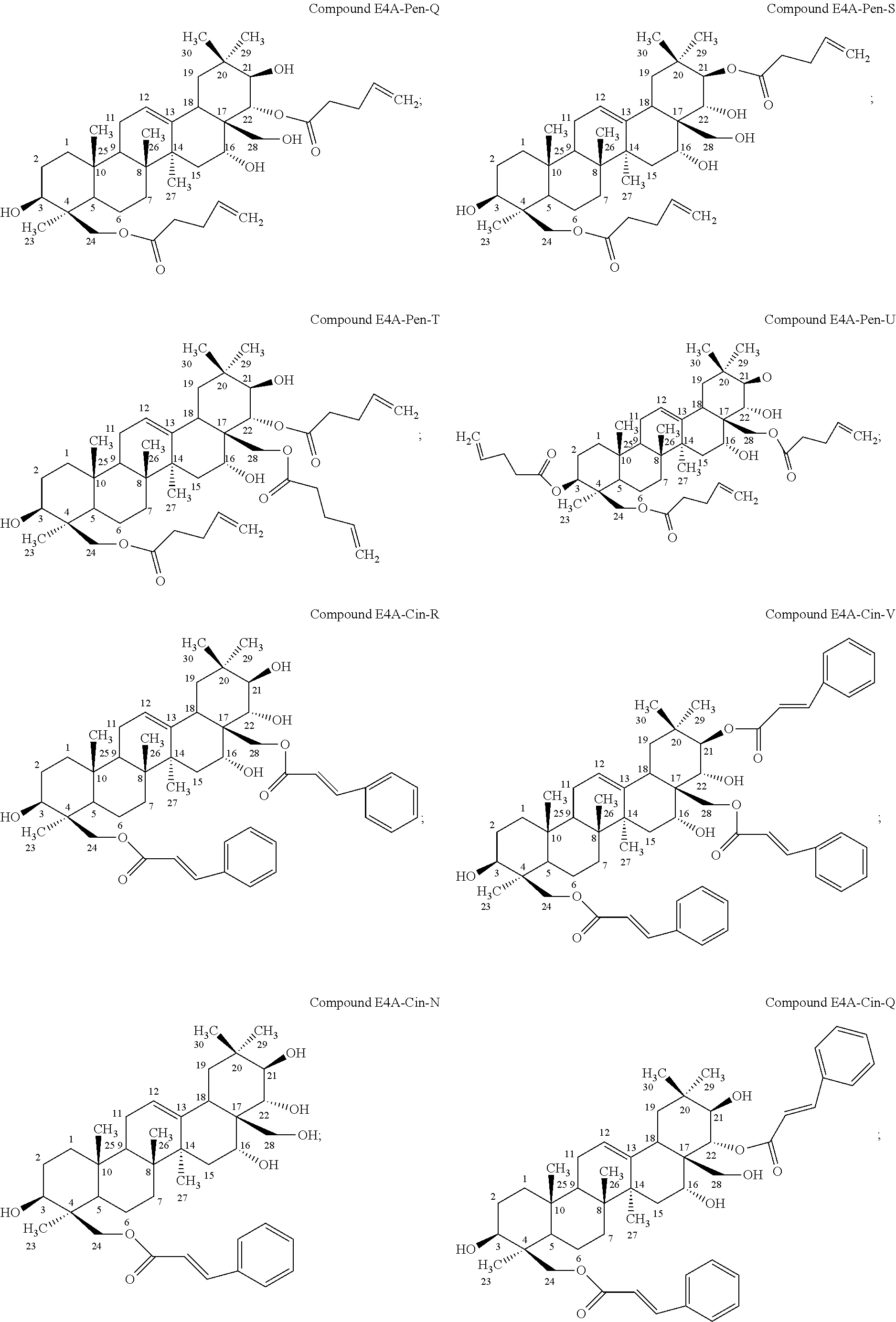

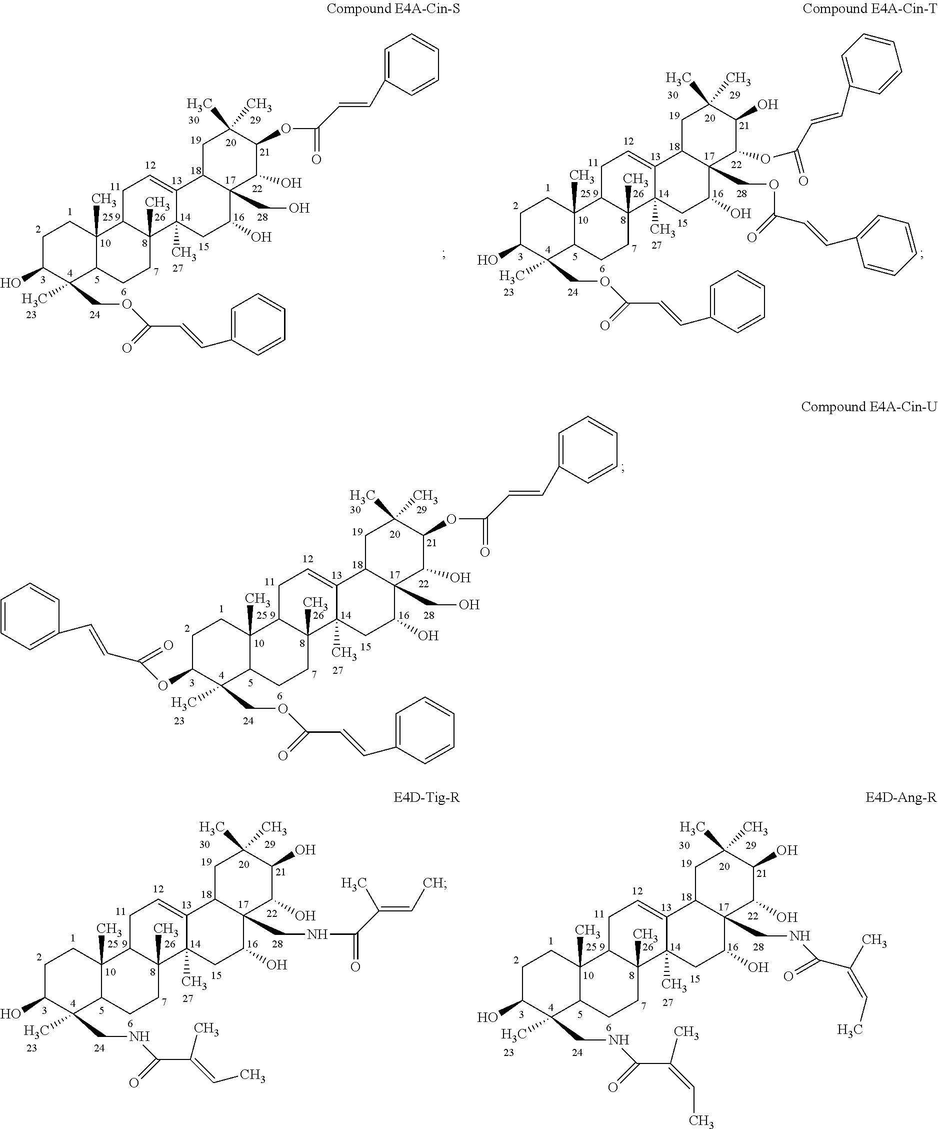

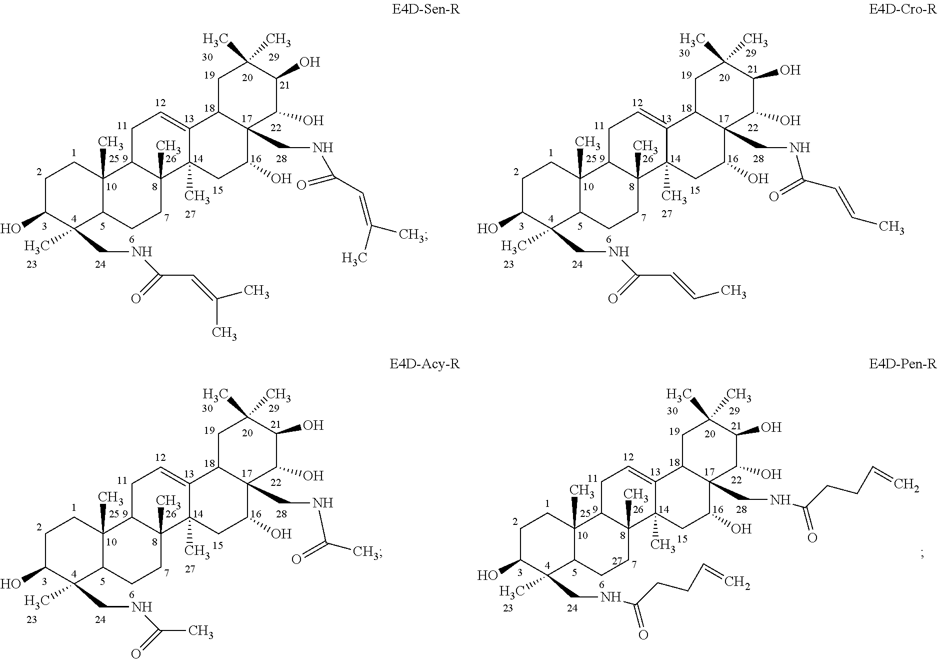

In embodiment, sugar moiety(ies) or acid moiety(ies) can be attached to the above compounds. In embodiment, the attachment of sugar moiety(ies) can be biosynthesized; In embodiment, the attachment of acid moiety can be biosynthesized; wherein the sugar moiety(ies) or acid moiety(ies) is/are included but not limited to a group of glucose, galactose, rhamnose, arabinose, xylose, fucose, allose, altrose, gulose, idose, lyxose, mannose, psicose, ribose, sorbose, tagatose, talose, fructose, alduronic acid, glucuronic acid, galacturonic acid, and derivatives or combinations thereof;

This invention provides compounds by esterification, amidation, amination, or sulfonamidation or methods of present invention of core compound (C), E4A, E4D1 or E4D in this application with acetyl chloride, angeloyl chloride, tigloyl chloride, senecioyl chloride, Crotonoyl chloride, O-3,3-Dimethylartyloyl chloride, Cinnamoyl chloride, Pentenoyl chloride, Hexanoyl chloride, benzoyl chloride, Ethylbutyryl chloride, and isolation of the compounds with HPLC, for treating cancer, inhibiting cancer growth, inhibiting cancer invasion, inhibiting cancer metastasis, modulating cell adhesion, modulating cell attachment, wherein the core compound selected from the following:

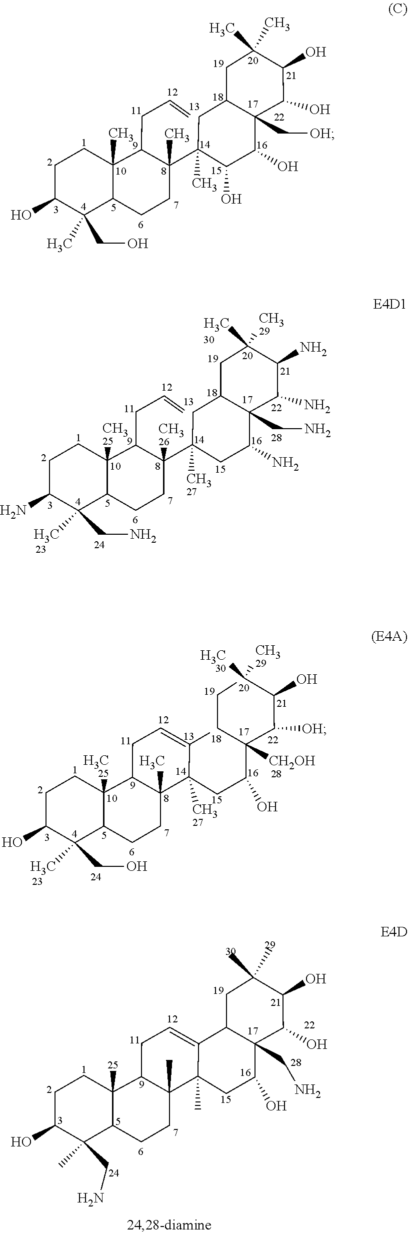

With experiments and methods in present application or esterification, amidation, amination, or sulfonamidation of compounds (C), E4A, E4D, or E4D1with acyl chloride, sulfonyl chloride, isocyanate, aldehyde, carboxylic acid, including Tigloyl chloride, angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, benzoyl chloride, Ethylbutyryl chloride, ethanoyl chloride, propanoyl chloride, propenoyl chloride, butanoyl chloride, butenoyl chloride, pentanoyl chloride, hexenoyl chloride, heptanoyl chloride, heptenoyl chloride, octanoyl chloride, octenoyl chloride, nonanoyl chloride, nonenoyl chloride, decanoyl chloride, decenoyl chloride, propionyl chloride, 2-propenoyl chloride, 2-butenoyl chloride, Isobutyryl chloride, 2-methylpropanoyl chloride, 2-ethylbutyryl chloride, ethylbutanoyl chloride, 2-ethylbutanoyl chloride, butyryl chloride, (E)-2,3-Dimethylacryloyl chloride, (E)-2-Methylcrotonoyl chloride, 3-cis-Methyl-methacryloyl chloride, 3-Methyl-2-butenoyl chloride, 3-Methylcrotonoyl chloride, 4-Pentenoyl chloride, (2E)-2-pentenoyl chloride, Caproyl chloride, 5-Hexenoyl chloride, Capryloyl chloride, Lauroyl chloride, Dodecanoyl chloride, Myristoyl chloride, Tetradecanoyl chloride, Oleoyl chloride, C(2-18) Acyl chloride. In an embodiment, the compound(s) is(are) in form in form of powder, liquid or crystal.

The compounds vary in composition when the time or temperature of the reaction is changed. The peaks, fractions and compounds are selected according to the activities of times studies and the changes of peaks. The compounds having strong to weak activities are selected and isolated. The anti cancer activities are the MTT studies of bone (U2OS), lung (H460), bladder (HTB-9), ovary (ES2), colon (HCT116), pancreas (Capan), ovary (OVCAR3), prostate (DU145), skin (SK-Mel-5), mouth (KB), kidney (A498), breast (MCF-7), liver (HepG2), brain (T98G), luekemia (K562), cervix (HeLa). The active esterification products are purified with HPLC. The reaction product of mixtures and individual compounds are tested with MTT Cytotoxic Assay. Details of method are in Experiment 3 of the present application. A second esterification of compound can be selected from the above experiment results to produce new active compounds. A partial esterification compound is selected from the above experiments to perform a second or repeated with a third esterification with different acyl chloride or methods in present application in order to produce new active compounds with the experiments in the present application, wherein the compound can be selected from K, (H1) or (H2):

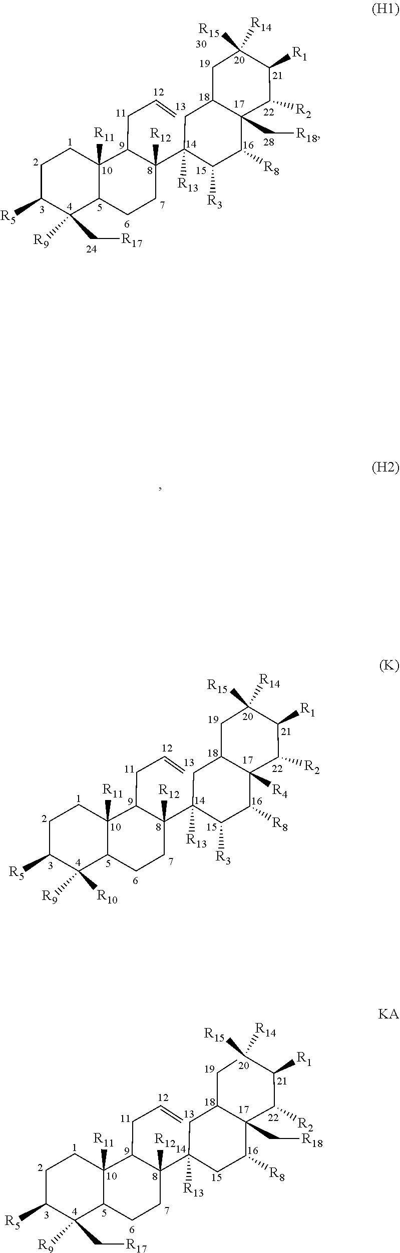

R1, R2, R3, R4, R5, R8, R9, R10, R11, R12, R13, R14, R15, R16, R17, R18 are independently selected from the group of CH3, CH2OH, COOH, hydrogen, hydroxyl, methyl, O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-alkane, O-alkene, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl; CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl;

CH2O-4-(dimethylamino)-2-methylbut-2-enoyl, CH2O-4-[(2-methoxyethyl)amino]-2-methyl-4oxobut-2-enoyl or wherein any 1 or 2 or 3 or 4 of R1, R2, R3, R4, R5, R8, R10, R16, R17, R18 is/are independently attached an 0-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl, CH2O-4-(dimethylamino)-2-methylbut-2-enoyl, CH2O-4-[(2-methoxyethyl)amino]-2-methyl-4oxobut-2-enoyl; R9, R11, R12, R13, R14, R15 are independently attached a CH3; or wherein R10 is attached an O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methylpropanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl, O-4-(dimethylamino)-2-methylbut-2-enoyl, and O-4-[(2-methoxyethyl)amino]-2-methyl-4oxobut-2-enoyl, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH3, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl; CH2O-4-(dimethylamino)-2-methylbut-2-enoyl, CH2O-4-[(2-methoxyethyl)amino]-2-methyl-4oxobut-2-enoyl or wherein R4 and R10 are independently attached an CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH2OH, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, CH2O-ethanoyl, CH2O-propanoyl, CH2O-propenoyl, CH2O-butanoyl, CH2O-butenoyl, CH2O-pentanoyl, CH2O-hexenoyl, CH2O-heptanoyl, CH2O-heptenoyl, CH2O-octanoyl, CH2O-octenoyl, CH2O-nonanoyl, CH2O-nonenoyl, CH2O-decanoyl, CH2O-decenoyl, CH2O-propionyl, CH2O-2-propenoyl, CH2O-2-butenoyl, CH2O-Isobutyryl, CH2O-2-methylpropanoyl, CH2O-2-ethylbutyryl, CH2O-ethylbutanoyl, CH2O-2-ethylbutanoyl, CH2O-butyryl, CH2O-(E)-2,3-Dimethylacryloyl, CH2O-(E)-2-Methylcrotonoyl, CH2O-3-cis-Methyl-methacryloyl, CH2O-3-Methyl-2-butenoyl, CH2O-3-Methylcrotonoyl, CH2O-4-Pentenoyl, CH2O-(2E)-2-pentenoyl, CH2O-Caproyl, CH2O-5-Hexenoyl, CH2O-Capryloyl, CH2O-Lauroyl, CH2O-Dodecanoyl, CH2O-Myristoyl, CH2O-Tetradecanoyl, CH2O-Oleoyl, CH2O—C(2-18) Acyl; CH2O-4-(dimethylamino)-2-methylbut-2-enoyl, CH2O-4-[(2-methoxyethyl)amino]-2-methyl-4oxobut-2-enoyl or wherein R17 and R18 are independently attached an O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl, O-ethanoyl, O-propanoyl, O-propenoyl, O-butanoyl, O-butenoyl, O-pentanoyl, O-hexenoyl, O-heptanoyl, O-heptenoyl, O-octanoyl, O-octenoyl, O-nonanoyl, O-nonenoyl, O-decanoyl, O-decenoyl, O-propionyl, O-2-propenoyl, O-2-butenoyl, O-Isobutyryl, O-2-methyl propanoyl, O-2-ethylbutyryl, O-ethylbutanoyl, O-2-ethylbutanoyl, O-butyryl, O-(E)-2,3-Dimethylacryloyl, O-(E)-2-Methylcrotonoyl, O-3-cis-Methyl-methacryloyl, O-3-Methyl-2-butenoyl, O-3-Methylcrotonoyl, O-4-Pentenoyl, O-(2E)-2-pentenoyl, O-Caproyl, O-5-Hexenoyl, O-Capryloyl, O-Lauroyl, O-Dodecanoyl, O-Myristoyl, O-Tetradecanoyl, O-Oleoyl, O—C(2-18) Acyl; wherein R3 is OH or H or absent; wherein R1, R2, R3, R5, R8 are OH or H or absent; wherein R9, R11, R12, R13, R14, and R15 are CH3; or wherein R1, R2, R5, R8 represent OH; R3 represents OH, H or absent; R4, R10 represent CH2Oangeloyl; R9, R11, R12, R13, R14, R15 represent CH3; or wherein R1, R2, R5, R8 represent OH or 0-tigloyl; R3 represents OH, H, or absent; R4, R10 represent CH2O tigloyl; R9, R11, R12, R13, R14, R15 represent CH3;

R1, R2, R3, R4, R5, R8, R9, R10, R11, R12, R13, R14, R15, R16, R17, R18 are independently selected from the group of OH, CH3, H, O, NH2, CH3, CH2OH, COOH NH-tigloyl, NH-angeloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl, NH-alkyl, NH-dibenzoyl, NH-benzoyl, NH-alkanoyl, NH-alkenoyl, NH-benzoyl alkyl substituted NH-alkanoyl, NH-alkanoyl substituted phenyl, NH-alkenoyl substituted phenyl, NH-aryl, NH-acyl, NH-heterocylic, NH-heteroraryl, NH-alkenylcarbonyl, NH-alkane, NH-alkene, NH-sugar moiety, NH-acid moiety, NH-ethanoyl, NH-propanoyl, NH-propenoyl, NH-butanoyl, NH-butenoyl, NH-pentanoyl, NH-hexenoyl, NH-heptanoyl, NH-heptenoyl, NH-octanoyl, NH-octenoyl, NH-nonanoyl, NH-nonenoyl, NH-decanoyl, NH-decenoyl, NH-propionyl, NH-2-propenoyl, NH-2-butenoyl, NH-Isobutyryl, NH-2-methylpropanoyl, NH-2-ethylbutyryl, NH-ethylbutanoyl, NH-2-ethylbutanoyl, NH-butyryl, NH-(E)-2,3-Dimethylacryloyl, NH-(E)-2-Methylcrotonoyl, NH-3-cis-Methyl-methacryloyl, NH-3-Methyl-2-butenoyl, NH-3-Methylcrotonoyl, NH-4-Pentenoyl, NH-(2E)-2-pentenoyl, NH-Caproyl, NH-5-Hexenoyl, NH-Capryloyl, NH-Lauroyl, NH-Dodecanoyl, NH-Myristoyl, NH-Tetradecanoyl, NH-Oleoyl, NH—C(2-18) Acyl, NH-ethanyl, NH-propanyl, NH-propenyl, NH-butanyl, NH-butenyl, NH-pentanyl, NH-hexenyl, NH-heptanyl, NH-heptenyl, NH-octanyl, NH-octenyl, NH-nonanyl, NH-nonenyl, NH-decanyl, NH-decenyl, NH-alkyl, NH-haloalkyl, NH-alkenyl, NH-alkynyl, NH-hydroxyalkyl, NH-alkylene-O-alkyl, NH-aryl, NH-alkylene-aryl, NH-heteroaryl, NH-alkylene-heteroaryl, NH-cycloalkyl, NH-heterocyclyl, and NH-alkylene-heterocyclyl, CH2NH-tigloyl, CH2NH-angeloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl, CH2NH-ethanyl, CH2NH-propanyl, CH2NH-propenyl, CH2NH-butanyl, CH2NH-butenyl, CH2NH-pentanyl, CH2NH-hexenyl, CH2NH-heptanyl, CH2NH-heptenyl, CH2NH-octanyl, CH2NH-octenyl, CH2NH-nonanyl, CH2NH-nonenyl, CH2NH-decanyl, CH2NH-decenyl, CH2NH-alkyl, CH2NH-haloalkyl, CH2NH-alkenyl, CH2NH-alkynyl, CH2NH-hydroxyalkyl, CH2NH-alkylene-O-alkyl, CH2NH-aryl, CH2NH-alkylene-aryl, CH2NH-heteroaryl, CH2NH-alkylene-heteroaryl, CH2NH-cycloalkyl, CH2NH-heterocyclyl, and CH2NH-alkylene-heterocyclyl,

CH2NHCH2-tigloyl, CH2NHCH2-angeloyl, CH2NHCH2-senecioyl, CH2NHCH2-acetyl, CH2NHCH2-Crotonoyl, CH2NHCH2-3,3-Dimethylartyloyl, CH2NHCH2-Cinnamoyl, CH2NHCH2-Pentenoyl, CH2NHCH2-Hexanoyl, CH2NHCH2-benzoyl, CH2NHCH2-Ethylbutyryl, CH2NHCH2-alkyl, CH2NHCH2-dibenzoyl, CH2NHCH2-benzoyl, CH2NHCH2-alkanoyl, CH2NHCH2-alkenoyl, CH2NHCH2-benzoyl alkyl substituted CH2NHCH2-alkanoyl, CH2NHCH2-alkanoyl substituted phenyl, CH2NHCH2-alkenoyl substituted phenyl, CH2NHCH2-aryl, CH2NHCH2-acyl, CH2NHCH2-heterocylic, CH2NHCH2-heteroraryl, CH2NHCH2-alkenylcarbonyl, CH2NHCH2-alkane, CH2NHCH2-alkene, CH2NHCH2-sugar moiety, CH2NHCH2-acid moiety, CH2NHCH2-ethanoyl, CH2NHCH2-propanoyl, CH2NHCH2-propenoyl, CH2NHCH2-butanoyl, CH2NHCH2-butenoyl, CH2NHCH2-pentanoyl, CH2NHCH2-hexenoyl, CH2NHCH2-heptanoyl, CH2NHCH2-heptenoyl, CH2NHCH2-octanoyl, CH2NHCH2-octenoyl, CH2NHCH2-nonanoyl, CH2NHCH2-nonenoyl, CH2NHCH2-decanoyl, CH2NH-decenoyl, CH2NHCH2-propionyl, CH2NHCH2-2-propenoyl, CH2NHCH2-2-butenoyl, CH2NHCH2-Isobutyryl, CH2NHCH2-2-methylpropanoyl, CH2NHCH2-2-ethylbutyryl, CH2NHCH2-ethylbutanoyl, CH2NHCH2-2-ethylbutanoyl, CH2NHCH2-butyryl, CH2NHCH2-(E)-2,3-Dimethylacryloyl, CH2NHCH2-(E)-2-Methylcrotonoyl, CH2NHCH2-3-cis-Methyl-methacryloyl, CH2NHCH2-3-Methyl-2-butenoyl, CH2NHCH2-3-Methylcrotonoyl, CH2NHCH2-4-Pentenoyl, CH2NHCH2-(2E)-2-pentenoyl, CH2NHCH2-Caproyl, CH2NHCH2-5-Hexenoyl, CH2NHCH2-Capryloyl, CH2NHCH2-Lauroyl, CH2NHCH2-Dodecanoyl, CH2NHCH2-Myristoyl, CH2NHCH2-Tetradecanoyl, CH2NHCH2-Oleoyl, CH2NHCH2-C(2-18) Acyl, CH2NHCH2-ethanyl, CH2NHCH2-propanyl, CH2NHCH2-propenyl, CH2NHCH2-butanyl, CH2NHCH2-butenyl, CH2NHCH2-pentanyl, CH2NHCH2-hexenyl, CH2NHCH2-heptanyl, CH2NHCH2-heptenyl, CH2NHCH2-octanyl, CH2NHCH2-octenyl, CH2NHCH2-nonanyl, CH2NHCH2-nonenyl, CH2NHCH2-decanyl, CH2NHCH2-decenyl, CH2NHCH2-alkyl, haloalkyl, CH2NHCH2-alkenyl, CH2NHCH2-alkynyl, CH2NHCH2-hydroxyalkyl, CH2NHCH2-alkylene-O-alkyl, CH2NHCH2-aryl, CH2NHCH2-alkylene-aryl, CH2NHCH2-heteroaryl, CH2NHCH2-alkylene-heteroaryl, CH2NHCH2-cycloalkyl, CH2NHCH2-heterocyclyl, and CH2NHCH2-alkylene-heterocyclyl,

CH2NHCO-tigloyl, CH2NHCO-angeloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl, CH2NHCO-alkyl, CH2NHCO-dibenzoyl, CH2NHCO-benzoyl, CH2NHCO-alkanoyl, CH2NHCO-alkenoyl, CH2NHCO-benzoyl alkyl substituted CH2NHCO-alkanoyl, CH2NHCO-alkanoyl substituted phenyl, CH2NHCO-alkenoyl substituted phenyl, CH2NHCO-aryl, CH2NHCO-acyl, CH2NHCO-heterocylic, CH2NHCO-heteroraryl, CH2NHCO-alkenylcarbonyl, CH2NHCO-alkane, CH2NHCO-alkene, CH2NHCO-sugar moiety, CH2NHCO-acid moiety, CH2NHCO-ethanoyl, CH2NHCO-propanoyl, CH2NHCO-propenoyl, CH2NHCO-butanoyl, CH2NHCO-butenoyl, CH2NHCO-pentanoyl, CH2NHCO-hexenoyl, CH2NHCO-heptanoyl, CH2NHCO-heptenoyl, CH2NHCO-octanoyl, CH2NHCO-octenoyl, CH2NHCO-nonanoyl, CH2NHCO-nonenoyl, CH2NHCO-decanoyl, CH2NHCO-decenoyl, CH2NHCO-propionyl, CH2NHCO-2-propenoyl, CH2NHCO-2-butenoyl, CH2NHCO-Isobutyryl, CH2NHCO-2-methylpropanoyl, CH2NHCO-2-ethylbutyryl, CH2NHCO-ethylbutanoyl, CH2NHCO-2-ethylbutanoyl, CH2NHCO-butyryl, CH2NHCO-(E)-2,3-Dimethylacryloyl, CH2NHCO-(E)-2-Methylcrotonoyl, CH2NHCO-3-cis-Methyl-methacryloyl, CH2NHCO-3-Methyl-2-butenoyl, CH2NHCO-3-Methylcrotonoyl, CH2NHCO-4-Pentenoyl, CH2NHCO-(2E)-2-pentenoyl, CH2NHCO-Caproyl, CH2NHCO-5-Hexenoyl, CH2NHCO-Capryloyl, CH2NHCO-Lauroyl, CH2NHCO-Dodecanoyl, CH2NHCO-Myristoyl, CH2NHCO-Tetradecanoyl, CH2NHCO-Oleoyl, CH2NHCO—C(2-18) Acyl, CH2NHCO-ethanyl, CH2NHCO-propanyl, CH2NHCO-propenyl, CH2NHCO-butanyl, CH2NHCO-butenyl, CH2NHCO-pentanyl, CH2NHCO-hexenyl, CH2NHCO-heptanyl, CH2NHCO-heptenyl, CH2NHCO-octanyl, CH2NHCO-octenyl, CH2NHCO-nonanyl, CH2NHCO-nonenyl, CH2NHCO-decanyl, CH2NHCO-decenyl, CH2NHCO-alkyl, CH2NHCO-haloalkyl, alkenyl, CH2NHCO-alkynyl, CH2NHCO-hydroxyalkyl, CH2NHCO-alkylene-O-alkyl, CH2NHCO-aryl, CH2NHCO-alkylene-aryl, CH2NHCO-heteroaryl, CH2NHCO-alkylene-heteroaryl, CH2NHCO-cycloalkyl, CH2NHCO-heterocyclyl, and CH2NHCO-alkylene-heterocyclyl,

CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl, CH2NHCONH-alkyl, CH2NHCONH-dibenzoyl, CH2NHCONH-benzoyl, CH2NHCONH-alkanoyl, CH2NHCONH-alkenoyl, CH2NHCONH-benzoyl alkyl substituted CH2NHCONH-alkanoyl, CH2NHCONH-alkanoyl substituted phenyl, CH2NHCONH-alkenoyl substituted phenyl, CH2NHCONH-aryl, CH2NHCONH-acyl, CH2NHCONH-heterocylic, CH2NHCONH-heteroraryl, CH2NHCONH-alkenylcarbonyl, CH2NHCONH-alkane, CH2NHCONH-alkene, CH2NHCONH-sugar moiety, CH2NHCONH-acid moiety, CH2NHCONH-ethanoyl, CH2NHCONH-propanoyl, CH2NHCONH-propenoyl, CH2NHCONH-butanoyl, CH2NHCONH-butenoyl, CH2NHCONH-pentanoyl, CH2NHCONH-hexenoyl, CH2NHCONH-heptanoyl, CH2NHCONH-heptenoyl, CH2NHCONH-octanoyl, CH2NHCONH-octenoyl, CH2NHCONH-nonanoyl, CH2NHCONH-nonenoyl, CH2NHCONH-decanoyl, CH2NHCONH-decenoyl, CH2NHCONH-propionyl, CH2NHCONH-2-propenoyl, CH2NHCONH-2-butenoyl, CH2NHCONH-Isobutyryl, CH2NHCONH-2-methylpropanoyl, CH2NHCONH-2-ethylbutyryl, CH2NHCONH-ethylbutanoyl, CH2NHCONH-2-ethylbutanoyl, CH2NHCONH-butyryl, CH2NHCONH-(E)-2,3-Dimethylacryloyl, CH2NHCONH-(E)-2-Methylcrotonoyl, CH2NHCONH-3-cis-Methyl-methacryloyl, CH2NHCONH-3-Methyl-2-butenoyl, CH2NHCONH-3-Methylcrotonoyl, CH2NHCONH-4-Pentenoyl, CH2NHCONH-(2E)-2-pentenoyl, CH2NHCONH-Caproyl, CH2NHCONH-5-Hexenoyl, CH2NHCONH-Capryloyl, CH2NHCONH-Lauroyl, CH2NHCONH-Dodecanoyl, CH2NHCONH-Myristoyl, CH2NHCONH-Tetradecanoyl, CH2NHCONH-Oleoyl, CH2NHCONH—C(2-18) Acyl, CH2NHCONH-ethanyl, CH2NHCONH-propanyl, CH2NHCONH-propenyl, CH2NHCONH-butanyl, CH2NHCONH-butenyl, CH2NHCONH-pentanyl, CH2NHCONH-hexenyl, CH2NHCONH-heptanyl, CH2NHCONH-heptenyl, CH2NHCONH-octanyl, CH2NHCONH-octenyl, CH2NHCONH-nonanyl, CH2NHCONH-nonenyl, CH2NHCONH-decanyl, CH2NHCONH-decenyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl),

NHSO2-tigloyl, NHSO2-senecioyl, NHSO2-acetyl, NHSO2-Crotonoyl, NHSO2-3,3-Dimethylartyloyl, NHSO2-Cinnamoyl, NHSO2-Pentenoyl, NHSO2-Hexanoyl, NHSO2-benzoyl, NHSO2-Ethylbutyryl, NHSO2-alkyl, NHSO2-dibenzoyl, NHSO2-benzoyl, NHSO2-alkanoyl, NHSO2-alkenoyl, NHSO2-benzoyl alkyl substituted NHSO2-alkanoyl, NHSO2-alkanoyl substituted phenyl, NHSO2-alkenoyl substituted phenyl, NHSO2-aryl, NHSO2-acyl, NHSO2-heterocylic, NHSO2-heteroraryl, NHSO2-alkenylcarbonyl, NHSO2-alkane, NHSO2-alkene, NHSO2-sugar moiety, NHSO2-acid moiety, NHSO2-ethanoyl, NHSO2-propanoyl, NHSO2-propenoyl, NHSO2-butanoyl, NHSO2-butenoyl, NHSO2-pentanoyl, NHSO2-hexenoyl, NHSO2-heptanoyl, NHSO2-heptenoyl, NHSO2-octanoyl, NHSO2-octenoyl, NHSO2-nonanoyl, NHSO2-nonenoyl, NHSO2-decanoyl, NHSO2-decenoyl, NHSO2-propionyl, NHSO2-2-propenoyl, NHSO2-2-butenoyl, NHSO2-Isobutyryl, NHSO2-2-methylpropanoyl, NHSO2-2-ethylbutyryl, NHSO2-ethylbutanoyl, NHSO2-2-ethylbutanoyl, NHSO2-butyryl, NHSO2-(E)-2,3-Dimethylacryloyl, NHSO2-(E)-2-Methylcrotonoyl, NHSO2-3-cis-Methyl-methacryloyl, NHSO2-3-Methyl-2-butenoyl, NHSO2-3-Methylcrotonoyl, NHSO2-4-Pentenoyl, NHSO2-(2E)-2-pentenoyl, NHSO2-Caproyl, NHSO2-5-Hexenoyl, NHSO2-Capryloyl, NHSO2-Lauroyl, NHSO2-Dodecanoyl, NHSO2-Myristoyl, NHSO2-Tetradecanoyl, NHSO2-Oleoyl, NHSO2-C(2-18) Acyl, NHSO2-ethanyl, NHSO2-propanyl, NHSO2-propenyl, NHSO2-butanyl, NHSO2-butenyl, NHSO2-pentanyl, NHSO2-hexenyl, NHSO2-heptanyl, NHSO2-heptenyl, NHSO2-octanyl, NHSO2-octenyl, NHSO2-nonanyl, NHSO2-nonenyl, NHSO2-decanyl, NHSO2-decenyl, NHSO2-alkyl, NHSO2-haloalkyl, NHSO2-alkenyl, NHSO2-alkynyl, NHSO2-hydroxyalkyl, NHSO2-alkylene-O-alkyl, NHSO2-aryl, NHSO2-alkylene-aryl, NHSO2-heteroaryl, NHSO2-alkylene-heteroaryl, NHSO2-cycloalkyl, NHSO2-heterocyclyl, NHSO2-alkylene-heterocyclyl, NHSO2-ethyl, NHSO2-(Z)-(2-methylbut-2-en-1-yl), NHSO2-(E)-prop-1-enyl, NHSO2-(E)-2-phenylethenyl, NHSO2-but-3-enyl,

CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl, CH2NHSO2-alkyl, CH2NHSO2-dibenzoyl, CH2NHSO2-benzoyl, CH2NHSO2-alkanoyl, CH2NHSO2-alkenoyl, CH2NHSO2-benzoyl alkyl substituted CH2NHSO2-alkanoyl, CH2NHSO2-alkanoyl substituted phenyl, CH2NHSO2-alkenoyl substituted phenyl, CH2NHSO2-aryl, CH2NHSO2-acyl, CH2NHSO2-heterocylic, CH2NHSO2-heteroraryl, CH2NHSO2-alkenylcarbonyl, CH2NHSO2-alkane, CH2NHSO2-alkene, CH2NHSO2-sugar moiety, CH2NHSO2-acid moiety, CH2NHSO2-ethanoyl, CH2NHSO2-propanoyl, CH2NHSO2-propenoyl, CH2NHSO2-butanoyl, CH2NHSO2-butenoyl, CH2NHSO2-pentanoyl, CH2NHSO2-hexenoyl, CH2NHSO2-heptanoyl, CH2NHSO2-heptenoyl, CH2NHSO2-octanoyl, CH2NHSO2-octenoyl, CH2NHSO2-nonanoyl, CH2NHSO2-nonenoyl, CH2NHSO2-decanoyl, CH2NHSO2-decenoyl, CH2NHSO2-propionyl, CH2NHSO2-2-propenoyl, CH2NHSO2-2-butenoyl, CH2NHSO2-Isobutyryl, CH2NHSO2-2-methylpropanoyl, CH2NHSO2-2-ethylbutyryl, CH2NHSO2-ethylbutanoyl, CH2NHSO2-2-ethylbutanoyl, CH2NHSO2-butyryl, CH2NHSO2-(E)-2,3-Dimethylacryloyl, CH2NHSO2-(E)-2-Methylcrotonoyl, CH2NHSO2-3-cis-Methyl-methacryloyl, CH2NHSO2-3-Methyl-2-butenoyl, CH2NHSO2-3-Methylcrotonoyl, CH2N HSO2-4-Pentenoyl, CH2NHSO2-(2E)-2-pentenoyl, CH2NHSO2-Caproyl, CH2NHSO2-5-Hexenoyl, CH2NHSO2-Capryloyl, CH2NHSO2-Lauroyl, CH2NHSO2-Dodecanoyl, CH2NHSO2-Myristoyl, CH2NHSO2-Tetradecanoyl, CH2NHSO2-Oleoyl, CH2NHSO2-C(2-18) Acyl, CH2NHSO2-ethanyl, CH2NHSO2-propanyl, CH2NHSO2-propenyl, CH2NHSO2-butanyl, CH2NHSO2-butenyl, CH2NHSO2-pentanyl, CH2NHSO2-hexenyl, CH2NHSO2-heptanyl, CH2NHSO2-heptenyl, CH2NHSO2-octanyl, CH2NHSO2-octenyl, CH2NHSO2-nonanyl, CH2NHSO2-nonenyl, CH2NHSO2-decanyl, CH2NHSO2-decenoyl, CH2NHSO2-alkyl, CH2NHSO2-haloalkyl, CH2NHSO2-alkenyl, CH2NHSO2-alkynyl, CH2NHSO2-hydroxyalkyl, CH2NHSO2-alkylene-O-alkyl, CH2NHSO2-aryl, CH2NHSO2-alkylene-aryl, CH2NHSO2-heteroaryl, CH2NHSO2-alkylene-heteroaryl, CH2NHSO2-cycloalkyl, CH2NHSO2-heterocyclyl, and CH2NHSO2-alkylene-heterocyclyl, CH2NHSO2-ethyl, CH2NHSO2-(Z)-(2-methylbut-2-en-1-yl), CH2NHSO2-(E)-prop-1-enyl, CH2NHSO2-(E)-2-phenylethenyl, CH2NHSO2-but-3-enyl,

In an embodiment, the compound(s) is(are) in form of powder, liquid, or crystal. In an embodiment, the compound(s) is(are) in form of salt, ester, amine, diamine, amide, sulfonamide, urea thereof, or metabolite thereof'

The compounds of present application on cells is arresting cells in the S-phase and blocking their entering into the G2/M phase of cell cycle. The compounds block the DNA synthesis of cancer cell This invention provides compounds and method for improving blood circulation; soothing stroke; Prevent plaque formation and promote their dissipated; improve blood viscosity; reducing cardiovascular; reducing cerebrovascular; reducing thrombosis, arteriosclerosis, coronary heart disease, hypertension, diabetes, thrombocytopenia purpura, hemoptysis, hematemesis; treating blood in the stool, uterine bleeding, traumatic bleeding, abdominal irritation, swelling, flutter, Blood circulation, swelling, pain; Treating bronchiectasis, tuberculosis and lung abscess caused by too hemoptysis; reducing bleeding, antitussive, expectorant and analgesic effect, dilate blood vessels; reducing blood pressure and the treatment of cerebral arteriosclerosis; elevated blood lipids and reduced cholesterol.

A composition comprising an effective amount of compound selected from the above formula or a salt, ester, amine, diamine, amide, sulfonamide, urea, metabolite or derivative thereof can be used as a medicament for blocking the invasion, migration, metastasis of cancer cells, inhibiting tumor or cancer cell growth and for treating cancer, wherein the cancers comprise breast cancer, leukocytic cancer, liver cancer, ovarian cancer, bladder cancer, prostatic cancer, skin cancer, bone cancer, brain cancer, leukemia cancer, lung cancer, colon cancer, CNS cancer, melanoma cancer, renal cancer, cervical cancer, esophageal cancer, testicular cancer, splenic cancer, kidney cancer, lymphatic cancer, pancreatic cancer, stomach cancer, eye cancer and thyroid cancer.

This invention provides a composition comprising the compounds provided in the invention for treating cancers; for inhibiting viruses; for preventing cerebral aging; for improving memory; improving cerebral functions; for curing enuresis, frequent micturition, urinary incontinence; dementia, Alzheimer's disease, autism, brain trauma, Parkinson's disease or other diseases caused by cerebral dysfunctions; for treating arthritis, rheumatism, poor circulation, arteriosclerosis, Raynaud's syndrome, angina pectoris, cardiac disorder, coronary heart disease, headache, dizziness, kidney disorder; diabetes; cerebrovascular diseasea; inhibiting NF-kappa B activation; for treating brain edema, severe acute respiratory syndrome, respiratory viral diseases, chronic venous insufficiency, hypertension, chronic venous disease, oedema, inflammation, hemonhoids, peripheral edema formation, varicose vein disease, flu, post traumatic edema and postoperative swelling; for inhibiting blood clots, for inhibiting ethanol absorption; for lowering blood sugar; for regulating adrenocorticotropin and corticosterone levels. This invention provides a composition for Anti-MS, anti-aneurysm, anti-asthmatic, anti-oedematous, anti-inflammatory, anti-bradykinic, anti-capillarihemorrhagic, anti-cephalagic, anti-cervicobrachialgic, anti-eclamptic, anti-edemic, anti-encaphalitic, anti-epiglottitic, anti-exudative, anti-flu, anti-fracture, anti-gingivitic, anti-hematomic, anti-herpetic, anti-histaminic, anti-hydrathritic, anti-meningitic, antioxidant, anti-periodontic, anti-phlebitic, anti-pleuritic, anti-raucedo, anti-rhinitic, anti-tonsilitic, anti-ulcer, anti-varicose, anti-vertiginous, cancerostatic, corticosterogenic, diuretic, fungicide, hemolytic, hyaluronidase inhibitor, lymphagogue, natriuretic, pesticide, pituitary stimulant, thymolytic, vasoprotective, inhibiting leishmaniases, modulating adhesion or angiogenesis of cells, anti-parasitic; increase the expression of the genes: ANGPT2, DDIT3, LIF and NFKB1Z, and manufacturing an adjuvant composition and venotonic treatment. The composition block the DNA synthesis of cancer cell.

This invention provides a method of synthesis compound, wherein the compound is selected from formula

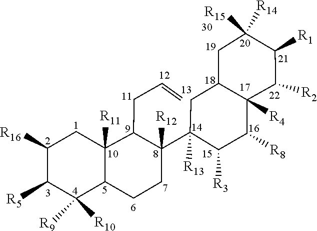

or salt, ester, diamine, amine, amide, sulfonamide or urea thereof, wherein R4 and R10 are selected from CH2O-tigloyl, NH-tigloyl, CH2NH-tigloyl, CH2NHCO-tigloyl, CH2NHCONH-tigloyl, CH2NHSO2-tigloyl; R1, R2, R3, R5, R8, R9, R11, R12, R13, R14, R15, R16 are independently selected from the group of H, O, OH, NH2, CH3, CH2OH, and COOH.

In an embodiment, the structure of above wherein R4 and R10 are selected from CH2O-tigloyl, NH-tigloyl, CH2NH-tigloyl, CH2NHCO-tigloyl, CH2NHCONH-tigloyl, CH2NHSO2-tigloyl; CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl; NH-angeloyl, NH-tigloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl; CH2NH-angeloyl, CH2NH-tigloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl; CH2NHCO-angeloyl, CH2NHCO-tigloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl; CH2NHCONH-angeloyl, CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl; CH2NHSO2-angeloyl, CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl; wherein R1, R2, R3, R5, R8, R9, R11, R12, R13, R14, R15, R16 are independently selected from the group of H, O, OH, NH2, CH3, CH2OH, and COOH; wherein the compound is in form in form of powder, liquid or crystal.

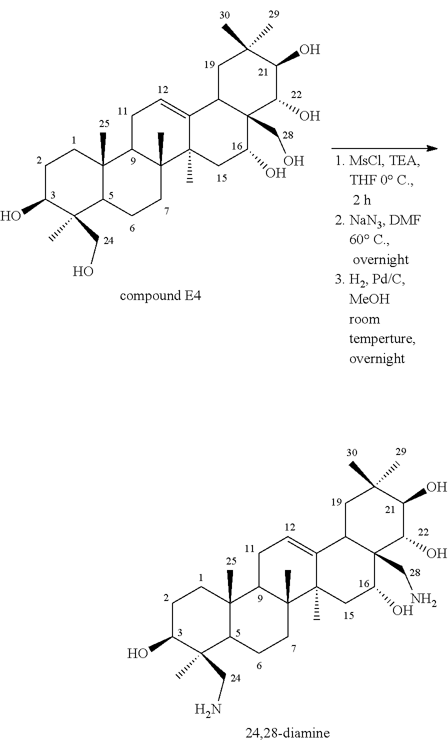

To a solution of 24,28-diamine(E4D) (0.1 mmol.) and aldehyde (RCHO, 0.3 mmol) in dimethylformamide (DMF, 1 mL) in a fritted plastic reaction tube was added SiliaBondCyanoborohydride (Si-CBH, 0.3 mmol). The reaction mixture was shaken at room temperature overnight. The reaction mixture was filtered into a fritted plastic reaction tube. Tosic acid functionalized silica (Si-SCX, 0.3 mmol) was added. The reaction mixture was shaken at room temperature overnight. The liquid was filtered off. A solution of 5% ammonia-methanol (1 mL) was added to the product captured silica. The mixture was shaken at room temperature overnight. The methanolic solution was filtered off and concentrated under reduced pressure to yield the desired amine analog.

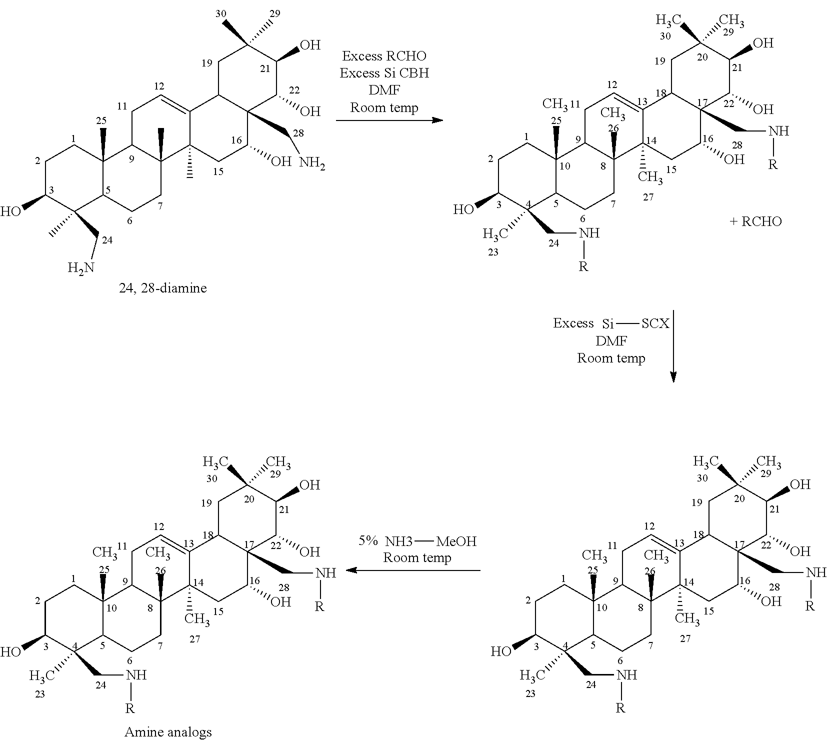

To a solution of 24,28-diamine(E4D) (0.1 mmol.) and carboxylic acid (RCO₂H, 0.3 mmol) in dimethylformamide (DMF, 1 mL) in a fritted plastic reaction tube was added EDC functionalized silica (Si-EDC, 0.3 mmol). The reaction mixture was shaken at room temperature overnight. Carbonate functionalized silica (Si—CO₃, 0.3 mmol) was added. The reaction mixture was shaken at room temperature overnight. The reaction mixture was filtered and concentrated under reduced pressure to yield the desired amide analog

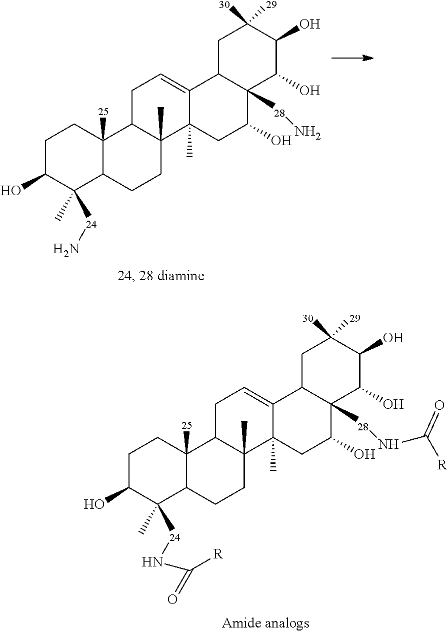

To a solution of 24,28-diamine(E4D) (0.1 mmol.) and sulfonyl chloride (RSO₂Cl, 0.3 mmol) in dimethylformamide (DMF, 1 mL) in a fritted plastic reaction tube was added triethylamine functionalized silica (Si-WAX-2, 0.3 mmol). The reaction mixture was shaken at room temperature overnight. Amine functionalized silica (Si-WAX, 0.3 mmol) was added. The reaction mixture was shaken at room temperature overnight. The reaction mixture was filtered and concentrated under reduced pressure to yield the desired sulfonamide analog.

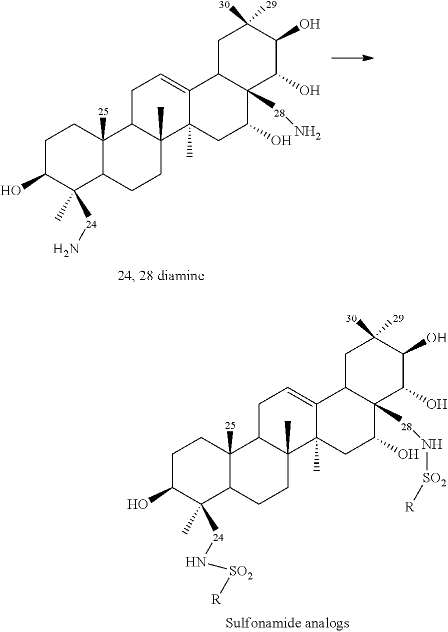

A solution of 24,28-diamine(E4D) (0.1 mmol.) and isocyanate (RNCO, 0.3 mmol) in dimethylformamide (DMF, 1 mL) in a fritted plastic reaction tube was shaken at room temperature overnight. Piperazine functionalized silica (Si-PPZ, 0.3 mmol) was added. The reaction mixture was shaken at room temperature overnight. The reaction mixture was filtered and concentrated under reduced pressure to yield the desired urea analog.

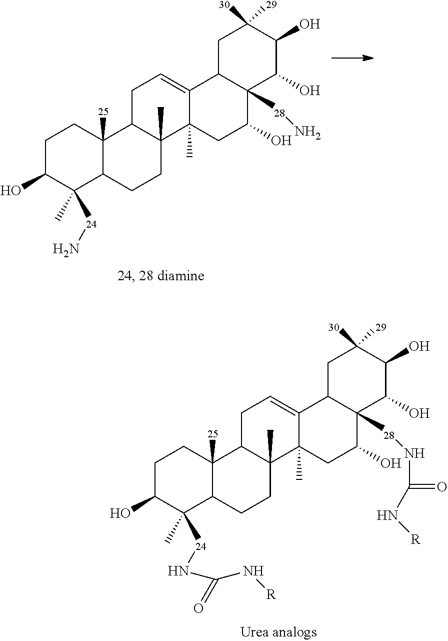

R group for amine, sulfonamides, amide analog, Urea Analogs, wherein sulfonyl chloride (RSO₂Cl), carboxylic acid (RCO₂H), isocyanate (RNCO), where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-hterocyclyl are unsubstituted or substituted with one or more groups of Z.

Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂; Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;

Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂-R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(akyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

Alkenyl means unsaturated linear or branched structures and combinations thereof, having formula R2C═CR2, one or more double bonds therein. Examples of alkenyl groups include vinyl, propenyl, isopropenyl, butenyl, s- and t-butenyl, pentenyl, hexenyl, butadienyl, pentadienyl, and hexadienyl. An aryl is a functional group of organic molecule derived from an aromatic compound such as benzene, a 6-14 membered carbocyclic aromatic ring system comprising 1-3 benzene rings. If two or more aromatic rings are present, then the rings are fused together, so that adjacent rings share a common bond. Examples include phenyl and naphthyl. The aryl group may be substituted with one or more substitutes independently selected from halogen, alkyl or alkoxy. Acyl is a functional group which can be obtained from an organic acid by the removal of the carboxyl. Acyl groups can be written using the general formula —COR, where there is a double bond between the carbon and oxygen. The names of acyl groups typically end in -yl, such as formyl, acetyl, propionyl, butyryl and benzoyl. Benzoyl is one of the acyls, C₆H₅COR, obtained from benzoic acid by the removal of the carboxyl. A heterocyclic compound is a compound containing a heterocyclic ring which refers to a non-aromatic ring having 1-4 heteroatoms, said ring being isolated or fused to a second ring selected from 3- to 7-membered alicyclic ring containing 0-4 heteroatoms, aryl and heteroaryl, wherein heterocyclic compounds include pyrrolidinyl, pipyrazinyl, morpholinyl, trahydrofuranyl, imidazolinyl, thiomorpholinyl, and the like. Heterocyclyl groups are derived from heteroarenes by removal of a hydrogen atom from any ring atom. Alkanoyl is the general name for an organic functional group RCO—, where R represents hydrogen or an alkyl group. Examples of alkanoyls are acetyl, propionoyl, butyryl, isobutyryl, pentanoyl and hexanoyl. Alkenoyl is an alkenylcarbonyl in which the alkenyl is defined above. Examples are pentenoyl (tigloyl) and pentenoyl (angeloyl). Alkyl is a radical containing only carbon and hydrogen atoms arranged in a chain, branched, cyclic or bicyclic structure or their combinations, having 1-18 carbon atoms. Examples include but are not limited to methyl, ethyl, propyl isopropyl, butyl, s- and t-butyl, pentyl, hexyl, heptyl, octyl, nonyl, undecyl, dodecyl, tridecyl, tetradecyl, pentadecyl, cyclopropyl, cyclobutyl, cyclopentyl and cyclohexyl. Benzoyl alkyl substituted alkanoyl refers to straight or branched alkanoyl substituted with at least one benzoyl and at least one alkyl, wherein the benzoyl is attached to a straight or branched alkyl. An example of a benzoyl alkyl substituted alkanoyl is benzoyl methyl isobutanoyl. A sugar moiety is a segment of molecule comprising one or more sugars or derivatives thereof or alduronic acid thereof.

(Y)Y3, Y and Y3 represent the same compound. YM and (ACH-Y) represent the same compound. Connecting moiety is a substructure or a group of atoms which connect the functional group to a core compound. Example: angeloyl group is connected by a sugar moiety to a triterpene core.

Acetyl=ethanoyl; Propionyl=methylpropanoyl; Crotonoyl=2-butenoyl; Isobutyryl=2-methylpropanoyl; 2-Ethylbutyryl=2-Ethylbutanoyl; Butyryl=n-Butyryl=butanoyl=C-4 Acyl; trans-2-Methyl-2-butenoyl=(E)-2,3-Dimethylacryloyl chloride=(E)-2-Methylcrotonoyl=3-cis-Methyl-methacryloyl=Tigloyl; 3,3-Dimethylacryloyl=3-Methyl-2-butenoyl=3-Methylcrotonoyl=Senecioyl; Propionyl chloride=methylpropanoyl; Hexanoyl=Caproyl; Heptanoyl=Enanthic=Oenanthic; Octanoyl=Capryloyl; Dodecanoyl=Lauroyl; Tetradecanoyl=Myristoyl; C(2-18)Acyl is an acyl group having 2 to 18 carbons.

ethanoyl is a C-2 Acyl, propanoyl is a C-3 Acyl, propenoyl is a C-3 Acyl, propionyl is a C-3 Acyl, butanoyl is a C-4 Acyl, butenoyl is a C-4 Acyl, crotonoyl is a C-4 Acyl, pentanoyl is a C-5 Acyl, pentenoyl is a C-5 Acyl, angeloyl is C-5 Acyl, tigloyl is C-5 Acyl, senecioyl is C-5 Acyl, hexanoyl is a C-6 Acyl, hexenoyl is a C-6 Acyl, heptanoyl is a C-7 Acyl, heptenoyl is a C-7 Acyl, octanoyl is a C-8 Acyl, octenoyl is a C-8 Acyl, nonanoyl is a C-9 Acyl, nonenoyl is a C-9 Acyl, decanoyl is a C-10 Acyl, decenoyl is a C-10 Acyl, lauroyl is a C-12 Acyl, dodecanoyl is a C-12 Acyl, myristoyl is a C-14 Acyl, oleoyl is a C-18 Acyl.

The building blocks used in the invention including triterpenes, hydroxylated triterpenes, acetyl, angeloyl, tigloyl, senecioyl, Crotonoyl, 3,3-Dimethylartyloyl, Cinnamoyl, Pentenoyl, Hexanoyl, benzoyl, Ethylbutyryl, alkyl, dibenzoyl, benzoyl, methylbutanoyl, methylpropanoyl, alkanoyl, alkenoyl, benzoyl alkyl substituted alkanoyl, alkanoyl substituted phenyl, alkenoyl substituted phenyl, aryl, acyl, heterocylic, heteroraryl, alkenylcarbonyl, ethanoyl, propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl and Oleoyl, 4-(dimethylamino)-2-methylbut-2-enoyl, 4-[(2-methoxyethyl)amino]-2-methyl-4oxobut-2-enoyl or halides thereof, or chloride.thereof.

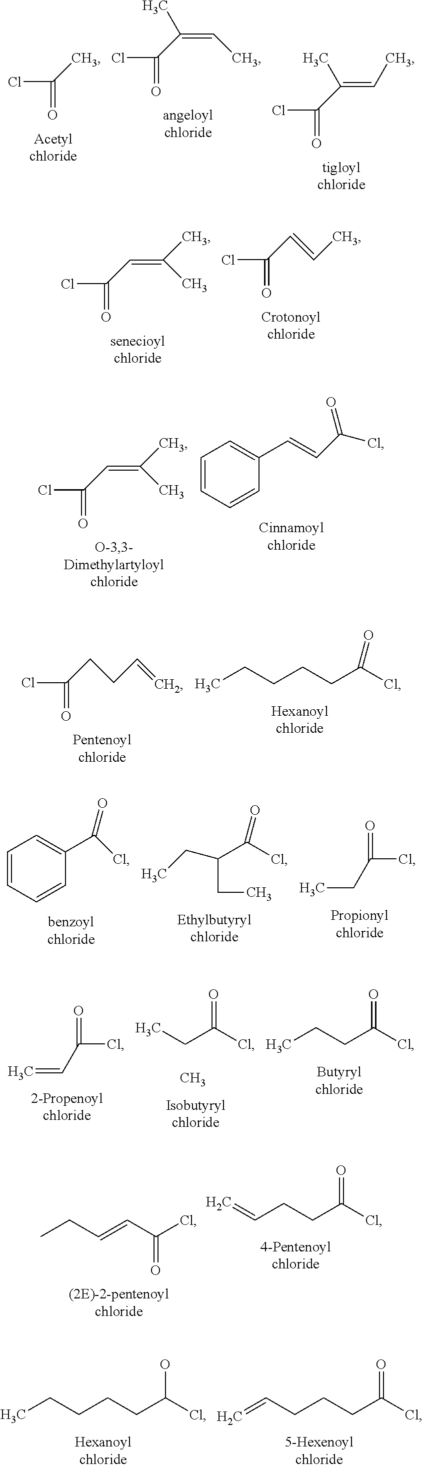

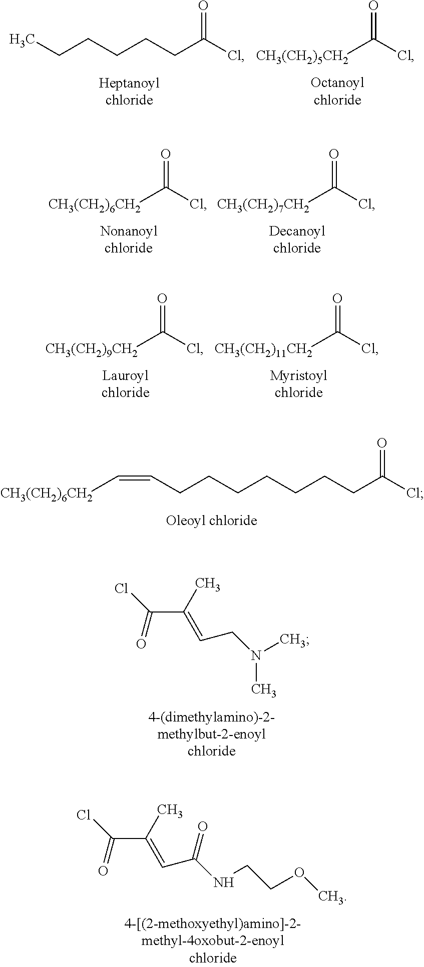

Acryloyl chloride [Synonym: 2-propenoly chloride]; Propionyl chloride [Synonym: methylpropanoyl chloride]; Crotonoyl chloride [Synonym: 2-butenoyl chloride]; Isobutyryl chloride [Synonym: 2-methylpropanoyl chloride]; 2-Ethylbutyryl chloride [Synonym: 2-Ethylbutanoyl chloride]; Butyryl chloride (Synonym: n-Butyryl chloride, butanoyl chloride, or C-4 Acyl halide); trans-2-Methyl-2-butenoyl chloride [Synonym: (E)-2,3-Dimethylacryloyl chloride, (E)-2-Methylcrotonoyl chloride, 3-cis-Methyl-methacryloyl chloride, Tigloyl chloride]; 3,3-Dimethylacryloyl chloride [Synonym: 3-Methyl-2-butenoyl chloride, 3-Methylcrotonoyl chloride, Senecioyl chloride ]; Hexanoyl chloride [Synonym: Caproyl chloride]; Heptanoyl chloride [Synonym: Enanthic chloride, Oenanthic chloride] Octanoyl chloride [Synonym: Capryloyl chloride].

In the presented experiments, concentrations of drug that inhibit 15% cell-growth or less (i.e. 85% of control or above) as compared to the no-drug control (DMSO) are considered non-cytotoxic concentrations. In an embodiment, the concentrations of drug that inhibit 10% cell-growth or less (i.e. 90% of control or above) as compared to the no-drug control (DMSO) are considered non-cytotoxic concentrations. In an embodiment, the concentrations of drug that inhibit 5% cell-growth or less (i.e. 95% of control or above) as compared to the no-drug control (DMSO) are considered non-cytotoxic concentrations. In an embodiment, the concentrations of drug that inhibit 20% cell-growth or less (i.e. 80% of control or above) as compared to the no-drug control (DMSO) are considered non-cytotoxic concentrations. In an embodiment, the concentrations of drug that inhibit 25% cell-growth or less (i.e. 75% of control or above) as compared to the no-drug control (DMSO) are considered non-cytotoxic concentrations. In an embodiment, the concentrations of drug that inhibit 30% cell-growth or less as compared to the no-drug control (DMSO) are considered non-cytotoxic concentrations. In an embodiment, the concentrations of drug that inhibit 45% cell-growth or less as compared to the no-drug control (DMSO) are considered non-cytotoxic concentrations.

The triterpene compound or compounds selected from this invention can be administered to a subject in need thereof, treating the subject, wherein including preventing cancer, or providing an adjuvant effect to the subject, or inhibiting the initation or promotion of cancer, or killing the cancer/tumor cells, or inhibiting cancer cell invasion. In an embodiment the compounds inhibit the activation of Nuclear Factor-kB, wherein inhibiting the localization or wherein binding the DNA. In an embodiment the compounds block the DNA synthesis. In an embodiment the compounds induce apoptosis in cancer cells.

The saponins are partially hydrolyzed into a mixture of products which can be separated by HPLC. Specific partial hydrolysis of saponins can also be achieved with enzymes. The glycosidases catalyze the hydrolysis of the glycosidic linkage. Galactosidase is an enzyme which catalyzes the hydrolysis of galactosides. Glucosidase is an enzyme which breaks glucose from saponin. Other enzyme examples are xylanases, lactase, amylase, chitinase, sucrase, maltase, and neuraminidase.

The sugar moiety of the triterpenoid saponin (example Xanifolia Y) can be removed by acid hydrolysis. The synthetic compound of ACH-Y is obtained. ACH-Y is a triterpene with acyl groups but no sugar moiety. The acyl group of the saponin (example Xanifolia Y) can be removed by alkaline hydrolysis. The synthetic compound AKOH-Y can be obtained. AKOH-Y is a pentacyclic triterpene with sugar moieties. A pentacyclic triterpene can be obtained by acid and alkaline hydroysis of saponins from natural sources. A pentacyclic triterpene can be obtained by synthetic methods (Reference: Surendra et al., Rapid and Enantioselective Synthetic Approaches to Germanicol and Other Pentacyclic Triterpenes, Journal of the American Chemical Society, 2008, 130(27), 8865-8869). Pentacyclic triterpenes with sugar moieties can also be obtained by synthesis (Reference: Ple et al., Synthesis of L-arabinopyranose containing hederagenin saponins, Tetrahedron 61 (2005) 4347-4362). Acylation is the process of adding an acyl group to a compound. The Friedel-Crafts reaction is an example of this process. An active compound can be obtained by acylating a pentacyclic triterpenes, or hydroxylated triterpenes. In an embodiment, acylating C24, C28, C21 and C22 of a pentacyclic triterpenes, or hydroxylated triterpenes produce compounds for inhibiting cancer growth, cancer invasion, cell invasion, cancer cell invasion, molecular cell invasion, cell attachment adhesion, or cell circulation. In an embodiment, the acyl group(s) may be at C3. In an embodiment, a sugar moiety is at C21, 22, or 28, wherein the sugar moiety is attached with 2 acyl groups. In an embodiment, acylating the compounds of (A), (B), (C), (D1), (D2), (F), (G), (H), produce the compounds for inhibiting cancer invasion, cells invasion or cancer cell invasion; cancer metastasis; or cancer growth. The building blocks in the present application are used to synthesise active saponins. In embodiment, the sugar moiety(ies) is/are included but not limited to a group of glucose, galactose, rhamnose, arabinose, xylose, fucose, allose, altrose, gulose, idose, lyxose, mannose, psicose, ribose, sorbose, tagatose, talose, fructose, alduronic acid, glucuronic acid, galacturonic acid, and derivatives or combinations thereof;

Acylating the compound (G) with angeloyl or tigloyl group gives the following compounds

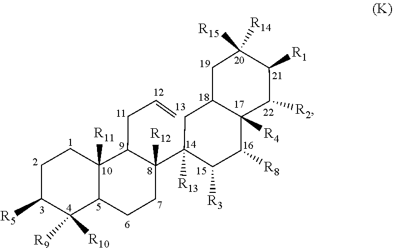

wherein R1, R2, R5, R8 represent OH or O-angeloyl; R3 represents OH, H or O-angeloyl; R4, R10 represent CH3, CH2OH or CH2Oangeloyl; R3 represents OH, H or O-angeloyl; R9, R11, R12, R13, R14, R15 represent CH3; or wherein R1, R2, R5, R8 represent OH or O-tigloyl; R3 represents OH, H or O-tigloyl; R4, R10 represent CH3, CH2OH or CH2O tigloyl; R9, R11, R12, R13, R14, R15 represent CH3; wherein the compounds inhibit cancer growth, cancer invasion, cells invasion or cancer cell invasion.

Acylating the compound (G) with angeloyl, tigloyl, senecioyl, acetyl, Crotonoyl, 3,3-Dimethylartyloyl, Cinnamoyl, Pentenoyl, Hexanoyl, benzoyl, Ethylbutyryl, alkyl, dibenzoyl, benzoyl, alkanoyl, alkenoyl, benzoyl alkyl substituted O-alkanoyl, alkanoyl substituted phenyl, alkenoyl substituted phenyl, aryl, acyl, heterocylic, heteroraryl, CH2O-alkenylcarbonyl, alkane, alkene give the compound (K) wherein R1, R2, R5, R8 represent OH, O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl; R4, R10 represent CH3, CH2OH, CH2O-angeloyl, CH2O-tigloyl, CH2O-senecioyl, CH2O-acetyl, CH2O-Crotonoyl, CH2O-3,3-Dimethylartyloyl, CH2O-Cinnamoyl, CH2O-Pentenoyl, CH2O-Hexanoyl, CH2O-benzoyl, CH2O-Ethylbutyryl, CH2O-alkyl, CH2O-dibenzoyl, CH2O-benzoyl, CH2O-alkanoyl, CH2O-alkenoyl, CH2O-benzoyl alkyl substituted O-alkanoyl, CH2O-alkanoyl substituted phenyl, CH2O-alkenoyl substituted phenyl, CH2O-aryl, CH2O-acyl, CH2O-heterocylic, CH2O-heteroraryl, CH2O-alkenylcarbonyl, alkane, alkene; R3 is absent of represents OH, H, O-angeloyl, O-tigloyl, O-senecioyl, O-acetyl, O-Crotonoyl, O-3,3-Dimethylartyloyl, O-Cinnamoyl, O-Pentenoyl, O-Hexanoyl, O-benzoyl, O-Ethylbutyryl, O-alkyl, O-dibenzoyl, O-benzoyl, O-alkanoyl, O-alkenoyl, O-benzoyl alkyl substituted O-alkanoyl, O-alkanoyl substituted phenyl, O-alkenoyl substituted phenyl, O-aryl, O-acyl, O-heterocylic, O-heteroraryl, O-alkenylcarbonyl; wherein R9, R11, R12, R13, R14, R15 represent CH3; wherein the compounds inhibit cancer growth, cancer invasion, cells invasion or cancer cell invasion; wherein the compound for use as mediator or inhibitor of adhesion protein or angiopoietin; wherein the compounds use as mediator modulating the secretion, expression, or synthesis of adhesion protein comprises reducing the fibronectin for inhibiting cell attachment, cell adhesion or cell circulation; wherein the adhesion proteins comprise fibronectin, integrins family, myosin, vitronectin, collagen, laminin, polyglycans, cadherin, heparin, tenascin, CD₅₄, and CAM; the compounds use for anti adhesion therapy and targeting adhesion molecules for therapy.

Applicant further states that anti-adhesion therapy and targeting adhesion molecules for therapy is a new direction for development of drugs. Some examples of anti-adhesion drugs in clinical trials are Efalizumab, Odulimomab, Alicaforsen, Aselizumab etc, which target varies adhesion proteins. Please see TEXT BOOK, Adhesion Molecules: Function and Inhibition, (Reference 2), edited by Klaus Ley page 289-291, 297.

Adhesion molecules in inflammatory disease, (Reference 4), Abstract, line 7-8 “Blockade of the function of expression of CAM has emerged as a new therapeutic target in inflammatory diseases”. Applicants' invention is an anti-adhesion therapy which is a new use of the compound as a mediator or inhibitor of adhesion proteins and angiopoietins. It inhibits excess adhesion and inhibits cell attachment.

In the present application, Applicants have used compounds selected from structure (2A) for anti adhesion therapy, as a mediator or inhibitor of adhesion proteins and angiopoietins, and modulation of the cell attachment, and cell adhesion.

This invention provide a synthetic method to obtain semi-natural compounds by chemically removing functional groups of complex natural products to the basic core structure before de-novo chemically adding on active groups directly or sequentially by reaction with the active group donating chemical under different reaction temperature and time to produce series of different active group modified core structure compounds that can be fractionated and easily structurally determined as well as screening for different bio-active efficacies and toxicities as potential new drug candidates.

The activities of compounds are tested with cancer of leukemia (CCRF-CEM, HL60(TB), K-562, MOLT-4, RPM18226, SR), lung(A549/ATCC, HOP-62, HOP92, NCI-H226, NCI-H322M, NCI-H460, colon (COLO205, HCC-2998, HCT-116, HCT-15, HT29, KM12, SW-620), CNS (SF-268, SF295, SF539, SNB-19, SNB-75, US51), melanoma (LOX IMVI, MALME-3M, M14, MDA-MB-3M, M14, MDA-MB-435, SK-MEL-2, SK-MEL-28, SK-MEL-5, UACC-257, UACC-62), ovary (ICTOV1, OVCAR-3, OVCAR-4, OVCAR-5, OVCAR-8, NCIADR-RES, SKOV3), renal (786-0, A498, ACHN, CAKI-1, SN12C, TK-10, UO-31), prostate (PC-3, DU-145), breast (MCF7, MDA-MB-231, HS578T, T47D, MDA-MB-468). The room temperature is 25 C in the present application.

Experimental Details

Experiment 1: Removal of the Sugar Moiety from Saponin by Acid Hydrolysis

15 mg saponin was dissolved in 1 ml of Methanol. 1 ml of 2N HCl was then added. The mixture was refluxed in 80 C water bath for 5 hours. The solution was then neutralized by adding 2 ml of 1N NaOH (to final pH 4-6). The aglycone was then extracted with ethylacetate 3 ml×2. The extracts were collected and pooled. Further isolation of aglycone (sugar-removed saponin) was achieved by HPLC with isocratic elution of 80-100% acetonitrile.

Experiment 2: Removal of the Acyl Group by Alkaline Hydrolysis

Methods: 20 mg of saponin was dissolved in 0.5 ml of 1N NaOH. The solution was incubated in 80C water bath for 4 hours. It was cooled to room temperature before neutralized with 0.5 ml 1N HCl (adjust pH to about 3). The mixture was extracted with 2 ml 1-butanol 3 times. The butanol fractions were collected and lyophilized. The hydrolyzed saponin with further purified with HPLC in a C-18 column eluted with 25% acetonitrile.

Experiment 3: Adding the Acyl Group to Triterpene by Esterification

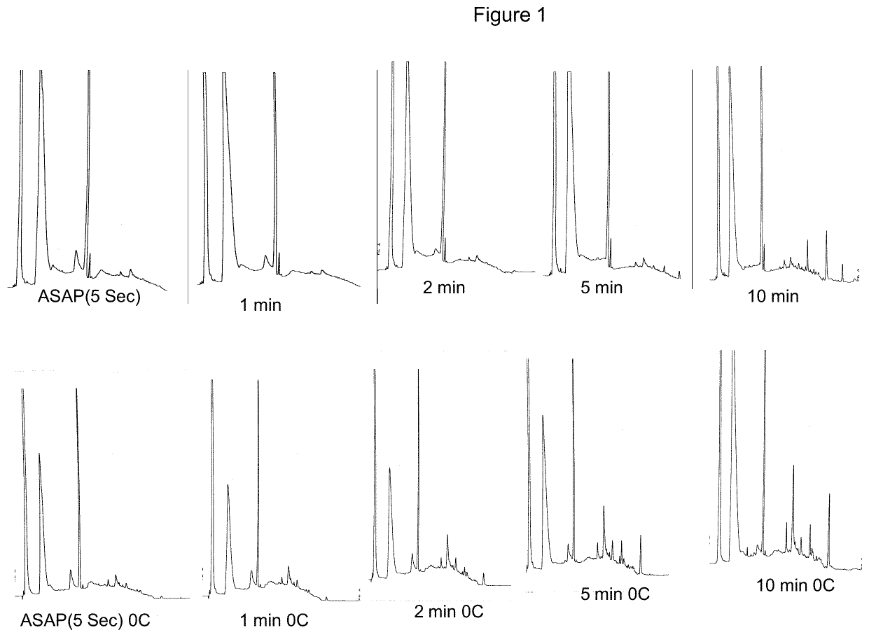

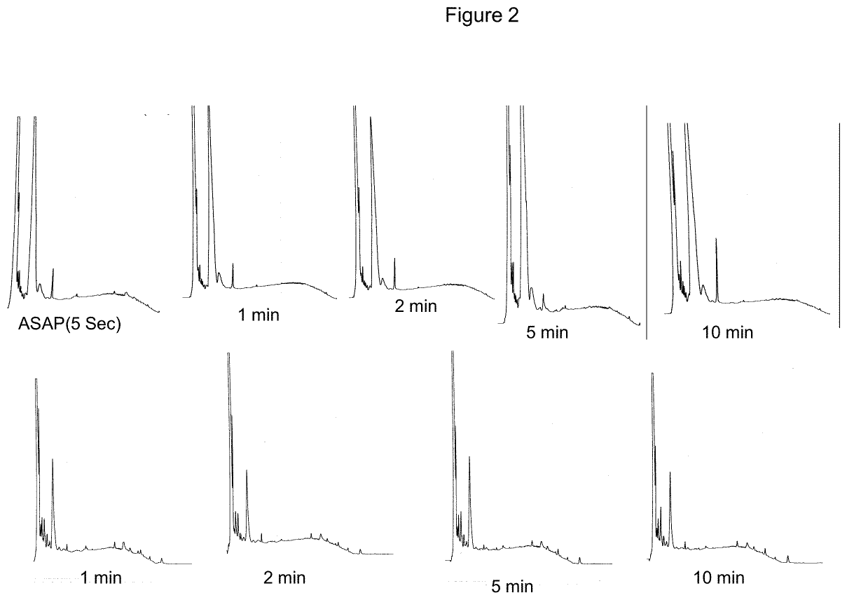

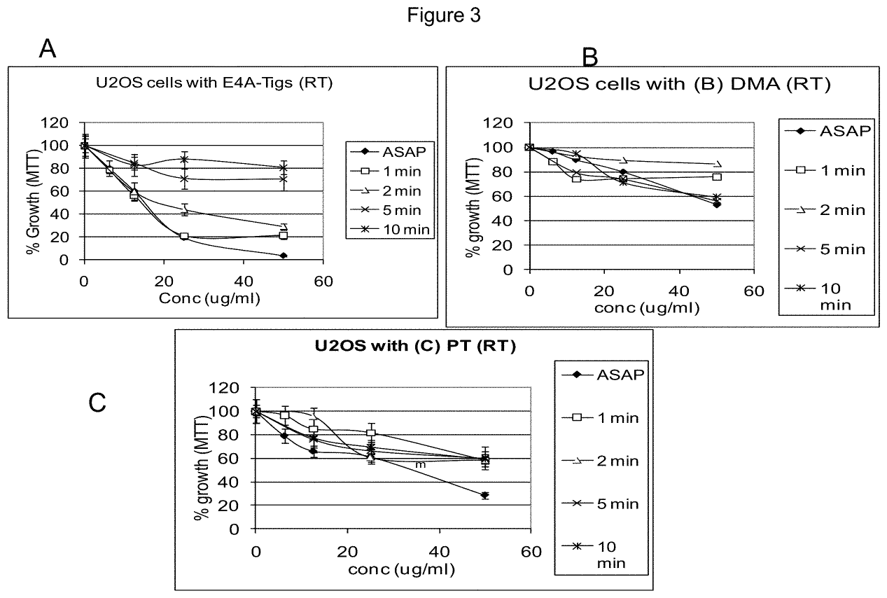

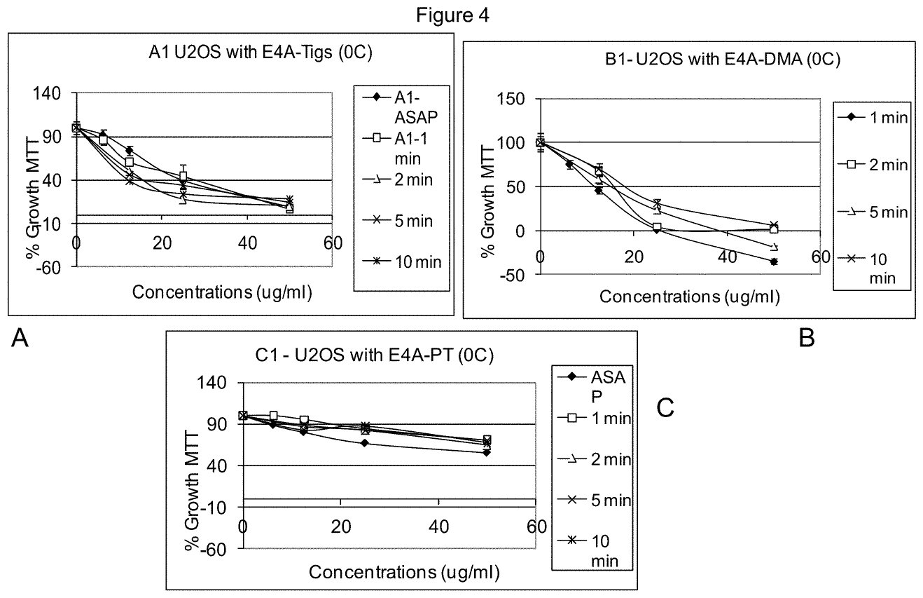

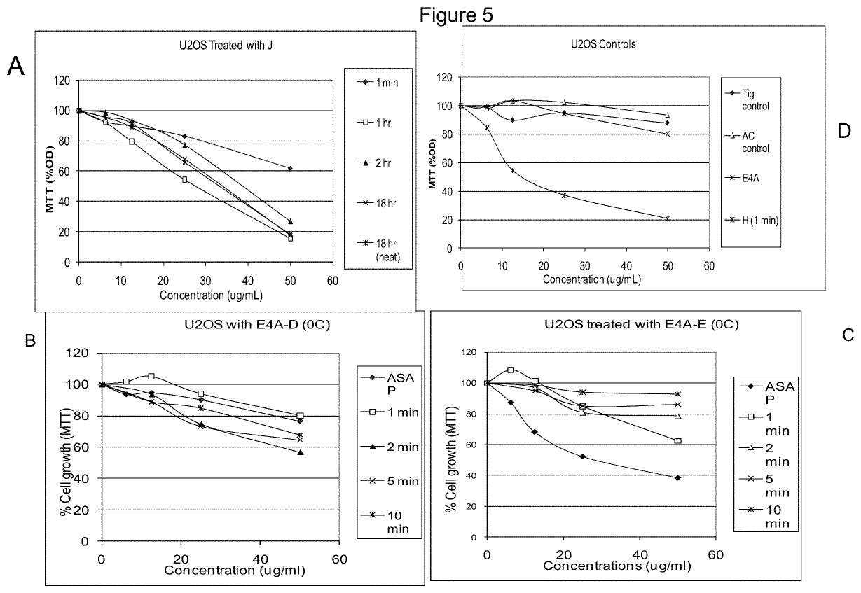

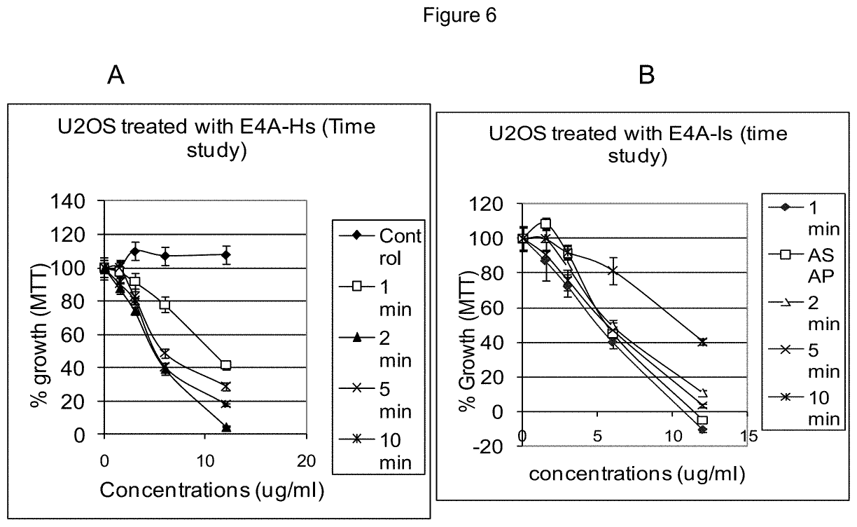

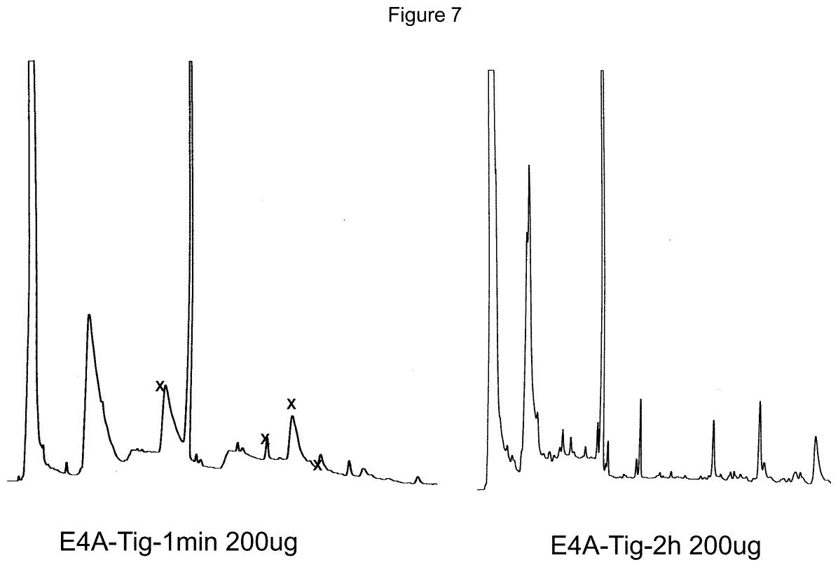

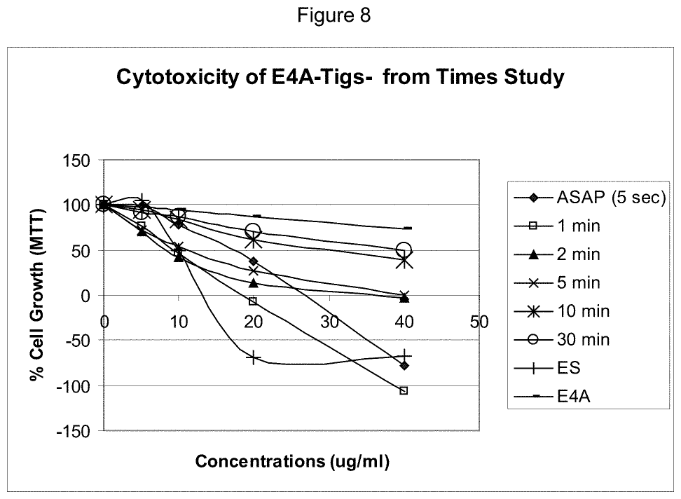

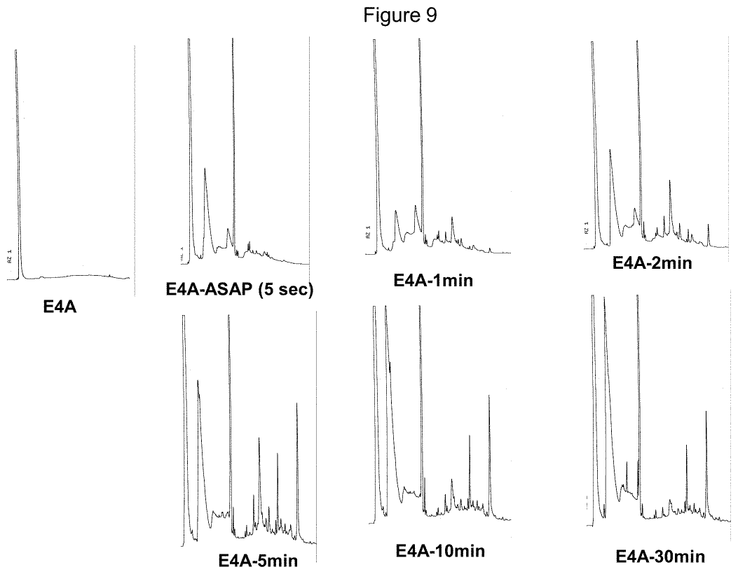

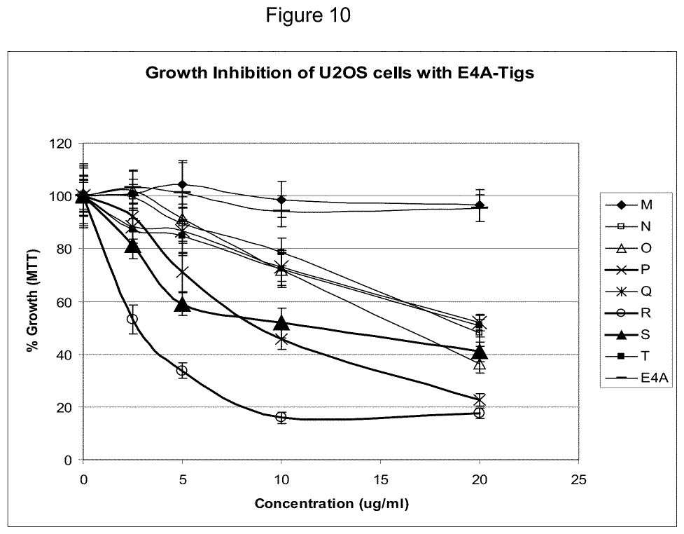

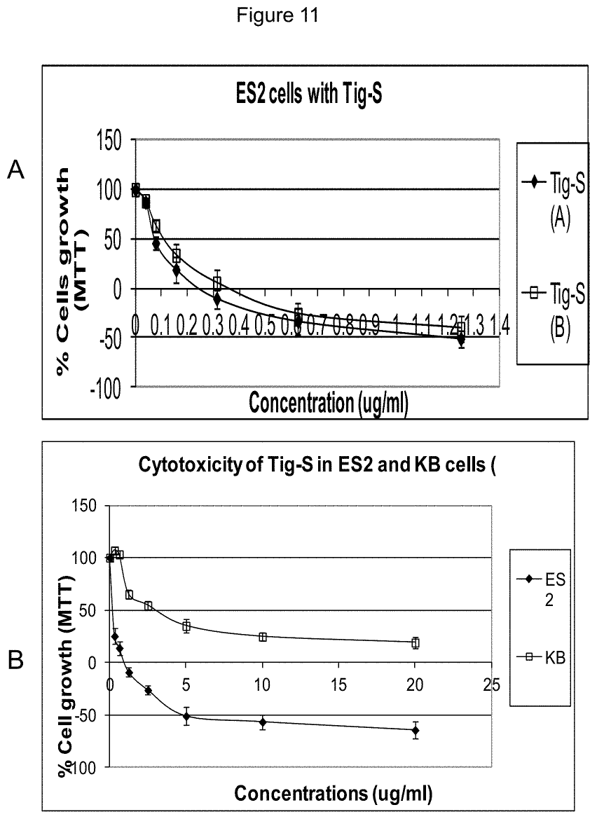

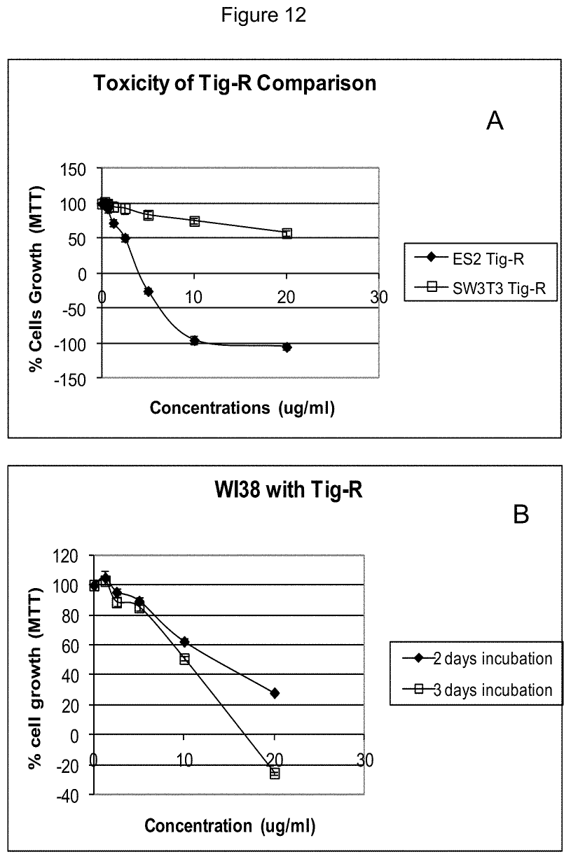

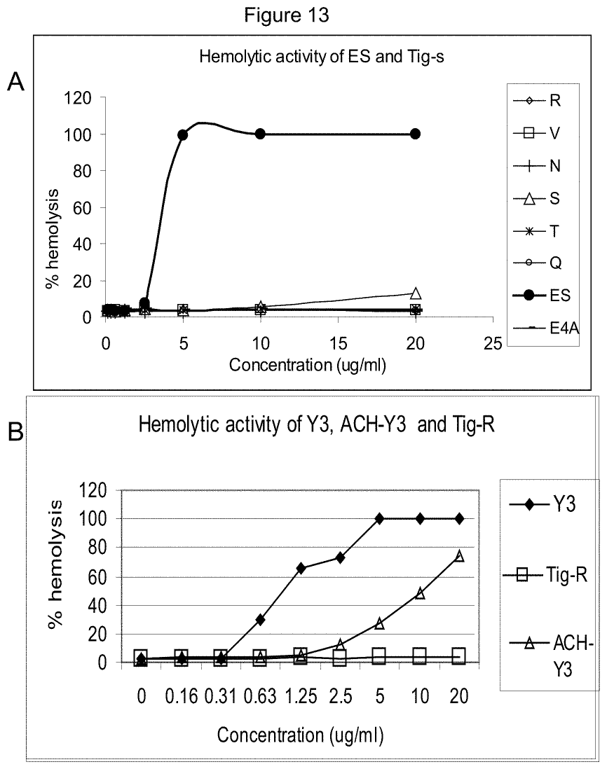

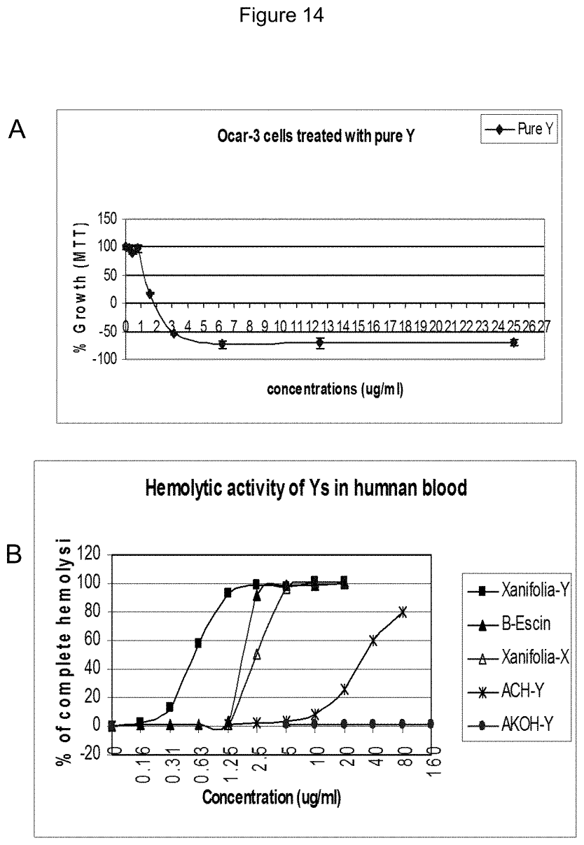

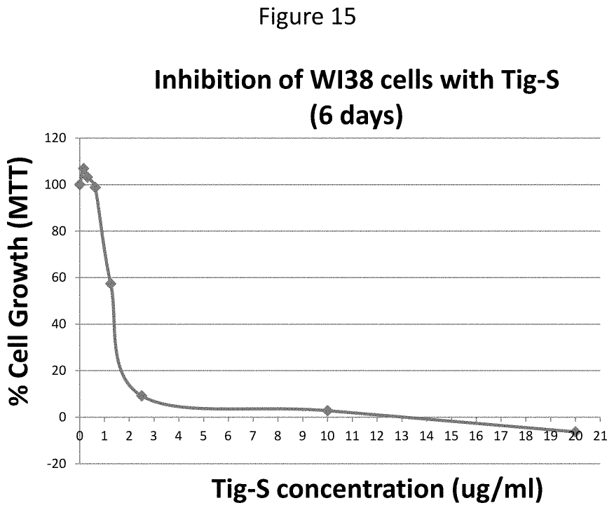

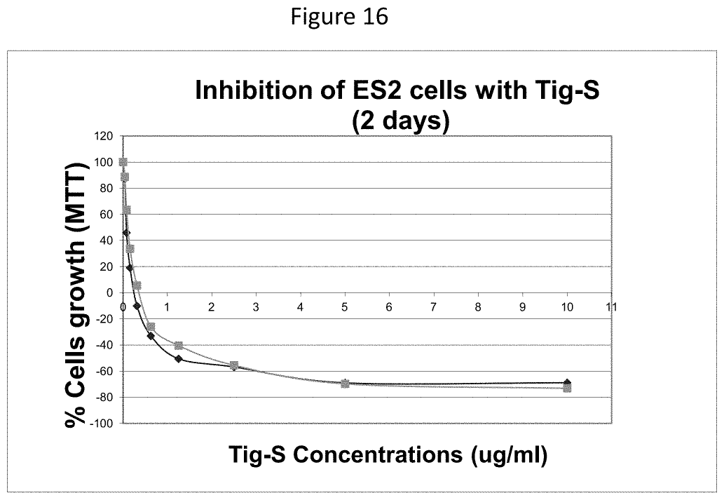

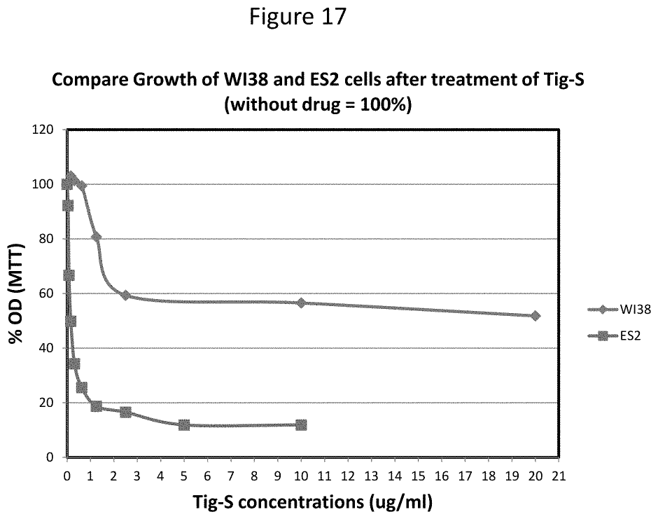

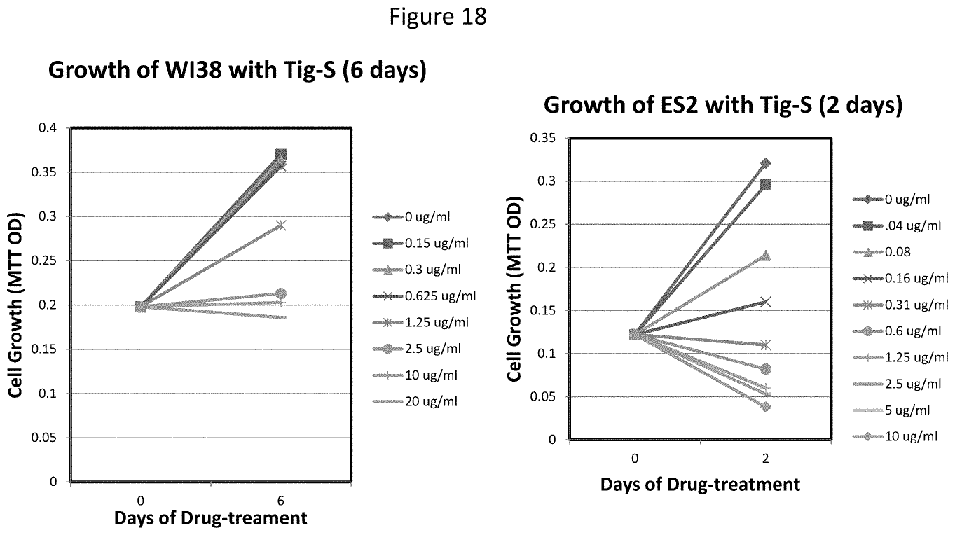

Method: 40 mg of triterpene core (fraction IV) was dissolved in 1 ml pyridine in a 50 ml tube. Reaction is started by adding 0.2 ml of acyl chloride (Tigloyl chloride, angeloyl chloride, Acetyl chloride, Crotonoyl chloride, 3,3-Dimethylartyloyl chloride(senecioyl chloride), Cinnamoyl chloride, Pentenoyl chloride, Hexanoyl chloride, benzoyl chloride or Ethylbutyryl chloride). The mixture is stirred for 5 sec, 1 min, 2 min, 5 min, 10 min, 30 min, 1 hr, 2 hr, 18 hr, 2 days or 3 days at 0 C, 25 C or 75 C temperature. At the end of reaction, 5 ml of 2N HCl or 1M NaHCO3 is added to the reaction mixture. The solution is then extracted 3 times with 10 ml of ethyl acetate which is then evaporated under vacuum and at 45 C and lyophilization. The reaction product is dissolved in 80% acetonitrile-0.005% Trifluoroacetic acid or DMSO; and was separated with HPLC. Selecting the HPLC fractions for isolation is according to the cytotoxic activity of the reaction product obtained at a specific reaction time. The active esterification products are purified with HPLC. The reaction product of mixtures and individual compounds are tested with MTT cytotoxic assay. Structures are determined with NMR. See examples FIGS. 1-12 (U.S. Ser. No. 14/313,080)

Experiment 4: Preparation of E4A
1. Beta-Escin dissolved in 1M NaOH (20 mg/ml) was incubated at 70 C for 5 hours.
2. The hydrolyzed solution was neutralized with HCl and the water was evaporated by lyophilization.
3. The product was dissolved in 50% methanol and 1N HCl. The mixture was incubated at 70 C for 5 hours.
4. The solution was neutralized with NaOH.
5. The hydrolyzed product was extracted with ethylacetate, which was subsequently removed by evaporation.
6. Further purification of the hydrolyzed product (E4A) was archived with FPLC chromatography in a C18 column equilibrated with 70% acetonitrile/TFA at the flow rate of 1 ml/min.

Experiment 5: Esterification of E4A with Tigloyl Chloride
1. 50 mg of E4A in 1 ml pyridine, stir gently in a 50 ml tube. Esterification was carried out at 25 C by adding 200 ul Tigloyl chloride.
2. Stir for 1 minute; then immediately add 5 ml of 2N HCl.
3. Stir for 1 hour and sit at room-Temp over night.
4. Extract the esterification products with 10 ml ethylacetate.
5. Evaporate the ethylacetate.
6. Dissolve the sample with 1 ml DMSO.
7. Fractionate the reaction products with HPLC.
8. Collect samples.

Experiment 6: Isolation of E4A-Tig Active Compounds with HPLC
1. Column: ZORBAX ODS 9.4×250 mm, 5 um
2. Solvents: A: 45% AN/TFA; B: 100% AN/TFA
3. Chromatography conditions: a) Elution: Solvent A to B in 80 min; then with solvent B for 40 min; b) flow rate: 1 ml/mim. c) Monitor OD: at 207 nm;

Experiment 7: MTT Experiment

Cells. HTB-9 (bladder), HeLa-S3 (cervix), DU145 (prostate), H460 (lung), MCF-7 (breast), K562 (leukemia), HCT116 (colon), HepG2 (liver), U2OS (bone), T98G (brain), SK-MEL-5 (Skin) and OVCAR 3, ES2 (ovary), Pancreas (Capan), Mouth (KB), Kidney (A498).

MTT Assay. The procedure for MTT assay followed the method described by Carmichael et al. (1987) with modifications. The cells were seeded into a 96-well plate at for 24 hours before drug-treatment. The cells were then exposed to the drugs for 48, 72, or 96 hours. After the drug-treatment, MTT (0.5 mg/mL) was added to cultures and incubated for an hour. The formazan (product of the reduction of tetrazolium by viable cells) formed and was dissolved with DMSO and the O.D. at 490 nm, and was measured by an ELISA reader. The MTT level of the cells before drug-treatment was also measured (T0). The % cell-growth (% G) is calculated as: % G=(TD−T0/TC−T0)×100(1), where TC or TD represents O.D. readings of control or drug-treated cells.

When T0>TD, then the cytotoxicity (LC) expressed as % of the control is calculated as: % LC=(TD−T0/T0)×100(2).

MTT Assay is performed to intermediate and final products from experiments.

Experiment 8: Chemical Synthesis, Isolation and Characterization of E4A-Tig-R

Chemical synthesis of E4A-Tig-R: 1. Preparation of E4A; 2. Esterification of E4A with Tigloyl Chloride; 3. Isolation of E4A-Tig-R with HPLC

Cytotoxic activity determination: 1. MTT assay

Chemical structure determination: 1. NMR analysis; 2. Mass Spectrum analysis

Compound E4A-Tig-R: 24,28-O-Tigloyl-3β,16α,21β,22α,24β,28-hexahydroxyolean-12-ene

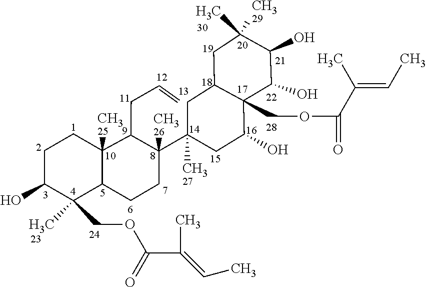

Experiment 9: Chemical Synthesis, Isolation and Characterization of E4A-Tig-N

Chemical synthesis of E4A-Tig-N: 1. Preparation of E4A; 2. Esterification of E4A with Tigloyl Chloride; 3. Isolation of E4A-Tig-N with HPLC

Cytotoxic activity determination: 1. MTT assay

Chemical structure determination: 1. NMR analysis; 2. Mass Spectrum analysis

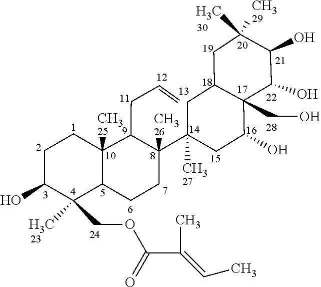

Experiment 10: Chemical Synthesis, Isolation and Characterization of E4A-Tig-Q

Chemical synthesis of E4A-Tig-Q: 1. Preparation of E4A; 2. Esterification of E4A with Tigloyl Chloride; 3. Isolation of E4A-Tig-Q with HPLC

Cytotoxic activity determination: 1. MTT assay

Chemical structure determination: 1. NMR analysis; 2. Mass Spectrum analysis

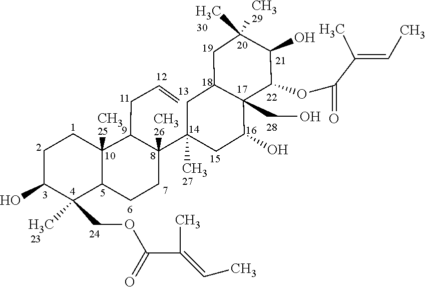

Experiment 11: Chemical Synthesis, Isolation and Characterization of E4A-Tig-V

Chemical synthesis of E4A-Tig-V: 1. Preparation of E4A; 2. Esterification of E4A with Tigloyl Chloride; 3. Isolation of E4A-Tig-V with HPLC

Cytotoxic activity determination: 1. MTT assay

Chemical structure determination: 1. NMR analysis; 2. Mass Spectrum analysis

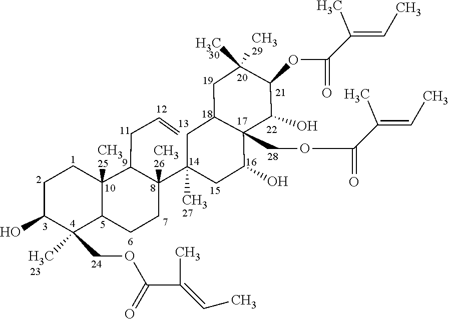

Experiment 12: Chemical Synthesis, Isolation and Characterization of E4A-Tig-T

Chemical synthesis of E4A-Tig-T: 1. Preparation of E4A; 2. Esterification of E4A with Tigloyl Chloride; 3. Isolation of E4A-Tig-T with HPLC

Cytotoxic activity determination: 1. MTT assay

Chemical structure determination: 1. NMR analysis; 2. Mass Spectrum analysis

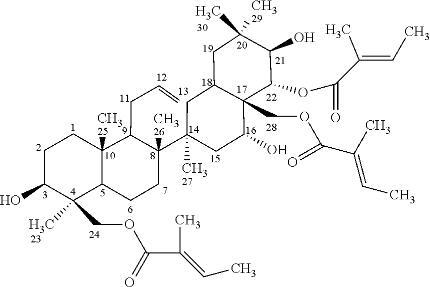

Experiment 13: Chemical Synthesis, Isolation and Characterization of E4A-Tig-U

Chemical synthesis of E4A-Tig-U: 1. Preparation of E4A; 2. Esterification of E4A with Tigloyl Chloride; 3. Isolation of E4A-Tig-U with HPLC

Cytotoxic activity determination: 1. MTT assay

Chemical structure determination: 1. NMR analysis; 2. Mass Spectrum analysis

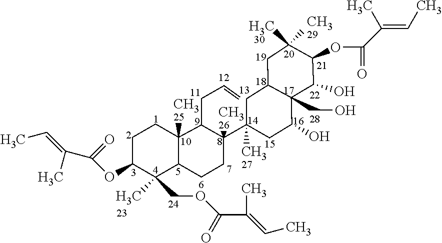

Experiment 14: Chemical Synthesis, Isolation and Characterization of E4A-Tig-S

Chemical synthesis of E4A-Tig-S: 1. Preparation of E4A; 2. Esterification of E4A with Tigloyl Chloride; 3. Isolation of E4A-Tig-S with HPLC

Cytotoxic activity determination: 1. MTT assay

Chemical structure determination: 1. NMR analysis; 2. Mass Spectrum analysis

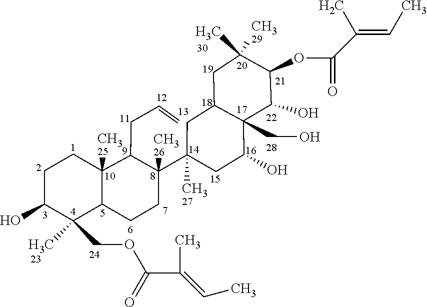

Experiment 15: Using method in Experiment 3, esterification of E4A with acetyl, angeloyl, tigloyl, senecioyl, Crotonoyl, Cinnamoyl, Pentenoyl gave the following compounds:

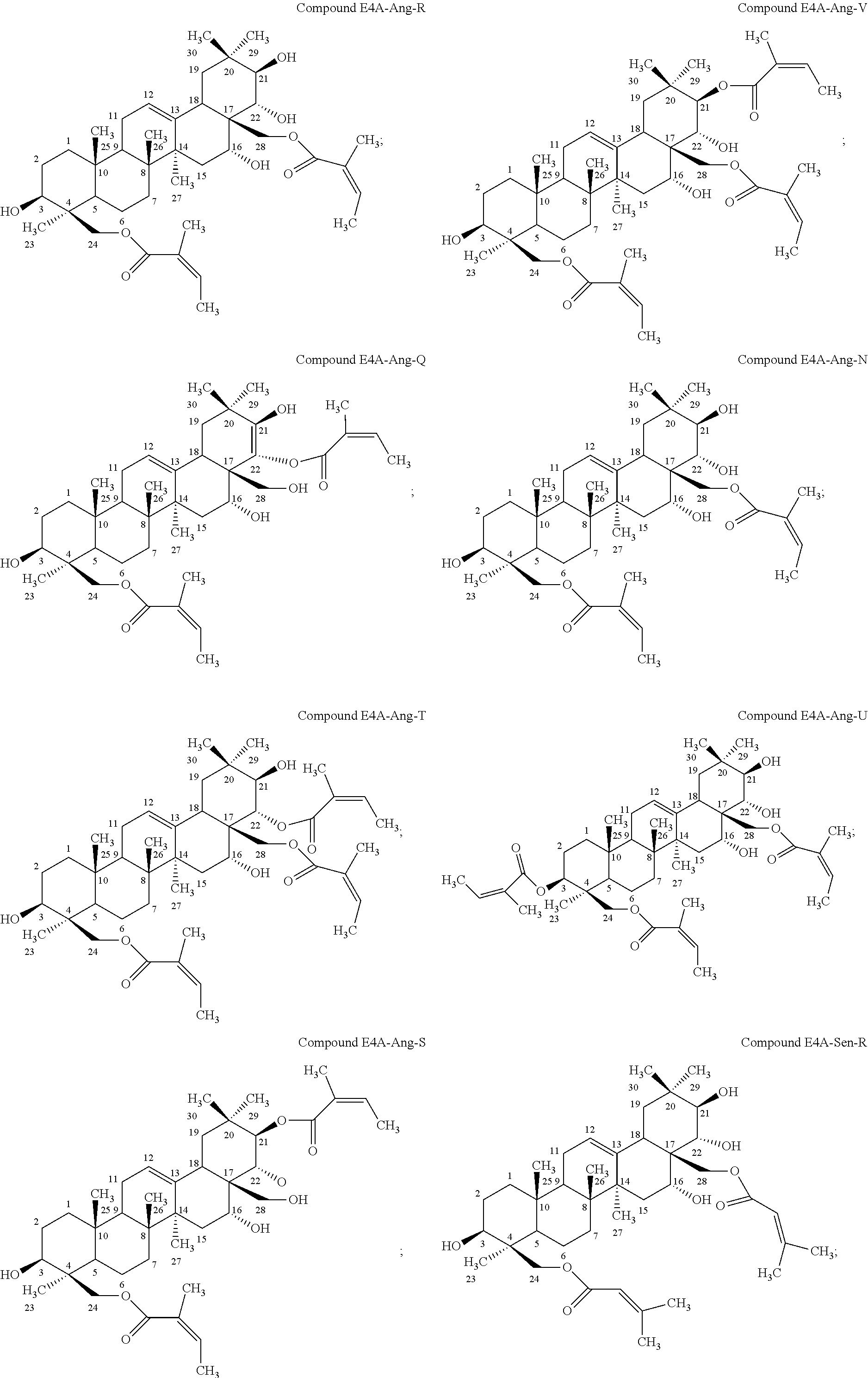

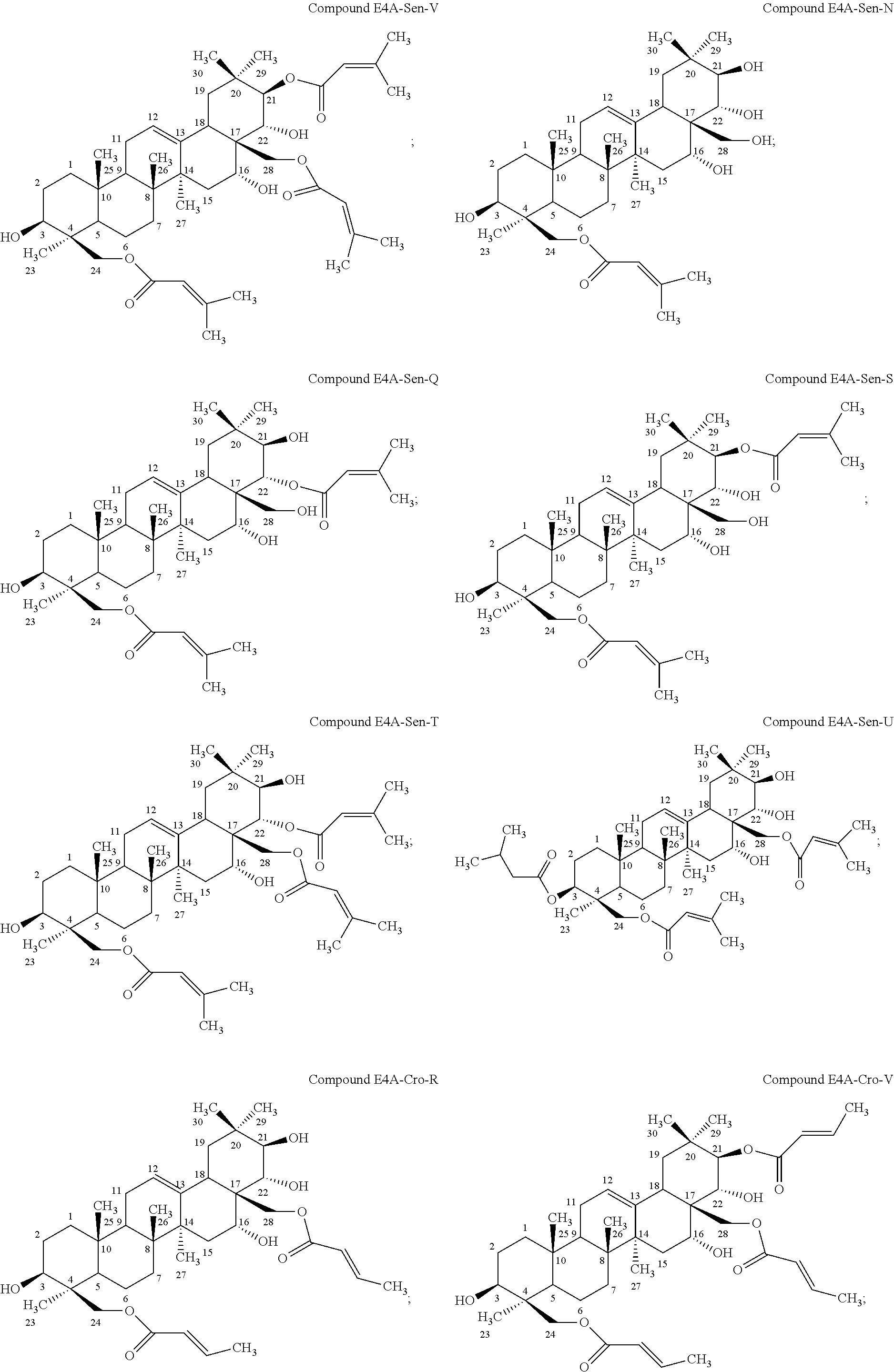

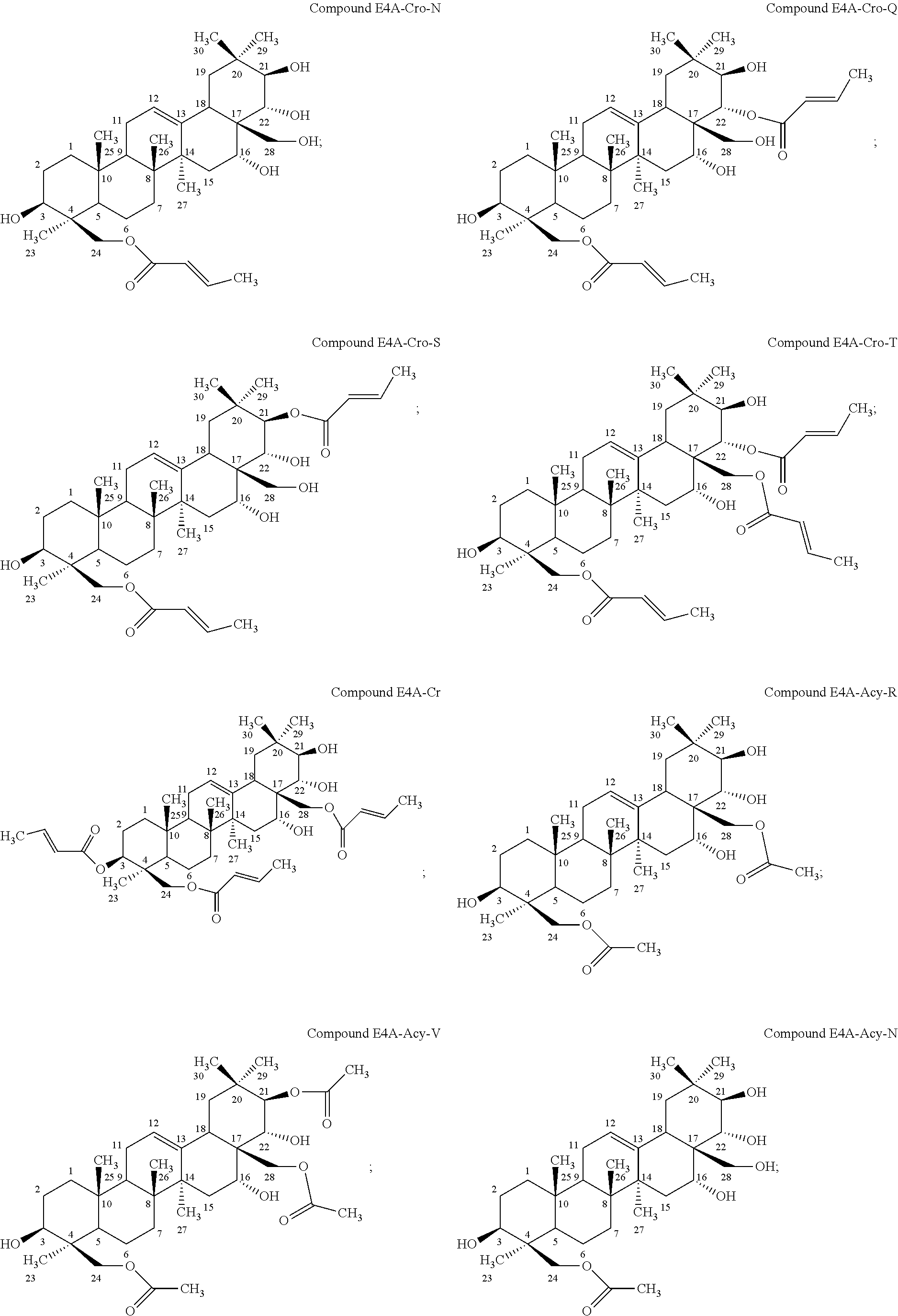

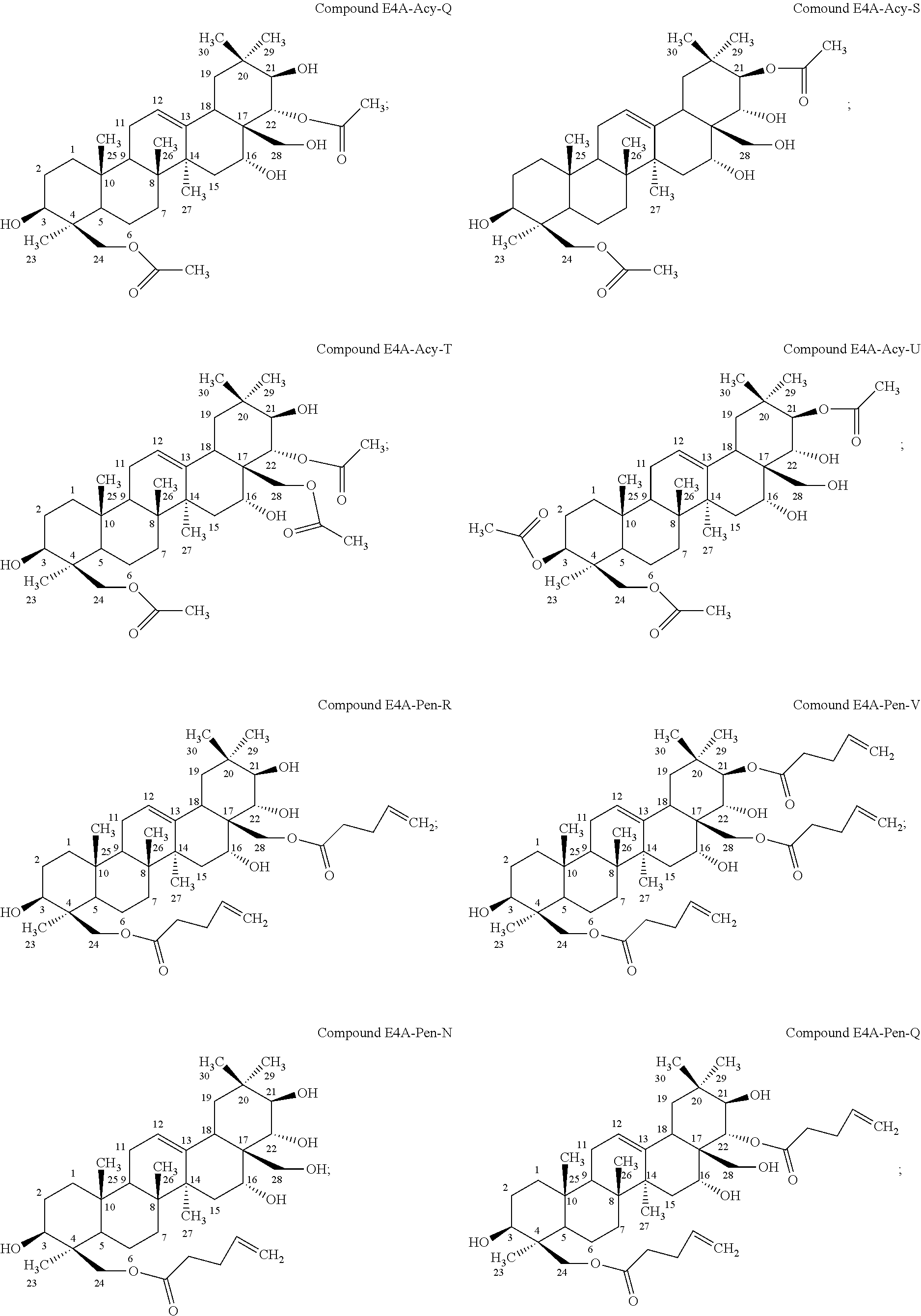

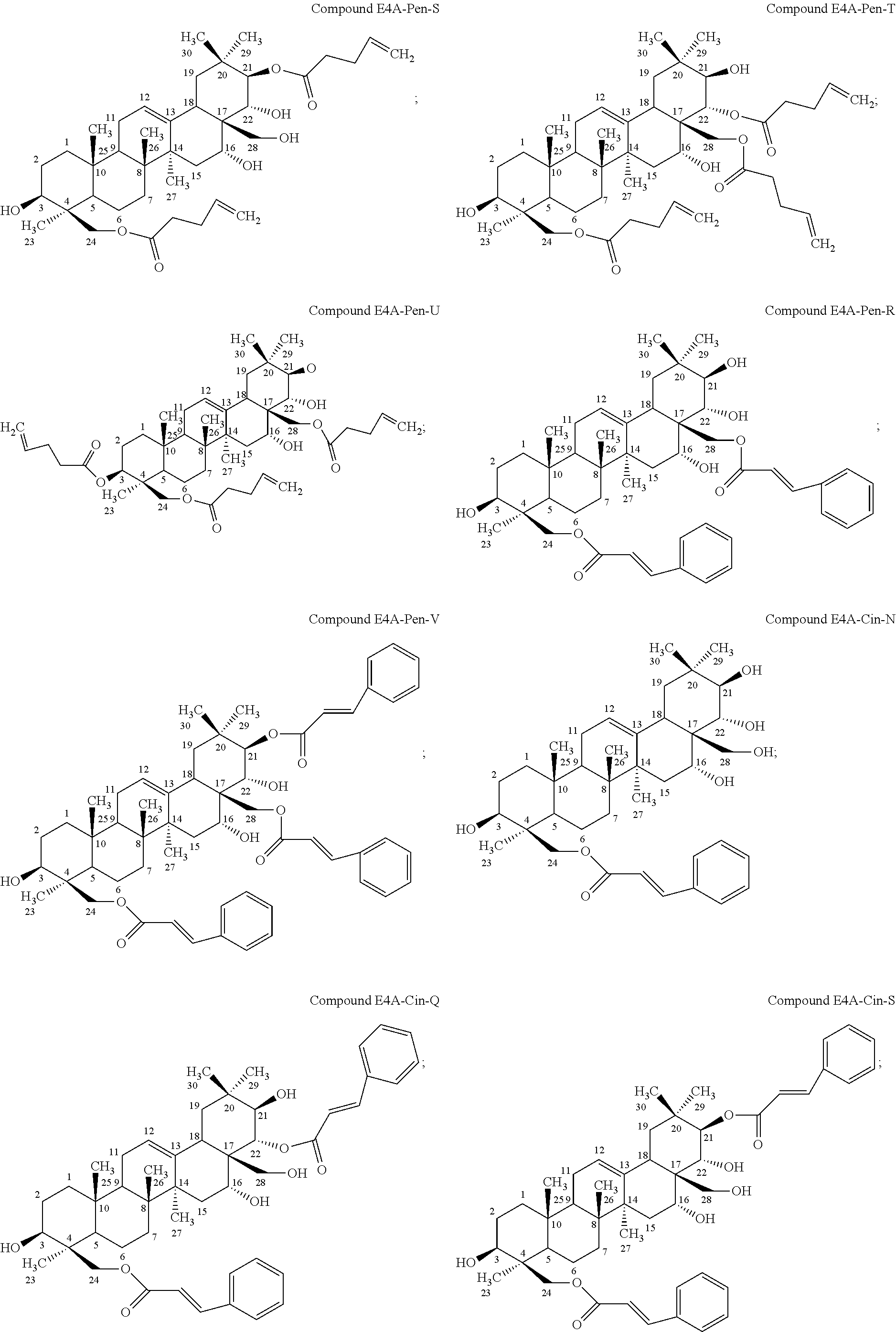

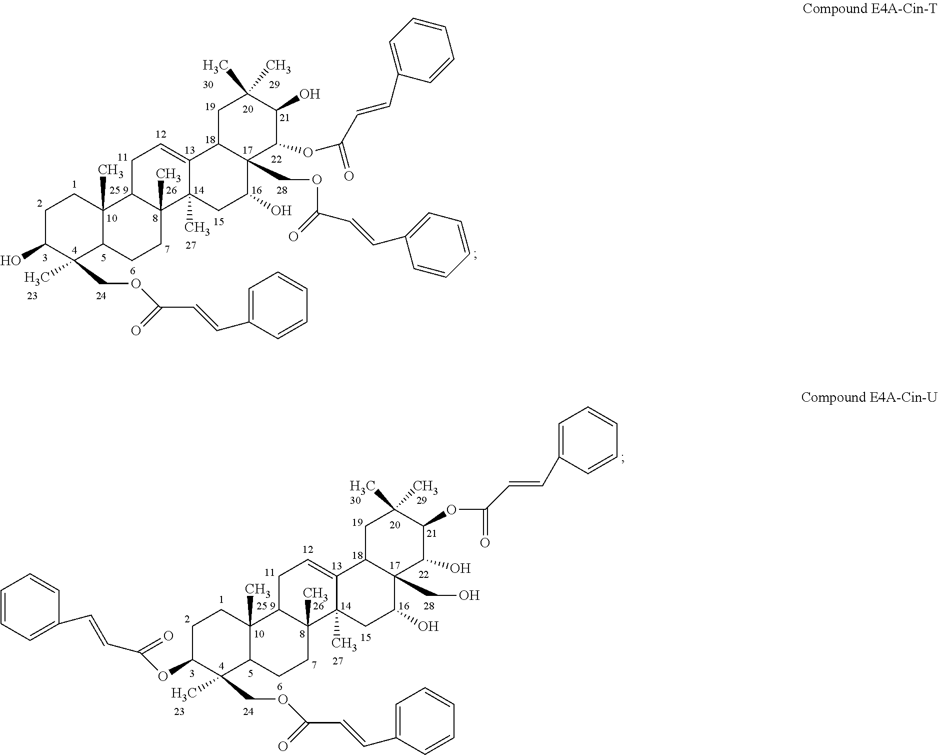

Experiment 16: Esterification of E4A-Tig-N with Senecioyl Chloride

Chemical synthesis of E4A-Tig-Sen-1: 1. Esterification of E4A-Tig-N with Senecioyl Chloride; 3. Isolation of E4A-Tig-Sen-1 with HPLC

Cytotoxic activity determination: 1. MTT assay

Chemical structure determination: 1. NMR analysis; 2. Mass Spectrum analysis

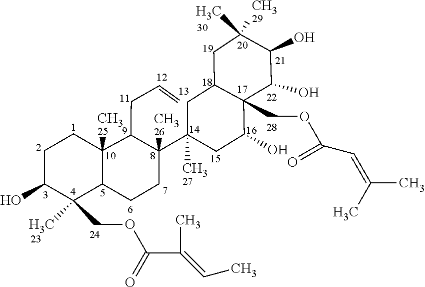

Experiment 17: Esterification of E4A-Tig-N with angeloyl chloride, Acetyl chloride, Crotonoyl chloride, 3,3-Dimethylartyloyl chloride, senecioyl chloride, Cinnamoyl chloride, Pentenoyl chloride, Hexanoyl chloride, Benzoyl chloride or Ethylbutyryl chloride; Isolation with HPLC; Cytotoxic activity determination; Chemical structure determination with the method of Experiment 8, gave the Following Compounds:

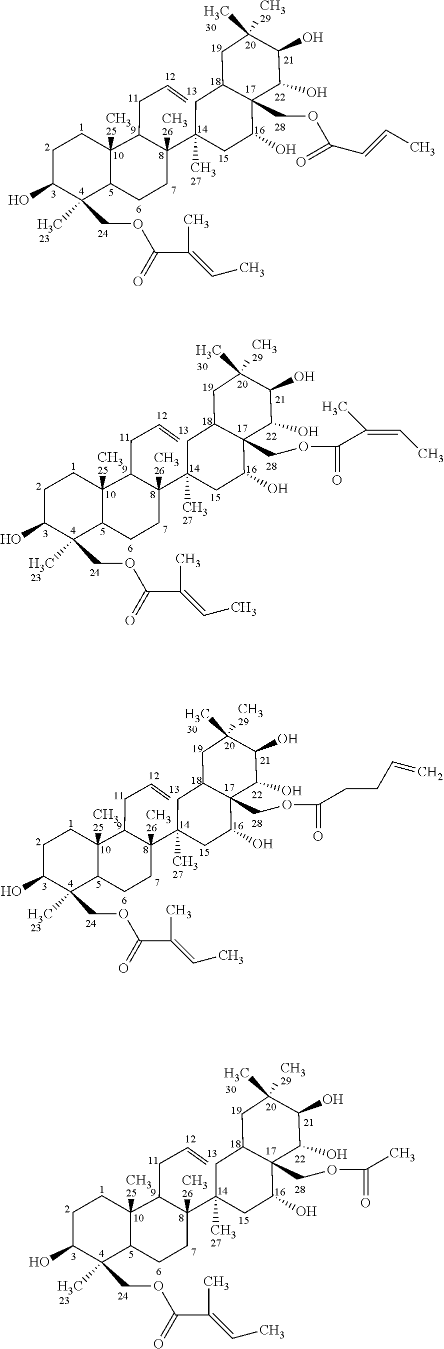

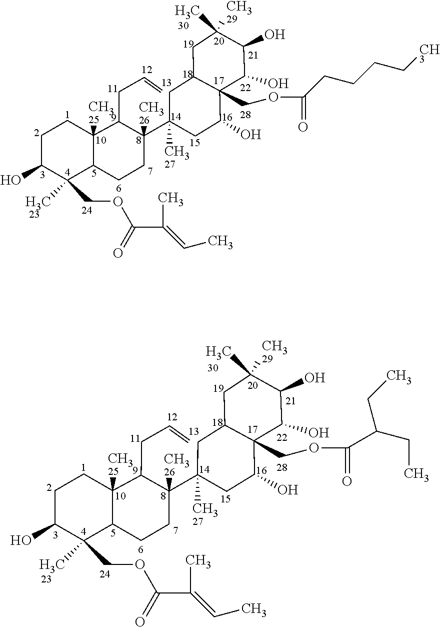

Experiment 20: Esterification of E4A with Propionyl Chloride

Methods: 50 mg of E4A in 1 ml pyridine, stir gently in a 50 ml tube. Esterification was carried out at 25 C by adding 200 ul Propionyl chloride, and immediately withdrawn 200 ul from the mixture and added to 1 ml of 2N HCl. (ASAP sample). At 1, 2, 5, 10 and 60 minutes afterward; 200 ul of reaction mixture was similarly withdrawn and add to 1 ml of 2N HCl. Mixtures were sit at room-Temp over night. Extract the esterification products with 2 ml ethylacetate. Evaporate the ethylacetate. Dissolve the sample with DMSO (final concentration of 40 mg/ml). Fractionate the reaction products with HPLC (C18 column, 1 ml/min).

HPLC condition: Column: C18 (9.4×250 mm, 5 um); Solvents: 80% Acetonitrile-0.005% TFA; Gradient: isocratic; Flow-rate: 1 ml/min; O.D.: 207 nm, AT=1024; Chart speed: 0.1 cm/min; Run time: 120 min; MTT assay (Cytotoxicity determination) condition: Cells: ES2 (ovarian cancer). Cell density: plate 10K cells per well over night before addition of drug. Drug incubation time: 2 days.

Experiment 21: Esterification of E4A with Isobutyryl Chloride

Methods: 52 mg of E4A in 1 ml pyridine, stir gently in a 50 ml tube. Esterification was carried out at 25° C. by adding 200 ul of isobutyryl chloride. 2 minute later, 4 ml 2N HCl was added to the reaction mixture. Mixtures were kept at room-Temp over night. Extract the esterification products with 5 ml ethyl acetate. Evaporate the ethyl acetate. Dissolve the sample with DMSO (final concentration of 40 mg/ml). Fractionate the reaction products with HPLC (C18 column).

HPLC condition: Column: C18 (9.4×250 mm, 5 um); Solvents: 80% Acetonitrile-0.005% TFA; Gradient: isocratic; Flow-rate: 1 ml/min; O.D.: 207 nm, AT=1024; Chart speed: 0.1 cm/min; Run time: 200 min.

MTT assay (Cytotoxicity determination) condition: Cells: ES2 (ovarian cancer); Cell density: plate 10K cells per well over night before addition of drug; Drug incubation time: 2 days.

Experiment 22: Esterification of E4A with 3,3-dimethylacryloly chloride from different times of esterification reaction. Reaction products obtained from each time of reaction (5 sec, 1 min, 2 min, 5 min, and 10 min) were fractionated by HPLC. The profile is plotted according to HPLC elution time and optical density of fractions. Reaction was performed at Room temperature and 0 C. Fractionate the reaction products with HPLC (C18 column). Cytotoxic activity is determined with MTT. Chemical structure determined with NMR.

Experiment 23: Esterification of E4A with Pentenoyl chloride-from different times of esterification reaction. Reaction products obtained from each time of reaction (5 sec, 1 min, 2 min, 5 min, and 10 min) were fractionated by HPLC. The profile is plotted according to HPLC elution time and optical density of fractions. Reaction was performed at Room temperature. Fractionate the reaction products with HPLC (C18 column). Cytotoxic activity is determined with MTT. Chemical structure determined with NMR.

Experiment 24: Esterification of E4A with Hexanoly chloride from different times of esterification reaction. Reaction products obtained from each time of reaction (5 sec, 1 min, 2 min, 5 min, and 10 min) were fractionated by HPLC. The profile is plotted according to HPLC elution time and optical density of fractions. Reaction was performed at 0 C. Fractionate the reaction products with HPLC (C18 column). Cytotoxic activity is determined with MTT. Chemical structure determined with NMR.

Experiment 25: Esterification of E4A with Acetyl chloride (H) from different times of esterification reaction. Reaction products obtained from each time of reaction (1 min, 2 min, 5 min and 10 min) were fractionated by HPLC. The profile is plotted according to HPLC elution time and optical density of fractions. Reaction was performed at Room temperature. Fractionate the reaction products with HPLC (C18 column). Cytotoxic activity is determined with MTT. Chemical structure determined with NMR.

Experiment 26: Esterification products of E4A with Crotonoyl chloride (I) from different times of esterification reaction. Reaction products obtained from each time of reaction (5 sec, 1 min, 2 min, 5 min and 10 min) were fractionated by HPLC. The profile is plotted according to HPLC elution time and optical density of fractions. Reaction was performed at Room temperature. Fractionate the reaction products with HPLC (C18 column). Cytotoxic activity is determined with MTT. Chemical structure determined with NMR.

Experiment 27: Esterification products of E4A with Cinnamoyl chloride (J) from different times of esterification reaction. Reaction products obtained from each time of reaction (1 min, 1hour, 2 hours, 18 hours, 18 hours(heat)) were fractionated by HPLC. The profile is plotted according to HPLC elution time and optical density of fractions. Reaction was performed at Room temperature and 75 C. Fractionate the reaction products with HPLC (C18 column). Cytotoxic activity is determined with MTT. Chemical structure determined with NMR.

Experiment 28: Esterification products of E4A with pentenoyl, hexanoyl, benzoyl, ethylbutyryl, propionyl, 2-propenoyl, isobutyryl, butyryl, (2E)-2-pentenoyl, 4-Pentenoyl, 5-hexenoyl, heptanoyl, octanoyl, nonanoyl, decanoyl, Lauroyl, myristoyl, from different times of esterification reaction. Reaction products obtained from each time of reaction were fractionated by HPLC. The profile is plotted according to HPLC elution time and optical density of fractions. Fractionate the reaction products with HPLC (C18 column). Cytotoxic activity is determined with MTT. Chemical structure determined with NMR.

Experiment 29: Esterification products of E4A with propanoyl, propenoyl, butanoyl, butenoyl, pentanoyl, hexenoyl, heptanoyl, heptenoyl, octanoyl, octenoyl, nonanoyl, nonenoyl, decanoyl, decenoyl, propionyl, 2-propenoyl, 2-butenoyl, Isobutyryl, 2-methylpropanoyl, 2-ethylbutyryl, ethylbutanoyl, 2-ethylbutanoyl, butyryl, (E)-2,3-Dimethylacryloyl, (E)-2-Methylcrotonoyl, 3-cis-Methyl-methacryloyl, 3-Methyl-2-butenoyl, 3-Methylcrotonoyl, 4-Pentenoyl, (2E)-2-pentenoyl, Caproyl, 5-Hexenoyl, Capryloyl, Lauroyl, Dodecanoyl, Myristoyl, Tetradecanoyl, Oleoyl from different times of esterification reaction. Reaction products obtained from each time of reaction were fractionated by HPLC. The profile is plotted according to HPLC elution time and optical density of fractions. Fractionate the reaction products with HPLC (C18 column). Cytotoxic activity is determined with MTT. Chemical structure determined with NMR.

Experiment 30: A Comparison of Non-Cancerous with Cancer Cells:
Method: A. Cells
WI38 is a Normal Lung fibroblast. Cells were grew in MEM medium supplemented with 10% FCS, antibiotics and glutamine. 20K cells were seeded per well (96-welled plate) for one day before drug-treatment
ES2 is a Ovary Clear cells carcinoma. Cells were grew in RPMI-1640 medium supplemented with 10% FCS, antibiotics and glutamine. 10K cells were seeded per well (96-welled plate) for one day before drug-treatment.

B. Drug Treatment:
Drug: Tig-S. stored as 1000× stock solution in DMSO.
   Drug concentration used: from 0.15-20 ug/ml.
Drug-treatment was carried out for 2 days (ES2) or 6 days (WI38). For the 6 days incubation, cells were fed with fresh medium (with drug) on day 3 and 5.

C. At the End of the Drug-Treatment, Cytotoxic Test was Performed with MTT Assay.

Conclusion:
For comparison between cancer cells (ES2) and non-cancerous cells (WI38), it is found that:
1. the IC₅₀ for ES2 cells (0.3 ug/ml) is lower than those of WI38 cells (1.5 ug/ml), and
2. The IC₁₀₀ for ES2 cells (0.15-0.3 ug/ml) is lower than those of WI38 cells (10 ug/ml)
3. With 10 ug/ml of Tig-S, about 90% of ES2 cells died while only 10% of WI38 cells died after the drug-treatment.
Based on these studies, it is concluded that the fast growing tumor cells (ES2) are more sensitive to Tig-S than the slow growing normal cells (WI38).

Experiment 31: Inhibition of Cell Cycling
1. Method: Cells: Human leukemia cells K562 cultured in RPMI1640 medium.
2. Drug: compound from present application or Tig-S (1000× stock solution in DMSO).
3. Start cells concentration: 500000/ml.
4. Cells were cultured with drug (0-20 ug/ml) for total of three days.
5. Cells were harvested by centrifugation (136×g, 6 minutes); fixed with 70% ethanol and kept in −20 C before staining.
6. Staining: Fixed cells were stained with Propidium iodide/RNase A/0.1% Triton X-100 in PBS.
7. Flow Cytometry Analysis was performed in Baylor Core Facility with a LSRII instrument.
8. Analysis: Single cell was gated and cell count—FL2-Area histogram were plotted.
9. Cell distribution in different cell-cycle phases (G0/G1, S, G2/M) was analyzed.

Results: Cells with no drug, or with 0.15 ug/ml and 0.3 ug/ml of Tig-S, have a similar (same) cell distribution in the G0/G1, S and G2/M phases of cell-cycle. With higher Tig-S concentrations, starting from 0.6 ug/ml, the cells in G2/M phase decrease. The decrease of G2/M cells correlated with higher drug concentrations (up to 20 ug/ml). These results indicate that drug-treated cells were arrested in the S-phase and unable to enter into the G2/M phase of the cell cycle.

Conclusion: The drug-effect of Tig-S on human leukemia K562 cells is arresting cells in the S-phase and blocking their entering into the G2/M phase of cell cycle. The Compound Tig-S block the DNA synthesis of cancer cell.

Experiment 32: Inhibition of H460 Cells Growth with Tig-S

Methods:

A. Cells
H460 cells are derived from a Human Lung large cell carcinoma. Cells were grew in RPMI 1640 medium supplemented with 10% FCS, antibiotics and glutamine. 5000 cells were seeded in a well (96-welled plate) for one day before drug-treatment

B. Drug Treatment:
Drug: Tig-S (stored as a 1000× stock solution in DMSO) was used.
Drug concentration used: from 0.15-20 ug/ml.
Drug-treatment was carried out for 1, 2 and 4 days. For the 4 days incubation, cells were fed with fresh medium (with drug) on day 2.

C. At the end of the drug-treatment, cytotoxic test was performed with MTT assay.

Results:
1. Tig-S inhibits H460 cells' growth with the IC50 of drug 3 ug/ml.
2. Minimum cells growth inhibition was observed beyond drug 5 ug/ml.
3. No dead cell was found at drug concentration in 20 ug/ml.

The results indicate that Tig-S inhibits the H460 cell's growth, but is not killing cells at high concentrations. Therefore, Tig-S is an effective drug for inhibition of cancer growth but has low toxicity.

Experiment 33: Inhibition of Human Leukemia K562 Cells by Tig-S

Method:
1. Cells: Human leukemia cells K562 in RPMI1640 medium.
2. Drug: Tig-S (1000× stock solution in DMSO).
3. Start cells concentration: 50×10K per ml (500000/ml).
4. Cells were cultured with or without drug for total of four days.
5. Cell number is doubled after 2 days of incubation. Fresh medium (equal volume, with or without drug) was then added to culture.
6. Cells were counted every day.

Conclusion:

Tig-S inhibits Leukemia K562 cells growth with IC50 about 0.6 ug/ml.

No grow (IC100) was observed beginning on day 2 at 2.5 ug/ml or higher.

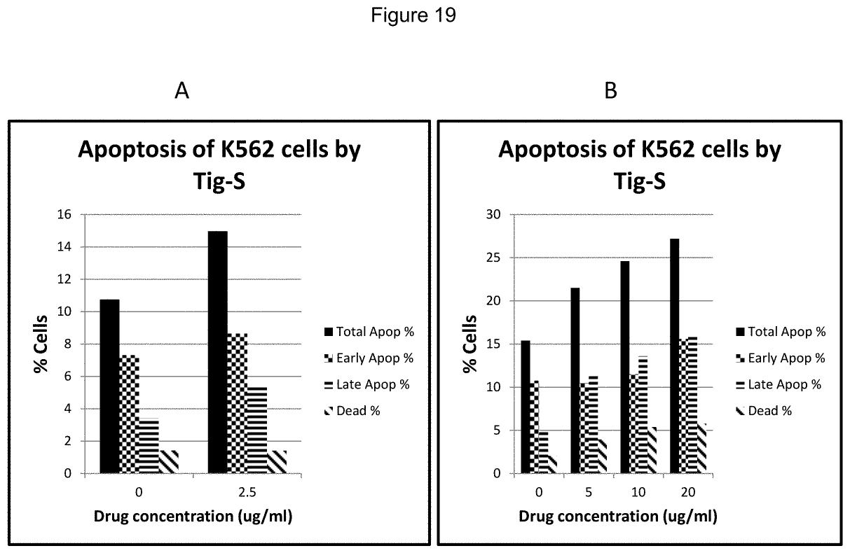

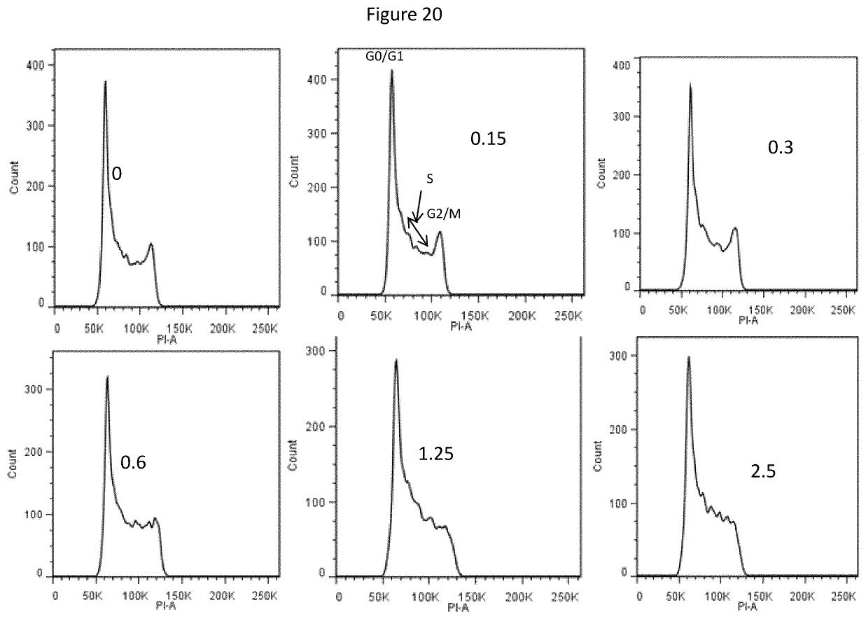

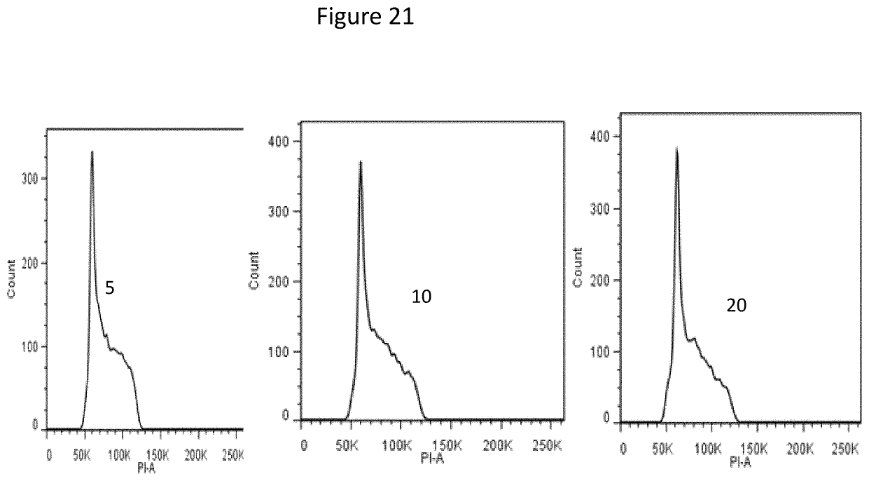

Experiment 34: Apoptosis in K562 Cells Induced by Tig-S
1. Cells: Human leukemia cells K562 cultured in RPMI1640 medium.
2. Drug: Tig-S (1000× stock solution in DMSO).
3. Start cells concentration: 500000/ml.
4. Cells were cultured with drug (0-20 ug/ml) for Two days.
5. Collect cells from culture (1-4 million) by centrifugation (136×g for 5 min. remove supernatant).
6. Wash cells with 1 ml of cold PBS, collect cells by centrifugation (136×g, 5 min, remove supernatant).
7. Re-suspend cells in 200 ul Binding buffer (10 mM HEPES, 140 mM NaCl, 2.5 mM CaCl2, pH 7.4).
8. Stained cells by adding 10 ul Annexin V (Alexa Fluor 488, Invitrogen cat #A13201), and 2 ul Propidium iodide (1 mg/ml in water) to cell suspension. Mix.
9. Sit at Room temperature for 15 min.
10. Wash cells with 1 ml binding buffer. Collect cells by centrifugation.
11. Re-suspend cells with in 1 ml of binding buffer for analysis by flow cytometry.
12. Flow Cytometry analysis was performed in Baylor Core Facility with a LSRII instrument.
13. Analysis: Control cells stained with PI or Annexin V-488 (Annexin V-FITC) alone were served as references. About 50000 cells were analyzed for each sample.
14. Cell distribution in following groups: live, early apoptosis, late-apoptosis, total apoptosis and dead cells were determined.
15. The percentage of cells in these groups is presented in the FIG. 19.

Results:

The background apoptosis level of K562 cells (no-drug control) is about 10-15%. After drug-treatment with Tig-S, the apoptotic cells population increased (from 15% to 27%) with increased drug concentration (from 2.5 ug/ml to 20 ug/ml).

Similarly, the dead cell population was also increase with the drug concentration.

Conclusion:

Tig-S induces cell-death by the apoptosis mechanism (not necrosis mechanism).

Experiment 35: The Haemolytic Assay

Erythrocytes (RBC) were isolated from human blood (EDTA whole blood, collected randomly). 50 ul of the 10% RBC suspension (in PBS) was added to 2 ml of sample solutions (concentration range from 0.1 ug/ml to 400 ug/ml) in PBS. The mixture was vortexed briefly and sat for 60 min at room temperature. The mixture was spun at 3K for 10 min and the relative amounts of lysed hemoglobin in the supernatant were measured at 540 nm. The synthetic compounds of present application were tested with this method.

Experiment 36: Animals Experiments

Methods
Athymic Nu/Nu mice are divided into two groups (A, and B).
On day 0, mice of group A and B were transplanted intra-peritoneally (i.p.) with ES2 (human ovarian cancer) cells.
On day 1 to 5, mice from A group received daily administration of 127 solvent by i.p. route.
On days 1 to 5, mice from B group received daily drug administration (tested drug Tig-S in 127 solvent) by i.p. route at dose of 100 mg/kg, twice daily.

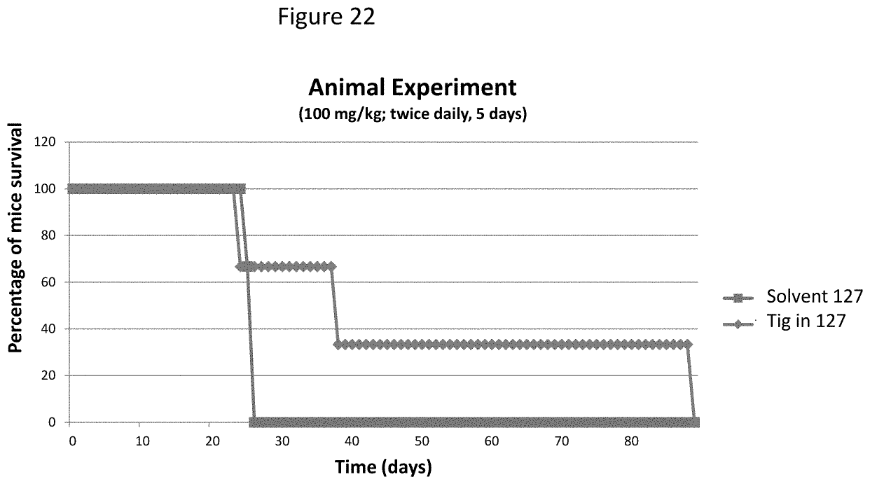

Result in FIG. 22

Experiment 37: Animals Experiments

Methods
Athymic Nu/Nu mice are divided into two groups (A, and B).
On day 0, mice of group A and B were transplanted intra-peritoneally (i.p.) with ES2 (human ovarian cancer) cells.
On day 1 to 5, mice from A group received daily administration of 127 solvent by i.p. route.
On days 1 to 5, mice from B group received daily drug administration (tested drug Tig-R in 127 solvent) by i.p. route at dose of 100 mg/kg, twice daily.

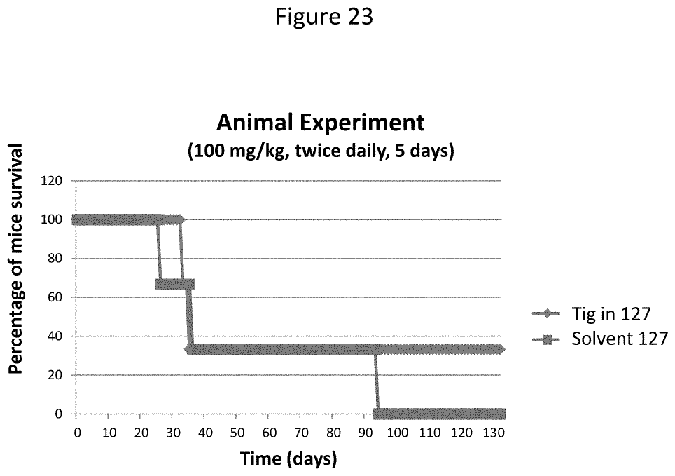

Result in FIG. 23

Experiment 38: Animals Experiments

Methods
Athymic Nu/Nu mice are divided into two groups (A, and B).
On day 0, mice of group A and B were transplanted intra-peritoneally (i.p.) with ES2 (human ovarian cancer) cells.
On day 1 to 5, mice from A group received daily administration of 127 solvent by i.p. route.
On days 1 to 10, mice from B group received daily drug administration (tested drug Tig-V in 127 solvent) by i.p. route at dose of 50 mg/kg, twice daily.

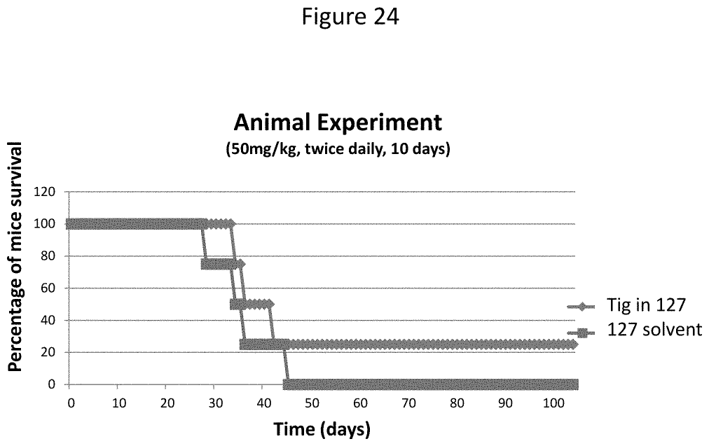

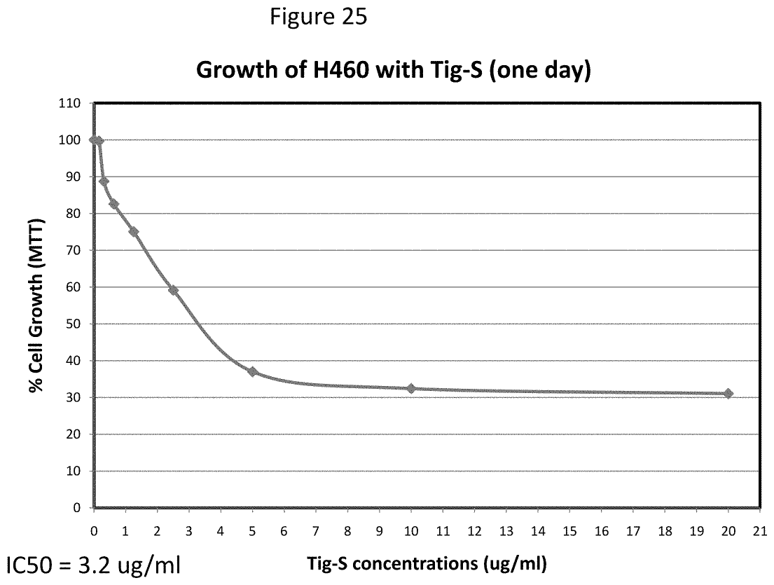

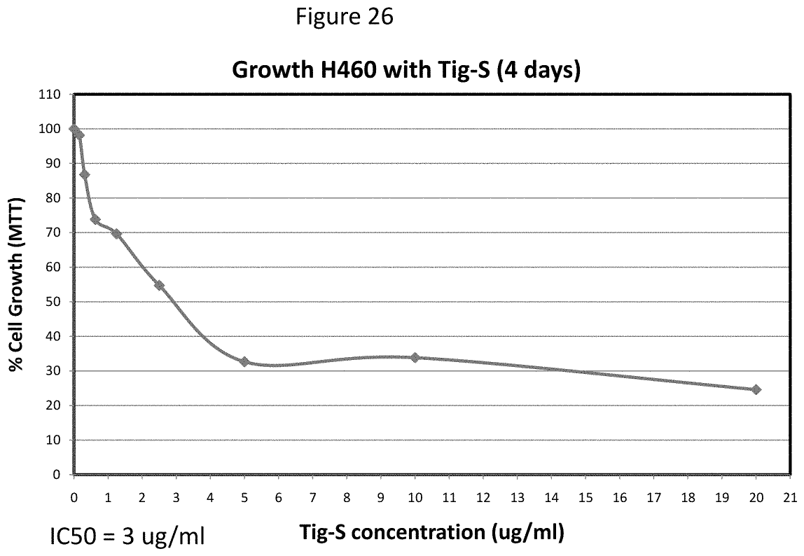

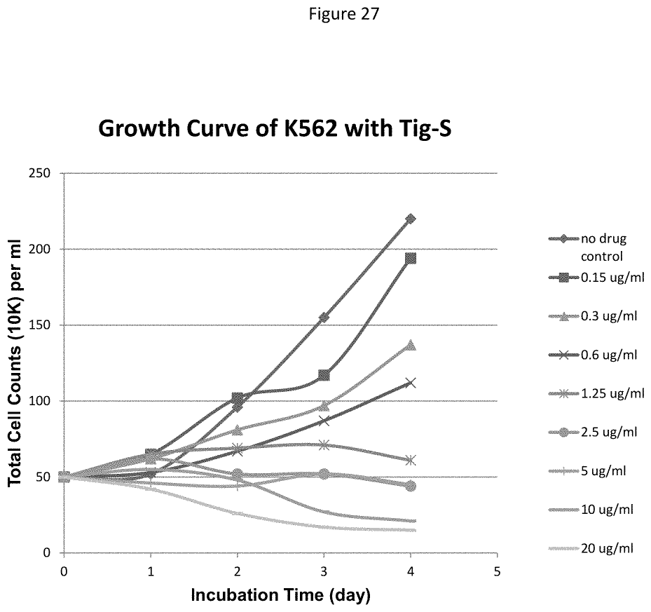

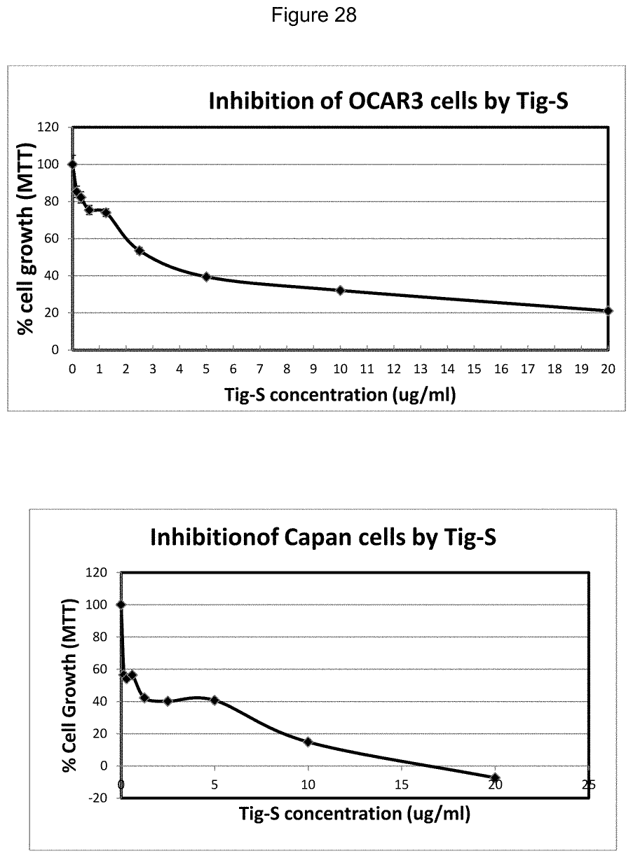

Result in FIG. 24 (U.S. Ser. No. 14/313,080)

Experiment 39: Inhibition of Capan Cells Growth by Tig-S

A. Cells: Capan cells are derived from Human pancreas carcinoma (pancreas). Cells were grew in RPMI 1640 medium supplemented with 10% FCS, antibiotics and glutamine. 10000 cells were seeded in a well (96-welled plate) for one day before drug-treatment

B. Drug Treatment:
Drug: Tig-S (stored as a 1000× stock solution in DMSO) was used.
Drug concentration used: from 0.15-20 ug/ml.
Drug-treatment was carried out for 3 days.

C. At the end of the drug-treatment, cytotoxic test was performed with MTT assay. The percentage of drug-treated cells' growth as compared to those of the no drug control is determined.

Experiment 40: Using method in Experiment 3, esterification of E4A2Y with acetyl, angeloyl, tigloyl, senecioyl, Crotonoyl, Cinnamoyl, Pentenoyl, 4-(dimethylamino)-2-methylbut-2-enoyl, and 4-[(2-methoxyethyl)amino]-2-methyl-4oxobut-2-enoyl gave the following compounds:

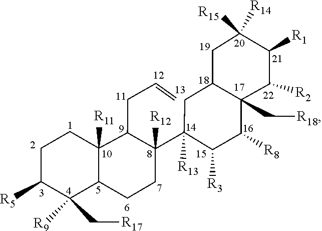

wherein:

1) R1, R2, R3, R5, R8 are OH; R17, R18 are O-acetyl;

2) R1, R2, R3, R5, R8 are OH; R17, R18 are O-angeloyl

3) R1, R2, R3, R5, R8 are OH; R17, R18 are O-tigloyl

4) R1, R2, R3, R5, R8 are OH; R17, R18 are O-senecioyl

5) R1, R2, R3, R5, R8 are OH; R17, R18 are O-Crotonoyl

6) R18, R2, R3, R5, R8 are OH; R17, R18 are O-Cinnamoyl

7) R18, R2, R3, R5, R8 are OH; R17, R18 are O-Pentenoyl

8) R18, R2, R3, R5, R8 are OH; R17, R18 are O-4-(dimethylamino)-2-methylbut-2-enoyl

9) R18, R2, R3, R5, R8 are OH; R17, R18 are O-4-[(2-methoxyethyl)amino]-2-methyl-4oxobut-2-enoyl

10) R18, R2, R3, R5, R8 are OH; R17, R1 are O-acetyl;

11) R18, R2, R3, R5, R8 are OH; R17, R1 are O-angeloyl

12) R18, R2, R3, R5, R8 are OH; R17, R1 are O-tigloyl

13) R18, R2, R3, R5, R8 are OH; R17, R1 are O-senecioyl

14) R18, R2, R3, R5, R8 are OH; R17, R1 are O-Crotonoyl

15) R18, R2, R3, R5, R8 are OH; R17, R1 are O-Cinnamoyl

16) R18, R2, R3, R5, R8 are OH; R17, R1 are O-Pentenoyl

17) R18, R2, R3, R5, R8 are OH; R17, R1 are O-4-(dimethylamino)-2-methylbut-2-enoyl

18) R18, R2, R3, R5, R8 are OH; R17, R1 are O-4-[(2-methoxyethyl)amino]-2-methyl-4oxobut-2-enoyl

19) R2, R3, R5, R8 are OH; R1, R17, R18 are O-acetyl;

20) R2, R3, R5, R8 are OH; R1, R17, R18 are O-angeloyl

21) R2, R3, R5, R8 are OH; R1, R17, R18 are O-tigloyl

22) R2, R3, R5, R8 are OH; R1, R17, R18 are O-senecioyl

23) R2, R3, R5, R8 are OH; R1, R17, R18 are O-Crotonoyl

24) R2, R3, R5, R8 are OH; R1, R17, R18 are O-Cinnamoyl

25) R2, R3, R5, R8 are OH; R1, R17, R18 are O-Pentenoyl

26) R2, R3, R5, R8 are OH; R1, R17, R18 are O-4-(dimethylamino)-2-methylbut-2-enoyl

27) R2, R3, R5, R8 are OH; R1, R17, R18 are O-4-[(2-methoxyethyl)amino]-2-methyl-4oxobut-2-enoyl

Experiment 41: Preparation of E4A(E4)
1. Beta-Escin dissolved in 1M NaOH (20 mg/ml) was incubated at 70 C for 5 hours.
2. The hydrolyzed solution was neutralized with HCl and the water was evaporated by lyophilization.
3. The product was dissolved in 50% methanol and 1N HCl. The mixture was incubated at 70 C for 5 hours.
4. The solution was neutralized with NaOH.
5. The hydrolyzed product was extracted with ethylacetate, which was subsequently removed by evaporation.
6. Further purification of the hydrolyzed product (E4A) was archived with FPLC chromatography in a C18 column equilibrated with 70% acetonitrile/TFA at the flow rate of 1 ml/min.

The product Structure E4A(E4)

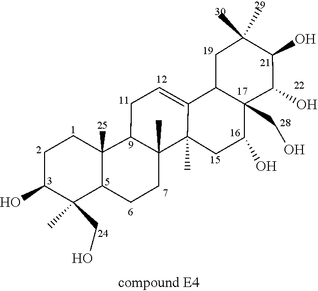

Experiment 42:

Method for the Preparation of 24,28-diamine

To a solution of compound (E4A)E4 (2 mmol) in tetrahydrofuran (THF, 10 mL) were added methanesulfonyl chloride (Ms-Cl, 2.2 mmol) and triethylamine (TEA, 3 mmol) at 0° C. and the resulting mixture was stirred for 2 hours. The reaction mixture was concentrated under reduced pressure, and the residue was dissolved in ethyl acetate (EtOAc) and washed with water. The organic layer was dried over anhydrous magnesium sulfate, filtered and concentrated under reduced pressure. The residue was dissolved in 10 mL of dimethylformamide (DMF), and then sodium azide (NaN3, 6 mmol) was added. After overnight stirring at 60° C., the reaction mixture was diluted with ethyl acetate and washed with water. The organic layer was dried over anhydrous magnesium sulfate, filtered and concentrated under reduced pressure. Then, the obtained compound was dissolved in methanol (MeOH), and 10% palladium on carbon (Pd—C, 0.2 mmol) was added. After overnight stirring under hydrogen atmosphere, the reaction mixture was filtered, washed with methanol and concentrated under reduced pressure to provide the desired 24,28-diamine(E4D).

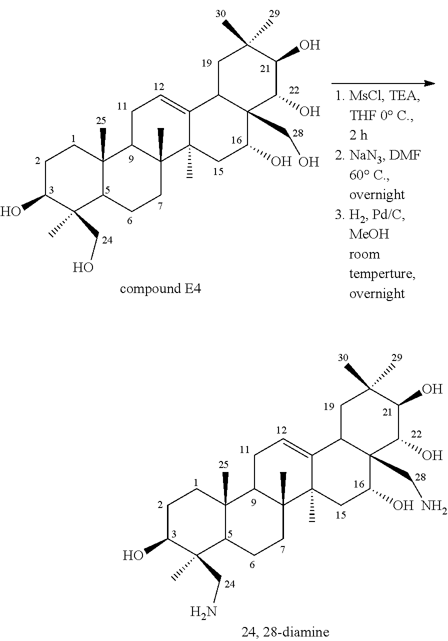

Experiment 43:

To a solution of 24,28-diamine(E4D) (0.1 mmol.) and aldehyde (RCHO, 0.3 mmol) in dimethylformamide (DMF, 1 mL) in a fritted plastic reaction tube was added SiliaBondCyanoborohydride (Si-CBH, 0.3 mmol). The reaction mixture was shaken at room temperature overnight. The reaction mixture was filtered into a fritted plastic reaction tube. Tosic acid functionalized silica (Si-SCX, 0.3 mmol) was added. The reaction mixture was shaken at room temperature overnight. The liquid was filtered off. A solution of 5% ammonia-methanol (1 mL) was added to the product captured silica. The mixture was shaken at room temperature overnight. The methanolic solution was filtered off and concentrated under reduced pressure to yield the desired amine analog.

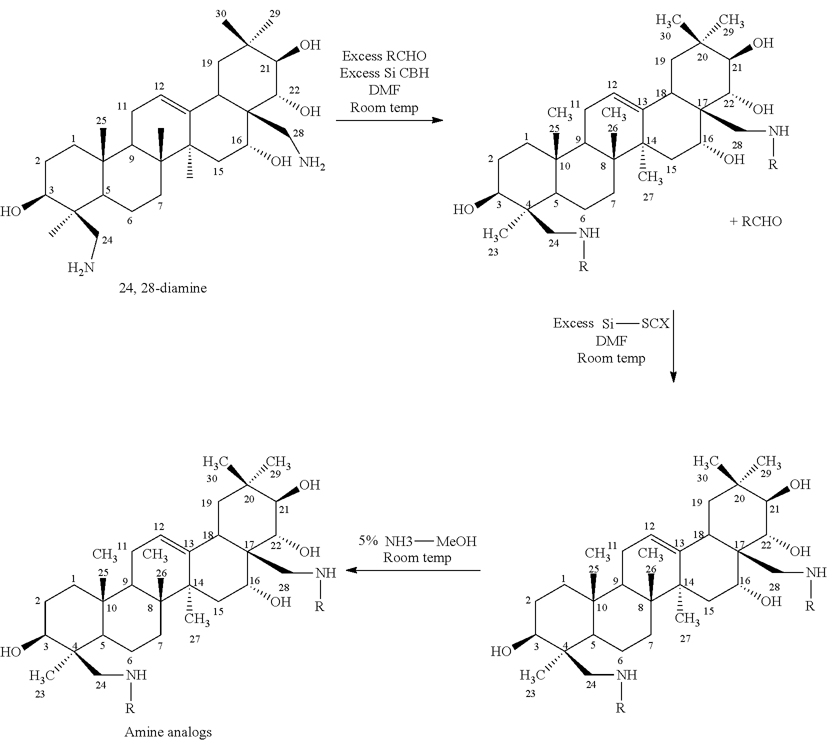

Experiment 44:

To a solution of 24,28-diamine(E4D) (0.1 mmol.) and carboxylic acid (RCO₂H, 0.3 mmol) in dimethylformamide (DMF, 1 mL) in a fritted plastic reaction tube was added EDC functionalized silica (Si-EDC, 0.3 mmol). The reaction mixture was shaken at room temperature overnight. Carbonate functionalized silica (Si—CO₃, 0.3 mmol) was added. The reaction mixture was shaken at room temperature overnight. The reaction mixture was filtered and concentrated under reduced pressure to yield the desired amide analog.

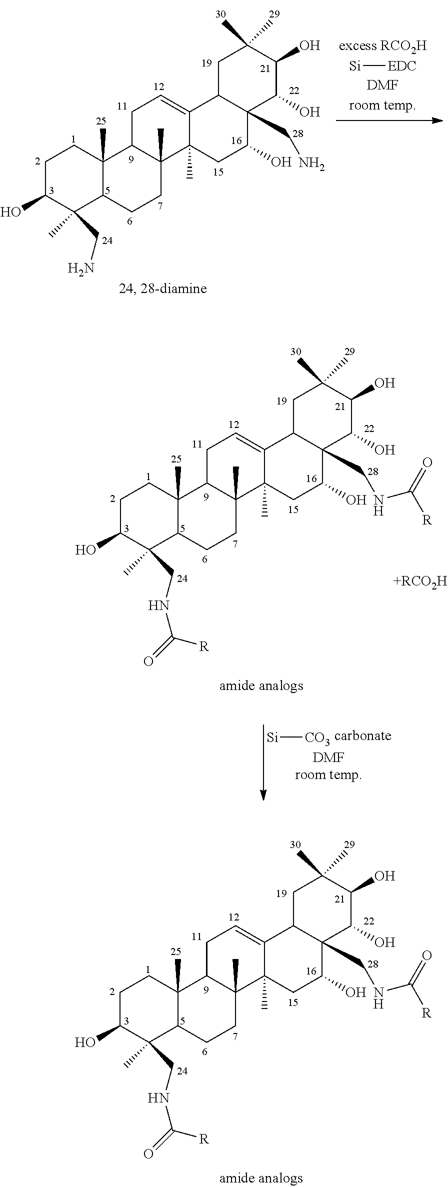

Experiment 45:

Method for the Preparation of Sulfonamide Analogs

To a solution of 24,28-diamine(E4D) (0.1 mmol.) and sulfonyl chloride (RSO₂Cl, 0.3 mmol) in dimethylformamide (DMF, 1 mL) in a fritted plastic reaction tube was added triethylamine functionalized silica (Si-WAX-2, 0.3 mmol). The reaction mixture was shaken at room temperature overnight. Amine functionalized silica (Si-WAX, 0.3 mmol) was added. The reaction mixture was shaken at room temperature overnight. The reaction mixture was filtered and concentrated under reduced pressure to yield the desired sulfonamide analog.

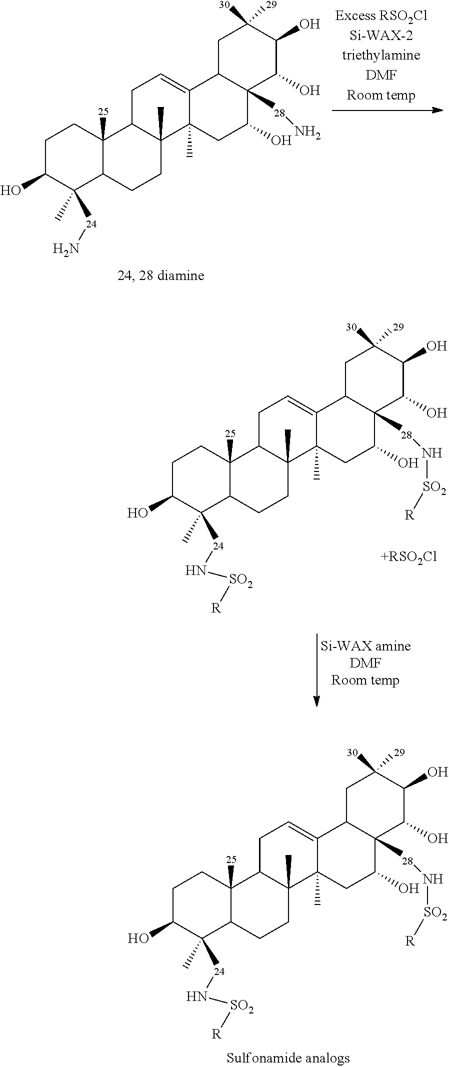

Experiment 46:

Method for the Preparation of Urea Analogs

A solution of 24,28-diamine(E4D) (0.1 mmol.) and isocyanate (RNCO, 0.3 mmol) in dimethylformamide (DMF, 1 mL) in a fritted plastic reaction tube was shaken at room temperature overnight. Piperazine functionalized silica (Si-PPZ, 0.3 mmol) was added. The reaction mixture was shaken at room temperature overnight. The reaction mixture was filtered and concentrated under reduced pressure to yield the desired urea analog.

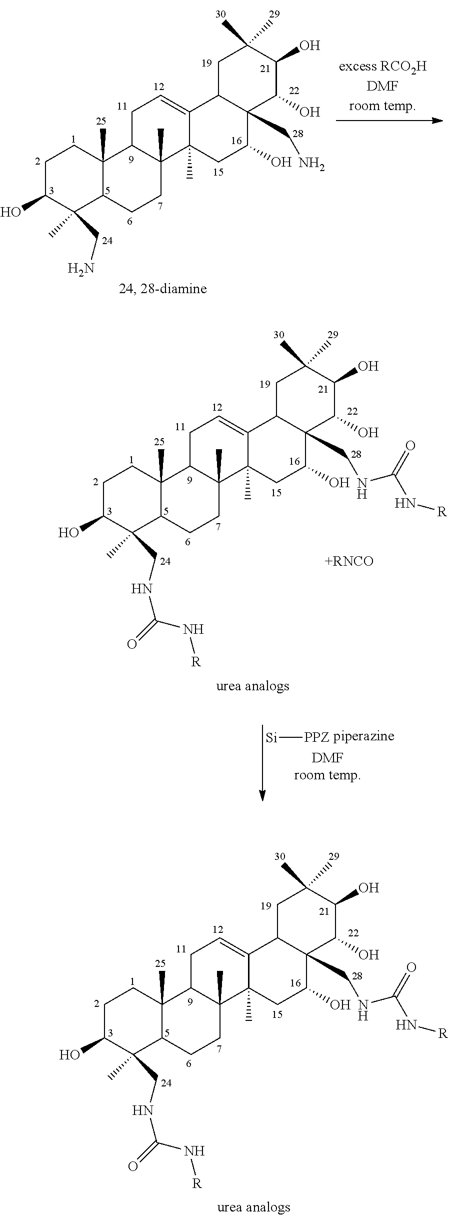

Experiment 47: Using method in Experiment 43, (E4D) with acetaldehyde, (Z)-2-methylbut-2-enal, (E)-2-methylbut-2-enal, 3-methylbut-2-enal, (E)-but-2-enal, cinnamaldehyde, pent-4-enal, (E)-4-(dimethylamino)but-2-enal gave the following compounds:

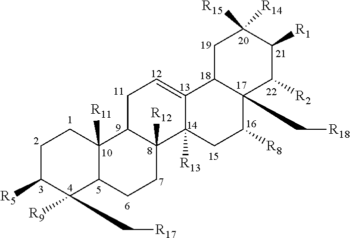

wherein:

1) R1, R2, R3, R5, R8 are OH; R17, R18 are NH-ethyl;

2) R1, R2, R3, R5, R8 are OH; R17, R18 are NH—(Z)-(2-methylbut-2-en-1-yl);

3) R1, R2, R3, R5, R8 are OH; R17, R18 are NH-(E)-(2-methylbut-2-en-1-yl);

4) R1, R2, R3, R5, R8 are OH; R17, R18 are NH-(3-methylbut-2-en-1-yl;

5) R1, R2, R3, R5, R8 are OH; R17, R18 are NH-(E)-(but-2-en-1-yl;

6) R18, R2, R3, R5, R8 are OH; R17, R18 are NH-cinnamyl;

7) R18, R2, R3, R5, R8 are OH; R17, R18 are NH-pent-4-en-1-yl;

8) R18, R2, R3, R5, R8 are OH; R17, R18 are NH-(E)-3-((4-(dimethylamino)but-2-en yl.

Experiment 48: Using Method in Experiment 46, (E4D) with isocyanatomethane, (Z) isocyanato-2-methylbut-2-ene, (E)-1-isocyanato-2-methylbut-2-ene, 1-isocyanato-3-methylbut-2-ene, (E)-1-isocyanatobut-2-ene, (E)-(3-isocyanatoprop-1-en-1-yl)benzene, 4-isocyanatobut-1-ene, (E)-4-isocyanato-N,N-dimethylbut-1-en-1-amine gave the following compounds:

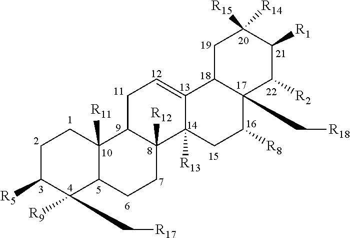

wherein:

1) R1, R2, R5, R8 are OH; R17, R18 are NH—CO—NH-ethyl;

2) R1, R2, R5, R8 are OH; R17, R18 are NH—CO—NH—(Z)-1-(2-methylbut-2-en-1-yl)

3) R1, R2, R5, R8 are OH; R17, R18 are NH—CO—NH-(E)-1-(2-methylbut-2-en-1-yl)

4) R1, R2, R5, R8 are OH; R17, R18 are NH—CO—NH-1-(3-methylbut-2-en-1-yl)

5) R1, R2, R5, R8 are OH; R17, R18 are NH—CO—NH-(E)-1-(but-2-en-1-yl)

6) R18, R2, R5, R8 are OH; R17, R18 are NH—CO—NH-1-cinnamyl

7) R18, R2, R5, R8 are OH; R17, R18 are NH—CO—NH-1-(but-3-en-1-yl)

8) R18, R2, R5, R8 are OH; R17, R18 are NH—CO—NH-(E)-1-(4-(dimethylamino)but-3-en-1-yl)

Experiment 49: Using method in Experiment 44, (E4D) with ethanoyl, angeloyl, tigloyl, senecioyl, Crotonoyl, Cinnamoyl, Pentenoyl, (E)-3-(dimethylamino)acryloyl, 4-[(2-methoxyethyl)amino]-2-methyl-4oxobut-2-enoyl gave the following compounds:

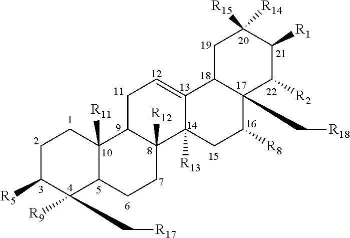

wherein:

1) R1, R2, R5, R8 are OH; R17, R18 are NH-acetyl;

2) R1, R2, R5, R8 are OH; R17, R18 are NH-angeloyl

3) R1, R2, R5, R8 are OH; R17, R18 are NH-tigloyl

4) R1, R2, R5, R8 are OH; R17, R18 are NH-senecioyl

5) R1, R2, R5, R8 are OH; R17, R18 are NH-Crotonoyl

6) R18, R2, R5, R8 are OH; R17, R18 are NH-Cinnamoyl

7) R18, R2, R5, R8 are OH; R17, R18 are NH-Pentenoyl

8) R18, R2, R5, R8 are OH; R17, R18 are NH-4-(dimethylamino)-2-methylbut-2-enoyl

9) R18, R2, R3, R5, R8 are OH; R17, R18 are NH-4-[(2-methoxyethyl)amino]-2-methyl-4oxobut-2-enoyl

Experiment 50:

Using method in Experiment 45, (E4D) with ethanesulfonyl, (Z)-but-2-ene-2-sulfonyl, (E)-prop-1-ene-1-sulfonyl, (E)-2-phenylethene-1-sulfonyl, but-3-ene-1-sulfonyl gave the following compounds:

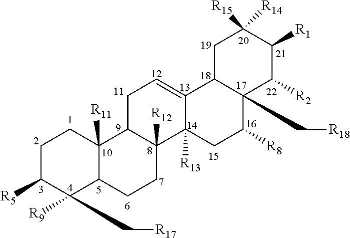

wherein:

1) R1, R2, R5, R8 are OH; R17, R18 are NHSO2-ethyl;

2) R1, R2, R5, R8 are OH; R17, R18 are NHSO2-(Z)-(2-methylbut-2-en-1-yl)

3) R1, R2, R5, R8 are OH; R17, R18 are NHSO2-(E)-prop-1-enyl

4) R18, R2, R5, R8 are OH; R17, R18 are NHSO2-(E)-2-phenylethenyl

5) R18, R2, R5, R8 are OH; R17, R18 are NHSO2-but-3-enyl

Experiment 51:

Acid Cl+Amine

The reaction of acid chlorides and amines to form amides. The reaction takes place at RT with a suitable base (ex. TEA or DIEA) in an aprotic solvent (ex. DCM, THF, or DMF).

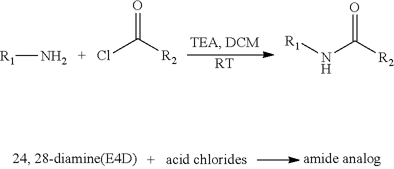

Experiment 52: Using method in Experiment 51, (E4D) with ethanoyl chlorides, angeloyl chlorides, tigloyl chlorides, senecioyl chlorides, Crotonoyl chlorides, Cinnamoyl chlorides, Pentenoyl chlorides, (E)-3-(dimethylamino)acryloyl chlorides, 4-[(2-methoxyethyl)amino]-2-methyl-4oxobut-2-enoyl chlorides gave the following compounds:

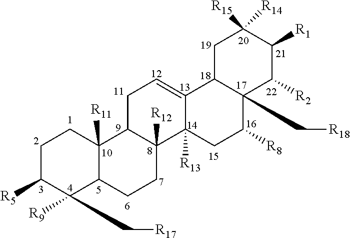

wherein:

1) R1, R2, R5, R8 are OH; R17, R18 are NH-acetyl;

2) R1, R2, R5, R8 are OH; R17, R18 are NH-angeloyl

3) R1, R2, R5, R8 are OH; R17, R18 are NH-tigloyl

4) R1, R2, R5, R8 are OH; R17, R18 are NH-senecioyl

5) R1, R2, R5, R8 are OH; R17, R18 are NH-Crotonoyl

6) R18, R2, R5, R8 are OH; R17, R18 are NH-Cinnamoyl

7) R18, R2, R5, R8 are OH; R17, R18 are NH-Pentenoyl

8) R18, R2, R5, R8 are OH; R17, R18 are NH-4-(dimethylamino)-2-methylbut-2-enoyl

9) R18, R2, R5, R8 are OH; R17, R18 are NH-4-[(2-methoxyethyl)amino]-2-methyl-4oxobut-2-enoyl

Experiment 53

Pharmacokinetic (PK) Study: Blood from Cutting Tail
Method:
Drug-Treatment:
   Mice: CD-1 female mice (6-7 weeks, 23-25 g)
   mice were injected (IP) with Test drug sample (10 mg/ml, in 127 (10% DMSO-20% Tween-80 in PBS) at a dose of 100 mg/kg.
   Blood was obtained from a mouse by cut-tail at 0.5, 2 and 4 hours after drug treatments.
   At 24 hours after the drug treatment, mice were anesthetized with isoflurane and blood was obtained with cardiac puncture. Animals were then euthanized by cervical dislocation.
Plasma Isolation:
   Blood (50-100 ul) was centrifuged (2000 g, 15 minutes). Plasma was collected from the top layer.
   1 volume of plasma was mixed with 3 volume of acetonitrile/0.005% TFA. Mixture was stored in refrigerator overnight.
   Sample was centrifuged (16000 g,10 minutes). Supernatant (called Plasma in 75% AN/TFA soluble fractions) was collected and subjected to HPLC analysis.
   HPLC Analysis Conditions:
   Column: C18 μBondapak
   Solvent: 60% acetonitrile/0.005% TFA (Trifluoroacetic acid)
   Gradient: isocratic
   Flow rate and run time: 1 ml/min for 30 minutes
   Detection wavelength: 207 nm
   O.D. range: 0.05 full scale
   Calculation:
   Standard cures of Test Drug is constructed (Area/3000K=1 ug Test Drug)
   Area of Test Drug Peak from Sample is Recorded
   Amount of Test Drug was calculated according to standard cures.

Results and conclusion: over 80% of Test Drug (E4A-TigR) in blood is cleared during the first 4 hours. The half-life is estimated to be about one hour; over 80% of Test Drug (E4D-Tig-R) in blood is cleared during the first 8 hours.

Experiment 54

Pharmacokinetic (PK) Study: Blood from Cardiac Puncture
Method:
Drug-Treatment:
   Mice: CD-1 female mice (8-10 weeks, 25-30 g)
   mice were injected (IP) with Test Drug (10 mg/ml, in 10% DMSO-20% Tween-80 in PBS) at a dose of 100 mg/kg.
   At 1 and 2 hours after drug treatments, individual animals were anesthetized with isoflurane.
   blood was obtained by cardiac puncture (between the chest-ribs bones). Animals were then euthanized by cervical dislocation.
Plasma Isolation:
   Blood (0.6-0.8 ml) was centrifuged (2000 g, 15 minutes). Plasma was collected from the top layer.
   1 volume of plasma was mixed with 3 volume of acetonitrile/0.005% TFA. Mixture was stored in −20° C. for 6 minutes.
   Sample was centrifuged (16000 g, 10 minutes). Supernatant was subjected to HPLC analysis.
   HPLC Analysis Conditions:
   Column: C18 μBondapak
   Solvent: 60% acetonitrile/0.005% TFA (Trifluoroacetic acid)
   Gradient: isocratic
   Flow rate and run time: 1 ml/min for 30 minutes
   Detection wavelength: 207 nm
   O.D. range: 0.05 full scale
Calculation:
   Standard cures of Test Drug is constructed (Area/3000K=1 ug Test Drug)
   Area of Test Drug peak from Sample is Recorded
   Amount of Test Drug was calculated according to standard cures.

Results and conclusion: over 80% of Test Drug (E4A-Tig-R) in blood is cleared during the first 4 hours. The half-life is estimated to be about one hour; over 80% of Test Drug (E4D-Tig-R) in blood is cleared during the first 8 hours.

Definations in Experiments 43, 44, 45 and 46

R group for amine, sulfonamides, amide analog, Urea Analogs, wherein sulfonyl chloride (RSO₂Cl), carboxylic acid (RCO₂H), isocyanate (RNCO), where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-hterocyclyl are unsubstituted or substituted with one or more groups of Z.

Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂;

Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;

Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂—R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —(NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(akyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

What is claimed is:

1. A compound having the structure: 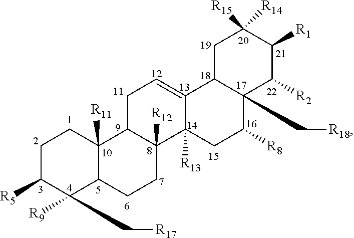
diamine, amine, amide, sulfonamide or urea thereof, wherein R1, R2, R3, R5, R8, R9, R11, R12, R13, R14, R15, R16 are independently selected from the group of H, O, OH, NH2, CH3, CH2OH, and COOH; R17 and R18 are selected from NH-ethyl, NH—(Z)-(2-methylbut-2-en-1-yl), NH-(E)-(2-methylbut-2-en-1-yl), NH-(3-methylbut-2-en-1-yl, NH-(E)-(but-2-en-1-yl, NH-cinnamyl, NH-pent-4-en-1-yl, NH-(E)-3-((4-(dimethylamino)but-2-en-1-yl, NH-acetyl, NH-angeloyl, NH-tigloyl, NH-senecioyl, NH-Crotonoyl, NH-Cinnamoyl, NH-Pentenoyl, NH-4-(dimethylamino)-2-methylbut-2-enoyl, NH-4-[(2-methoxyethyl)amino]-2-methyl-4oxobut-2-enoyl, NHSO2-ethyl, NHSO2-(Z)-(2-methylbut-2-en-1-yl), NHSO2-(E)-prop-1-enyl, NHSO2-(E)-2-phenylethenyl, NHSO2-but-3-enyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl); or wherein the R17 and R18 are selected from NH-ethanyl, NH-propanyl, NH-propenyl, NH-butanyl, NH-butenyl, NH-pentanyl, NH-hexenyl, NH-heptanyl, NH-heptenyl, NH-octanyl, NH-octenyl, NH-nonanyl, NH-nonenyl, NH-decanyl, NH-decenyl, NH-alkyl, NH-haloalkyl, NH-alkenyl, NH-alkynyl, NH-hydroxyalkyl, NH-alkylene-O-alkyl, NH-aryl, NH-alkylene-aryl, NH-heteroaryl, NH-alkylene-heteroaryl, NH-cycloalkyl, NH-heterocyclyl, and NH-alkylene-heterocyclyl, NH-tigloyl, NH-angeloyl, NH-senecioyl, NH-acetyl, NH-Crotonoyl, NH-3,3-Dimethylartyloyl, NH-Cinnamoyl, NH-Pentenoyl, NH-Hexanoyl, NH-benzoyl, NH-Ethylbutyryl, NH-alkyl, NH-dibenzoyl, NH-benzoyl, NH-alkanoyl, NH-alkenoyl, NH-benzoyl alkyl substituted NH-alkanoyl, NH-alkanoyl substituted phenyl, NH-alkenoyl substituted phenyl, NH-aryl, NH-acyl, NH-heterocylic, NH-heteroraryl, NH-alkenylcarbonyl, NH-alkane, NH-alkene, NH-sugar moiety, NH-acid moiety, NH-ethanoyl, NH-propanoyl, NH-propenoyl, NH-butanoyl, NH-butenoyl, NH-pentanoyl, NH-hexenoyl, NH-heptanoyl, NH-heptenoyl, NH-octanoyl, NH-octenoyl, NH-nonanoyl, NH-nonenoyl, NH-decanoyl, NH-decenoyl, NH-propionyl, NH-2-propenoyl, NH-2-butenoyl, NH-Isobutyryl, NH-2-methylpropanoyl, NH-2-ethylbutyryl, NH-ethylbutanoyl, NH-2-ethylbutanoyl, NH-butyryl, NH-(E)-2,3-Dimethylacryloyl, NH-(E)-2-Methylcrotonoyl, NH-3-cis-Methyl-methacryloyl, NH-3-Methyl-2-butenoyl, NH-3-Methylcrotonoyl, NH-4-Pentenoyl, NH-(2E)-2-pentenoyl, NH-Caproyl, NH-5-Hexenoyl, NH-Capryloyl, NH-Lauroyl, NH-Dodecanoyl, NH-Myristoyl, NH-Tetradecanoyl, NH-Oleoyl, NH—C(2-18) Acyl,
CH2NH-tigloyl, CH2NH-angeloyl, CH2NH-senecioyl, CH2NH-acetyl, CH2NH-Crotonoyl, CH2NH-3,3-Dimethylartyloyl, CH2NH-Cinnamoyl, CH2NH-Pentenoyl, CH2NH-Hexanoyl, CH2NH-benzoyl, CH2NH-Ethylbutyryl, CH2NH-alkyl, CH2NH-dibenzoyl, CH2NH-benzoyl, CH2NH-alkanoyl, CH2NH-alkenoyl, CH2NH-benzoyl alkyl substituted CH2NH-alkanoyl, CH2NH-alkanoyl substituted phenyl, CH2NH-alkenoyl substituted phenyl, CH2NH-aryl, CH2NH-acyl, CH2NH-heterocylic, CH2NH-heteroraryl, CH2NH-alkenylcarbonyl, CH2NH-alkane, CH2NH-alkene, CH2NH-sugar moiety, CH2NH-acid moiety, CH2NH-ethanoyl, CH2NH-propanoyl, CH2NH-propenoyl, CH2NH-butanoyl, CH2NH-butenoyl, CH2NH-pentanoyl, CH2NH-hexenoyl, CH2NH-heptanoyl, CH2NH-heptenoyl, CH2NH-octanoyl, CH2NH-octenoyl, CH2NH-nonanoyl, CH2NH-nonenoyl, CH2NH-decanoyl, CH2NH-decenoyl, CH2NH-propionyl, CH2NH-2-propenoyl, CH2NH-2-butenoyl, CH2NH-Isobutyryl, CH2NH-2-methylpropanoyl, CH2NH-2-ethylbutyryl, CH2NH-ethylbutanoyl, CH2NH-2-ethylbutanoyl, CH2NH-butyryl, CH2NH-(E)-2,3-Dimethylacryloyl, CH2NH-(E)-2-Methylcrotonoyl, CH2NH-3-cis-Methyl-methacryloyl, CH2NH-3-Methyl-2-butenoyl, CH2NH-3-Methylcrotonoyl, CH2NH-4-Pentenoyl, CH2NH-(2E)-2-pentenoyl, CH2NH-Caproyl, CH2NH-5-Hexenoyl, CH2NH-Capryloyl, CH2NH-Lauroyl, CH2NH-Dodecanoyl, CH2NH-Myristoyl, CH2NH-Tetradecanoyl, CH2NH-Oleoyl, CH2NH—C(2-18) Acyl,
CH2NHCO-tigloyl, CH2NHCO-angeloyl, CH2NHCO-senecioyl, CH2NHCO-acetyl, CH2NHCO-Crotonoyl, CH2NHCO-3,3-Dimethylartyloyl, CH2NHCO-Cinnamoyl, CH2NHCO-Pentenoyl, CH2NHCO-Hexanoyl, CH2NHCO-benzoyl, CH2NHCO-Ethylbutyryl, CH2NHCO-alkyl, CH2NHCO-dibenzoyl, CH2NHCO-benzoyl, CH2NHCO-alkanoyl, CH2NHCO-alkenoyl, CH2NHCO-benzoyl alkyl substituted CH2NHCO-alkanoyl, CH2NHCO-alkanoyl substituted phenyl, CH2NHCO-alkenoyl substituted phenyl, CH2NHCO-aryl, CH2NHCO-acyl, CH2NHCO-heterocylic, CH2NHCO-heteroraryl, CH2NHCO-alkenylcarbonyl, CH2NHCO-alkane, CH2NHCO-alkene, CH2NHCO-sugar moiety, CH2NHCO-acid moiety, CH2NHCO-ethanoyl, CH2NHCO-propanoyl, CH2NHCO-propenoyl, CH2NHCO-butanoyl, CH2NHCO-butenoyl, CH2NHCO-pentanoyl, CH2NHCO-hexenoyl, CH2NHCO-heptanoyl, CH2NHCO-heptenoyl, CH2NHCO-octanoyl, CH2NHCO-octenoyl, CH2NHCO-nonanoyl, CH2NHCO-nonenoyl, CH2NHCO-decanoyl, CH2NHCO-decenoyl, CH2NHCO-propionyl, CH2NHCO-2-propenoyl, CH2NHCO-2-butenoyl, CH2NHCO-Isobutyryl, CH2NHCO-2-methylpropanoyl, CH2NHCO-2-ethylbutyryl, CH2NHCO-ethylbutanoyl, CH2NHCO-2-ethylbutanoyl, CH2NHCO-butyryl, CH2NHCO-(E)-2,3-Dimethylacryloyl, CH2NHCO-(E)-2-Methylcrotonoyl, CH2NHCO-3-cis-Methyl-methacryloyl, CH2NHCO-3-Methyl-2-butenoyl, CH2NHCO-3-Methylcrotonoyl, CH2NHCO-4-Pentenoyl, CH2NHCO-(2E)-2-pentenoyl, CH2NHCO-Caproyl, CH2NHCO-5-Hexenoyl, CH2NHCO-Capryloyl, CH2NHCO-Lauroyl, CH2NHCO-Dodecanoyl, CH2NHCO-Myristoyl, CH2NHCO-Tetradecanoyl, CH2NHCO-Oleoyl, CH2NHCO—C(2-18) Acyl,
CH2NHCONH-tigloyl, CH2NHCONH-senecioyl, CH2NHCONH-acetyl, CH2NHCONH-Crotonoyl, CH2NHCONH-3,3-Dimethylartyloyl, CH2NHCONH-Cinnamoyl, CH2NHCONH-Pentenoyl, CH2NHCONH-Hexanoyl, CH2NHCONH-benzoyl, CH2NHCONH-Ethylbutyryl, CH2NHCONH-alkyl, CH2NHCONH-dibenzoyl, CH2NHCONH-benzoyl, CH2NHCONH-alkanoyl, CH2NHCONH-alkenoyl, CH2NHCONH-benzoyl alkyl substituted CH2NHCONH-alkanoyl, CH2NHCONH-alkanoyl substituted phenyl, CH2NHCONH-alkenoyl substituted phenyl, CH2NHCONH-aryl, CH2NHCONH-acyl, CH2NHCONH-heterocylic, CH2NHCONH-heteroraryl, CH2NHCONH-alkenylcarbonyl, CH2NHCONH-alkane, CH2NHCONH-alkene, CH2NHCONH-sugar moiety, CH2NHCONH-acid moiety, CH2NHCONH-ethanoyl, CH2NHCONH-propanoyl, CH2NHCONH-propenoyl, CH2NHCONH-butanoyl, CH2NHCONH-butenoyl, CH2NHCONH-pentanoyl, CH2NHCONH-hexenoyl, CH2NHCONH-heptanoyl, CH2NHCONH-heptenoyl, CH2NHCONH-octanoyl, CH2NHCONH-octenoyl, CH2NHCONH-nonanoyl, CH2NHCONH-nonenoyl, CH2NHCONH-decanoyl, CH2NHCONH-decenoyl, CH2NHCONH-propionyl, CH2NHCONH-2-propenoyl, CH2NHCONH-2-butenoyl, CH2NHCONH-Isobutyryl, CH2NHCONH-2-methylpropanoyl, CH2NHCONH-2-ethylbutyryl, CH2NHCONH-ethylbutanoyl, CH2NHCONH-2-ethylbutanoyl, CH2NHCONH-butyryl, CH2NHCONH-(E)-2,3-Dimethylacryloyl, CH2NHCONH-(E)-2-Methylcrotonoyl, CH2NHCONH-3-cis-Methyl-methacryloyl, CH2NHCONH-3-Methyl-2-butenoyl, CH2NHCONH-3-Methylcrotonoyl, CH2NHCONH-4-Pentenoyl, CH2NHCONH-(2E)-2-pentenoyl, CH2NHCONH-Caproyl, CH2NHCONH-5-Hexenoyl, CH2NHCONH-Capryloyl, CH2NHCONH-Lauroyl, CH2NHCONH-Dodecanoyl, CH2NHCONH-Myristoyl, CH2NHCONH-Tetradecanoyl, CH2NHCONH-Oleoyl, CH2NHCONH—C(2-18) Acyl, NHSO2-tigloyl, NHSO2-senecioyl, NHSO2-acetyl, NHSO2-Crotonoyl, NHSO2-3,3-Dimethylartyloyl, NHSO2-Cinnamoyl, NHSO2-Pentenoyl, NHSO2-Hexanoyl, NHSO2-benzoyl, NHSO2-Ethylbutyryl, NHSO2-alkyl, NHSO2-dibenzoyl, NHSO2-benzoyl, NHSO2-alkanoyl, NHSO2-alkenoyl, NHSO2-benzoyl alkyl substituted NHSO2-alkanoyl, NHSO2-alkanoyl substituted phenyl, NHSO2-alkenoyl substituted phenyl, NHSO2-aryl, NHSO2-acyl, NHSO2-heterocylic, NHSO2-heteroraryl, NHSO2-alkenylcarbonyl, NHSO2-alkane, NHSO2-alkene, NHSO2-sugar moiety, NHSO2-acid moiety, NHSO2-ethanoyl, NHSO2-propanoyl, NHSO2-propenoyl, NHSO2-butanoyl, NHSO2-butenoyl, NHSO2-pentanoyl, NHSO2-hexenoyl, NHSO2-heptanoyl, NHSO2-heptenoyl, NHSO2-octanoyl, NHSO2-octenoyl, NHSO2-nonanoyl, NHSO2-nonenoyl, NHSO2-decanoyl, NHSO2-decenoyl, NHSO2-propionyl, NHSO2-2-propenoyl, NHSO2-2-butenoyl, NHSO2-Isobutyryl, NHSO2-2-methylpropanoyl, NHSO2-2-ethylbutyryl, NHSO2-ethylbutanoyl, NHSO2-2-ethylbutanoyl, NHSO2-butyryl, NHSO2-(E)-2,3-Dimethylacryloyl, NHSO2-(E)-2-Methylcrotonoyl, NHSO2-3-cis-Methyl-methacryloyl, NHSO2-3-Methyl-2-butenoyl, NHSO2-3-Methylcrotonoyl, NHSO2-4-Pentenoyl, NHSO2-(2E)-2-pentenoyl, NHSO2-Caproyl, NHSO2-5-Hexenoyl, NHSO2-Capryloyl, NHSO2-Lauroyl, NHSO2-Dodecanoyl, NHSO2-Myristoyl, NHSO2-Tetradecanoyl, NHSO2-Oleoyl, NHSO2-C(2-18) Acyl,
CH2NHSO2-tigloyl, CH2NHSO2-senecioyl, CH2NHSO2-acetyl, CH2NHSO2-Crotonoyl, CH2NHSO2-3,3-Dimethylartyloyl, CH2NHSO2-Cinnamoyl, CH2NHSO2-Pentenoyl, CH2NHSO2-Hexanoyl, CH2NHSO2-benzoyl, CH2NHSO2-Ethylbutyryl, CH2NHSO2-alkyl, CH2NHSO2-dibenzoyl, CH2NHSO2-benzoyl, CH2NHSO2-alkanoyl, CH2NHSO2-alkenoyl, CH2NHSO2-benzoyl alkyl substituted CH2NHSO2-alkanoyl, CH2NHSO2-alkanoyl substituted phenyl, CH2NHSO2-alkenoyl substituted phenyl, CH2NHSO2-aryl, CH2NHSO2-acyl, CH2NHSO2-heterocylic, CH2NHSO2-heteroraryl, CH2NHSO2-alkenylcarbonyl, CH2NHSO2-alkane, CH2NHSO2-alkene, CH2NHSO2-sugar moiety, CH2NHSO2-acid moiety, CH2NHSO2-ethanoyl, CH2NHSO2-propanoyl, CH2NHSO2-propenoyl, CH2NHSO2-butanoyl, CH2NHSO2-butenoyl, CH2NHSO2-pentanoyl, CH2NHSO2-hexenoyl, CH2NHSO2-heptanoyl, CH2NHSO2-heptenoyl, CH2NHSO2-octanoyl, CH2NHSO2-octenoyl, CH2NHSO2-nonanoyl, CH2NHSO2-nonenoyl, CH2NHSO2-decanoyl, CH2NHSO2-decenoyl, CH2NHSO2-propionyl, CH2NHSO2-2-propenoyl, CH2NHSO2-2-butenoyl, CH2NHSO2-Isobutyryl, CH2NHSO2-2-methylpropanoyl, CH2NHSO2-2-ethylbutyryl, CH2NHSO2-ethylbutanoyl, CH2NHSO2-2-ethylbutanoyl, CH2NHSO2-butyryl, CH2NHSO2-(E)-2,3-Dimethylacryloyl, CH2NHSO2-(E)-2-Methylcrotonoyl, CH2NHSO2-3-cis-Methyl-methacryloyl, CH2NHSO2-3-Methyl-2-butenoyl, CH2NHSO2-3-Methylcrotonoyl, CH2NHSO2-4-Pentenoyl, CH2NHSO2-(2E)-2-pentenoyl, CH2NHSO2-Caproyl, CH2NHSO2-5-Hexenoyl, CH2NHSO2-Capryloyl, CH2NHSO2-Lauroyl, CH2NHSO2-Dodecanoyl, CH2NHSO2-Myristoyl, CH2NHSO2-Tetradecanoyl, CH2NHSO2-Oleoyl, CH2NHSO2-C(2-18) Acyl, wherein the compound is in form in form of powder, liquid or crystal.

2. The compound of claim 1 having the structure: 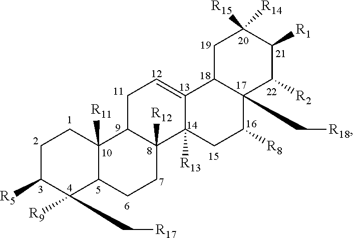
or amine,
wherein:
1) R1, R2, R3, R5, R8 are OH; R17, R18 are NH-ethyl;
2) R1, R2, R3, R5, R8 are OH; R17, R18 are NH—(Z)-(2-methylbut-2-en-1-yl);
3) R1, R2, R3, R5, R8 are OH; R17, R18 are NH-(E)-(2-methylbut-2-en-1-yl);
4) R1, R2, R3, R5, R8 are OH; R17, R18 are NH-(3-methylbut-2-en-1-yl;
5) R1, R2, R3, R5, R8 are OH; R17, R18 are NH-(E)-(but-2-en-1-yl;
6) R18, R2, R3, R5, R8 are OH; R17, R18 are NH-cinnamyl;
7) R18, R2, R3, R5, R8 are OH; R17, R18 are NH-pent-4-en-1-yl;
8) R18, R2, R3, R5, R8 are OH; R17, R18 are NH-(E)-3-((4-(dimethylamino)but-2-en-1-yl.

3. The compound of claim 1 having the structure: 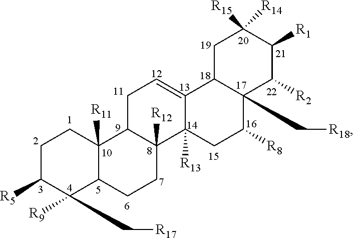
or amide,
wherein:
1) R1, R2, R5, R8 are OH; R17, R18 are NH-acetyl;
2) R1, R2, R5, R8 are OH; R17, R18 are NH-angeloyl;
3) R1, R2, R5, R8 are OH; R17, R18 are NH-tigloyl;
4) R1, R2, R5, R8 are OH; R17, R18 are NH-senecioyl;
5) R1, R2, R5, R8 are OH; R17, R18 are NH-Crotonoyl;
6) R18, R2, R5, R8 are OH; R17, R18 are NH-Cinnamoyl;
7) R18, R2, R5, R8 are OH; R17, R18 are NH-Pentenoyl;
8) R18, R2, R5, R8 are OH; R17, R18 are NH-4-(dimethylamino)-2-methylbut-2-enoyl;
9) R18, R2, R5, R8 are OH; R17, R18 are NH-4-[(2-methoxyethyl)amino]-2-methyl-4oxobut-2-enoyl.

4. The compound of claim 1 having the structure: 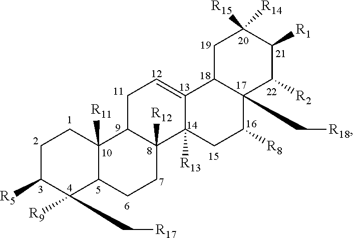
or sulfonamide,
wherein:
1) R1, R2, R3, R5, R8 are OH; R17, R18 are NHSO2-ethyl;
2) R1, R2, R3, R5, R8 are OH; R17, R18 are NHSO2-(Z)-(2-methylbut-2-en-1-yl);
3) R1, R2, R3, R5, R8 are OH; R17, R18 are NHSO2-(E)-prop-1-enyl;
4) R18, R2, R3, R5, R8 are OH; R17, R18 are NHSO2-(E)-2-phenylethenyl;
5) R18, R2, R3, R5, R8 are OH; R17, R18 are NHSO2-but-3-enyl.

5. The compound of claim 1 having the structure: 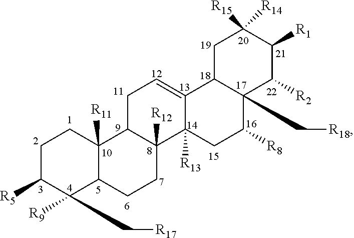
or urea,
wherein:
1) R1, R2, R5, R8 are OH; R17, R18 are NH—CO—NH-ethyl;
2) R1, R2, R5, R8 are OH; R17, R18 are NH—CO—NH—(Z)-1-(2-methylbut-2-en-1-yl);
3) R1, R2, R5, R8 are OH; R17, R18 are NH—CO—NH-(E)-1-(2-methylbut-2-en-1-yl);
4) R1, R2, R5, R8 are OH; R17, R18 are NH—CO—NH-1-(3-methylbut-2-en-1-yl);
5) R1, R2, R5, R8 are OH; R17, R18 are NH—CO—NH-(E)-1-(but-2-en-1-yl);
6) R18, R2, R5, R8 are OH; R17, R18 are NH—CO—NH-1-cinnamyl;
7) R18, R2, R5, R8 are OH; R17, R18 are NH—CO—NH-1-(but-3-en-1-yl);
8) R18, R2, R5, R8 are OH; R17, R18 are NH—CO—NH-(E)-1-(4-(dimethylamino)but-3-en-1-yl).

6. A Method for the preparation of compound of 24,28-diamine(E4D) with E4:
1) To a solution of triterpene or compound (E4)E4A (2 mmol) in tetrahydrofuran (THF, 10 mL) were added methanesulfonyl chloride (Ms-Cl, 2.2 mmol) and triethylamine (TEA, 3 mmol) at 0° C. and the resulting mixture was stirred for 2 hours;
2) The reaction mixture was concentrated under reduced pressure, and the residue was dissolved in ethyl acetate (EtOAc) and washed with water;
3) The organic layer was dried over anhydrous magnesium sulfate, filtered and concentrated under reduced pressure;
4) The residue was dissolved in 10 mL of dimethylformamide (DMF), and then sodium azide (NaN3, 6 mmol) was added. After overnight stirring at 60° C., the reaction mixture was diluted with ethyl acetate and washed with water;
5) The organic layer was dried over anhydrous magnesium sulfate, filtered and concentrated under reduced pressure;
6) Then, the obtained compound was dissolved in methanol (MeOH), and 10% palladium on carbon (Pd—C, 0.2 mmol) was added. After overnight stirring under hydrogen atmosphere, the reaction mixture was filtered, washed with methanol and concentrated under reduced pressure to provide the desired 24,28-diamine(E4D);

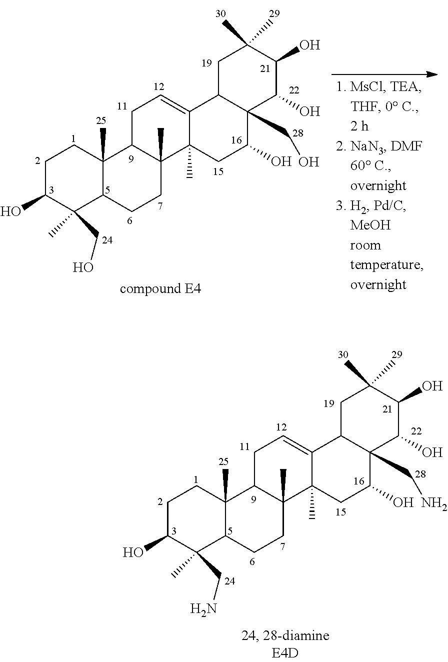

7. The method of claim 6, wherein the (E4D)24,28-diamine is preparation of compound of amide analog:
1) To a solution of 24,28-diamine (0.1 mmol.) and carboxylic acid (RCO₂H, 0.3 mmol) in dimethylformamide (DMF, 1 mL) in a fritted plastic reaction tube was added EDC functionalized silica (Si-EDC, 0.3 mmol);
2) The reaction mixture was shaken at room temperature overnight. Carbonate functionalized silica (Si—CO₃, 0.3 mmol) was added. The reaction mixture was shaken at room temperature overnight;
3) The reaction mixture was filtered and concentrated under reduced pressure to yield the desired amide analog. 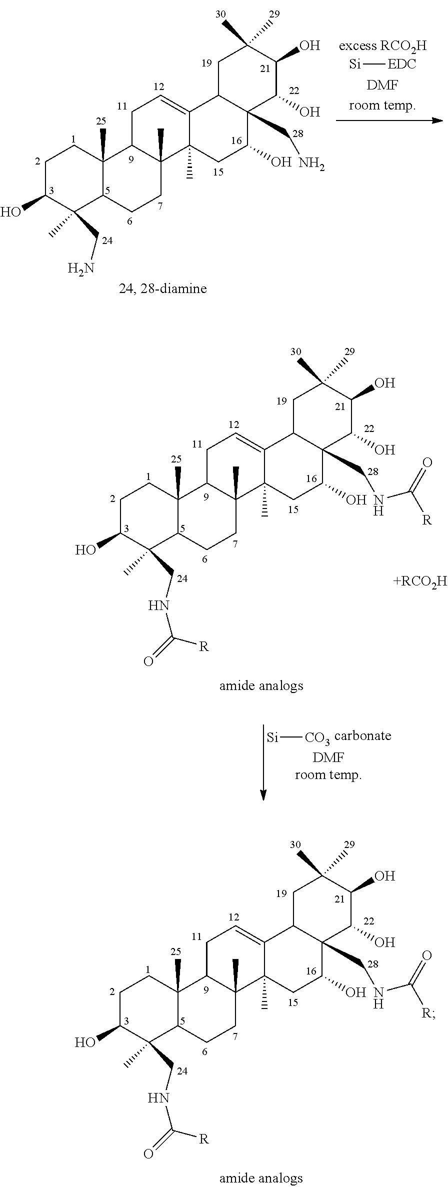
wherein R group for amide analog, carboxylic acid (RCO₂H), where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-heterocyclyl are unsubstituted or substituted with one or more groups of Z.
Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂;
Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;
Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂—R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(akyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

8. The Method of claim 6, wherein the (E4D)24,28-diamine is preparation of compound of sulfonamide analog:
1) To a solution of 24,28-diamine (0.1 mmol.) and sulfonyl chloride (RSO₂Cl, 0.3 mmol) in dimethylformamide (DMF, 1 mL) in a fritted plastic reaction tube was added triethylamine functionalized silica (Si-WAX-2, 0.3 mmol);
2) The reaction mixture was shaken at room temperature overnight. Amine functionalized silica (Si-WAX, 0.3 mmol) was added;
3) The reaction mixture was shaken at room temperature overnight. The reaction mixture was filtered and concentrated under reduced pressure to yield the desired sulfonamide analog, 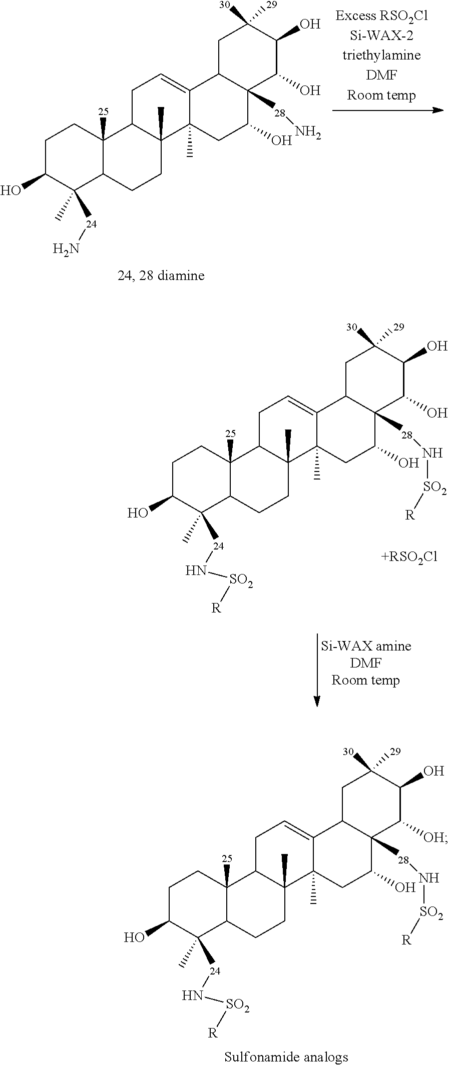
wherein R group for sulfonamides, and sulfonyl chloride (RSO₂Cl), where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-hterocyclyl are unsubstituted or substituted with one or more groups of Z.
Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂;
Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;
Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂—R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(akyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

9. The Method of claim 6, wherein the (E4D)24,28-diamine is for the Preparation of compound of urea analog:
1) A solution of 24,28-diamine (0.1 mmol.) and isocyanate (RNCO, 0.3 mmol) in dimethylformamide (DMF, 1 mL) in a fritted plastic reaction tube was shaken at room temperature overnight;
2) Piperazine functionalized silica (Si-PPZ, 0.3 mmol) was added;
3) The reaction mixture was shaken at room temperature overnight;
4) The reaction mixture was filtered and concentrated under reduced pressure to yield the desired urea analog. 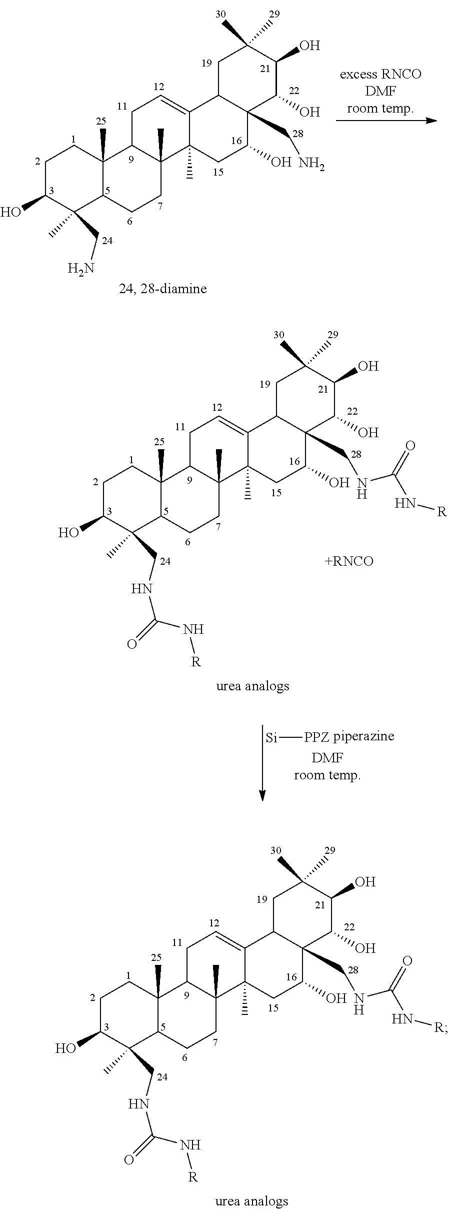
wherein R group for urea analogs, and isocyanate (RNCO), where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-hterocyclyl are unsubstituted or substituted with one or more groups of Z.
Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂;
Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;
Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂—R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(alkyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

10. The Method of claim 6, wherein the (E4D)24,28-diamine is for the Preparation of compound of amine analog:
1) To a solution of 24,28-diamine(E4D) (0.1 mmol.) and aldehyde (RCHO, 0.3 mmol) in dimethylformamide (DMF, 1 mL) in a fritted plastic reaction tube was added SiliaBondCyanoborohydride (Si-CBH, 0.3 mmol). The reaction mixture was shaken at room temperature overnight.
2) The reaction mixture was filtered into a fritted plastic reaction tube. Tosic acid functionalized silica (Si-SCX, 0.3 mmol) was added. The reaction mixture was shaken at room temperature overnight.
3) The liquid was filtered off. A solution of 5% ammonia-methanol (1 mL) was added to the product captured silica. The mixture was shaken at room temperature overnight.
4) The methanolic solution was filtered off and concentrated under reduced pressure to yield the desired amine analog. 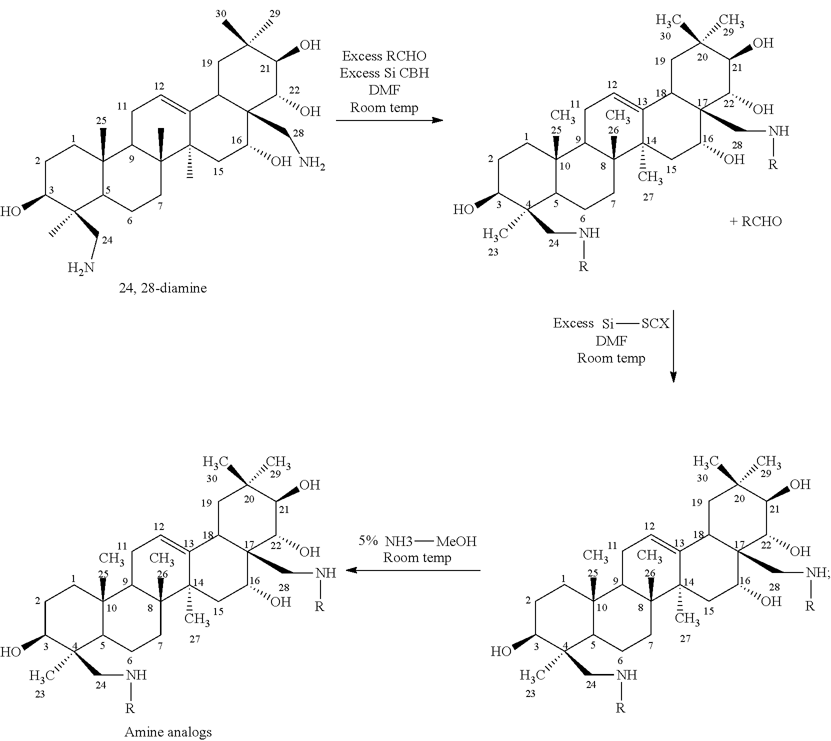
wherein R group for amine and RCHO, where is selected from the group consisting of alkyl, haloalkyl, alkenyl, alkynyl, hydroxyalkyl, -alkylene-O-alkyl, aryl, -alkylene-aryl, heteroaryl, -alkylene-heteroaryl, cycloalkyl, heterocyclyl, and -alkylene-heterocyclyl, wherein said aryl, heteroaryl, the aryl portion of said -alkylene-aryl, or the heteroaryl portion of said -alkylene-heteroaryl are unsubstituted or substituted with one or more groups Y which are selected; said heterocyclyl or the heterocyclyl portion of said -alkylene-hterocyclyl are unsubstituted or substituted with one or more groups of Z.
Y is one or more substituents independently selected from the group consisting of halogen, alkyl, haloalkyl, aryl, -alkylene-aryl, —OH, —CN, —N(R′)₂, —N(R′)—C(O)—R′, —N(R′)—C(O)—(N′)₂, —C(O)N(R′)₂, —C(O)OH, —C(O)O-alkyl, N(R′)—S(O)₂—(R′) and —S(O)₂N(R′)₂;
Each R′ is independently selected from the group consisting of H, alkyl, cyloalkyl, haloalkyl, heterocyclyl, -alkelene-hterocyclyl, aryl, -alkylene-aryl, heteroaryl, -alkylene-heteroaryl;
Z is one or more substituents independently selected from the group consisting of alkyl, one or more hydroxy substituted alkyl, aryl, -alkylene-aryl, -alkylene-O-alkyl, -alkylene-O-alkylene-aryl, -alkylene-O-aryl, -alkylene-O-alkyl, -alkylene-O-alkylene-aryl, -alkylene-O-aryl, —CN, haloalkyl, -alkylene-C(O)—N(R″)₂, —C(O)—N(R″)₂, —C(O)OH, —C(O)O-alkyl, —N(R″)₂, and -alkylene-N(R″)₂, —S(O)₂—N(R″)₂, -alkylene-S(O)₂—N(R″)₂, —N(R″)—C(O)—R″, —N(R″)—C(O)—R″, —N(R″)—C(O)—N(R″)₂, -alkylene-N(R″)—C(O)—N(R″)₂, -alkylene-N(R″)—C(O)—R″, -alkylene-S(O)₂—R″, —N(R″)—S(O)₂—R″, and -alkylene-N(R″)—S(O)₂—R″, cycloalkyl, heterocyclyl, -alkylene-heterocyclyl, heteroaryl and -alkylene-heteroaryl, or wherein two Z substituents on adjacent carbon atoms, on a carbon atom and an adjacent heteroatom, or on a single carbon atom, together with carbon atom(s) and/or the combination of the carbon atom and the adjacent heteroatom to which said Z substituents are attached form a four to seven-membered cycloalkyl, cyloalkeny, heterocyclyl, aryl or heteroaryl ring, wherein said aryl, heteroaryl, the aryl portion of said -alkylene-aryl, -alkylene-O-alkylene-aryl, -alkylene-O-alkylene-aryl, -alkylene-O-aryl, and the heteroaryl portion of said -alkylene-heteroaryl are unsubstituted or substituted with one or more R* substituents which are independently selected; and R* is one or more substituents independently selected from the group consisting of halogen, cyano, alkyl, alkoxy, haloalkyl, haloalkoxy, nitro, —NH₂, —NH(alkyl), —N(alkyl)₂, —NH-alkylene-aryl, —N(alkyl)-alkylene-aryl, -alkylene-aryl, —C(O)NH₂, —C(O)NH(alkyl), —C(O)N(alkyl)₂, —S(O)₂NH₂, —S(O)₂NH(akyl), —S(O)₂N(alkyl)₂, —NHC(O)-alkyl, —N(alkyl)C(O)-alkyl, —NHC(O)-aryl, —N(alkyl)C(O)-aryl, —NH—S(O)₂-alkyl, —N(alkyl)-S(O)₂-alkyl, —NH—S(O)₂-aryl, and —N(alkyl)-S(O)₂-aryl.

11: The method of claim 10, (E4D) with acetaldehyde, (Z)-2-methylbut-2-enal, (E)-2-methylbut-2-enal, 3-methylbut-2-enal, (E)-but-2-enal, cinnamaldehyde, pent-4-enal, (E)-4-(dimethylamino)but-2-enal gave the following compounds: 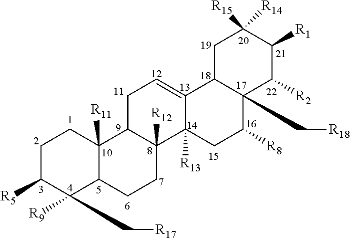
wherein:
1) R1, R2, R3, R5, R8 are OH; R17, R18 are NH-ethyl;
2) R1, R2, R3, R5, R8 are OH; R17, R18 are NH—(Z)-(2-methylbut-2-en-1-yl);
3) R1, R2, R3, R5, R8 are OH; R17, R18 are NH-(E)-(2-methylbut-2-en-1-yl);
4) R1, R2, R3, R5, R8 are OH; R17, R18 are NH-(3-methylbut-2-en-1-yl;
5) R1, R2, R3, R5, R8 are OH; R17, R18 are NH-(E)-(but-2-en-1-yl;
6) R18, R2, R3, R5, R8 are OH; R17, R18 are NH-cinnamyl;
7) R18, R2, R3, R5, R8 are OH; R17, R18 are NH-pent-4-en-1-yl;
8) R18, R2, R3, R5, R8 are OH; R17, R18 are NH-(E)-3-((4-(dimethylamino)but-2-en-1-yl.

12. The method of claim 8, (E4D) with isocyanatomethane, (Z)-1-isocyanato methylbut-2-ene, (E)-1-isocyanato-2-methylbut-2-ene, 1-isocyanato-3-methylbut-2-ene, (E)-1-isocyanatobut-2-ene, (E)-(3-isocyanatoprop-1-en-1-yl)benzene, 4-isocyanatobut-1-ene, (E)-4-isocyanato-N,N-dimethylbut-1-en-1-amine gave the following compounds: 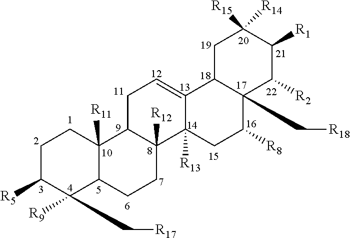
wherein:
1) R1, R2, R5, R8 are OH; R17, R18 are NH—CO—NH-ethyl;
2) R1, R2, R5, R8 are OH; R17, R18 are NH—CO—NH—(Z)-1-(2-methylbut-2-en-1-yl);
3) R1, R2, R5, R8 are OH; R17, R18 are NH—CO—NH-(E)-1-(2-methylbut-2-en-1-yl);
4) R1, R2, R5, R8 are OH; R17, R18 are NH—CO—NH-1-(3-methylbut-2-en-1-yl);
5) R1, R2, R5, R8 are OH; R17, R18 are NH—CO—NH-(E)-1-(but-2-en-1-yl);
6) R18, R2, R5, R8 are OH; R17, R18 are NH—CO—NH-1-cinnamyl;
7) R18, R2, R5, R8 are OH; R17, R18 are NH—CO—NH-1-(but-3-en-1-yl);
8) R18, R2, R5, R8 are OH; R17, R18 are NH—CO—NH-(E)-1-(4-(dimethylamino)but-3-en-1-yl).

13. The method of claim 7, wherein (E4D) with ethanoyl, angeloyl, tigloyl, senecioyl, Crotonoyl, Cinnamoyl, Pentenoyl, (E)-3-(dimethylamino)acryloyl, 4-[(2-methoxyethyl)amino]-2-methyl-4oxobut-2-enoyl gave the following compounds: 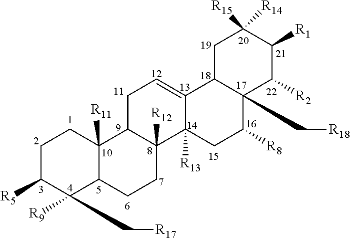
wherein:
1) R1, R2, R5, R8 are OH; R17, R18 are NH-acetyl;
2) R1, R2, R5, R8 are OH; R17, R18 are NH-angeloyl;
3) R1, R2, R5, R8 are OH; R17, R18 are NH-tigloyl;
4) R1, R2, R5, R8 are OH; R17, R18 are NH-senecioyl;
5) R1, R2, R5, R8 are OH; R17, R18 are NH-Crotonoyl;
6) R18, R2, R5, R8 are OH; R17, R18 are NH-Cinnamoyl;
7) R18, R2, R5, R8 are OH; R17, R18 are NH-Pentenoyl;
8) R18, R2, R5, R8 are OH; R17, R18 are NH-4-(dimethylamino)-2-methylbut-2-enoyl;
9) R18, R2, R5, R8 are OH; R17, R18 are NH-4-[(2-methoxyethyl)amino]-2-methyl-4oxobut-2-enoyl.

14. The method of claim 8, wherein (E4D) with ethanesulfonyl, (Z)-but-2-ene-2-sulfonyl, (E)-prop-1-ene-1-sulfonyl, (E)-2-phenylethene-1-sulfonyl, but-3-ene-1-sulfonyl gave the following compounds: 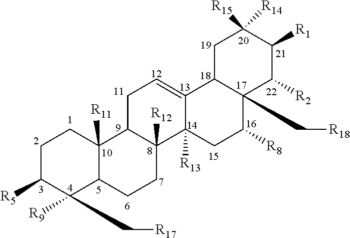
wherein:
1) R1, R2, R5, R8 are OH; R17, R18 are NHSO2-ethyl;
2) R1, R2, R5, R8 are OH; R17, R18 are NHSO2-(Z)-(2-methylbut-2-en-1-yl);
3) R1, R2, R5, R8 are OH; R17, R18 are NHSO2-(E)-prop-1-enyl;
4) R18, R2, R5, R8 are OH; R17, R18 are NHSO2-(E)-2-phenylethenyl;
5) R18, R2, R5, R8 are OH; R17, R18 are NHSO2-but-3-enyl.

15. The compound is selected from claim 1 as medicament.

16. The compound is selected from claim 3 as medicament.

17. A method of treating cancer in a subject, comprising administering to the subject a therapeutically effective amount of a compound having the structure: 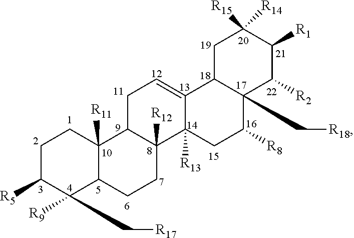
wherein R1, R2, R5, R8 are OH; R17, R18 are selected from NH-acetyl, NH-angeloyl, NH-tigloyl, NH-senecioyl, NH-Crotonoyl, NH-Cinnamoyl, NH-Pentenoyl, NH-4-(dimethylamino)-2-methylbut-2-enoyl, NH-4-[(2-methoxyethyl)amino]-2-methyl-4oxobut-2-enoyl, NHSO2-ethyl, NHSO2-(Z)-(2-methylbut-2-en-1-yl), NHSO2-(E)-prop-1-enyl, NHSO2-(E)-2-phenylethenyl, NHSO2-but-3-enyl, NH—CO—NH-ethyl, NH—CO—NH—(Z)-1-(2-methylbut-2-en-1-yl), NH—CO—NH-(E)-1-(2-methylbut-2-en-1-yl), NH—CO—NH-1-(3-methylbut-2-en-1-yl), NH—CO—NH-(E)-1-(but-2-en-1-yl), NH—CO—NH-1-cinnamyl, NH—CO—NH-1-(but-3-en-1-yl), NH—CO—NH-(E)-1-(4-(dimethylamino)but-3-en-1-yl), NH-ethyl, NH—(Z)-(2-methylbut-2-en-1-yl), NH-(E)-(2-methylbut-2-en-1-yl), NH-(3-methylbut-2-en-1-yl, NH-(E)-(but-2-en-1-yl, NH-cinnamyl, NH-pent-4-en-1-yl, NH-(E)-3-((4-(dimethylamino)but-2-en-1-yl.

18. The method of claim 17, wherein the compound is selected from following:
1) R1, R2, R5, R8 are OH; R17, R18 are NH-acetyl;
2) R1, R2, R5, R8 are OH; R17, R18 are NH-angeloyl;
3) R1, R2, R5, R8 are OH; R17, R18 are NH-tigloyl;
4) R1, R2, R5, R8 are OH; R17, R18 are NH-senecioyl;
5) R1, R2, R5, R8 are OH; R17, R18 are NH-Crotonoyl;
6) R18, R2, R5, R8 are OH; R17, R18 are NH-Cinnamoyl;
7) R18, R2, R5, R8 are OH; R17, R18 are NH-Pentenoyl;
8) R18, R2, R5, R8 are OH; R17, R18 are NH-4-(dimethylamino)-2-methylbut-2-enoyl;
9) R18, R2, R5, R8 are OH; R17, R18 are NH-4-[(2-methoxyethyl)amino]-2-methyl-4oxobut-2-enoyl.

19. A composition comprising one or more compounds selected from claim 1 with pharmaceutical carrier.
